(19) United States
(12) Patent Application Publication
(10) Pub. No.: US 2023/0405117 A1
(43) Pub. Date: Dec. 21, 2023

(54) METHODS AND SYSTEMS FOR CLASSIFICATION AND TREATMENT OF SMALL CELL LUNG CANCER

(71) Applicant: BOARD OF REGENTS, THE UNIVERSITY OF TEXAS SYSTEM, Austin, TX (US)

(72) Inventors: Elizabeth M PARK, Houston, TX (US); Lauren A. BYERS, Houston, TX (US); Carl M. GAY, Houston, TX (US); John V. HEYMACH, Houston, TX (US)

(73) Assignee: BOARD OF REGENTS, THE UNIVERSITY OF TEXAS SYSTEM, Austin, TX (US)

(21) Appl. No.: 18/252,005

(22) PCT Filed: Nov. 5, 2021
(86) PCT No.: PCT/US2021/058218

§ 371 Date: May 5, 2023

Related U.S. Application Data
(60) Provisional application No. 63/110,664, filed on Nov. 6, 2020.

(51) Int. Cl. A61K 39/00; A61K 35/17; A61P 35/00

(52) U.S. Cl. CPC A61K 39/4611; A61K 39/4631; A61K 2239/55; A61K 35/17; A61P 35/00; A61K 39/464482

(57) ABSTRACT

Aspects of the present disclosure are directed to methods for classification and treatment of small cell lung cancer (SCLC). Certain aspects pertain to treatment of a subject having SCLC using a targeting agent for a cell surface protein, where a targeting agent is selected based on a subtype classification of the SCLC. Disclosed are methods for identifying a subject as having an SCLC subtype (e.g., SCLC-A, SCLC-N, SCLC-P, SCLC-I) and administering a targeting agent configured to target a cell surface protein associated with the identified SCLC subtype. Also disclosed are compositions comprising targeting agents for treatment of SCLC.

This application claims benefit of priority of U.S. Provisional Patent Application No. 63/110,664 filed Nov. 6, 2020, which is hereby incorporated by reference in its entirety.

This invention was made with government support under grant number R01 CA207295 awarded by the National Institutes of Health. The government has certain rights in the invention.

### BACKGROUND

### I. Field of the Invention

Aspects of this invention relate to at least the fields of cancer biology and medicine.

### II. Background

Small cell lung cancer (SCLC) is a highly aggressive form of lung cancer for which there exist a very limited number of therapeutics and minimal improvements made over the past 30 years¹⁻³. As a result, the 5-year survival rate is less than 7% across all stages of SCLC^{2,3}. The National Cancer Institute has declared SCLC to be a recalcitrant malignancy with urgent need for a deeper mechanistic understanding of resistance development and identification and targeting of unique vulnerabilities^{4,5}.

SCLC has previously been considered and managed as a homogenous disease, with nearly ubiquitous loss of tumor protein 53 (TP53) and RB Transcriptional Corepressor 1 (RBI) expression resulting in high rates of mutation (tumor mutational burden, or TMB)₆. Unfortunately, neither of these ubiquitous mutations are targetable by currently available therapeutics. The current standard of care (SOC) for patients is platinum-based chemotherapy often in combination with immunotherapy^{7,8}. Chemotherapy alone usually results in a response, but is followed by rapid relapse of resistant disease, and the addition of immunotherapy results in only modest improvements in survival^{9,10}. Second-line treatment consisted solely of topotecan until mid-2020, at which time lurbinectedin was approved for patients with relapsed SCLC^{11,12}. However, both of these treatments have only modest success against relapsed disease¹³. The treatment of SCLC as a single disease ignores a potentially striking avenue for therapeutic development. Current treatment does not consider disease heterogeneity, which could explain the disappointing results from clinical trials and SOC in unselected populations. In contrast to SCLC, NSCLC has seen striking advances in patient care and survival by targeting specific tumor vulnerabilities, as exemplified by treatment of EGFR-mutant patients^{14,15}. Similarly, exploiting different genetic and proteomic vulnerabilities within SCLC tumors could allow for the development of novel, targeted therapeutic reagents based on the tumor's unique signature. Shifting SCLC treatment from a “one-treatment-fits-all” to a more tailored approach will allow for targeted therapeutic reagents to be developed specific to tumor vulnerabilities, providing more effective care and ultimately improving overall survival rates among patients.

SCLC has been surprisingly unresponsive to immune checkpoint blockade (ICB), especially when compared to other cancers with similarly high TMB levels²⁰⁻²². For example, the addition of the anti-PD-L1 compounds atezolizumab or durvalumab to chemotherapy showed median improvement of only one month compared with chemotherapy alone^{9,10}. There is a need in the art for new and improved methods and compositions for treatment of patients with SCLC, including targeted and immune-based therapeutics.

### SUMMARY

The present disclosure fulfils certain needs in the field of cancer medicine by the identification of novel, targetable, surface-expressed targets within each SCLC subtype (SCLC-A, SCLC-N, SCLC-P, and SCLC-I). Identified herein are numerous differentially expressed surface proteins between the four subtypes of SCLC for therapeutic targeting. Embodiments of the disclosure are directed to methods for treatment of a subject determined to have SCLC-A, SCLC-N, SCLC-P, or SCLC-I using a targeting agent configured to target one or more surface proteins associated with the subject's SCLC subtype.

Embodiments of the present disclosure include methods for detecting SCLC, methods for treating SCLC, methods for classifying a subject with SCLC, methods for identifying an SCLC subtype, methods for treating a subject having SCLC-A, methods for treating a subject having SCLC-N, methods for treating a subject having SCLC-P, methods for treating a subject having SCLC-I, methods for targeting a surface marker associated with SCLC-A, methods for targeting a surface marker associated with SCLC-N, methods for targeting a surface marker associated with SCLC-P, and methods for targeting a surface marker associated with SCLC-I. Methods of the disclosure can include at least 1, 2, 3, 4 or more of the following steps: classifying a subject as having SCLC-A, classifying a subject as having SCLC-N, classifying a subject as having SCLC-P, classifying a subject as having SCLC-I, sequencing DNA from a tumor sample from a subject, measuring an expression level of ASCU in a biological sample from a subject, measuring an expression level of NEUROD1 in a biological sample from a subject, measuring an expression level of POU2F3 in a biological sample from a subject, measuring methylation levels of one or more methylation sites from a nucleic acid sample from a subject, and administering a targeting agent to a subject.

Disclosed herein, in some embodiments, is a method for treating a subject for small cell lung cancer (SCLC), the method comprising administering a targeting agent capable of specifically binding to one or more of the proteins of Table 1, Table 2, or Table 3 to a subject determined to have SCLC-A. In some embodiments, the one or more proteins are one or more proteins of Table 1. In some embodiments, the one or more proteins are one or more proteins of Table 2. In some embodiments, the one or more proteins are one or more proteins of Table 3. In some embodiments, the targeting agent is capable of specifically binding to DLL3. In some embodiments, the targeting agent is capable of specifically binding to CEACAM5. In some embodiments, the targeting agent is capable of specifically binding to SCNN1A. In some embodiments, the subject was determined to have SCLC-A by detecting expression of ASCL1 from cancer cells from the subject.

Disclosed herein, in some embodiments, is a method for treating a subject for small cell lung cancer (SCLC), the method comprising administering a targeting agent capable of specifically binding to one or more of the proteins of Table 4, Table 5, or Table 6 to a subject determined to have SCLC-N. In some embodiments, the one or more proteins are one or more proteins of Table 4. In some embodiments, the one or more proteins are one or more proteins of Table 5. In some embodiments, the one or more proteins are one or more proteins of Table 6. In some embodiments, the targeting agent is capable of specifically binding to SSTR2. In some embodiments, the targeting agent is capable of specifically binding to SEMA6D. In some embodiments, the targeting agent is capable of specifically binding to SGCD. In some embodiments, the subject was determined to have SCLC-N by detecting expression of NEUROD1 from cancer cells from the subject.

Disclosed herein, in some embodiments, is a method for treating a subject for small cell lung cancer (SCLC), the method comprising administering a targeting agent capable of specifically binding to one or more of the proteins of Table 7, Table 8, or Table 9 to a subject determined to have SCLC-P. In some embodiments, the one or more proteins are one or more proteins of Table 7. In some embodiments, the one or more proteins are one or more proteins of Table 8. In some embodiments, the one or more proteins are one or more proteins of Table 9. In some embodiments, the targeting agent is capable of specifically binding to MICA. In some embodiments, the targeting agent is capable of specifically binding to TMEM87A. In some embodiments, the targeting agent is capable of specifically binding to ART3. In some embodiments, the subject was determined to have SCLC-P by detecting expression of POU2F3 from cancer cells from the subject.

Disclosed herein, in some embodiments, is a method for treating a subject for small cell lung cancer (SCLC), the method comprising administering a targeting agent capable of specifically binding to one or more of the proteins of Table 10, Table 11, or Table 12 to a subject determined to have SCLC-I. In some embodiments, the one or more proteins are one or more proteins of Table 10. In some embodiments, the one or more proteins are one or more proteins of Table 11. In some embodiments, the one or more proteins are one or more proteins of Table 12. In some embodiments, the targeting agent is capable of specifically binding to SLAMF8. In some embodiments, the targeting agent is capable of specifically binding to MRC2. In some embodiments, the targeting agent is capable of specifically binding to PIEZO1. In some embodiments, the subject was determined to have SCLC-I by determining cancer cells from the subject not to express any of ASCL1, NEUROD1, or POU2F3.

In some embodiments, the targeting agent comprises an antibody or fragment thereof. In some embodiments, the targeting agent is a bispecific T-cell engager. In some embodiments, a cell comprising the targeting agent is administered to the subject. In some embodiments, the cell is an immune cell. In some embodiments, the immune cell is a T cell. In some embodiments, the targeting agent is a chimeric antigen receptor. In some embodiments, the targeting agent is a T cell receptor. In some embodiments, the targeting agent is operatively linked to a therapeutic agent. In some embodiments, the therapeutic agent is a chemotherapeutic. In some embodiments, the therapeutic agent is a toxin. In some embodiments, the therapeutic agent is a therapeutic nucleic acid. In some embodiments, the targeting agent is an antibody-drug conjugate. In some embodiments, the targeting agent is an antibody-oligonucleotide conjugate.

Disclosed here, in some embodiments, is a method for treating a subject for SCLC, the method comprising administering a DLL3-binding protein to a subject determined to have SCLC-A.

Disclosed here, in some embodiments, is a method for treating a subject for SCLC, the method comprising administering a CEACAM5-binding protein to a subject determined to have SCLC-A.

Disclosed here, in some embodiments, is a method for treating a subject for SCLC, the method comprising administering a SCNN1A-binding protein to a subject determined to have SCLC-A.

Disclosed here, in some embodiments, is a method for treating a subject for SCLC, the method comprising administering a SSTR2-binding protein to a subject determined to have SCLC-N.

Disclosed here, in some embodiments, is a method for treating a subject for SCLC, the method comprising administering a SEMA6D-binding protein to a subject determined to have SCLC-N.

Disclosed here, in some embodiments, is a method for treating a subject for SCLC, the method comprising administering a SGCD-binding protein to a subject determined to have SCLC-N.

Disclosed here, in some embodiments, is a method for treating a subject for SCLC, the method comprising administering a MICA-binding protein to a subject determined to have SCLC-P.

Disclosed here, in some embodiments, is a method for treating a subject for SCLC, the method comprising administering a TMEM87A-binding protein to a subject determined to have SCLC-P.

Disclosed here, in some embodiments, is a method for treating a subject for SCLC, the method comprising administering a ART3-binding protein to a subject determined to have SCLC-P.

Disclosed here, in some embodiments, is a method for treating a subject for SCLC, the method comprising administering a SLAMF8-binding protein to a subject determined to have SCLC-I.

Disclosed here, in some embodiments, is a method for treating a subject for SCLC, the method comprising administering a MRC2-binding protein to a subject determined to have SCLC-I.

Disclosed here, in some embodiments, is a method for treating a subject for SCLC, the method comprising administering a PIEZO1-binding protein to a subject determined to have SCLC-I.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows differential gene expression from RNASeq data (Cell Line, George) and microarray data (Sato) of surfaceome proteins across all four SCLC subtypes.

FIGS. 2A-2D shows example hits for SCLC-A (FIG. 2A), SCLC-N (FIG. 2B), SCLC-P (FIG. 2C), and SCLC-I (FIG. 2D). Shown is the analysis from the George et al dataset; an identical analysis was performed for the cell line and Sato datasets

FIGS. 3A-3D show mean expression of the cell surface protein encoding gene SSTR2 in multiple datasets (FIGS. 3A-3C) along with flow cytometry analysis of proportion of analyzed cells that express SSTR2 protein in subtyped cell lines (FIG. 3D).

FIGS. 4A-4C show differential mean expression between subtypes of genes encoding MICA for the George et al. tumor mRNA (FIG. 4A), cell line mRNA (FIG. 4B), and Sato et al. tumor mRNA (FIG. 4C).

FIGS. 5A-5C show differential mean expression between subtypes of genes encoding CEACAM5 for the George et al. tumor mRNA (FIG. 5A), cell line mRNA (FIG. 5B), and Sato et al. tumor mRNA (FIG. 5C).

FIGS. 6A and 6B show western blot analysis of cell lines of each of the four SCLC subtypes. (FIG. 6A) shows analysis of SSTR2 expression. A ratio of band intensity of SSTR2 to GAPDH, arbitrarily normalized to the first lane (H82), is shown below the membrane. (FIG. 6B) shows analysis of CEACAM5 and MICA expression.

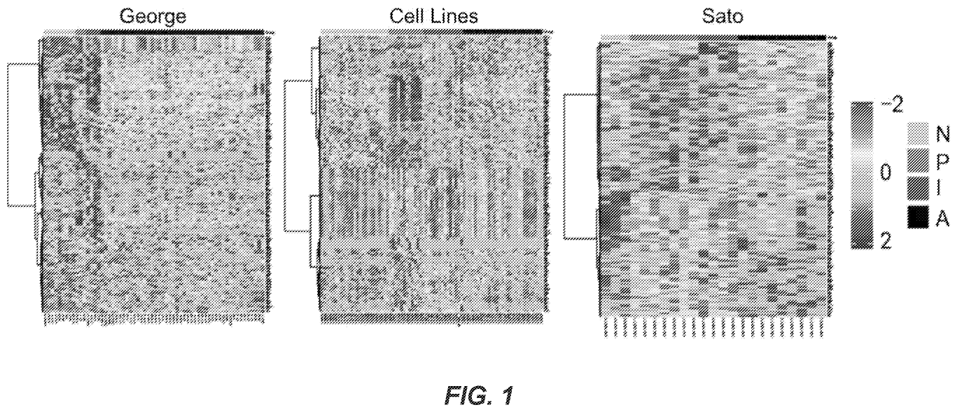

### DETAILED DESCRIPTION OF THE INVENTION

Using mRNA gene expression patterns, tumors from SCLC patients can be classified into four major subtypes of SCLC. Three of them are defined by differential expression of the transcription factors ASCL1 (SCLC-A), NEUROD1 (SCLC-N), and POU2F3 (SCLC-P), and a fourth group is characterized for having high expression of inflammatory-related genes (SCLC-I). Importantly, these subtypes have distinct therapeutic vulnerabilities and show differential response patterns to standard of care and investigation agents. Certain methods for such classification and treatment of SCLC are described in U.S. Patent Application Publication No. US 2021/0062274, incorporated by reference herein in its entirety.

The present disclosure is based, at least in part, on the identification of surface markers (also “cell surface proteins” or “surface proteins”) associated with (i.e., preferentially expressed in) each of the four SCLC subtypes. These associated surface markers may be used to select targeting agents (e.g., antibodies, antibody fragments, CAR T cells, etc.) for use in treatment of each subtype. For example, a targeting agent configured to bind to a surface marker associated with SCLC-A may be used to treat a subject who has been identified to have the SCLC-A subtype. Similarly, a targeting agent configured to bind to a surface marker associated with SCLC-N, SCLC-P, or SCLC-I may be used to treat a subject who has been identified to have the SCLC-N, SCLC-P, or SCLC-I subtype, respectively.

Certain aspects of the disclosure are directed to methods for treatment of a subject with SCLC comprising administering a targeting agent configured to bind to a surface protein of Table 1, Table 2, and/or Table 3, where the subject was determined to have SCLC-A. In some embodiments, the targeting agent is a DLL3-binding protein. In some embodiments, the targeting agent is a CEACAM5-binding protein. In some embodiments, the targeting agent is a SCNN1A-binding protein.

Further aspects of the disclosure are directed to methods for treatment of a subject with SCLC comprising administering a targeting agent configured to bind to a surface protein of Table 4, Table 5, and/or Table 6, where the subject was determined to have SCLC-N. In some embodiments, the targeting agent is a SSTR2-binding protein. In some embodiments, the targeting agent is a SEMA6D-binding protein. In some embodiments, the targeting agent is a SGCD-binding protein.

Further aspects of the disclosure are directed to methods for treatment of a subject with SCLC comprising administering a targeting agent configured to bind to a surface protein of Table 7, Table 8, and/or Table 9, where the subject was determined to have SCLC-P. In some embodiments, the targeting agent is a MICA-binding protein. In some embodiments, the targeting agent is a TMEM87-binding protein. In some embodiments, the targeting agent is a ART3-binding protein.

Further aspects of the disclosure are directed to methods for treatment of a subject with SCLC comprising administering a targeting agent configured to bind to a surface protein of Table 10, Table 11, and/or Table 12, where the subject was determined to have SCLC-I. In some embodiments, the targeting agent is a SLAMF8-binding protein. In some embodiments, the targeting agent is a MRC2-binding protein. In some embodiments, the targeting agent is a PIEZO1-binding protein.

### I. Treatment Methods

Aspects of the present disclosure include methods of treating a patient with small cell lung cancer (SCLC). Certain aspects are directed to methods for treatment of a subject for SCLC, where the treatment is selected based on the SCLC subtype of the subject. As described herein, a subject may have SCLC, where the SCLC can be classified as one of four subtypes: SCLC-A, SCLC-N, SCLC-P, or SCLC-I. In some embodiments, the treatment is a treatment with a targeting agent disclosed herein, wherein the targeting agent is configured to bind to a surface protein identified with an SCLC subtype.

In some embodiments, the subject is identified as having an SCLC subtype based on the expression or methylation status of ASCU, NEUROD1, and POU2F3 in nucleic acid from cancer tissue from the subject. SCLC-A may be identified based on expression of ASCU and lack of expression of NEUROD1 or POU2F3. SCLC-N may be identified based on expression of NEUROD1 and lack of expression of either ASCU or POU2F3. SCLC-P may be identified based on expression of POU2F3 and lack of expression of either ASCU or NEUROD1. SCLC-I may be identified based on lack of expression of any of ASCL1, NEUROD1, and POU2F3.

A treatment for the subject may be determined based on the subtype determination. Such treatment may also be in combination with another therapeutic regime, such as chemotherapy or immunotherapy. In addition, the treatment may be in combination due to a subject's cancer falling into more than one subtype, such as, for example, if one portion of the cancer cells fall into the SCLC-A subtype (e.g., express ASCL1) and another portion of the cancer cells fall into the SCLC-N subtype (e.g., express NEUROD1). The type and/or subtype of a given cancer may change over time, and in some embodiments the present methods regarding identifying the type and/or subtype and selecting an appropriate treatment are performed more than once, such as repeating the methods after a patient develops resistance to a selected therapy, or after a predetermined period of time, and modifying the therapy accordingly.

In some embodiments, a subject is or was determined to have a cancer of the SCLC-A subtype. In some embodiments, the subject is administered a B-cell lymphoma 2 (BCL-2) inhibitor. A BCL-2 inhibitor may describe any agent, molecule, or compound capable of inhibiting the activity of a BCL-2 family protein. Examples of BCL-2 inhibitors include ABT-737, ABT-263 (navitoclax), ABT-199 (venetoclax), GX15-070 (obatoclax), HA14-1, TW-37, AT101, and BI-97C1 (sabutoclax). In some embodiments, the BCL-2 inhibitor is ABT-737 or navitoclax. In some embodiments, the subject is administered a DLL3-targeted therapeutic. A DLL3-targeted therapeutic may describe any agent, molecule, or compound capable of binding to a DLL3 protein. In some embodiments, the DLL3-targeted therapeutic is an anti-DLL3 antibody or fragment thereof. In some embodiments, the DLL3-targeted therapeutic is rovalpituzumab. In some embodiments, the DLL3-targeted therapeutic is an antibody-drug conjugate. In some embodiments, the DLL3-targeted therapeutic is rovalpituzumab tesirine. In some embodiments, the subject having a cancer of the SCLC-A subtype is administered a targeting agent configured to bind to one or more of the proteins of Table 1, Table 2, and/or Table 3. In some embodiments, the subject is administered a targeting agent configured to bind to one or more of the proteins of Table 1. In some embodiments, the subject is administered a targeting agent configured to bind to one or more of the proteins of Table 2. In some embodiments, the subject is administered a targeting agent configured to bind to one or more of the proteins of Table 3. In some embodiments, the subject is administered a targeting agent configured to bind to DLL3 (e.g., a DLL3-binding protein). In some embodiments, the subject is administered a targeting agent configured to bind to CEACAM5 (e.g., a CEACAM5-binding protein). In some embodiments, the subject is administered a targeting agent configured to bind to SCNN1A (e.g., a SCNN1A-binding protein).

In some embodiments, a subject is or was determined to have a cancer of the SCLC-N subtype. In some embodiments, the subject is administered an Aurora kinase (AURK) inhibitor, a JAK inhibitor, or a c-Met inhibitor. In some embodiments, the subject is administered an AURK inhibitor. Examples of AURK inhibitors include alisertib, ZM447439, hesperidin, ilorasertib, VX-680, CCT 137690, lestaurtinib, NU 6140, PF 03814735, SNS 314 mesylate, TC-A 2317 hydrochloride, TAK-901, AMG-900, AS-703569, AT-9283, CYC-116, SCH-1473759, and TC-S 7010. In some embodiments, the AURK inhibitor is CYC-116, alisertib, or AS-703569. Examples of JAK inhibitors include ruxolitinib, tofacitinib, oclacitinib, baricitinib, peficitinib, fedratinib, upadacitinib, filgotinib, cerdulatinib, gandotinib, lestaurtinib, momelotinib, pacritinib, and PF-04975842. Examples of c-Met inhibitors include BMS-777607, cabozantinib, MK-2461, AMG-458, JNJ-38877605, PF-04217903, and GSK-1363089. Other drugs to which subjects having a cancer of the SCLC-N subtype may be sensitive include PF-562271, VS-507, KW-2449, pimozide, CB-64D, AC-220, omacetaxine mepasuccinate, XL-888, XL-880, ifosfamide, SL-0101, GW-5074, letrozole, CYC-202, and BIM-46187. In some embodiments, the subject having a cancer of the SCLC-N subtype is administered a targeting agent configured to bind to one or more of the proteins of Table 4, Table 5, and/or Table 6. In some embodiments, the subject is administered a targeting agent configured to bind to one or more of the proteins of Table 4. In some embodiments, the subject is administered a targeting agent configured to bind to one or more of the proteins of Table 5. In some embodiments, the subject is administered a targeting agent configured to bind to one or more of the proteins of Table 6. In some embodiments, the subject is administered a targeting agent configured to bind to SSTR2 (e.g., a SSTR2-binding protein). In some embodiments, the subject is administered a targeting agent configured to bind to SEMA6D (e.g., a SEMA6D-binding protein). In some embodiments, the subject is administered a targeting agent configured to bind to SGCD (e.g., a SGCD-binding protein).

In some embodiments, a subject is or was determined to have a cancer of the SCLC-P subtype. In some embodiments, the subject is administered a PARP inhibitor, an AKT inhibitor, a Sky inhibitor, a JAK inhibitor, a SRC inhibitor, a BET inhibitor, an ERK inhibitor, an mTor inhibitor, an HSP90 inhibitor, a PI3K inhibitor, a CDK inhibitor, a topoisomerase inhibitor, a nucleoside analogue, an anti-metabolite, or a platinum-containing chemotherapeutic agent. Examples of PARP inhibitors include olaparib, rucaparib, niraparib, talazoparib, veliparib, pamiparib, CEP 9722, E7016, iniparib, AZD2461, and 3-aminobenzamide. In some embodiments, the PARP inhibitor is talazoparib, olaparib, niraparib, AZD-2461, or rucaparib. Examples of AKT inhibitors include CCT-128930, GSK690693, MK 2206, SC79, capivasertib, ipatasertib, borussertib, uprosertib, perifosine, AZD-5363, and A-443654. Examples of Syk inhibitors include R-406, R-788 (fostamatinib), BAY 61-3606, and nilvadipine. Examples of JAK inhibitors include ruxolitinib, tofacitinib, oclacitinib, baricitinib, peficitinib, fedratinib, upadacitinib, filgotinib, cerdulatinib, gandotinib, lestaurtinib, momelotinib, pacritinib, AZD-1480, XL-019, SB-1578, WL-1034, and PF-04975842. Examples of SRC inhibitors include dasatinib, AZD-0530, KX2-391, bosutinib, saracatinib, and quercetin. Examples of BET inhibitors include GSK1210151A, GSK525762, (+)-JQ1, OTX-015, TEN-010, CPI-203, CPI-0610, LY294002, AZD5153, MT-1, and MS645. Examples of ERK inhibitors include SC-1 (pluripotin), AX 15836, BIX 02189, ERK5-IN-1, FR 180204, TCS ERK 11e, TMCB, and XMD 8-92. Examples of CDK inhibitors include R-547, palbociclib, LY-2835219, CYC-202, ribociclib, abemaciclib, and trilaciclib. Examples of mTor inhibitors include PF-04212384, OSI-027, rapamycin, AZD-2014, RG-7603, BGT-226, PI-103, GS K-2126458, everolimus, temsirolimus, ridaforolimus, sirolimus, dactolisib, and sapanisertib. Examples of anti-metabolites and nucleoside analogues include teriflunomide, pemetrexed, ONX-0801, fluorouracil, cladribine, methotrexate, mercaptopurine, gemcitabine, capecitabine, hydroxyurea, fludarabine, 2-fluoroadenosine, pralatrexate, nelarabine, cladribine, clofarabine, decitabine, azacitidine, cytarabine, floxuridine, and thioguanine. In some embodiments, the anti-metabolite is pemetrexed, methotrexate, or pralatrexate. In some embodiments, the nucleoside analog is floxuridine, cytarabine, clofarabine, or fludarabine. Examples of platinum-containing chemotherapeutic agents include cisplatin, carboplatin, oxaliplatin, nedaplatin, picoplatin, and satraplatin. In some embodiments, the platinum-containing chemotherapeutic agent is cisplatin, carboplatin, oxaliplatin, nedaplatin, picoplatin, or satraplatin. Other drugs to which patients having a cancer of the SCLC-P subtype may be sensitive include ENMD-2076, HPI-1, CP-868596, TL-32711, FGF inhibitor, AS-703569, vandetanib, CYC-116, KW-2499, GSK-2334470, BMS-582664, AEG-40730, ICG-001, CB-64D, SCH-1473759, MK-2461, CH-5132799, dovitinib, AM-2282, PP-242, ZSTK-474, crizotinib, apitolisib, AT-9283, MPC-3100, alisertib, LOR-253, INK-128, AZD-8055, omacetaxine mepasuccinate, everolimus, XL-888, XL-880, PF-04929113, PF-4942847, dactolisib, PF-04691502, TAK-901, CUDC-305, tretinoin, GSK-461364, BAY-80-6946, danorubicin, doxorubicin, valrubicin, YK-4-279, PF-4176340, BKM-120, APO-866, EB-1627, axitinib, XR-5944, XR-5000, BX-912, mitoxantrone, LY-294002, ixabepilone, GDC-0941, BMS-536924, 3-AP, thiotepa, belinostat, and ABT-348. In some embodiments, the subject having a cancer of the SCLC-P subtype is administered a targeting agent configured to bind to one or more of the proteins of Table 7, Table 8, and/or Table 9. In some embodiments, the subject is administered a targeting agent configured to bind to one or more of the proteins of Table 7. In some embodiments, the subject is administered a targeting agent configured to bind to one or more of the proteins of Table 8. In some embodiments, the subject is administered a targeting agent configured to bind to one or more of the proteins of Table 9. In some embodiments, the subject is administered a targeting agent configured to bind to MICA (e.g., a MICA-binding protein). In some embodiments, the subject is administered a targeting agent configured to bind to TMEM87A (e.g., a TMEM87A-binding protein). In some embodiments, the subject is administered a targeting agent configured to bind to ART3 (e.g., a ART3-binding protein).

In some embodiments, a subject is or was determined to have a cancer of the SCLC-I subtype. These cells may express immune checkpoint proteins, inflammatory markers, STING pathway proteins, CCL5, CXCL10, MHC proteins, CD274 (PD-L1), LAG3, C10orf54 (VISTA), ID01, CD38, and ICOS. In this case, the patient is selected for treatment with an immune checkpoint inhibitor, a BTK inhibitor, a Syk inhibitor, a multikinase inhibitor, an ERK inhibitor, an VEGFR inhibitor, a MEK inhibitor, a FGFR inhibitor. Examples of BTK inhibitors include ibrutinib, LCB 03-0110, LFM-A13, PCI 29732, PF 06465469, and terreic acid. Examples of Syk inhibitors include R-406, R-788 (fostamatinib), BAY 61-3606, and nilvadipine. Examples of multikinase inhibitors include LY-2801653, ENMD-2076, ponatinib, and pazopanib. Examples of ERK inhibitors include SC-1 (pluripotin), AX 15836, BIX 02189, ERK5-IN-1, FR 180204, TCS ERK 11e, TMCB, and XMD 8-92. Examples of VEGFR inhibitors include ASP-4130 (tivozanib), lenvatinib, RG-7167, sorafenib, sunitinib, bevacizumab, cabozantinib, regorafenib, nintedanib, and apatinib. Examples of MEK inhibitors include RO-5126766, AZD-8330, TAK-733, XL-518, PD-0325901, ARRY-162, trametinib, pimasertib, cobimetinib, binimetinib, and selumetinib. Examples of FGFR inhibitors include AZD-4547, PD-173074, LY-2874455, BGJ-398, ponatinib, nintedanib, dovitinib, danusertib, and brivanib. Other drugs to which patients having a cancer of the SCLC-I subtype may be sensitive include AZD-1480, AZD-0530, ASP-3026, fulvestrant, SCH-1473759, MK-2461, LY-2090314, PP-242, 17-AAG, BPR1J-097, INK-128, AZD-8055, omacetaxine mepasuccinate, everolimus, XL-888, XL-880, dactolisib, PF-04691502, OSI-027, rapamycin, CUDC-305, and bleomycin. In some embodiments, the subject having a cancer of the SCLC-I subtype is administered a targeting agent configured to bind to one or more of the proteins of Table 10 Table 11, and/or Table 12. In some embodiments, the subject is administered a targeting agent configured to bind to one or more of the proteins of Table 10. In some embodiments, the subject is administered a targeting agent configured to bind to one or more of the proteins of Table 11. In some embodiments, the subject is administered a targeting agent configured to bind to one or more of the proteins of Table 12. In some embodiments, the subject is administered a targeting agent configured to bind to SLAMF8 (e.g., a SLAMF8-binding protein). In some embodiments, the subject is administered a targeting agent configured to bind to MRC2 (e.g., a MRC2-binding protein). In some embodiments, the subject is administered a targeting agent configured to bind to PIEZO1 (e.g., a PIEZO1-binding protein).

### II. Targeting Agents

Aspects of the present disclosure comprise targeting agents. A “targeting agent” of the present disclosure describes a molecule capable of specifically binding to a cell surface protein. In some embodiments, a targeting agent is an antigen-binding protein. An antigen-binding protein describes a protein capable of specifically binding to an antigen. Examples of antigen-binding proteins include antibodies, antibody fragments (e.g., scFv, Fab, etc.), antibody-like molecules (e.g., bispecific T-cell engagers), chimeric antigen receptors, and ligands (e.g., natural ligands, synthetic ligands). In some embodiments, a targeting agent comprises an antibody. Various agents capable of specifically binding to a cell surface protein are known in the art and are contemplated herein.

Targeting agents of the present disclosure include molecules comprising an antigen-binding protein and one or more additional components. In some embodiments, an antigen-binding protein is operatively linked (e.g., covalently linked, non-covalently linked) to one or more therapeutic agents. In some embodiments, the therapeutic agent is a chemotherapeutic. In some embodiments, the therapeutic agent is a toxin (e.g., MMAE, DM1, tesirine, etc.). In some embodiments, the therapeutic agent is a therapeutic nucleic acid (e.g., antisense oligonucleotide, small interfering RNA, small hairpin RNA, etc.). In some embodiments, a targeting molecule of the present disclosure is an antibody-drug conjugate (ADC). In some embodiments, a targeting molecule of the present disclosure is an antibody-oligonucleotide conjugate (AOC).

A targeting agent of the disclosure may be a molecule capable of specifically binding to one or more surface markers of any of Tables 1-12. In some embodiments, a targeting agent is capable of specifically binding to a surface marker of Table 1. In some embodiments, a targeting agent is capable of specifically binding to a surface marker of Table 2. In some embodiments, a targeting agent is capable of specifically binding to a surface marker of Table 3. In some embodiments, a targeting agent is capable of specifically binding to a surface marker of Table 4. In some embodiments, a targeting agent is capable of specifically binding to a surface marker of Table 5. In some embodiments, a targeting agent is capable of specifically binding to a surface marker of Table 6. In some embodiments, a targeting agent is capable of specifically binding to a surface marker of Table 7. In some embodiments, a targeting agent is capable of specifically binding to a surface marker of Table 8. In some embodiments, a targeting agent is capable of specifically binding to a surface marker of Table 9. In some embodiments, a targeting agent is capable of specifically binding to a surface marker of Table 10. In some embodiments, a targeting agent is capable of specifically binding to a surface marker of Table 11. In some embodiments, a targeting agent is capable of specifically binding to a surface marker of Table 12.

### III. Cell Surface Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

As used herein, a “cell surface protein,” (also “surface protein” or “surface marker”) describes a protein which may be expressed on a surface (e.g., cell membrane) of a cell. A cell surface protein may be attached to a membrane of a cell. A cell surface protein may be embedded in a membrane of a cell. A cell surface protein may comprise one or more transmembrane regions. In some embodiments, cell surface proteins associated with (i.e. preferentially expressed in) SCLC subtypes are contemplated. Also contemplated are methods of targeting cell surface proteins for treatment of SCLC. A cell surface protein may be targeted, e.g., via an antibody or antibody fragment, for delivery of a therapeutic to SCLC cells. For example, a cell surface protein may be targeted using an antibody for delivery of a toxin or other therapeutic to SCLC cells expressing the cell surface protein. Examples of cell surface proteins which may be targeted using methods and compositions of the present disclosure include Delta Like Canonical Notch Ligand 3 (DLL3), CEA Cell Adhesion Molecule 5 (CEACAM5), Sodium Channel Epithelial 1 Subunit Alpha (SCNN1A), Somatostatin receptor type 2 (SSTR2), Semaphorin 6D (SEMAD6), Sarcoglycan Delta (SGCD), MHC Class I Polypeptide-Related Sequence A (MICA), Transmembrane Protein 87A (TMEM87A), ADP-Ribosyltransferase 3 (ARTS), SLAM Family Member 8 (SLAMF8), Mannose Receptor C Type 2 (MRC2), and Piezo Type Mechanosensitive Ion Channel Component 1 (PIEZO1).

A. DLL3

Delta Like Canonical Notch Ligand 3 (DLL3), also known as SCDO1, is an inhibitory Notch ligand highly expressed in neuroendocrine tumors. A complete mRNA sequence of human DLL3 has the Genbank accession number NM 016941. DLL3 expression is driven by ASCL1 and, accordingly, as demonstrated herein, DLL3 is preferentially expressed in SCLC-A. A novel antibody-drug conjugate (ADC) targeting DLL3, rovalpituzumab tesirine (Rova-T), showed activity in DLL3-expressing patient-derived xenograft (PDX) models. In clinical trials, clinical activity of Rova-T was observed in a subset of patients, but further clinical development was stopped due to ADC payload toxicity and lower-than-expected response rates in relapsed SCLC. Despite disappointing results with Rova-T, other DLL3 approaches appear promising and are being investigated, including DLL3 CAR-T (NCT03392064), the first CAR-T therapy trial for SCLC. Additionally, DLL3 is the target of bispecific T cell engager (BiTE) immuno-oncology therapy AMG 757 because the cessation of Rova-T appears to be a result of ADC toxicity effects and not DLL3-specific effects.

In some embodiments, disclosed are methods comprising administering to a subject with SCLC a DLL3-binding protein, where the subject was determined to have SCLC-A. Certain non-limiting examples of DLL3-binding proteins of the disclosure include anti-DLL3 antibodies, anti-DLL3 antibody fragments, anti-DLL3 antibody drug conjugates, anti-DLL3 bispecific T cell engagers (BiTEs), and anti-DLL3 chimeric antigen receptors. In some embodiments, the DLL3-binding protein is or comprises rovalpituzumab. In some embodiments, the DLL3-binding protein is rovalpituzumab tesirine. In some embodiments, the DLL3-binding protein is AMG 119. In some embodiments, the DLL3-binding protein is AMG 757.

B. CEACAM5

CEA Cell Adhesion Molecule 5 (CEACAM5), also known as CD66e, is a cell adhesion molecule overexpressed in gastrointestinal and breast cancers as well as in NSCLC. A complete mRNA sequence of human CEACAM5 has the Genbank accession number NM_001291484. CEACAM is the target of labetuzumab govitecan, an ADC in clinical investigation for patients with refractory metastatic colorectal cancer, as well as a CAR T-cell.

In some embodiments, disclosed are methods comprising administering to a subject with SCLC a CEACAM5-binding protein, where the subject was determined to have SCLC-A. Certain non-limiting examples of CEACAM5-binding proteins of the disclosure include anti-CEACAM5 antibodies, anti-CEACAM5 antibody fragments, anti-CEACAM5 antibody drug conjugates, anti-CEACAM5 bispecific T cell engagers (BiTEs), and anti-CEACAM5 chimeric antigen receptors. In some embodiments, the CEACAM5-binding protein is or comprises labetuzumab. In some embodiments, the CEACAM5-binding protein is labetuzumab govitecan.

C. SCNN1A

Sodium Channel Epithelial 1 Subunit Alpha (SCNN1A), also known as BESC2, is a nonvoltage-gated, amiloride-sensitive, sodium channels. A complete mRNA sequence of human SCNN1A has the Genbank accession number NM_001038.

In some embodiments, disclosed are methods comprising administering to a subject with SCLC a SCNN1A-binding protein, where the subject was determined to have SCLC-A. Certain non-limiting examples of CEACAM5-binding proteins of the disclosure include anti-SCNN1A antibodies, anti-SCNN1A antibody fragments, anti-SCNN1A antibody drug conjugates, anti-SCNN1A bispecific T cell engagers (BiTEs), and anti-SCNN1A chimeric antigen receptors.

D. SSTR2

Somatostatin receptor type 2 (SSTR2) is a seven transmembrane receptor. SSTR2 is a well-established target expressed in low- and intermediate-grade neuroendocrine tumors (NETs), in which somatostatin analogues, such as octreotide and lanreotide, which bind SSTR2, are routinely used therapeutically. SSTR2 is also the target of an ADC, PEN-221. A complete mRNA sequence of human SSTR2 has the Genbank accession number NM_001050.

In some embodiments, disclosed are methods comprising administering to a subject with SCLC a SSTR2-binding protein, where the subject was determined to have SCLC-N. Certain non-limiting examples of SSTR2-binding proteins of the disclosure include anti-SSTR2 antibodies, anti-SSTR2 antibody fragments, anti-SSTR2 antibody drug conjugates, anti-SSTR2 bispecific T cell engagers (BiTEs), and anti-SSTR2 chimeric antigen receptors. In some embodiments, the SSTR2-binding protein is PEN-221.

E. SEMAD6

Semaphorin 6D (SEMAD6) is a cell surface protein. A complete mRNA sequence of human SEMAD6 has the Genbank accession number NM_020858.

In some embodiments, disclosed are methods comprising administering to a subject with SCLC a SEMAD6-binding protein, where the subject was determined to have SCLC-N. Certain non-limiting examples of SEMAD6-binding proteins of the disclosure include anti-SEMAD6 antibodies, anti-SEMAD6 antibody fragments, anti-SEMAD6 antibody drug conjugates, anti-SEMAD6 bispecific T cell engagers (BiTEs), and anti-SEMAD6 chimeric antigen receptors.

F. SGCD

Sarcoglycan Delta (SGCD) is a component of the sarcoglycan complex. A complete mRNA sequence of human SGCD has the Genbank accession number NM_000337.

In some embodiments, disclosed are methods comprising administering to a subject with SCLC a SGCD-binding protein, where the subject was determined to have SCLC-N. Certain non-limiting examples of SGCD-binding proteins of the disclosure include anti-SGCD antibodies, anti-SGCD antibody fragments, anti-SGCD antibody drug conjugates, anti-SGCD bispecific T cell engagers (BiTEs), and anti-SGCD chimeric antigen receptors.

G. MICA

MHC Class I Polypeptide-Related Sequence A (MICA) is a cell surface protein. MICA normally acts as the ligand for Natural Killer Group 2 (NKG2D) receptor activation, however prolonged NKG2D activation can ultimately suppress Natural Killer (NK) cell and CD8+ T-cell activity, allowing for immune evasion. A complete mRNA sequence of human MICA has the Genbank accession number NM_000247.

In some embodiments, disclosed are methods comprising administering to a subject with SCLC a MICA-binding protein, where the subject was determined to have SCLC-P. Certain non-limiting examples of MICA-binding proteins of the disclosure include anti-MICA antibodies, anti-MICA antibody fragments, anti-MICA antibody drug conjugates, anti-MICA bispecific T cell engagers (BiTEs), and anti-MICA chimeric antigen receptors. In some embodiments, the MICA-binding protein is IPH43.

H. TMEM87A

Transmembrane Protein 87A (TMEM87A) is a cell surface protein. A complete mRNA sequence of human TMEM87A has the Genbank accession number NM_015497.

In some embodiments, disclosed are methods comprising administering to a subject with SCLC a TMEM87A-binding protein, where the subject was determined to have SCLC-P. Certain non-limiting examples of TMEM87A-binding proteins of the disclosure include anti-TMEM87A antibodies, anti-TMEM87A antibody fragments, anti-TMEM87A antibody drug conjugates, anti-TMEM87A bispecific T cell engagers (BiTEs), and anti-TMEM87A chimeric antigen receptors.

I. ART3

ADP-Ribosyltransferase 3 (ART3) is a cell surface protein. A complete mRNA sequence of human ART3 has the Genbank accession number NM_001130016.

In some embodiments, disclosed are methods comprising administering to a subject with SCLC a ART3-binding protein, where the subject was determined to have SCLC-P. Certain non-limiting examples of ART3-binding proteins of the disclosure include anti-ART3 antibodies, anti-ART3 antibody fragments, anti-ART3 antibody drug conjugates, anti-ART3 bispecific T cell engagers (BiTEs), and anti-ART3 chimeric antigen receptors.

J. SLAMF8

SLAM Family Member 8 (SLAMF8), also known as CD353, is a member of the CD2 family of cell surface proteins involved in lymphocyte activation. A complete mRNA sequence of human SLAMF8 has the Genbank accession number NM_020125.

In some embodiments, disclosed are methods comprising administering to a subject with SCLC a SLAMF8-binding protein, where the subject was determined to have SCLC-I. Certain non-limiting examples of SLAMF8-binding proteins of the disclosure include anti-SLAMF8 antibodies, anti-SLAMF8 antibody fragments, anti-SLAMF8 antibody drug conjugates, anti-SLAMF8 bispecific T cell engagers (BiTEs), and anti-SLAMF8 chimeric antigen receptors.

K. MRC2

Mannose Receptor C Type 2 (MRC2), also known as CD280, is a member of the mannose receptor family of proteins. A complete mRNA sequence of human MRC2 has the Genbank accession number NM_006039.

In some embodiments, disclosed are methods comprising administering to a subject with SCLC a MRC2-binding protein, where the subject was determined to have SCLC-I. Certain non-limiting examples of MRC2-binding proteins of the disclosure include anti-MRC2 antibodies, anti-MRC2 antibody fragments, anti-MRC2 antibody drug conjugates, anti-MRC2 bispecific T cell engagers (BiTEs), and anti-MRC2 chimeric antigen receptors.

L. PIEZO1

Piezo Type Mechanosensitive Ion Channel Component 1 (PIEZO1) is a mechanically-activated ion channel. A complete mRNA sequence of human PIEZO1 has the Genbank accession number NM_001142864.

In some embodiments, disclosed are methods comprising administering to a subject with SCLC a PIEZO1-binding protein, where the subject was determined to have SCLC-I. Certain non-limiting examples of PIEZO1-binding proteins of the disclosure include anti-PIEZO1 antibodies, anti-PIEZO1 antibody fragments, anti-PIEZO1 antibody drug conjugates, anti-PIEZO1 bispecific T cell engagers (BiTEs), and anti-PIEZO1 chimeric antigen receptors.

### IV. Antibodies

Aspects of the disclosure relate to antibodies or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (t), delta (6), gamma (γ), alpha (a), or epsilon (c) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the extrenal surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

A. Functional Antibody Fragments and Antigen-Binding Fragments

1. Antigen-Binding Fragments

Certain aspects relate to antibody fragments, such as antibody fragments that bind to a cell surface protein on a cancer cell. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, theylack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

2. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

B. Polypeptides with antibody CDRs & Scaffolding Domains that Display the CDs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### V. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy, or skin biopsy. In certain embodiments the sample is obtained from a biopsy from lung tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, plasma, serum, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. A sample may also include a sample devoid of cells, for example a cell-free sample comprising cell-free nucleic acid, such as a serum sample. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, blood collection, plasma collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple lung samples or multiple blood or plasma samples, may be obtained for diagnosis by the methods described herein. In other cases, multiple samples, such as one or more samples from one tissue type (for example lung) and one or more samples from another specimen (for example serum) may be obtained for diagnosis by the methods. In some cases, multiple samples such as one or more samples from one tissue type (e.g. lung) and one or more samples from another specimen (e.g. serum) may be obtained at the same or different times.

In some embodiments, a biological sample analyzed hereis is a liquid sample. In some embodiments, the sample is a blood sample. In some embodiments, the sample is a plasma sample. In some embodiments, the sample is a serum sample. A liquid sample may comprise tumor DNA. Tumor DNA from a liquid sample may be cell-free DNA (cfDNA) and/or DNA from circulating tumor cells. As described herein, “circulating tumor DNA,” or “ctDNA” describes tumor DNA obtained from blood or a blood component (e.g., plasma, serum) from a subject. Tumor DNA, including circulating tumor DNA (ctDNA), may be isolated from a sample and analyzed as disclosed herein (e.g., by sequencing such as bisulfite sequencing).

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VI. Assay Methods

A. Detection of Methylated DNA

Aspects of the methods include assaying nucleic acids (e.g., tumor DNA) to determine expression levels and/or methylation levels of nucleic acids. In some embodiments, disclosed are methods comprising determining a methylation status of one or more methylation sites from methylated DNA. The disclosed methods may comprise determining a subject (i.e., DNA from a subject such as tumor DNA) to have differential methylation at one or more methylation sites. As used herein, “differential methylation” of a methylation site describes a significant difference in methylation status of the methylation site in a sample (e.g., a sample comprising tumor DNA from a subject having cancer) as compared to a control or reference (e.g., DNA from a healthy subject). For example, in some embodiments, a methylation site from a sample comprising tumor DNA has significantly increased methylation levels compared to the same methylation site from control (e.g., healthy, non-tumor) DNA. In some embodiments, a methylation site from a sample comprising tumor DNA has significantly decreased methylation levels compared to the same methylation site from control (e.g., healthy, non-tumor) DNA. Assays for the detection of methylated DNA are known in the art. Methylated DNA includes, for example, methylated circulating tumor DNA. Certain, non-limiting examples of such methods are described herein.

1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5 mC) present in a hydrolysed DNA sample. The method includes hydrolyzing the DNA into its constituent nucleoside bases, the 5 mC and dC bases are separated chromatographically and, then, the fractions are measured. Then, the 5 mC/dC ratio can be calculated for each sample, and this can be compared between the experimental and control samples.

2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay—LINE-1, available from Active Motif; 5-mC DNA ELISA Kit, available from Zymo Research; MethylFlash Methylated DNAS-mC Quantification Kit and MethylFlash Methylated DNAS-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody; and then (3) colorimetric/fluorometric detection reagents.

The Global DNA Methylation Assay—LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analysing a fraction of the genome, it is possible to achieve better accuracy in quantification.

4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ˜5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme Mspl, which will cut at all CCGG sites. The enzyme EcoRI is included in both reactions as an internal control. Both Mspl and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/Mspl ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5 mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after Mspl digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

8. Methods that Exclude Bisulfite Conversion

In some aspects, direct detection of modified bases without bisulfite conversion may be used to detect methylation. For example, Pacific Biosciences company has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include nanopore-based single-molecule real-time sequencing technology (SMRT), which is able to detect modified bases directly (described in Laszlo A. H. et al., Proc. Natl. Acad. Sci. USA. 2013 and Schreiber J., et al., Proc. Natl. Acad. Sci. USA. 2013, which are herein incorporated by reference).

9. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.0R Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR, 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

10. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or Mmel. These ways, small fragments are generated that are located in close proximity to the original HpaII site. Then, NGS and mapping to the genome are performed. The number of reads for each HpaII site correlates with its methylation level.

A number of restriction enzymes have been discovered that use methylated DNA as a substrate (methylation-dependent endonucleases). Most of them were discovered and are sold by SibEnzyme: Bisl, BlsI, Glal. GluI, Krol, Mtel, Pcsl, Pkrl. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other. This is a very useful tool for isolation of methylated CpG islands that again can be combined with NGS. Being bisulfite-free, these three approaches have a great potential for quick whole genome methylome profiling.

B. Sequencing

In some embodiments, the methods of the disclosure include a sequencing method. Example sequencing methods include those described below.

1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine. The essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing.

3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### VII. Cancer Therapy

In some embodiments, the method further comprises administering a cancer therapy to the patient. The cancer therapy may be chosen based on expression level measurements, alone or in combination with a clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered. The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

A. Chemotherapies

In some embodiments, methods of the disclosure comprise administering a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent. Other suitable chemotherapeutic agents include antimicrotubule agents, e.g., Paclitaxel (“Taxol”) and doxorubicin hydrochloride (“doxorubicin”).

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m 2. Further, 5-FU dosing schedules may be for a variety of time periods, for example up to six weeks, or as determined by one of ordinary skill in the art to which this disclosure pertains.

The amount of the chemotherapeutic agent delivered to the patient may be variable. In one suitable embodiment, the chemotherapeutic agent may be administered in an amount effective to cause arrest or regression of the cancer in a host. In other embodiments, the chemotherapeutic agent may be administered in an amount that is anywhere between 2 to fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

B. Surgery

In some embodiments, the disclosed methods comprise surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an anti-cancer therapy, such as a chemotherapeutic. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

C. Immunotherapy

In some embodiments, the disclosed methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below. In some embodiments, a cancer immunotherapy is administered to a subject having been determined to have a cancer of the SCLC-I subtype. In some embodiments, a cancer immunotherapy is administered to a subject in combination with one or more additional cancer therapies.

1. Checkpoint Inhibitors and Combination Treatment

Embodiments of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM 12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tc 1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

2. Activation of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### VIII. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about μM to 50 μM; or about 25 μM to 150 μM; or about 25 μM to 100 μM; or about 25 μM to μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. Pharmaceutical Compositions

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising targeting agents) are administered to a subject intravenously.

The manner of application may be varied widely. Any of the conventional methods for administration of pharmaceutical compositions are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Surfaceome Analysis of SCLC Subtypes

### Methods

Data sets: Three datasets were examined to identify surfaceome targets: published RNA-Seq data from 81 treatment-naïve patient tumors (George data set)¹⁸, published microarray data from 23 cancerous and 42 normal patient samples (Sato data set)¹⁷, and RNA-Seq data from an in-house collection of 63 SCLC cell lines (Cell Line data set)^{16,23,24}.

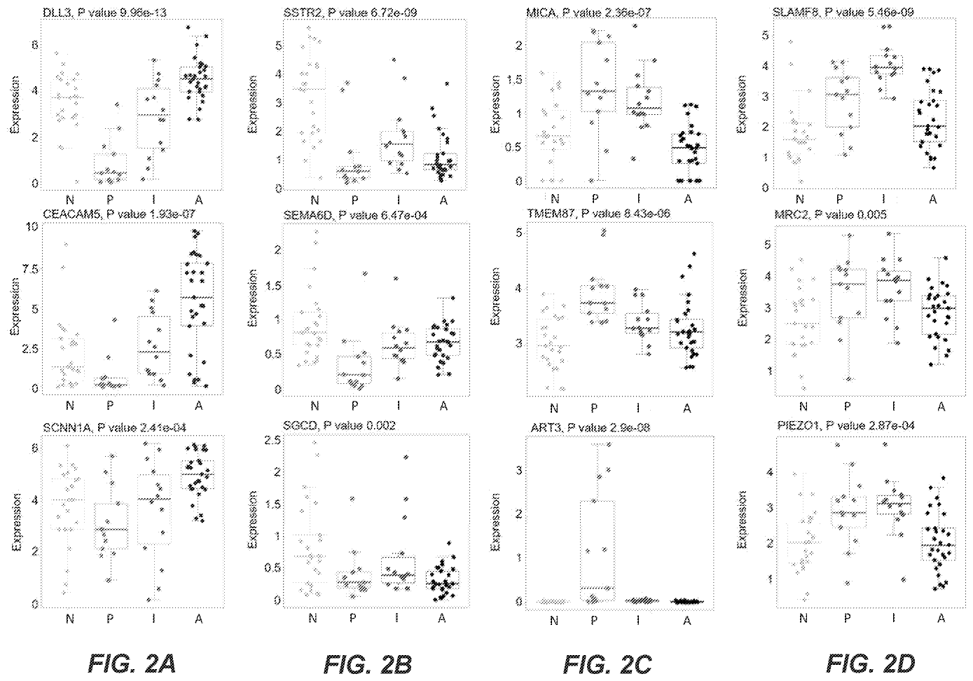

Statistical analysis: Each tumor or cell line sample was sorted into its respective subtype using a 1300-gene signature¹⁹, then compared expression of the surfaceome²⁵ transcripts between the subtypes in each of the three data sets utilizing ANOVA p-values, characterizing hits as a p-value less than 0.05 based on a FDR of 0.01 for the Sato and cell line sets and a FDR of 0.0001 for the George data set (FIG. 2). The FDR was adjusted for the George data set because, as the largest data set, it gave many more significant genes using the established FDR of 0.01. To ensure rigor in the hits, the FDR was lowered accordingly. In parallel, the inventors examined the percent difference of transcript expression between each subtype within each data set. Each transcript was binned into the subset for which it exhibits the highest expression, and the percent difference in expression across subtypes was calculated. Any gene for which the expression in its highest subtype was 10% or more than any other subtype was considered a hit. Hits from both analysis methods across all three data sets were consolidated into a list of target candidates, shown in Tables 1-12. Targets associated with the SCLC-A subtype are shown in Tables 1-3. Targets associated with the SCLC-N subtype are shown in Tables 4-6. Targets associated with the SCLC-P subtype are shown in Tables 7-9. Targets associated with the SCLC-I subtype are shown in Tables 10-12. To be considered a hit (i.e. associated with a particular subtype), a candidate transcript had to either have a) an ANOVA p value less than or equal to 0.05 or b) have an increase in expression by at least 10% in the subtype for which the transcript exhibits the highest expression. Importantly, the inventors classified proteins as surface-expressed based on a report detailing the in silico human surfaceome²⁵.

### Results

The hits analysis revealed a list of 2373 surface-expressed transcripts that are differentially expressed across the four different subtypes of SCLC (Tables A-I). A subset of candidates are listed below and are shown in FIGS. 2A-2D.

SCLC-A: DLL3 was previously shown to be predominantly expressed in SCLC-A, and this analysis correlated these findings. The inventors additionally found CEA Cell Adhesion Molecule 5 (CEACAM5) and Sodium Channel Epithelial 1 Subunit Alpha (SCNN1A) to be strongly expressed in SCLC-A.

SCLC-N: Somatostatin receptor type 2 (SSTR2), Semaphorin 6D (SEMAD6) and Sarcoglycan Delta (SGCD) are strongly expressed in SCLC-N.

SCLC-P: MHC Class I Polypeptide-Related Sequence A (MICA), Transmembrane Protein 87A (TMEM87A) and ADP-Ribosyltransferase 3 (ART3) are strongly expressed in SCLC-P.

SCLC-I: SLAM Family Member 8 (SLAMF8), Mannose Receptor C Type 2 (MRC2), and Piezo Type Mechanosensitive Ion Channel Component 1 (PIEZO1) are highly expressed in SCLC-I.

TABLE 1
Surface proteins associated with the SCLC-A subtype from the George et al. dataset

| Surface Protein | ANOVA P value | A vs N | A vs P | A vs I |
|---|---|---|---|---|
| ROBO1 | 2.84105E−15 | 0.513318 | 65.52% | 41.66% |
| SCN3A | 3.94687E−13 | 0.477538 | 89.30% | 51.39% |
| PTPRN2 | 7.98399E−13 | 0.223642 | 72.06% | 37.88% |
| DLL3 | 9.9566E−13 | 0.22315 | 81.31% | 36.96% |
| SCN2A | 1.3185E−12 | 0.72742 | 96.86% | 71.38% |
| SEZ6L2 | 3.03851E−09 | 0.167686 | 62.51% | 38.45% |
| EPOR | 6.58233E−09 | 0.109658 | 44.58% | 30.25% |
| SGCE | 1.4434E−08 | 0.107614 | 43.60% | 24.78% |
| ALCAM | 2.27058E−08 | 0.474648 | 36.19% | 40.92% |
| JAM3 | 2.50974E−08 | 0.140215 | 45.45% | 22.18% |
| PTPRH | 5.79499E−08 | 0.518246 | 69.24% | 76.74% |
| SLC6A17 | 9.06298E−08 | 0.217736 | 78.00% | 46.12% |
| BDKRB1 | 1.4514E−07 | 0.382055 | 77.14% | 47.77% |
| LRP11 | 1.54942E−07 | 0.120421 | 24.43% | 11.95% |
| KCNMB2 | 1.6141E−07 | 0.161155 | 81.80% | 50.54% |
| CEACAM5 | 1.92784E−07 | 0.602428 | 88.52% | 51.91% |
| DNER | 1.94019E−07 | 0.108691 | 79.60% | 39.20% |
| GPR6 | 2.04931E−07 | 0.111667 | 88.53% | 54.81% |
| GJB1 | 4.11699E−07 | 0.622747 | 59.82% | 65.16% |
| MFSD12 | 7.75528E−07 | 0.226654 | 25.44% | 11.62% |
| SLC7A2 | 1.3774E−06 | 0.685447 | 38.09% | 54.71% |
| NPC1L1 | 1.78602E−06 | 0.591312 | 87.93% | 65.71% |
| GRIN2C | 1.93207E−06 | 0.48594 | 78.53% | 36.29% |
| PTPRN | 2.91319E−06 | 0.150986 | 85.16% | 50.66% |
| CALY | 3.26088E−06 | 0.676083 | 92.31% | 79.19% |
| SLC36A4 | 4.15749E−06 | 0.121561 | 32.42% | 12.06% |
| SLC2A12 | 4.69219E−06 | 0.392637 | 51.60% | 31.32% |
| TMEM154 | 8.21862E−06 | 0.584381 | 44.31% | 26.01% |
| CLDN12 | 9.05944E−06 | 0.248436 | 19.94% | 26.53% |
| FAM174A | 1.2936E−05 | 0.197447 | 25.50% | 18.90% |
| TMEM184A | 1.40777E−05 | 0.293346 | 58.31% | 19.06% |
| NLGN1 | 1.84516E−05 | 0.30062 | 71.46% | 56.87% |
| TNFRSF11A | 1.97931E−05 | 0.353285 | 53.16% | 36.22% |
| CDH1 | 3.73036E−05 | 0.224274 | 11.44% | 20.78% |
| CPD | 4.07671E−05 | 0.217368 | 26.62% | 25.05% |
| ILDR1 | 5.77417E−05 | 0.430687 | 39.11% | 43.25% |
| CCR10 | 6.25666E−05 | 0.428066 | 55.65% | 38.61% |
| CLDN7 | 8.61026E−05 | 0.289436 | 10.60% | 22.28% |
| SLC7A4 | 0.000102933 | 0.162167 | 65.25% | 40.36% |
| SPINT2 | 0.000113296 | 0.108017 | 16.78% | 15.31% |
| GPR111 | 0.000119261 | 0.802409 | 95.08% | 74.09% |
| SLC39A14 | 0.00013616 | 0.183869 | 31.59% | 20.17% |
| KIAA1644 | 0.000150509 | 0.334594 | 78.28% | 45.61% |
| BCAM | 0.000151493 | 0.26964 | 24.32% | 25.53% |
| TAAR1 | 0.00016684 | 0.831891 | 96.97% | 89.12% |
| HTR3A | 0.000175471 | 0.524177 | 81.02% | 69.83% |
| NMUR2 | 0.000195944 | 0.805154 | 83.86% | 71.98% |
| ABCA3 | 0.00020242 | 0.156733 | 35.63% | 17.30% |
| SCNN1A | 0.000240791 | 0.243577 | 37.52% | 27.11% |
| RXFP3 | 0.000262175 | 0.138332 | 94.50% | 82.36% |
| ENTPD3 | 0.000273885 | 0.226454 | 61.17% | 38.28% |
| CD320 | 0.000302997 | 0.186224 | 14.27% | 16.31% |
| SV2B | 0.000420383 | 0.402711 | 56.07% | 55.39% |
| GPR152 | 0.000426642 | 0.531875 | 67.95% | 56.92% |
| SLCO3A1 | 0.0005138 | 0.239712 | 18.42% | 19.49% |
| DPP10 | 0.000542401 | 0.401299 | 75.13% | 45.96% |
| APP | 0.00059644 | 0.102058 | 14.20% | 14.64% |
| SLC18A1 | 0.000598735 | 0.360389 | 87.06% | 83.53% |
| SDK2 | 0.000668337 | 0.216239 | 42.42% | 46.03% |
| PKD1L3 | 0.000677872 | 0.705778 | 87.82% | 52.10% |
| ANO9 | 0.0006877 | 0.29025 | 18.17% | 29.43% |
| NKAIN2 | 0.000691291 | 0.272325 | 58.62% | 52.84% |
| IL17RE | 0.000720317 | 0.30643 | 38.50% | 40.86% |
| GRIN1 | 0.000721706 | 0.701276 | 92.56% | 70.58% |
| GLRA3 | 0.000810376 | 0.744026 | 90.13% | 79.86% |
| TSPAN1 | 0.00081067 | 0.308206 | 44.58% | 41.76% |
| SLC5A2 | 0.000882596 | 0.270054 | 70.63% | 63.71% |
| IL20RA | 0.000979405 | 0.666634 | 20.55% | 42.30% |
| SPACA4 | 0.000989342 | 0.693894 | 54.32% | 64.00% |
| SHISA8 | 0.00100122 | 0.14828 | 74.43% | 43.82% |
| EPHA7 | 0.001092927 | 0.281773 | 49.57% | 34.01% |
| TMEM116 | 0.001113645 | 0.135198 | 27.06% | 15.94% |
| DLL1 | 0.00112921 | 0.283026 | 33.02% | 19.35% |
| ZP1 | 0.001561421 | 0.257511 | 86.96% | 69.24% |
| LRFN3 | 0.001566479 | 0.101462 | 25.75% | 14.84% |
| CDH8 | 0.001633859 | 0.671096 | 48.60% | 53.86% |
| LRIG1 | 0.001657351 | 0.114158 | 20.55% | 10.86% |
| EPHB1 | 0.002010498 | 0.2352 | 53.22% | 37.33% |
| TSPAN8 | 0.002121172 | 0.470899 | 31.34% | 42.28% |
| GFRA3 | 0.002175194 | 0.605733 | 56.86% | 54.19% |
| SLC26A6 | 0.002260401 | 0.225542 | 33.52% | 21.22% |
| CLDN18 | 0.002408984 | 0.735808 | 31.77% | 32.65% |
| FZD5 | 0.002557049 | 0.169006 | 29.95% | 29.23% |
| UGT8 | 0.00262277 | 0.270944 | 14.21% | 30.72% |
| NIPAL1 | 0.002640345 | 0.367205 | 14.44% | 59.31% |
| PTH2R | 0.002718192 | 0.572014 | 85.11% | 22.22% |
| CCKBR | 0.002950713 | 0.348152 | 80.04% | 44.82% |
| SLC4A10 | 0.003102873 | 0.836458 | 61.29% | 83.55% |
| HEPACAM2 | 0.003364408 | 0.288534 | 11.89% | 37.82% |
| OR51E1 | 0.003670912 | 0.529062 | 64.78% | 56.07% |
| BAMBI | 0.003937506 | 0.124444 | 32.72% | 23.27% |
| TMEM25 | 0.005076015 | 0.113137 | 16.13% | 22.82% |
| GPR27 | 0.00595835 | 0.107616 | 48.84% | 25.40% |
| CD47 | 0.005971164 | 0.155759 | 16.43% | 16.99% |
| LYPD6B | 0.006667331 | 0.515922 | 16.45% | 32.77% |
| TPBG | 0.006918377 | 0.317762 | 15.23% | 22.57% |
| SLC22A23 | 0.007282072 | 0.254882 | 14.87% | 32.56% |
| KIRREL2 | 0.007553205 | 0.197278 | 90.11% | 62.60% |
| SLC2A13 | 0.008133276 | 0.367651 | 17.22% | 17.06% |
| ACVRIC | 0.008198981 | 0.549338 | 76.39% | 62.72% |
| DLL4 | 0.008400227 | 0.172189 | 22.50% | 14.75% |
| UNC93A | 0.008486685 | 0.548395 | 87.31% | 67.17% |
| CDH7 | 0.008507328 | 0.525223 | 86.31% | 28.86% |
| SLITRK5 | 0.009181416 | 0.409606 | 81.31% | 58.10% |
| NEGR1 | 0.009654704 | 0.368091 | 59.26% | 46.39% |
| SLITRK6 | 0.010256541 | 0.249755 | 53.01% | 52.15% |
| SLC37A1 | 0.010299059 | 0.154556 | 20.04% | 12.30% |
| PCDHA1 | 0.010469816 | 0.200406 | 77.07% | 73.88% |
| LRFN2 | 0.011657549 | 0.313324 | 14.95% | 67.99% |
| GPR115 | 0.012887629 | 0.724504 | 64.64% | 63.34% |
| BCAN | 0.013082657 | 0.705165 | 58.46% | 71.33% |
| GP2 | 0.014390617 | 0.870841 | 63.97% | 87.05% |
| KISS1R | 0.014691855 | 0.631176 | 59.36% | 46.96% |
| NCAM2 | 0.01495719 | 0.574525 | 13.22% | 13.53% |
| JAG2 | 0.015320507 | 0.123251 | 28.91% | 17.83% |
| PRPH2 | 0.015938273 | 0.218703 | 59.46% | 56.88% |
| OR8G1 | 0.016332738 | 0.812903 | 100.00% | 100.00% |
| PTGER1 | 0.016428746 | 0.464843 | 48.69% | 44.24% |
| OR51G2 | 0.017016783 | 0.610332 | 90.24% | 92.32% |
| NRN1 | 0.017114781 | 0.296517 | 41.60% | 17.56% |
| LPHN3 | 0.017398384 | 0.105528 | 48.07% | 36.49% |
| NPHS1 | 0.018038642 | 0.391864 | 92.90% | 66.15% |
| KIAA1324 | 0.019204802 | 0.144505 | 42.06% | 18.99% |
| PCDHB14 | 0.01989985 | 0.23259 | 30.09% | 24.50% |
| SLC45A2 | 0.020204874 | 0.146653 | 56.00% | 65.47% |
| SCARB1 | 0.02091067 | 0.215994 | 18.81% | 14.26% |
| SLC5A1 | 0.023194202 | 0.352354 | 65.18% | 41.98% |
| AMIGO2 | 0.023669182 | 0.250072 | 15.46% | 36.48% |
| GLRB | 0.023978169 | 0.250079 | 41.21% | 36.23% |
| ENPP1 | 0.025333073 | 0.202569 | 55.34% | 31.01% |
| GABRA2 | 0.02581235 | 0.898768 | 83.82% | 95.94% |
| CADM2 | 0.026724848 | 0.262917 | 88.15% | 68.40% |
| SLITRK3 | 0.02779597 | 0.419223 | 65.59% | 54.37% |
| OR51E2 | 0.027850862 | 0.601597 | 84.61% | 76.27% |
| MET | 0.029443618 | 0.163592 | 28.79% | 33.81% |
| PKD2L1 | 0.033329961 | 0.597712 | 74.03% | 52.20% |
| OR51S1 | 0.03443262 | 0.695061 | 100.00% | 100.00% |
| CEACAM3 | 0.034736354 | 0.627567 | 43.70% | 13.53% |
| SLC7A9 | 0.034866161 | 0.132988 | 61.12% | 42.60% |
| ABCA12 | 0.036267598 | 0.659658 | 18.44% | 70.41% |
| CDHR3 | 0.036445654 | 0.179738 | 65.97% | 54.34% |
| OR51F1 | 0.037238532 | 0.560063 | 100.00% | 100.00% |
| OR51L1 | 0.037274686 | 0.912869 | 100.00% | 67.13% |
| SLC1A5 | 0.038122893 | 0.131175 | 13.31% | 14.63% |
| SLC4A5 | 0.039046047 | 0.171187 | 10.99% | 19.33% |
| DAG1 | 0.039359211 | 0.156186 | 11.94% | 13.56% |
| OR8D1 | 0.044146729 | 0.906771 | 100.00% | 100.00% |
| CD24 | 0.04478253 | 0.126977 | 15.70% | 10.14% |
| GJB4 | 0.045238901 | 0.368761 | 79.41% | 48.66% |
| SLITRK1 | 0.045845498 | 0.649526 | 61.33% | 40.37% |
| SCN9A | 0.046290464 | 0.319877 | 24.27% | 51.12% |
| PTGDR2 | 0.046940022 | 0.556022 | 48.74% | 59.95% |
| SSTR5 | 0.049548712 | 0.428603 | 97.85% | 80.92% |
| LYPD5 | 0.050390723 | 0.168328 | 11.83% | 39.82% |
| FZD3 | 0.050840924 | 0.121895 | 16.82% | 28.70% |
| LINGO2 | 0.051488414 | 0.497153 | 61.92% | 85.78% |
| SLC39A4 | 0.05155746 | 0.230646 | 22.47% | 21.11% |
| SLC8A1 | 0.055683173 | 0.2683 | 19.48% | 17.76% |
| CEACAM20 | 0.05645749 | 0.94661 | 97.66% | 98.38% |
| MANSC1 | 0.060362259 | 0.126194 | 12.53% | 16.79% |
| IL5RA | 0.061991206 | 0.292696 | 47.81% | 35.85% |
| OR8A1 | 0.064199917 | 0.947314 | 90.89% | 79.37% |
| OR56A5 | 0.066441287 | 0.757234 | 34.32% | 95.44% |
| SLC8A3 | 0.070473098 | 0.295359 | 87.15% | 71.17% |
| OR52B4 | 0.070833748 | 1 | 17.16% | 100.00% |
| SLCO1A2 | 0.071664988 | 0.484353 | 48.62% | 69.20% |
| NCR2 | 0.081128593 | 0.599899 | 45.53% | 78.69% |
| GPC3 | 0.082397966 | 0.226161 | 33.74% | 31.63% |
| OR51A7 | 0.085724844 | 0.757758 | 85.91% | 100.00% |
| GPR56 | 0.085744928 | 0.203505 | 20.94% | 19.17% |
| CHL1 | 0.085856017 | 0.499857 | 35.93% | 22.98% |
| OR1J2 | 0.086388495 | 0.813048 | 16.42% | 49.95% |
| SGCZ | 0.086457707 | 0.721765 | 55.72% | 73.90% |
| PCDHA13 | 0.087239323 | 0.492689 | 88.37% | 54.59% |
| ZACN | 0.088033335 | 0.305461 | 79.39% | 41.84% |
| MUC12 | 0.091755769 | 0.4751 | 84.60% | 67.64% |
| PCDHB8 | 0.091950401 | 0.336234 | 33.55% | 48.22% |
| UTS2R | 0.09236842 | 0.236408 | 77.45% | 20.44% |
| HTR1E | 0.098631893 | 0.510789 | 86.27% | 96.13% |
| SYNPR | 0.099424732 | 0.424215 | 49.20% | 70.90% |
| ACPT | 0.101996953 | 0.172541 | 81.16% | 61.02% |
| MFSD2B | 0.104647085 | 0.199805 | 51.22% | 44.30% |
| TMC7 | 0.109715468 | 0.191021 | 16.52% | 26.12% |
| MS4A15 | 0.110898446 | 0.746087 | 52.09% | 24.32% |
| OR51F2 | 0.114529932 | 0.698836 | 100.00% | 65.11% |
| GRIA2 | 0.115696545 | 0.502523 | 37.59% | 56.31% |
| GALR1 | 0.120909898 | 0.420528 | 76.19% | 56.16% |
| SLC32A1 | 0.121131002 | 0.738544 | 97.93% | 78.76% |
| FFAR2 | 0.129810246 | 0.119595 | 55.02% | 36.60% |
| SLC13A3 | 0.131090881 | 0.436619 | 19.72% | 49.03% |
| RNF150 | 0.133545778 | 0.237511 | 37.88% | 34.97% |
| CHRM5 | 0.134954761 | 0.643328 | 19.81% | 47.44% |
| OR52R1 | 0.135308114 | 0.743844 | 91.81% | 81.51% |
| NETO1 | 0.136383182 | 0.435375 | 85.26% | 33.43% |
| SLC5A12 | 0.138407917 | 0.451971 | 48.11% | 62.46% |
| GRIK1 | 0.13890028 | 0.774247 | 40.96% | 61.64% |
| ADAM29 | 0.139801139 | 0.641763 | 24.58% | 63.82% |
| SLCO5A1 | 0.141583549 | 0.316595 | 27.90% | 23.05% |
| ADAM21 | 0.147660529 | 0.148199 | 51.40% | 50.57% |
| GPR37 | 0.148497131 | 0.335368 | 45.21% | 34.83% |
| PCDHB16 | 0.14964025 | 0.253341 | 17.74% | 39.49% |
| OR56B4 | 0.155286966 | 0.416374 | 36.04% | 69.88% |
| CNTNAP3B | 0.156215375 | 0.198115 | 68.70% | 38.14% |
| SLC38A11 | 0.165681894 | 0.598921 | 78.94% | 45.42% |
| HTR4 | 0.166055241 | 0.414149 | 68.27% | 81.77% |
| MST1R | 0.171722942 | 0.134391 | 16.71% | 31.26% |
| PMEL | 0.172241872 | 0.233045 | 31.54% | 42.33% |
| SLC15A5 | 0.177222694 | 0.435818 | 80.62% | 100.00% |
| MEP1B | 0.179479737 | 0.209065 | 57.17% | 63.18% |
| ART1 | 0.184855852 | 0.512029 | 77.07% | 78.94% |
| LGR5 | 0.190299628 | 0.214657 | 56.92% | 32.17% |
| GABRR1 | 0.191134594 | 0.935444 | 97.84% | 100.00% |
| OR51T1 | 0.192575362 | 0.534236 | 73.02% | 71.37% |
| PIGR | 0.195634422 | 0.381963 | 23.44% | 14.43% |
| PCDHA7 | 0.198105226 | 0.152205 | 59.22% | 59.88% |
| SLC2A7 | 0.200172164 | 0.389601 | 63.39% | 55.87% |
| OR2T2 | 0.202845671 | 0.540744 | 100.00% | 100.00% |
| PLXNA2 | 0.205565774 | 0.148909 | 17.56% | 20.13% |
| CHRNA4 | 0.21323047 | 0.422938 | 97.08% | 53.37% |
| MRGPRX2 | 0.215983064 | 0.855124 | 55.81% | 62.84% |
| CHRM3 | 0.217633548 | 0.23049 | 25.72% | 53.53% |
| GPR148 | 0.219300333 | 0.525458 | 74.64% | 91.55% |
| SLC44A5 | 0.236569238 | 0.293996 | 31.83% | 32.39% |
| PLD5 | 0.237947047 | 0.463887 | 62.94% | 30.98% |
| TGFBR3 | 0.238729359 | 0.223404 | 33.42% | 12.27% |
| OR1J4 | 0.238749781 | 0.47034 | 63.96% | 50.28% |
| OR1Q1 | 0.241675133 | 0.404288 | 19.54% | 64.39% |
| BEST2 | 0.244576766 | 0.592918 | 78.39% | 83.86% |
| OR1F1 | 0.247679011 | 0.716483 | 93.01% | 82.59% |
| OR14I1 | 0.254154812 | 0.654576 | 87.93% | 81.23% |
| TACR3 | 0.256873797 | 0.138153 | 48.76% | 74.79% |
| SLC20A2 | 0.260899684 | 0.47988 | 36.02% | 20.25% |
| TBXA2R | 0.261240502 | 0.22995 | 35.56% | 16.61% |
| GPR101 | 0.262437498 | 0.576105 | 83.17% | 61.26% |
| OR2G2 | 0.263274302 | 0.711543 | 100.00% | 64.36% |
| OR10P1 | 0.264740003 | 0.285346 | 100.00% | 68.65% |
| ZP3 | 0.265528678 | 0.115483 | 19.26% | 21.51% |
| HTR3C | 0.271703182 | 0.868092 | 36.92% | 32.33% |
| SLC5A7 | 0.273328515 | 0.419321 | 95.31% | 90.15% |
| RXFP1 | 0.274291063 | 0.640113 | 31.88% | 29.69% |
| CDHR4 | 0.275830566 | 0.445499 | 52.06% | 61.96% |
| GABRA3 | 0.276147578 | 0.510692 | 37.78% | 43.02% |
| PCDHB10 | 0.279015447 | 0.202287 | 25.58% | 20.18% |
| BEST4 | 0.279529035 | 0.252307 | 29.91% | 27.63% |
| OR2F2 | 0.285177702 | 0.832881 | 100.00% | 81.99% |
| OR51Q1 | 0.287759935 | 0.801022 | 54.15% | 100.00% |
| LHFPL5 | 0.293518396 | 0.21691 | 30.72% | 34.51% |
| GPR37L1 | 0.298958368 | 0.38006 | 20.98% | 30.12% |
| PCDHB6 | 0.303185061 | 0.139004 | 23.24% | 54.67% |
| SLC12A1 | 0.304749154 | 0.821557 | 60.97% | 89.86% |
| CFC1 | 0.311391988 | 0.990789 | 100.00% | 89.23% |
| GPR119 | 0.311805215 | 0.457402 | 100.00% | 77.97% |
| IL1RAPL1 | 0.3136187 | 0.471525 | 62.18% | 75.23% |
| BRS3 | 0.317560714 | 0.275005 | 91.46% | 64.32% |
| OR2L3 | 0.325542542 | 0.791928 | 62.09% | 100.00% |
| NMBR | 0.32639682 | 0.138086 | 51.40% | 36.03% |
| SLC34A3 | 0.343822185 | 0.369108 | 87.55% | 43.72% |
| ZNRF4 | 0.351218852 | 0.505083 | 95.65% | 74.00% |
| CHRNA2 | 0.352021588 | 0.852557 | 89.06% | 88.78% |
| GALR3 | 0.353632174 | 0.586378 | 27.76% | 57.48% |
| GIPR | 0.354815735 | 0.213851 | 50.07% | 22.04% |
| GABRA4 | 0.358534778 | 0.768526 | 86.57% | 74.43% |
| PTPRZ1 | 0.364759277 | 0.633729 | 21.92% | 41.20% |
| P2RX6 | 0.372284329 | 0.184313 | 29.40% | 46.40% |
| OR51G1 | 0.374237004 | 0.514699 | 100.00% | 84.28% |
| CNTN5 | 0.383557411 | 0.676637 | 56.38% | 64.09% |
| OR1C1 | 0.388589636 | 1 | 56.15% | 62.78% |
| GABRB1 | 0.390463253 | 0.618987 | 58.03% | 49.04% |
| OR51B2 | 0.394225873 | 1 | 54.28% | 17.48% |
| TACR2 | 0.394580056 | 0.21082 | 51.40% | 10.06% |
| SLC6A18 | 0.394713425 | 0.263789 | 100.00% | 61.92% |
| OR8G2 | 0.396345118 | 0.545095 | 100.00% | 100.00% |
| OR1L1 | 0.397354663 | 0.83068 | 70.82% | 73.51% |
| OR10AG1 | 0.401198599 | 0.52291 | 84.67% | 100.00% |
| OR2S2 | 0.41170195 | 0.587798 | 58.68% | 73.04% |
| CCR9 | 0.417804595 | 0.393008 | 57.09% | 66.87% |
| GJB5 | 0.421998357 | 0.180577 | 50.83% | 20.16% |
| CACNG3 | 0.425279062 | 0.230333 | 97.52% | 86.12% |
| OR2L13 | 0.426916519 | 0.7394 | 10.94% | 78.54% |
| NUP210L | 0.430722039 | 0.157832 | 61.64% | 67.34% |
| SLC26A3 | 0.432045118 | 0.738256 | 75.74% | 46.73% |
| GPRC6A | 0.446303254 | 0.982045 | 87.55% | 95.09% |
| OR7D4 | 0.455205742 | 0.603949 | 100.00% | 83.33% |
| MUC17 | 0.46122426 | 0.32458 | 96.89% | 53.84% |
| OR1L3 | 0.468035802 | 0.58562 | 46.99% | 68.65% |
| LRRC4C | 0.47309339 | 0.790211 | 53.14% | 75.68% |
| RXFP2 | 0.47356422 | 1 | 49.20% | 100.00% |
| NPBWR1 | 0.478981981 | 0.384959 | 19.76% | 55.23% |
| OR7C2 | 0.482071482 | 0.928642 | 73.97% | 100.00% |
| GCGR | 0.484284551 | 0.392066 | 89.68% | 83.91% |
| OR8B12 | 0.495204567 | 0.199983 | 100.00% | 100.00% |
| PCDH10 | 0.49819273 | 0.238572 | 47.43% | 66.75% |
| OR2A25 | 0.50715683 | 0.360482 | 73.54% | 100.00% |
| GLRA2 | 0.512056674 | 0.661314 | 66.68% | 95.27% |
| TAAR5 | 0.521170378 | 0.14502 | 100.00% | 51.66% |
| OTOP2 | 0.527633573 | 0.756464 | 98.53% | 57.66% |
| TMPRSS15 | 0.529964199 | 0.613393 | 67.52% | 54.53% |
| OR13F1 | 0.530608489 | 0.742802 | 82.56% | 68.24% |
| OR5A1 | 0.534414833 | 0.458599 | 100.00% | 36.84% |
| ASGR2 | 0.534688 | 0.3959 | 43.47% | 14.70% |
| GHSR | 0.536780388 | 0.887473 | 91.92% | 88.46% |
| KCNK18 | 0.548471162 | 0.761076 | 100.00% | 38.39% |
| OR8K1 | 0.567010311 | 0.291298 | 100.00% | 71.70% |
| LRRTM4 | 0.571301144 | 0.331909 | 21.06% | 70.71% |
| CDH10 | 0.591179559 | 0.533814 | 32.21% | 31.48% |
| OR2T10 | 0.592870688 | 1 | 14.66% | 85.27% |
| NTNG1 | 0.592879926 | 0.240935 | 60.32% | 22.78% |
| LRTM1 | 0.597870931 | 0.157215 | 80.49% | 81.47% |
| TACR1 | 0.59816171 | 0.26735 | 36.79% | 32.95% |
| OR11A1 | 0.598998302 | 0.295719 | 89.97% | 58.08% |
| SLC17A1 | 0.599370324 | 0.502156 | 100.00% | 60.50% |
| PCDHGB6 | 0.599663955 | 0.21695 | 30.34% | 46.95% |
| OR10G3 | 0.611490713 | 0.74702 | 100.00% | 20.64% |
| OR4D9 | 0.613727779 | 0.398394 | 48.70% | 100.00% |
| SLC22A6 | 0.615993812 | 0.327099 | 90.60% | 22.74% |
| TYR | 0.622900631 | 0.788116 | 65.92% | 85.10% |
| EGFR | 0.627012522 | 0.173057 | 29.98% | 21.94% |
| PKD1L2 | 0.627138204 | 0.170139 | 54.02% | 29.57% |
| SLC22A14 | 0.627836328 | 0.837303 | 64.23% | 72.07% |
| DRD3 | 0.628693142 | 0.319338 | 59.32% | 12.29% |
| GPC5 | 0.636579657 | 0.400156 | 42.14% | 34.80% |
| OR6A2 | 0.639116497 | 0.808372 | 93.39% | 100.00% |
| OR4D6 | 0.644465007 | 0.89489 | 45.90% | 100.00% |
| CACNA1I | 0.647825326 | 0.482865 | 61.76% | 35.82% |
| OR4X2 | 0.659557232 | 0.795779 | 100.00% | 100.00% |
| SLC44A4 | 0.669722054 | 0.181155 | 13.03% | 15.58% |
| PTCHD3 | 0.675579697 | 0.889982 | 57.94% | 60.90% |
| AQP10 | 0.680129188 | 0.439666 | 15.42% | 38.32% |
| OR7G3 | 0.683683855 | 0.2655 | 100.00% | 51.56% |
| CRB1 | 0.691759031 | 0.171984 | 48.30% | 47.33% |
| ADAM30 | 0.69755855 | 0.540237 | 65.31% | 77.62% |
| OPN5 | 0.704011413 | 0.828511 | 96.92% | 50.32% |
| OR9Q1 | 0.709790119 | 0.620133 | 73.87% | 71.28% |
| LRRTM3 | 0.722653288 | 0.105728 | 79.85% | 42.52% |
| SLC2A2 | 0.750041962 | 0.557398 | 20.44% | 72.05% |
| TMEM114 | 0.761434161 | 0.206386 | 90.87% | 50.03% |
| KCNJ3 | 0.773441601 | 0.242145 | 47.19% | 34.73% |
| OR1K1 | 0.778808459 | 0.278018 | 46.73% | 50.89% |
| MUC21 | 0.784645007 | 0.347383 | 35.29% | 54.94% |
| GP5 | 0.787311346 | 0.230306 | 31.92% | 30.11% |
| GYPA | 0.79142846 | 0.682098 | 34.95% | 34.81% |
| SLC16A12 | 0.800712751 | 0.102803 | 42.54% | 33.24% |
| PTPRR | 0.819079688 | 0.192063 | 12.44% | 22.47% |
| NTSR2 | 0.821965712 | 0.483076 | 51.32% | 62.11% |
| OR5B12 | 0.834986035 | 0.758609 | 64.29% | 50.95% |
| GPR112 | 0.874242494 | 0.363501 | 29.65% | 48.44% |
| TAAR8 | 0.905692412 | 0.361322 | 27.52% | 49.90% |
| SLC9A7 | 0.931945612 | 0.410788 | 31.75% | 14.70% |
| ADRA1D | 0.933185867 | 0.230098 | 23.28% | 23.30% |
| OR2A14 | 0.935214939 | 0.251381 | 45.19% | 34.10% |
| OR52K2 | 0.993735068 | 0.260296 | 29.90% | 19.22% |
| OR10J5 | 0.996448953 | 0.201452 | 16.77% | 13.86% |
| CNTNAP2 | 9.47371E−12 | 0.045283 | 70.31% | 26.44% |
| SYP | 1.25311E−11 | 0.013618 | 48.43% | 21.91% |
| SEZ6 | 6.94855E−11 | 0.050499 | 66.22% | 35.83% |
| IL10RB | 1.53692E−07 | 0.197341 | 11.50% | 0.33% |
| VLDLR | 1.90564E−07 | 0.388037 | 1.81% | 47.00% |
| CHRNB2 | 1.50541E−06 | 0.050225 | 52.96% | 21.24% |
| QSOX2 | 3.19787E−06 | 0.071822 | 27.20% | 7.78% |
| NPTN | 4.11171E−06 | 0.214732 | 8.05% | 10.15% |
| SV2A | 4.87056E−06 | 0.022505 | 27.04% | 18.72% |
| SIGIRR | 1.66864E−05 | 0.267117 | 4.46% | 20.89% |
| ITFG1 | 2.99819E−05 | 0.145539 | 2.23% | 5.25% |
| CADM1 | 3.06039E−05 | 0.064151 | 38.80% | 10.40% |
| SLC29A4 | 5.87081E−05 | 0.001259 | 55.04% | 20.99% |
| LRRC24 | 6.84993E−05 | 0.01443 | 47.89% | 25.21% |
| TSPAN31 | 7.94379E−05 | 0.176463 | 5.43% | 5.55% |
| PVR | 0.000131708 | 0.296659 | 1.80% | 33.59% |
| PODXL2 | 0.000171054 | 0.051888 | 19.39% | 13.20% |
| FAM171B | 0.000348213 | 0.090878 | 48.37% | 27.73% |
| ENPP4 | 0.000556761 | 0.214433 | 7.41% | 17.90% |
| PIGT | 0.000621992 | 0.13698 | 1.94% | 13.38% |
| SGCB | 0.000624351 | 0.009972 | 23.21% | 9.15% |
| RET | 0.000700213 | 0.060464 | 65.72% | 53.26% |
| LGR4 | 0.000718549 | 0.057668 | 26.04% | 22.82% |
| ADCY5 | 0.000768431 | 0.031072 | 58.69% | 31.66% |
| CLDN3 | 0.000786408 | 0.043539 | 25.59% | 20.60% |
| SLC17A5 | 0.000819867 | 0.17861 | 16.12% | 6.95% |
| LRP12 | 0.00085377 | 0.01955 | 22.90% | 16.08% |
| GGT7 | 0.000985048 | 0.034975 | 5.67% | 16.80% |
| TMEM182 | 0.001067196 | 0.289553 | 2.98% | 25.17% |
| LMBR1 | 0.001082348 | 0.066479 | 16.03% | 8.41% |
| PAM | 0.001181299 | 0.018784 | 29.92% | 26.30% |
| IFNAR1 | 0.001600572 | 0.10435 | 8.97% | 1.88% |
| SERINC3 | 0.00186261 | 0.101053 | 1.84% | 5.67% |
| SERINC1 | 0.001934137 | 0.061776 | 8.85% | 5.34% |
| MFSD6 | 0.002322247 | 0.012274 | 15.95% | 11.68% |
| SSR1 | 0.00248723 | 0.109119 | 2.80% | 2.75% |
| P2RX4 | 0.002513039 | 0.146105 | 14.51% | 0.66% |
| TMEM8B | 0.002606002 | 0.050811 | 19.10% | 28.71% |
| CRB3 | 0.002738949 | 0.290113 | 6.60% | 29.70% |
| TMEM161A | 0.003043568 | 0.10575 | 5.63% | 14.62% |
| LIFR | 0.003323269 | 0.021622 | 47.05% | 9.40% |
| SLC37A3 | 0.00334168 | 0.068352 | 13.79% | 12.12% |
| PVRL2 | 0.003954317 | 0.19781 | 0.97% | 10.46% |
| ATP13A3 | 0.004788394 | 0.103693 | 7.02% | 10.99% |
| CXADR | 0.006731902 | 0.067652 | 19.39% | 25.99% |
| SLC26A5 | 0.00719155 | 0.020972 | 66.27% | 27.82% |
| OPRD1 | 0.008009037 | 0.025836 | 63.53% | 47.62% |
| GPR160 | 0.009903187 | 0.299374 | 18.19% | 6.12% |
| HIAT1 | 0.010748544 | 0.074637 | 5.61% | 3.31% |
| ADAM28 | 0.011485543 | 0.426139 | 24.99% | 3.02% |
| SPPL2B | 0.011489005 | 0.028025 | 19.47% | 12.83% |
| PTTG1IP | 0.012178726 | 0.050763 | 6.79% | 3.73% |
| NUP210 | 0.013228414 | 0.1221 | 11.14% | 9.53% |
| EPCAM | 0.013861589 | 0.066145 | 7.79% | 12.85% |
| TMEM8A | 0.014941666 | 0.140918 | 9.54% | 7.66% |
| LRRC37B | 0.015292209 | 0.070554 | 11.60% | 15.48% |
| DIRC2 | 0.015750615 | 0.049001 | 21.44% | 3.46% |
| TMEM219 | 0.015837944 | 0.099902 | 3.06% | 1.95% |
| STT3B | 0.01697836 | 0.076165 | 0.77% | 0.49% |
| IGSF8 | 0.017926295 | 0.156815 | 6.89% | 10.06% |
| TMEM123 | 0.018384773 | 0.10213 | 3.42% | 7.79% |
| DAGLA | 0.018990173 | 0.062827 | 32.83% | 23.95% |
| FZD9 | 0.019025909 | 0.121833 | 64.78% | 8.59% |
| SLC7A5 | 0.019735889 | 0.20518 | 11.27% | 9.68% |
| SLC39A9 | 0.021042591 | 0.013011 | 13.62% | 9.70% |
| FREM2 | 0.021128353 | 0.40561 | 8.44% | 53.92% |
| ACVR1 | 0.02233146 | 0.164949 | 1.40% | 4.97% |
| SLC9A6 | 0.023756876 | 0.07958 | 17.01% | 15.55% |
| BSG | 0.024425203 | 0.088992 | 7.17% | 4.48% |
| SEMA4F | 0.026736167 | 0.08673 | 23.60% | 15.68% |
| TMEM108 | 0.028703465 | 0.110059 | 6.47% | 29.28% |
| SLC2A11 | 0.028949765 | 0.084674 | 34.19% | 23.56% |
| SLC41A3 | 0.031855762 | 0.046125 | 14.27% | 16.88% |
| PPAP2C | 0.03277155 | 0.195446 | 7.35% | 27.08% |
| SLC2A8 | 0.036955992 | 0.122282 | 14.68% | 5.91% |
| TMEM179B | 0.03701416 | 0.084872 | 0.88% | 0.36% |
| CLDN4 | 0.037642954 | 0.001793 | 14.24% | 10.28% |
| TM9SF1 | 0.038196952 | 0.083833 | 10.12% | 5.67% |
| NETO2 | 0.039255946 | 0.160351 | 8.82% | 18.80% |
| RNF167 | 0.039610855 | 0.06436 | 1.73% | 0.71% |
| TSPAN15 | 0.040748581 | 0.064938 | 14.20% | 11.65% |
| GABRG3 | 0.043676947 | 0.094642 | 88.03% | 5.54% |
| SLC3A2 | 0.046660579 | 0.105099 | 0.078519 | 0.056853 |

TABLE 2
Surface proteins associated with the SCLC-A subtype from the cell line dataset

| Surface Protein | ANOVA P value | A vs N | A vs P | A vs I |
|---|---|---|---|---|
| CLDN3 | 2.42551E−13 | 0.719746 | 59.47% | 81.14% |
| NKAIN2 | 3.38063E−13 | 0.462799 | 20.37% | 36.96% |
| SCNN1A | 6.71207E−11 | 0.619875 | 36.18% | 66.62% |
| PTPRN2 | 3.57913E−09 | 0.260752 | 45.18% | 40.09% |
| DNER | 5.17956E−09 | 0.563469 | 34.00% | 32.34% |
| FAM174B | 7.17107E−09 | 0.215947 | 29.42% | 61.31% |
| GPR56 | 9.74635E−09 | 0.177952 | 27.01% | 42.15% |
| DLL4 | 1.44489E−08 | 0.342869 | 48.18% | 62.62% |
| KCNMB2 | 2.35185E−08 | 0.276975 | 14.63% | 29.58% |
| CLDN4 | 2.51125E−08 | 0.373644 | 52.95% | 65.47% |
| KIAA1324 | 8.52893E−08 | 0.202579 | 32.53% | 47.27% |
| OR51F1 | 2.5326E−07 | 0.741796 | 54.03% | 52.07% |
| OR51T1 | 3.15478E−07 | 0.95222 | 73.14% | 86.02% |
| F11R | 8.6059E−07 | 0.393838 | 10.45% | 26.21% |
| GPR6 | 9.43586E−07 | 0.62593 | 69.64% | 89.85% |
| TMEM116 | 9.49245E−07 | 0.116633 | 10.12% | 11.75% |
| TMEM154 | 1.03977E−06 | 0.488411 | 56.73% | 42.52% |
| OR51G1 | 1.18153E−06 | 0.894596 | 74.63% | 79.00% |
| BAMBI | 1.30824E−06 | 0.425267 | 16.33% | 29.25% |
| OR51F2 | 1.76533E−06 | 0.684157 | 60.86% | 65.69% |
| SDK2 | 2.79986E−06 | 0.215895 | 38.16% | 36.37% |
| MTNR1B | 2.85489E−06 | 0.718696 | 70.73% | 61.65% |
| CXCR4 | 3.01882E−06 | 0.105171 | 31.48% | 43.83% |
| SLC36A4 | 3.59191E−06 | 0.148415 | 18.74% | 16.36% |
| OR52R1 | 5.08417E−06 | 0.62271 | 56.16% | 62.29% |
| OR51H1P | 6.71689E−06 | 0.835138 | 72.51% | 70.11% |
| OR51S1 | 1.23112E−05 | 0.856013 | 54.78% | 83.03% |
| CLDN7 | 1.36784E−05 | 0.445548 | 14.30% | 75.36% |
| GRM8 | 1.56799E−05 | 0.255458 | 28.23% | 57.22% |
| CEACAM5 | 1.67657E−05 | 0.456221 | 65.01% | 41.14% |
| GPR39 | 2.0823E−05 | 0.708464 | 54.81% | 73.64% |
| OR51L1 | 2.76272E−05 | 0.944632 | 86.72% | 71.37% |
| OR51A4 | 2.78456E−05 | 0.850676 | 60.23% | 75.82% |
| OR51A2 | 3.46569E−05 | 0.883599 | 70.15% | 77.76% |
| FRAS1 | 3.57098E−05 | 0.152946 | 16.43% | 25.65% |
| OR51A7 | 5.13974E−05 | 0.926519 | 75.45% | 88.77% |
| CEACAM6 | 5.9763E−05 | 0.697588 | 75.50% | 61.56% |
| LYPD1 | 6.97097E−05 | 0.576418 | 70.68% | 71.19% |
| JAM3 | 7.10708E−05 | 0.100866 | 14.53% | 14.09% |
| ATP4B | 0.000124845 | 0.566772 | 53.35% | 28.01% |
| KL | 0.000150403 | 0.316534 | 44.43% | 62.89% |
| TMEM255B | 0.000210166 | 0.447391 | 48.21% | 37.19% |
| OR52J3 | 0.00028157 | 0.858534 | 99.46% | 79.47% |
| MUC1 | 0.000281706 | 0.418791 | 32.71% | 11.27% |
| PTH2R | 0.000394211 | 0.528961 | 70.03% | 53.52% |
| CEACAM7 | 0.000401652 | 0.824287 | 99.43% | 81.31% |
| DSCAML1 | 0.000428411 | 0.13678 | 19.10% | 55.37% |
| NMUR2 | 0.000465995 | 0.531378 | 50.69% | 62.11% |
| PROM1 | 0.000514814 | 0.321741 | 11.00% | 25.20% |
| ABCB5 | 0.000650776 | 0.493423 | 65.55% | 39.92% |
| TMEM132D | 0.000699523 | 0.634272 | 21.33% | 80.07% |
| TIGIT | 0.000711936 | 0.318193 | 16.58% | 37.50% |
| OR52A4 | 0.000743244 | 0.80462 | 82.39% | 85.20% |
| NLGN1 | 0.000762407 | 0.12472 | 28.25% | 33.92% |
| OR51G2 | 0.000815476 | 0.931449 | 80.30% | 74.63% |
| PLXNA2 | 0.000883663 | 0.166267 | 27.84% | 22.94% |
| PLB1 | 0.000886089 | 0.173229 | 60.91% | 34.00% |
| TMEM211 | 0.000903827 | 0.451466 | 51.58% | 56.14% |
| ADCY2 | 0.000924544 | 0.267393 | 48.20% | 54.76% |
| FNDC9 | 0.001027742 | 0.163174 | 13.25% | 37.72% |
| SLC6A17 | 0.001171438 | 0.113421 | 70.34% | 20.86% |
| CSMD1 | 0.001541352 | 0.440894 | 18.89% | 42.04% |
| TSPAN8 | 0.001664174 | 0.483321 | 58.42% | 11.17% |
| MPZ | 0.001960371 | 0.47327 | 32.86% | 57.35% |
| SLC45A2 | 0.002061415 | 0.374183 | 42.86% | 50.51% |
| CHL1 | 0.002097906 | 0.371243 | 24.96% | 33.93% |
| IL5RA | 0.002227456 | 0.335831 | 70.63% | 52.82% |
| TMEM184A | 0.002336914 | 0.178724 | 16.50% | 18.00% |
| SLC2A12 | 0.002557362 | 0.208757 | 18.09% | 20.29% |
| PCDHB1 | 0.002591703 | 0.675631 | 44.57% | 50.61% |
| LIFR | 0.003185162 | 0.13777 | 15.37% | 20.51% |
| SPPL2B | 0.003313453 | 0.16231 | 13.59% | 15.28% |
| SLC5A9 | 0.003729648 | 0.328247 | 21.84% | 21.12% |
| STEAP4 | 0.004297974 | 0.285109 | 25.12% | 28.78% |
| TMEM178B | 0.004738132 | 0.118263 | 23.28% | 22.80% |
| SLC18A1 | 0.005078518 | 0.538404 | 59.10% | 82.23% |
| SCN2A | 0.005183802 | 0.203847 | 43.44% | 18.98% |
| GPR158 | 0.00559701 | 0.182099 | 33.50% | 11.00% |
| TSPAN11 | 0.005935321 | 0.105031 | 10.63% | 32.52% |
| OR51E2 | 0.006425491 | 0.464681 | 60.55% | 42.55% |
| GRIN3A | 0.006427088 | 0.166567 | 29.03% | 27.53% |
| KLRC3 | 0.006460354 | 0.827042 | 10.83% | 36.94% |
| HEPHL1 | 0.0066345 | 0.398492 | 17.78% | 38.57% |
| OR52A5 | 0.006876442 | 0.861014 | 40.41% | 72.27% |
| CADM2 | 0.006912669 | 0.156053 | 36.21% | 16.89% |
| BTLA | 0.007819411 | 0.218228 | 64.45% | 35.83% |
| GALR1 | 0.007873528 | 0.545259 | 87.08% | 72.16% |
| THBD | 0.008144678 | 0.836794 | 78.35% | 83.67% |
| SLC6A5 | 0.008733233 | 0.218806 | 66.07% | 50.55% |
| OR52E2 | 0.008875518 | 0.697298 | 65.95% | 88.60% |
| TM4SF1 | 0.009828902 | 0.582092 | 99.69% | 11.90% |
| PANX2 | 0.011500501 | 0.450012 | 22.26% | 34.62% |
| RET | 0.013323451 | 0.473224 | 29.09% | 34.46% |
| SCN3A | 0.015665583 | 0.133641 | 14.93% | 26.18% |
| SLITRK4 | 0.017598956 | 0.266781 | 56.08% | 40.87% |
| TNFSF15 | 0.017811031 | 0.565279 | 35.26% | 20.46% |
| OR2AE1 | 0.019078549 | 0.565892 | 31.70% | 14.00% |
| MS4A1 | 0.019332289 | 0.520081 | 79.02% | 45.30% |
| OR52A1 | 0.019887299 | 0.842151 | 98.90% | 100.00% |
| CD200 | 0.02032333 | 0.382683 | 28.45% | 45.42% |
| FFAR4 | 0.020384015 | 0.493797 | 83.70% | 50.67% |
| TSPAN1 | 0.020737739 | 0.479562 | 52.59% | 21.83% |
| EPHB1 | 0.025803765 | 0.16633 | 23.58% | 30.74% |
| PCDH1 | 0.025925525 | 0.367423 | 26.26% | 15.43% |
| CNTN4 | 0.030821929 | 0.222679 | 22.61% | 29.36% |
| GPR143 | 0.031196622 | 0.170402 | 16.21% | 19.95% |
| OR2AG2 | 0.031381897 | 0.559685 | 59.72% | 100.00% |
| AVPRIA | 0.031769305 | 0.315882 | 67.64% | 72.54% |
| GPR144 | 0.031815936 | 0.520263 | 79.86% | 75.54% |
| GPR114 | 0.033564891 | 0.266623 | 99.60% | 43.90% |
| TLR10 | 0.033774749 | 0.63845 | 57.09% | 50.25% |
| GJB1 | 0.034227595 | 0.7153 | 99.37% | 66.03% |
| TLR1 | 0.037289655 | 0.603098 | 57.60% | 13.88% |
| MAMDC4 | 0.038907212 | 0.314596 | 61.24% | 14.98% |
| SLC24A3 | 0.039275276 | 0.129541 | 77.30% | 27.28% |
| ICAM2 | 0.046963398 | 0.82728 | 98.97% | 26.99% |
| CDH17 | 0.049357256 | 0.149412 | 33.39% | 10.67% |
| LRTM1 | 0.049881692 | 0.254531 | 56.65% | 26.87% |
| OR51E1 | 0.057873303 | 0.39426 | 36.42% | 53.85% |
| HTR5A | 0.060966983 | 0.781424 | 98.81% | 73.90% |
| NETO1 | 0.061280475 | 0.124161 | 63.66% | 37.61% |
| HTR1D | 0.062424675 | 0.276345 | 44.16% | 33.15% |
| OR10AD1 | 0.063295452 | 0.547734 | 11.47% | 31.82% |
| SCNN1G | 0.064272807 | 0.428267 | 74.48% | 31.38% |
| TLR6 | 0.065104936 | 0.373262 | 54.56% | 23.45% |
| P2RY12 | 0.068122543 | 0.112945 | 16.74% | 18.63% |
| SLC34A2 | 0.07384138 | 0.525343 | 52.77% | 32.40% |
| GLRA3 | 0.076258206 | 0.362804 | 13.84% | 56.15% |
| SLC7A9 | 0.076612149 | 0.233394 | 40.58% | 27.54% |
| CCKAR | 0.077838652 | 0.451114 | 45.93% | 45.25% |
| CHRNA10 | 0.079366153 | 0.490539 | 50.10% | 11.94% |
| KIAA1644 | 0.081777828 | 0.213633 | 28.13% | 57.82% |
| OR1L6 | 0.082566594 | 0.61398 | 59.91% | 60.94% |
| LCT | 0.083929719 | 0.214314 | 11.57% | 26.17% |
| LRRN4 | 0.084831855 | 0.335084 | 20.92% | 25.28% |
| GRPR | 0.093599699 | 0.329395 | 23.64% | 37.35% |
| FCRL6 | 0.094053988 | 0.512733 | 78.37% | 18.41% |
| PCDH17 | 0.094280193 | 0.138028 | 20.28% | 29.82% |
| OR2C1 | 0.105489764 | 0.691383 | 98.84% | 22.78% |
| GPR139 | 0.109840931 | 0.563193 | 99.15% | 100.00% |
| AQP9 | 0.114420742 | 0.268749 | 77.44% | 60.90% |
| CSMD2 | 0.117920936 | 0.127842 | 37.00% | 24.12% |
| GABRP | 0.119858515 | 0.285521 | 79.33% | 37.78% |
| GP2 | 0.12109813 | 0.697751 | 63.22% | 60.18% |
| SLC2A9 | 0.127552251 | 0.461735 | 12.09% | 34.53% |
| CCR8 | 0.129503819 | 0.608588 | 50.07% | 63.52% |
| GPR115 | 0.139420627 | 0.199964 | 63.64% | 74.58% |
| PRSS8 | 0.143302283 | 0.20152 | 22.70% | 56.78% |
| PCDHA7 | 0.149025155 | 0.21111 | 21.37% | 22.73% |
| TEX101 | 0.151357321 | 0.740308 | 48.34% | 75.46% |
| MUC4 | 0.151998995 | 0.17201 | 14.73% | 30.74% |
| NTM | 0.152298627 | 0.173338 | 35.80% | 52.44% |
| CHRM4 | 0.154316689 | 0.310639 | 32.01% | 39.36% |
| NAALADL1 | 0.155664822 | 0.273845 | 53.82% | 25.66% |
| KIRREL3 | 0.160339999 | 0.102269 | 47.57% | 18.30% |
| BTNL10 | 0.161012809 | 0.321967 | 23.15% | 44.76% |
| GLRA4 | 0.161339509 | 0.104277 | 33.06% | 44.28% |
| GJB3 | 0.163399011 | 0.383729 | 77.33% | 23.67% |
| ADAM28 | 0.163421263 | 0.180578 | 21.58% | 30.85% |
| OR51D1 | 0.167235034 | 0.977668 | 98.09% | 100.00% |
| TAAR1 | 0.169676454 | 0.830415 | 70.38% | 64.32% |
| LRRC4C | 0.172968676 | 0.303982 | 78.07% | 20.22% |
| CASR | 0.180071376 | 0.72817 | 10.51% | 78.51% |
| TMPRSS13 | 0.180606284 | 0.106 | 18.38% | 73.91% |
| MPL | 0.181982745 | 0.132992 | 16.82% | 18.75% |
| RXFP3 | 0.195262717 | 0.974637 | 97.83% | 100.00% |
| PLP1 | 0.196855329 | 0.202745 | 19.78% | 12.92% |
| UMOD | 0.203472941 | 0.984645 | 98.68% | 16.14% |
| NPC1L1 | 0.208111162 | 0.177569 | 50.34% | 59.47% |
| SLC16A12 | 0.211359962 | 0.384344 | 26.26% | 46.91% |
| ADAM29 | 0.219564057 | 0.674931 | 11.48% | 15.95% |
| TPO | 0.230214212 | 0.410634 | 73.11% | 34.47% |
| CLDN8 | 0.230826406 | 0.975655 | 97.91% | 100.00% |
| SLAMF9 | 0.239443665 | 0.803716 | 45.21% | 60.42% |
| OR56A4 | 0.24526769 | 0.712488 | 16.24% | 53.43% |
| GRIA1 | 0.247999743 | 0.118381 | 41.28% | 47.55% |
| GPR61 | 0.269250031 | 0.147287 | 98.77% | 27.41% |
| TACSTD2 | 0.270576747 | 0.843263 | 29.07% | 100.00% |
| GPR111 | 0.270949315 | 0.27181 | 45.49% | 53.23% |
| RXFP4 | 0.275641874 | 0.756933 | 43.73% | 59.35% |
| SSTR5 | 0.285760023 | 0.621207 | 47.10% | 100.00% |
| ZP1 | 0.286417239 | 0.275397 | 52.04% | 45.41% |
| LHFPL1 | 0.290557161 | 0.130155 | 64.05% | 67.56% |
| TREM1 | 0.291276251 | 0.529909 | 60.56% | 29.12% |
| P2RY6 | 0.293812431 | 0.761492 | 98.18% | 100.00% |
| SLITRK3 | 0.298052908 | 0.114055 | 24.70% | 40.92% |
| OR2AG1 | 0.298981285 | 0.317617 | 19.95% | 57.95% |
| CX3CR1 | 0.308359296 | 0.379878 | 99.10% | 35.66% |
| MUC15 | 0.322581996 | 0.102786 | 38.91% | 32.52% |
| CDH9 | 0.336510422 | 0.111464 | 82.27% | 23.50% |
| DCSTAMP | 0.346386452 | 0.428186 | 98.10% | 100.00% |
| PCDH11X | 0.350927663 | 0.228486 | 35.53% | 15.12% |
| TRAT1 | 0.356444465 | 0.292886 | 98.95% | 72.35% |
| OR13C4 | 0.363448784 | 0.250674 | 98.37% | 100.00% |
| GPR97 | 0.364496471 | 0.241852 | 98.68% | 56.91% |
| GPR50 | 0.366015279 | 0.253139 | 80.25% | 18.80% |
| DRD1 | 0.373528691 | 0.198974 | 57.45% | 48.01% |
| CHRM5 | 0.390903944 | 0.323917 | 26.60% | 22.80% |
| PVRL4 | 0.395927064 | 0.471404 | 98.64% | 18.51% |
| SCN1A | 0.40052708 | 0.133629 | 25.29% | 15.61% |
| SLC22A7 | 0.417435959 | 0.421769 | 29.45% | 26.09% |
| TREML2 | 0.429760152 | 0.654546 | 97.80% | 51.60% |
| CDH26 | 0.442764199 | 0.170486 | 13.77% | 18.62% |
| CLDN9 | 0.448499624 | 0.121858 | 98.73% | 51.50% |
| OR6A2 | 0.464019468 | 0.375298 | 10.04% | 30.22% |
| OR52N4 | 0.46837279 | 0.543369 | 21.42% | 16.84% |
| SORCS3 | 0.470945076 | 0.334882 | 29.37% | 37.19% |
| SELL | 0.482522576 | 0.270109 | 20.95% | 18.33% |
| OR51B2 | 0.49050313 | 0.238092 | 39.15% | 75.52% |
| VIPR1 | 0.493166622 | 0.174589 | 25.55% | 13.23% |
| SLC44A4 | 0.493325335 | 0.124078 | 23.96% | 52.04% |
| PKD1L2 | 0.499836618 | 0.260788 | 73.53% | 17.54% |
| DRD5 | 0.501647631 | 0.443843 | 67.66% | 31.39% |
| OR2G6 | 0.521852047 | 0.624042 | 98.44% | 29.89% |
| OR52E4 | 0.528009097 | 0.414756 | 53.62% | 30.49% |
| CCR7 | 0.528589134 | 0.284491 | 70.83% | 17.59% |
| OR52E8 | 0.543144092 | 0.457579 | 99.12% | 11.80% |
| OR52N2 | 0.54364032 | 0.469246 | 36.87% | 29.28% |
| MS4A6A | 0.56272475 | 0.468199 | 98.61% | 43.98% |
| GPR37L1 | 0.569660997 | 0.454327 | 32.00% | 29.86% |
| OR5AK2 | 0.571029371 | 0.52337 | 98.81% | 25.01% |
| OR52L1 | 0.581079191 | 0.651071 | 18.47% | 66.06% |
| OR6C70 | 0.589966926 | 0.385499 | 98.03% | 55.10% |
| CEACAM21 | 0.591307598 | 0.512154 | 97.78% | 51.31% |
| GRM1 | 0.603547953 | 0.199233 | 26.29% | 15.28% |
| PTPRO | 0.605148475 | 0.117836 | 10.83% | 12.97% |
| OR56A3 | 0.605413463 | 0.233456 | 33.07% | 70.48% |
| FCGR1B | 0.611416363 | 0.157712 | 98.35% | 26.50% |
| OR52N1 | 0.616368764 | 0.316361 | 27.49% | 37.74% |
| OR51B4 | 0.625043893 | 0.212658 | 36.16% | 63.84% |
| SIT1 | 0.670741496 | 0.454638 | 97.67% | 47.24% |
| RNF128 | 0.698191117 | 0.168108 | 26.79% | 25.13% |
| OR1E1 | 0.765370966 | 0.403748 | 96.91% | 100.00% |
| OR2Z1 | 0.772339568 | 0.551807 | 97.14% | 100.00% |
| MSLN | 0.832716302 | 0.335762 | 31.86% | 55.59% |
| TRGC2 | 0.877961037 | 0.308061 | 47.81% | 24.39% |
| OR51Q1 | 0.89044125 | 0.216263 | 13.16% | 40.13% |
| CD2 | 0.901949109 | 0.257533 | 96.63% | 10.98% |
| OR3A3 | 0.918956986 | 0.355678 | 93.70% | 100.00% |
| OR51I1 | 0.928903846 | 0.118784 | 12.11% | 48.30% |
| OR51I2 | 0.933934547 | 0.234344 | 29.46% | 23.59% |
| NPY1R | 0.954579812 | 0.203248 | 20.47% | 10.79% |
| NRXN1 | 2.81822E−13 | 0.000138 | 41.20% | 32.71% |
| SLC37A1 | 1.73598E−10 | 0.282594 | 3.80% | 54.72% |
| ILDR1 | 3.81693E−10 | 0.591516 | 9.63% | 71.40% |
| CADM1 | 2.05992E−08 | 0.097347 | 18.55% | 37.36% |
| CNTNAP2 | 1.93462E−07 | 0.080886 | 27.79% | 36.81% |
| KIAA0319 | 1.99897E−06 | 0.268488 | 40.00% | 9.84% |
| PLXNB1 | 2.56777E−06 | 0.193511 | 2.88% | 6.29% |
| MANSC1 | 4.95483E−06 | 0.077139 | 11.71% | 17.51% |
| CPM | 5.92894E−06 | 0.35473 | 9.41% | 34.60% |
| VSIG10 | 6.98693E−06 | 0.156953 | 5.37% | 2.76% |
| PCNXL2 | 8.89734E−06 | 0.03691 | 6.30% | 18.41% |
| TNFSF13B | 1.08318E−05 | 0.572241 | 6.52% | 38.67% |
| NCAM1 | 1.26863E−05 | 0.032152 | 0.36% | 32.60% |
| TNFRSF11A | 1.7428E−05 | 0.195698 | 6.01% | 47.87% |
| LAMP3 | 3.27086E−05 | 0.349462 | 7.97% | 24.67% |
| SLCO3A1 | 4.23488E−05 | 0.133386 | 1.16% | 20.47% |
| CD164 | 5.88986E−05 | 0.110949 | 4.87% | 1.26% |
| LRRC19 | 6.40915E−05 | 0.132696 | 7.69% | 12.78% |
| DLL3 | 6.99064E−05 | 0.021607 | 26.61% | 38.03% |
| CELSR3 | 7.84076E−05 | 0.043083 | 21.81% | 3.79% |
| SPINT2 | 8.432E−05 | 0.463714 | 0.12% | 27.87% |
| GPR98 | 0.000111511 | 0.080098 | 12.09% | 30.63% |
| DSCAM | 0.000142465 | 0.084568 | 37.30% | 66.39% |
| MFSD6 | 0.000155596 | 0.078808 | 3.37% | 11.82% |
| NRCAM | 0.000241684 | 0.01606 | 4.02% | 21.51% |
| CDHR2 | 0.000417751 | 0.048345 | 19.16% | 19.90% |
| CD274 | 0.000435901 | 0.361162 | 2.78% | 31.17% |
| SLC26A5 | 0.000457813 | 0.229818 | 6.87% | 18.86% |
| SCN8A | 0.00046048 | 0.030231 | 13.89% | 6.97% |
| SLC19A2 | 0.000471901 | 0.017147 | 2.97% | 14.87% |
| TMEM30A | 0.00047847 | 0.049206 | 2.10% | 1.18% |
| LTBR | 0.000601952 | 0.558914 | 3.06% | 45.93% |
| CNTNAP5 | 0.000944472 | 0.047497 | 55.74% | 72.69% |
| GPR137B | 0.001001296 | 0.160058 | 3.96% | 15.19% |
| SLCO4C1 | 0.001355291 | 0.027975 | 25.08% | 27.10% |
| CD226 | 0.00148 | 0.327766 | 7.32% | 7.98% |
| SLC15A1 | 0.001495143 | 0.488211 | 0.18% | 73.74% |
| IL17RE | 0.001650383 | 0.549996 | 5.20% | 9.29% |
| FAM174A | 0.001831702 | 0.125693 | 11.96% | 3.65% |
| OR13D1 | 0.002084051 | 0.482306 | 4.38% | 37.76% |
| MEP1B | 0.002138482 | 0.079696 | 25.53% | 30.89% |
| SELP | 0.002486472 | 0.329723 | 4.22% | 52.38% |
| GPR171 | 0.00257369 | 0.130563 | 8.54% | 33.36% |
| SV2B | 0.002605544 | 0.14781 | 1.58% | 58.45% |
| CDH2 | 0.002945579 | 0.005672 | 8.15% | 17.29% |
| NIPAL2 | 0.003184126 | 0.292185 | 20.99% | 5.70% |
| PCDHAC1 | 0.004493167 | 0.096666 | 10.94% | 17.64% |
| SLC24A5 | 0.004530771 | 0.00781 | 2.82% | 12.80% |
| EPHA7 | 0.00591625 | 0.105303 | 1.14% | 23.73% |
| LRP11 | 0.006693531 | 0.057794 | 6.75% | 3.65% |
| CADM4 | 0.007260938 | 0.165916 | 0.04% | 12.91% |
| HTR4 | 0.007408214 | 0.785335 | 9.56% | 61.17% |
| GPR107 | 0.007843444 | 0.045573 | 6.16% | 2.48% |
| PAM | 0.008245061 | 0.103585 | 8.72% | 8.04% |
| SLC4A8 | 0.008671296 | 0.014193 | 20.01% | 12.26% |
| NIPAL3 | 0.008955911 | 0.085266 | 1.17% | 13.35% |
| GPR19 | 0.009621378 | 0.018444 | 25.07% | 18.52% |
| ATP13A3 | 0.010339182 | 0.033086 | 2.61% | 6.69% |
| CLCA4 | 0.011761077 | 0.365154 | 8.42% | 36.31% |
| LMBRD2 | 0.013003468 | 0.052016 | 3.03% | 4.46% |
| P2RY14 | 0.015391254 | 0.08558 | 9.26% | 21.00% |
| DPP10 | 0.016109634 | 0.066815 | 15.21% | 44.03% |
| SLC16A7 | 0.01830618 | 0.058351 | 7.28% | 36.64% |
| SDK1 | 0.018394001 | 0.097402 | 5.86% | 26.61% |
| KLRC2 | 0.0199057 | 0.665188 | 2.29% | 6.94% |
| CHRNB2 | 0.019970912 | 0.009941 | 11.02% | 15.33% |
| PGAP1 | 0.020448682 | 0.020757 | 6.72% | 9.19% |
| SIDT1 | 0.020510928 | 0.07108 | 5.70% | 24.67% |
| LDLRAD4 | 0.020833278 | 0.239747 | 8.54% | 16.19% |
| CD5 | 0.021375005 | 0.025398 | 37.68% | 56.44% |
| ABCA12 | 0.021687914 | 0.108906 | 12.30% | 8.86% |
| KCNMB3 | 0.022103167 | 0.116531 | 6.05% | 7.32% |
| HCAR1 | 0.023805255 | 0.02454 | 6.50% | 5.35% |
| GPR137C | 0.024235948 | 0.111747 | 8.34% | 12.96% |
| OR13C3 | 0.024985001 | 0.331062 | 6.73% | 79.87% |
| SLC39A6 | 0.029919702 | 0.020042 | 6.16% | 3.60% |
| GPR87 | 0.030600045 | 0.129285 | 9.52% | 15.34% |
| P2RY13 | 0.032230889 | 0.126106 | 9.27% | 17.95% |
| TMEM9B | 0.039014426 | 0.058556 | 2.24% | 1.22% |
| NEGR1 | 0.0390838 | 0.021542 | 8.20% | 30.64% |
| SLAMF8 | 0.039703797 | 0.570583 | 28.66% | 9.38% |
| SEMA4D | 0.041428257 | 0.023521 | 4.84% | 13.37% |
| ZDHHC11 | 0.042993938 | 0.093724 | 17.59% | 28.47% |
| SLC44A1 | 0.047262548 | 0.045964 | 0.62% | 4.88% |
| ERMP1 | 0.048015557 | 0.058031 | 1.04% | 4.27% |
| TMCO3 | 0.049898976 | 0.060559 | 1.78% | 6.22% |

TABLE 3
Surface proteins associated with the SCLC-A subtype from the Sato dataset

| Surface Protein | ANOVA p value | A vs N | A vs P | A vs I |
|---|---|---|---|---|
| SLC36A4 | 3.6209E−17 | 0.15502 | 22.308% | 22.861% |
| KCNMB2 | 2.12475E−11 | 0.147835 | 54.918% | 52.805% |
| DLL3 | 4.95184E−11 | 0.174969 | 54.054% | 43.414% |
| SCN3A | 8.2927E−10 | 0.3364 | 63.826% | 73.498% |
| CNTNAP2 | 9.35797E−10 | 0.149454 | 60.412% | 45.824% |
| EPHA7 | 4.09629E−06 | 0.103841 | 60.054% | 27.894% |
| NCAM1 | 1.0331E−05 | 0.193353 | 38.981% | 43.300% |
| GPR6 | 3.04057E−05 | 0.206156 | 38.466% | 47.680% |
| TMEM178B | 6.39697E−05 | 0.102242 | 45.414% | 38.991% |
| USH2A | 0.000202096 | 0.339776 | 45.994% | 47.909% |
| SLC10A4 | 0.000248882 | 0.274111 | 26.806% | 30.060% |
| FZD9 | 0.000341704 | 0.24411 | 42.544% | 32.753% |
| CSMD2 | 0.000359364 | 0.222779 | 26.009% | 40.148% |
| GABRQ | 0.000475856 | 0.384127 | 50.287% | 51.070% |
| NKAIN2 | 0.000487651 | 0.370731 | 46.408% | 75.324% |
| BAI3 | 0.000507217 | 0.264845 | 63.729% | 50.032% |
| SLC13A3 | 0.000514257 | 0.497167 | 32.557% | 37.717% |
| SLITRK6 | 0.000760758 | 0.410857 | 19.282% | 29.530% |
| GABRB3 | 0.000836048 | 0.233776 | 55.244% | 32.639% |
| GRIN3A | 0.000988034 | 0.102544 | 39.707% | 33.547% |
| TSPAN11 | 0.001296991 | 0.331247 | 40.427% | 39.169% |
| SLC6A20 | 0.001507455 | 0.287331 | 58.519% | 52.405% |
| IMPG2 | 0.002094285 | 0.656235 | 40.016% | 48.251% |
| SLC38A11 | 0.002154783 | 0.384341 | 51.780% | 39.107% |
| OR51B4 | 0.002313114 | 0.336505 | 44.933% | 38.853% |
| KIAA0319 | 0.002483783 | 0.11467 | 36.340% | 38.323% |
| SCNN1A | 0.002953916 | 0.232782 | 22.755% | 16.122% |
| HTR3A | 0.003313948 | 0.179301 | 32.946% | 25.480% |
| MEGF11 | 0.005261409 | 0.369124 | 31.185% | 57.438% |
| SLCO5A1 | 0.005309677 | 0.154419 | 19.329% | 22.456% |
| OR51E2 | 0.005385784 | 0.498221 | 43.895% | 50.589% |
| SCN2A | 0.005615783 | 0.579349 | 56.037% | 69.463% |
| RXFP2 | 0.006741993 | 0.326676 | 48.979% | 47.403% |
| PCDHB14 | 0.007987304 | 0.147473 | 20.708% | 17.263% |
| MRGPRX2 | 0.008177888 | 0.570892 | 21.686% | 56.823% |
| ZACN | 0.008212913 | 0.523969 | 35.080% | 36.087% |
| TMPRSS6 | 0.009348823 | 0.297175 | 33.980% | 24.019% |
| SDK2 | 0.009660435 | 0.235089 | 26.388% | 16.965% |
| EPHA8 | 0.011240564 | 0.370718 | 42.163% | 38.458% |
| KIRREL3 | 0.011666252 | 0.469852 | 72.765% | 67.381% |
| OPRD1 | 0.013540907 | 0.447248 | 42.558% | 11.970% |
| OR51I1 | 0.014025586 | 0.376518 | 38.102% | 35.813% |
| EPHB1 | 0.014465982 | 0.172161 | 33.125% | 46.403% |
| TAS2R19 | 0.014590558 | 0.153296 | 39.139% | 29.226% |
| TMEM132D | 0.016265486 | 0.21735 | 28.160% | 32.928% |
| GML | 0.016952953 | 0.24852 | 16.805% | 31.682% |
| CCR10 | 0.017943645 | 0.30845 | 14.425% | 42.166% |
| CLDN9 | 0.019362449 | 0.165337 | 27.569% | 20.463% |
| CHRM4 | 0.020057719 | 0.320558 | 41.741% | 22.390% |
| GHRHR | 0.025395908 | 0.473286 | 36.531% | 36.426% |
| OR2C1 | 0.025539295 | 0.226252 | 15.137% | 23.532% |
| LRRC52 | 0.025718703 | 0.347979 | 23.744% | 29.467% |
| CLDN19 | 0.027568501 | 0.183315 | 34.096% | 29.990% |
| NPC1L1 | 0.027664883 | 0.650431 | 45.735% | 45.030% |
| LIFR | 0.028042227 | 0.14005 | 34.930% | 20.756% |
| KCNG4 | 0.035325371 | 0.323904 | 38.633% | 30.792% |
| CADM2 | 0.03592761 | 0.170513 | 60.254% | 66.949% |
| OR6A2 | 0.048402304 | 0.241793 | 47.158% | 29.900% |
| PCDHB4 | 0.04915665 | 0.140332 | 12.870% | 30.033% |
| DSCAM | 0.049749306 | 0.442161 | 42.429% | 38.937% |
| GPR3 | 0.053308509 | 0.370976 | 42.227% | 11.756% |
| ZNRF4 | 0.054720302 | 0.254887 | 42.294% | 17.092% |
| PCDHGC3 | 0.055137741 | 0.273287 | 36.717% | 34.037% |
| SLC18A1 | 0.056847155 | 0.117628 | 29.841% | 48.985% |
| PTH2R | 0.057035473 | 0.200735 | 18.643% | 21.415% |
| TMEM255B | 0.058967754 | 0.122116 | 26.501% | 21.509% |
| SSTR1 | 0.061635122 | 0.108684 | 39.195% | 39.539% |
| ANO9 | 0.063449988 | 0.115157 | 22.862% | 22.304% |
| OR51E1 | 0.064203033 | 0.184966 | 33.475% | 22.616% |
| TNFRSF11A | 0.071568554 | 0.158845 | 24.040% | 22.870% |
| SLITRK3 | 0.072970747 | 0.130772 | 42.610% | 55.146% |
| CACNG4 | 0.075295546 | 0.229757 | 26.340% | 31.059% |
| PCDH8 | 0.075536069 | 0.137834 | 28.815% | 38.716% |
| LRRC37A2 | 0.077267382 | 0.383611 | 29.502% | 42.495% |
| MTNR1B | 0.079530838 | 0.476711 | 21.629% | 41.123% |
| SLC7A9 | 0.085711186 | 0.12475 | 10.928% | 25.188% |
| RRH | 0.088449346 | 0.172084 | 22.950% | 31.787% |
| MLNR | 0.093877884 | 0.246308 | 11.933% | 67.321% |
| SSTR4 | 0.096475655 | 0.44798 | 33.174% | 60.206% |
| SHISA8 | 0.104907471 | 0.26363 | 27.706% | 25.560% |
| GNRHR2 | 0.105712198 | 0.302476 | 25.311% | 30.786% |
| CHRNA6 | 0.107053817 | 0.135801 | 23.847% | 20.954% |
| HTR1F | 0.110049933 | 0.243619 | 25.437% | 22.688% |
| PCDHB16 | 0.114264115 | 0.154052 | 28.878% | 23.171% |
| OR5K1 | 0.11843297 | 0.534206 | 36.538% | 10.684% |
| OR2J2 | 0.124912351 | 0.303661 | 47.232% | 47.637% |
| CEACAM3 | 0.125467587 | 0.14594 | 33.235% | 25.602% |
| FASLG | 0.141651826 | 0.432049 | 10.029% | 16.818% |
| FSHR | 0.154384245 | 0.324573 | 31.452% | 35.872% |
| OR10J1 | 0.155816912 | 0.36456 | 20.329% | 22.753% |
| SLC2A9 | 0.157908097 | 0.233686 | 30.791% | 32.036% |
| MAMDC4 | 0.159285638 | 0.271523 | 32.617% | 40.752% |
| GRIA1 | 0.164707109 | 0.456342 | 50.002% | 33.319% |
| OR5P2 | 0.168607105 | 0.386843 | 24.427% | 35.804% |
| CCKBR | 0.16991124 | 0.22797 | 39.953% | 30.549% |
| SLC17A8 | 0.176197732 | 0.393595 | 10.957% | 41.542% |
| OR12D3 | 0.193758767 | 0.477544 | 38.168% | 22.809% |
| OR1C1 | 0.197981615 | 0.226152 | 40.991% | 37.681% |
| EPHA10 | 0.217205955 | 0.137644 | 14.704% | 36.381% |
| TMEM26 | 0.231754999 | 0.33384 | 13.250% | 25.896% |
| RPRML | 0.232422987 | 0.310727 | 31.976% | 48.797% |
| ERVK13-1 | 0.246852182 | 0.285272 | 34.595% | 37.679% |
| FCAMR | 0.257489198 | 0.473894 | 10.823% | 50.472% |
| TAS2R9 | 0.258115839 | 0.128005 | 32.008% | 28.559% |
| OR51B5 | 0.272525891 | 0.358403 | 13.408% | 23.503% |
| SLC22A12 | 0.282201725 | 0.394495 | 21.378% | 39.099% |
| PLXNA4 | 0.287514564 | 0.272268 | 30.092% | 27.292% |
| CATSPERG | 0.287622709 | 0.123575 | 29.005% | 41.241% |
| CNTNAP3 | 0.291475048 | 0.13097 | 16.321% | 40.176% |
| SLC28A1 | 0.306710197 | 0.536389 | 19.634% | 46.421% |
| GPR111 | 0.315373938 | 0.120573 | 34.760% | 44.541% |
| OR7E24 | 0.330202419 | 0.147504 | 10.179% | 17.082% |
| MRGPRX3 | 0.337045286 | 0.326367 | 14.929% | 44.917% |
| LYPD4 | 0.342132517 | 0.231182 | 29.842% | 34.120% |
| UPK2 | 0.376496111 | 0.259384 | 31.322% | 13.881% |
| PMEL | 0.378874334 | 0.162151 | 17.241% | 34.662% |
| OR8B2 | 0.383388425 | 0.407016 | 38.383% | 36.739% |
| NTSR1 | 0.400107773 | 0.243023 | 28.527% | 15.852% |
| P2RX6 | 0.403276605 | 0.216562 | 18.287% | 39.600% |
| GP2 | 0.421840526 | 0.331034 | 19.146% | 33.302% |
| KIR2DL2 | 0.484815762 | 0.246266 | 16.226% | 37.669% |
| SLC22A16 | 0.496766455 | 0.354668 | 31.261% | 31.710% |
| DCT | 0.498290319 | 0.106322 | 37.415% | 35.608% |
| GPR150 | 0.502744828 | 0.258241 | 11.544% | 20.003% |
| GPR52 | 0.506798691 | 0.215444 | 17.122% | 26.294% |
| ADRA2B | 0.508365779 | 0.115223 | 13.314% | 20.612% |
| GRIN2C | 0.508812967 | 0.515733 | 26.706% | 46.268% |
| SYNPR | 0.531601558 | 0.153175 | 45.827% | 20.640% |
| NPBWR2 | 0.531726436 | 0.174621 | 26.632% | 13.088% |
| LHFPL5 | 0.551998942 | 0.347731 | 26.548% | 31.810% |
| SLC17A1 | 0.552893145 | 0.245995 | 20.117% | 38.775% |
| OR10D3 | 0.579559865 | 0.306504 | 22.682% | 27.642% |
| KIR3DL3 | 0.633760997 | 0.363982 | 12.189% | 21.245% |
| SLC12A5 | 0.634871755 | 0.131703 | 34.205% | 27.162% |
| AQP8 | 0.66424754 | 0.318738 | 21.762% | 43.638% |
| GALR1 | 0.707401413 | 0.139824 | 22.635% | 30.150% |
| CACNG2 | 0.72884106 | 0.244945 | 30.076% | 24.906% |
| OR1J2 | 0.745542617 | 0.1659 | 21.786% | 20.816% |
| LRRC19 | 0.757790628 | 0.114687 | 20.488% | 35.039% |
| GALR2 | 0.786281062 | 0.20244 | 25.392% | 10.962% |
| KIR2DS3 | 0.839037134 | 0.212415 | 15.550% | 24.822% |
| TMEM194A | 1.99109E−17 | 0.008766 | 3.285% | 12.698% |
| FLVCR1 | 9.54416E−15 | 0.010361 | 6.615% | 6.187% |
| GPR180 | 1.67834E−12 | 0.095857 | 5.913% | 1.526% |
| GPR137C | 5.94514E−12 | 0.010676 | 22.977% | 33.222% |
| PGAP1 | 9.93308E−09 | 0.027223 | 19.227% | 22.341% |
| OPRK1 | 2.46524E−08 | 0.029869 | 42.001% | 35.326% |
| FRAS1 | 1.61818E−07 | 0.016696 | 22.242% | 30.497% |
| RET | 4.51872E−07 | 0.045272 | 65.408% | 44.954% |
| CDH2 | 4.37339E−06 | 0.055668 | 33.107% | 47.125% |
| DPP10 | 9.64044E−06 | 0.028249 | 57.339% | 47.124% |
| IL17RB | 4.05664E−05 | 0.029719 | 0.358% | 22.944% |
| NCR3LG1 | 4.55152E−05 | 0.201455 | 20.909% | 66.651% |
| GPC2 | 5.95108E−05 | 0.044895 | 0.566% | 9.423% |
| CXADR | 6.75458E−05 | 0.01686 | 7.438% | 8.463% |
| DNER | 7.50498E−05 | 0.00785 | 67.506% | 50.615% |
| CEACAM5 | 0.000117654 | 0.435062 | 59.590% | 3.722% |
| PTCHD2 | 0.000155344 | 0.040558 | 21.106% | 31.835% |
| JAG2 | 0.000169434 | 0.060764 | 25.767% | 25.102% |
| GPR19 | 0.000186837 | 0.137129 | 8.450% | 16.650% |
| OR10A5 | 0.000263957 | 0.373593 | 3.957% | 16.729% |
| ABCA5 | 0.000303787 | 0.021574 | 13.007% | 16.271% |
| UNC5A | 0.000444148 | 0.077637 | 74.208% | 73.358% |
| L1CAM | 0.000470882 | 0.014761 | 44.031% | 44.644% |
| SLC35A5 | 0.000489255 | 0.041868 | 1.928% | 7.461% |
| ACVR2A | 0.000518356 | 0.054361 | 17.915% | 18.037% |
| JAM3 | 0.000628884 | 0.140538 | 25.566% | 21.465% |
| NRCAM | 0.000647028 | 0.003451 | 18.231% | 20.282% |
| KITLG | 0.000827928 | 0.065612 | 20.623% | 12.737% |
| ZP4 | 0.000873488 | 0.083478 | 23.152% | 29.585% |
| C1orf159 | 0.000929423 | 0.067247 | 11.866% | 16.926% |
| CLDN3 | 0.001973181 | 0.013 | 1.869% | 21.120% |
| FAT3 | 0.002040177 | 0.094829 | 43.475% | 52.371% |
| VSIG10 | 0.002186823 | 0.11297 | 7.153% | 25.989% |
| PTPRN | 0.002502007 | 0.021962 | 34.813% | 40.511% |
| ACPT | 0.003274664 | 0.033291 | 13.466% | 16.473% |
| UGT8 | 0.003431098 | 0.001063 | 6.485% | 23.446% |
| SCN8A | 0.003916188 | 0.071417 | 39.470% | 35.926% |
| LRP6 | 0.005394716 | 0.047986 | 1.142% | 6.706% |
| PTPRN2 | 0.005602401 | 0.005449 | 52.180% | 31.704% |
| EPOR | 0.005607685 | 0.08905 | 31.528% | 50.431% |
| SCN10A | 0.005978571 | 0.163899 | 8.624% | 9.264% |
| LYNX1 | 0.006849684 | 0.118617 | 32.773% | 5.580% |
| TMEM63C | 0.006996996 | 0.017557 | 47.104% | 25.882% |
| CD24 | 0.00729205 | 0.075986 | 1.734% | 0.299% |
| LRP11 | 0.008244906 | 0.073493 | 8.653% | 8.958% |
| ILDR1 | 0.008245603 | 0.21415 | 6.931% | 9.684% |
| LPAR2 | 0.008734435 | 0.02919 | 12.803% | 10.538% |
| LRRN3 | 0.008850131 | 0.029899 | 32.449% | 27.768% |
| IGSF3 | 0.009066078 | 0.001982 | 11.764% | 1.074% |
| FZD2 | 0.010682578 | 0.229173 | 27.342% | 2.980% |
| LDLRAD3 | 0.011317141 | 0.141225 | 3.271% | 16.778% |
| SLC38A1 | 0.011998963 | 0.004467 | 6.662% | 10.621% |
| ABCC5 | 0.012124386 | 0.009045 | 12.557% | 5.227% |
| PCDHB2 | 0.012378101 | 0.042525 | 2.905% | 5.919% |
| TENM3 | 0.013055662 | 0.063878 | 34.445% | 47.559% |
| AVPR1B | 0.01539388 | 0.0787 | 25.662% | 21.399% |
| SCN9A | 0.015639788 | 0.328849 | 46.227% | 0.169% |
| HRH3 | 0.016266498 | 0.084254 | 63.985% | 48.046% |
| TGFBR1 | 0.017602758 | 0.026832 | 2.109% | 0.454% |
| TECTB | 0.017653043 | 0.262742 | 7.922% | 22.696% |
| PCDH17 | 0.017795987 | 0.221154 | 15.208% | 9.992% |
| SLC38A2 | 0.020800535 | 0.002456 | 1.595% | 0.816% |
| NKAIN1 | 0.024737509 | 0.078601 | 45.852% | 27.510% |
| OR2B6 | 0.025670547 | 0.055965 | 26.933% | 11.751% |
| ZDHHC11 | 0.026038949 | 0.079895 | 12.171% | 26.230% |
| TMEM104 | 0.029689108 | 0.092341 | 6.091% | 7.052% |
| ANKH | 0.029725716 | 0.185744 | 25.519% | 27.744% |
| MC1R | 0.02985773 | 0.094628 | 21.850% | 10.537% |
| SLC46A1 | 0.030028088 | 0.492967 | 35.040% | 38.739% |
| TPBGL | 0.031590253 | 0.079668 | 26.967% | 19.166% |
| DISP2 | 0.036826576 | 0.088057 | 56.065% | 43.614% |
| BDKRB1 | 0.037433263 | 0.057829 | 33.314% | 6.038% |
| TMEM158 | 0.037449315 | 0.01588 | 38.869% | 22.698% |
| EDAR | 0.039067345 | 0.058796 | 17.509% | 17.615% |
| LRP8 | 0.039867424 | 0.000954 | 2.261% | 2.149% |
| PCDHA9 | 0.042449507 | 0.05559 | 20.069% | 28.474% |
| SLC4A8 | 0.043513887 | 0.083987 | 16.097% | 8.934% |
| OR5112 | 0.046049029 | 0.33085 | 8.386% | 19.753% |
| CRB1 | 0.048117789 | 0.011209 | 54.617% | 75.610% |

TABLE 4
Surface proteins associated with the SCLC-N subtype from the George et al. dataset

| Surface Protein | ANOVA p value | N vs P | N vs I | N vs A |
|---|---|---|---|---|
| CELSR3 | 6.1925E−12 | 0.573759 | 27.31% | 10.29% |
| ATP2B3 | 5.06576E−11 | 0.916577 | 70.82% | 73.79% |
| LRFN5 | 1.72877E−10 | 0.905313 | 51.65% | 57.69% |
| PRIMA1 | 8.69651E−10 | 0.698271 | 53.42% | 70.49% |
| UNC5A | 2.83215E−09 | 0.732281 | 41.62% | 31.43% |
| SEMA6A | 4.14523E−09 | 0.575409 | 44.51% | 61.17% |
| CHRNA3 | 5.28743E−09 | 0.836309 | 42.61% | 58.35% |
| SSTR2 | 6.71534E−09 | 0.683883 | 44.88% | 65.50% |
| EFNB1 | 1.51695E−08 | 0.138497 | 10.89% | 36.24% |
| ADAM22 | 2.59859E−08 | 0.595686 | 46.14% | 26.59% |
| CHRM4 | 2.64172E−08 | 0.774461 | 35.51% | 16.15% |
| GRM2 | 4.28391E−08 | 0.723294 | 54.88% | 40.05% |
| SORCS2 | 1.02917E−07 | 0.66894 | 54.02% | 59.16% |
| SLC7A14 | 1.32126E−07 | 0.811933 | 48.58% | 22.88% |
| PTCHD2 | 1.32495E−07 | 0.662233 | 29.70% | 36.92% |
| SLC17A6 | 1.60444E−07 | 0.831928 | 55.20% | 89.15% |
| INSR | 1.65814E−07 | 0.387762 | 34.54% | 20.85% |
| SLC22A17 | 3.83495E−07 | 0.492411 | 28.13% | 27.84% |
| SCN8A | 4.49606E−07 | 0.500287 | 40.53% | 24.47% |
| AJAP1 | 4.69171E−07 | 0.88978 | 57.28% | 63.75% |
| PCDH8 | 5.12598E−07 | 0.823214 | 51.39% | 53.43% |
| FNDC5 | 6.51458E−07 | 0.644115 | 17.52% | 62.74% |
| MDGA1 | 2.12746E−06 | 0.620897 | 19.08% | 53.44% |
| NRXN1 | 2.45923E−06 | 0.713869 | 38.37% | 12.54% |
| LHFPL4 | 2.79358E−06 | 0.815741 | 54.64% | 18.12% |
| NRXN3 | 3.07513E−06 | 0.737151 | 54.88% | 35.34% |
| ALK | 3.36763E−06 | 0.701165 | 43.01% | 93.07% |
| BAI3 | 3.66247E−06 | 0.624551 | 61.51% | 10.54% |
| GRIK3 | 3.82855E−06 | 0.777522 | 30.37% | 19.97% |
| GRM8 | 4.24999E−06 | 0.781647 | 41.08% | 27.83% |
| CNTNAP5 | 4.38812E−06 | 0.962856 | 53.24% | 27.84% |
| CLDN11 | 5.95214E−06 | 0.602525 | 47.81% | 38.43% |
| MEGF11 | 6.10315E−06 | 0.196511 | 66.73% | 54.63% |
| CHRNB4 | 8.05596E−06 | 0.720094 | 44.93% | 72.54% |
| SLC4A8 | 8.58488E−06 | 0.556254 | 34.02% | 25.39% |
| SDK1 | 9.45195E−06 | 0.481539 | 16.48% | 19.30% |
| FLRT1 | 9.98115E−06 | 0.667204 | 54.99% | 53.55% |
| DISP2 | 1.05317E−05 | 0.608779 | 31.72% | 14.02% |
| PGAP1 | 1.08883E−05 | 0.340334 | 24.89% | 15.62% |
| GABRB3 | 1.09213E−05 | 0.795586 | 39.54% | 19.03% |
| GPR137C | 1.24293E−05 | 0.329891 | 37.01% | 23.75% |
| NFASC | 1.56363E−05 | 0.569673 | 39.02% | 11.93% |
| ITGA2B | 2.35216E−05 | 0.540112 | 61.10% | 53.56% |
| TMEM132E | 3.28842E−05 | 0.866342 | 64.00% | 71.53% |
| PTPRS | 3.31876E−05 | 0.480181 | 29.78% | 24.19% |
| SLC6A3 | 3.84958E−05 | 0.876632 | 41.33% | 38.10% |
| ADCY2 | 4.07443E−05 | 0.690115 | 39.83% | 20.23% |
| ATP2B2 | 4.20568E−05 | 0.600252 | 57.74% | 85.06% |
| CDH15 | 5.61025E−05 | 0.837607 | 56.88% | 78.12% |
| GPR144 | 7.18573E−05 | 0.977342 | 74.92% | 50.88% |
| ATP1A3 | 7.5315E−05 | 0.646923 | 25.04% | 15.72% |
| CDH23 | 8.0446E−05 | 0.586608 | 68.71% | 65.78% |
| INSRR | 0.000153184 | 0.755215 | 71.90% | 54.16% |
| CACNA1G | 0.000202034 | 0.972712 | 68.33% | 58.74% |
| BVES | 0.0002091 | 0.162455 | 50.31% | 56.05% |
| CADM3 | 0.000241401 | 0.432176 | 23.09% | 70.31% |
| NTNG2 | 0.000249909 | 0.602432 | 28.97% | 60.52% |
| PKDREJ | 0.000266154 | 0.596165 | 51.05% | 48.95% |
| EDA | 0.000271187 | 0.579058 | 20.61% | 82.46% |
| SLC7A3 | 0.000306704 | 0.654907 | 81.88% | 74.69% |
| THSD7B | 0.000312704 | 0.709072 | 37.16% | 71.42% |
| CLSTN2 | 0.00035872 | 0.607084 | 29.01% | 11.28% |
| GPR173 | 0.000394965 | 0.331256 | 22.46% | 15.11% |
| LINGO4 | 0.000418643 | 0.852769 | 46.75% | 60.74% |
| CACNG5 | 0.000424004 | 0.821964 | 57.53% | 51.77% |
| OXER1 | 0.000429125 | 0.177194 | 25.55% | 52.17% |
| EFNB3 | 0.000439341 | 0.650232 | 28.31% | 24.45% |
| CDH24 | 0.000469432 | 0.260252 | 19.13% | 19.41% |
| GPC1 | 0.000510653 | 0.286168 | 17.02% | 14.91% |
| SEMA6D | 0.000646567 | 0.618302 | 29.84% | 28.25% |
| GRIN2A | 0.000650613 | 0.61281 | 83.43% | 86.03% |
| DRD2 | 0.000696344 | 0.427341 | 38.67% | 14.41% |
| PROKR1 | 0.000699718 | 0.77653 | 58.40% | 64.44% |
| PCDH19 | 0.000719942 | 0.254658 | 32.07% | 72.44% |
| DSCAML1 | 0.000745893 | 0.452443 | 48.67% | 30.73% |
| TMEM132C | 0.00077889 | 0.26231 | 73.32% | 83.45% |
| NLGN4X | 0.000819876 | 0.458666 | 36.19% | 53.09% |
| FRAS1 | 0.00084398 | 0.579842 | 42.06% | 18.13% |
| PTPRU | 0.000920643 | 0.567345 | 38.90% | 10.40% |
| TSPAN5 | 0.000938159 | 0.259173 | 23.48% | 34.34% |
| CDH4 | 0.000984619 | 0.587127 | 66.87% | 55.81% |
| HCRTR1 | 0.000989857 | 0.671068 | 45.68% | 75.85% |
| L1CAM | 0.001036463 | 0.571241 | 29.52% | 13.94% |
| OPRK1 | 0.001049209 | 0.776458 | 33.10% | 51.70% |
| CDH20 | 0.001085102 | 0.830887 | 63.49% | 69.78% |
| ADCYAP1R1 | 0.001095909 | 0.696811 | 39.19% | 83.25% |
| LRRC37A | 0.00112235 | 0.33307 | 23.59% | 23.82% |
| KCNJ12 | 0.001148316 | 0.668503 | 56.52% | 63.77% |
| GABRB2 | 0.001229154 | 0.59384 | 62.92% | 83.16% |
| LPPR4 | 0.001234492 | 0.476717 | 32.76% | 64.18% |
| CSPG5 | 0.001245776 | 0.595644 | 44.15% | 30.60% |
| SLITRK2 | 0.001354932 | 0.807261 | 85.42% | 95.56% |
| PCDHAC1 | 0.001439138 | 0.672744 | 41.03% | 30.24% |
| GABRD | 0.001458697 | 0.643466 | 41.17% | 53.89% |
| PLXNA1 | 0.001533472 | 0.293294 | 16.17% | 13.83% |
| DCHS1 | 0.001573525 | 0.181226 | 21.19% | 24.88% |
| GPR179 | 0.001585972 | 0.729298 | 42.97% | 52.29% |
| LRRC37A2 | 0.001591585 | 0.236561 | 22.36% | 18.21% |
| CLDN24 | 0.001598158 | 0.890572 | 81.36% | 73.96% |
| GRIK5 | 0.001613968 | 0.509031 | 20.18% | 15.81% |
| BOC | 0.001619979 | 0.28387 | 13.57% | 37.33% |
| TSHR | 0.001767578 | 0.588054 | 18.57% | 82.58% |
| SGCD | 0.001972312 | 0.505073 | 21.76% | 62.76% |
| GPR98 | 0.002112942 | 0.769006 | 43.65% | 21.31% |
| IGSF1 | 0.002133039 | 0.855788 | 58.27% | 76.85% |
| ADRA2A | 0.0025547 | 0.492519 | 42.01% | 53.18% |
| PCDHGC4 | 0.002558076 | 0.760026 | 36.69% | 24.75% |
| SLC10A4 | 0.002734957 | 0.589003 | 23.01% | 12.44% |
| CNTFR | 0.003007624 | 0.610219 | 56.94% | 49.56% |
| GPR12 | 0.003164611 | 0.511798 | 74.50% | 44.89% |
| SV2C | 0.003228604 | 0.685839 | 56.96% | 85.39% |
| PKD1 | 0.00377731 | 0.285687 | 24.31% | 11.31% |
| GFRA2 | 0.003789973 | 0.632558 | 48.50% | 62.45% |
| LRTM2 | 0.003804776 | 0.878045 | 47.05% | 33.95% |
| GRM7 | 0.003870244 | 0.863609 | 61.35% | 68.19% |
| PLXNA3 | 0.003954812 | 0.178186 | 21.09% | 13.59% |
| DRD5 | 0.003987268 | 0.921329 | 72.11% | 52.18% |
| FAT2 | 0.004082827 | 0.514623 | 39.27% | 78.58% |
| PTCH1 | 0.004296687 | 0.466503 | 31.54% | 17.59% |
| GPR162 | 0.004390574 | 0.394175 | 30.07% | 32.00% |
| ADAM11 | 0.004402101 | 0.560331 | 32.64% | 44.46% |
| DCC | 0.004625318 | 0.717 | 49.85% | 49.46% |
| ANO1 | 0.004855082 | 0.242015 | 12.52% | 24.04% |
| CSMD2 | 0.004989606 | 0.720491 | 24.87% | 16.54% |
| LGR6 | 0.005071809 | 0.467987 | 28.87% | 62.90% |
| GABRQ | 0.00536001 | 0.662686 | 56.27% | 46.48% |
| GRID1 | 0.005829422 | 0.671908 | 45.20% | 15.36% |
| SLC9A3 | 0.005943468 | 0.851469 | 39.41% | 40.49% |
| RHO | 0.006311015 | 0.605136 | 61.85% | 54.93% |
| KREMEN1 | 0.006666046 | 0.335346 | 20.81% | 28.75% |
| LYPD1 | 0.007059685 | 0.563758 | 23.82% | 15.52% |
| ISLR2 | 0.007463654 | 0.785404 | 26.51% | 31.74% |
| GRIN2D | 0.007544549 | 0.504649 | 16.73% | 46.36% |
| GFRA1 | 0.007650674 | 0.492506 | 44.83% | 77.03% |
| LYNX1 | 0.007677437 | 0.633419 | 42.72% | 30.35% |
| FZD10 | 0.008095895 | 0.525546 | 42.02% | 54.09% |
| SLC24A3 | 0.008104277 | 0.410185 | 20.07% | 29.26% |
| TMEM235 | 0.008171478 | 0.949558 | 40.09% | 90.08% |
| ANO5 | 0.008677809 | 0.463405 | 58.16% | 20.06% |
| OR2B2 | 0.008711431 | 0.78781 | 91.23% | 80.23% |
| TSPAN7 | 0.008954136 | 0.264206 | 16.67% | 15.43% |
| ASTN1 | 0.009088081 | 0.532796 | 40.82% | 52.19% |
| TECTA | 0.010110951 | 0.473739 | 37.27% | 25.38% |
| CDHR1 | 0.010144136 | 0.733111 | 34.51% | 40.51% |
| TMEM132B | 0.010155423 | 0.495876 | 51.12% | 68.27% |
| GLRA4 | 0.010404238 | 0.831564 | 64.29% | 67.53% |
| ADRA2C | 0.010641811 | 0.549583 | 52.72% | 28.47% |
| UMODL1 | 0.01074962 | 0.705974 | 40.71% | 68.13% |
| CEACAM7 | 0.011097401 | 0.947309 | 29.25% | 29.07% |
| LRRTM2 | 0.011193361 | 0.686155 | 47.72% | 50.61% |
| MRGPRE | 0.011685277 | 0.693856 | 57.07% | 10.50% |
| GRM4 | 0.01182794 | 0.659113 | 44.03% | 15.64% |
| HTR1D | 0.011978359 | 0.599101 | 61.72% | 49.53% |
| AMHR2 | 0.012854106 | 0.680105 | 79.32% | 77.95% |
| GABRG1 | 0.012961128 | 0.994729 | 97.24% | 45.07% |
| IGSF9B | 0.014781834 | 0.494546 | 42.48% | 34.31% |
| IGDCC3 | 0.015128595 | 0.315368 | 38.83% | 67.00% |
| SLC1A2 | 0.015955141 | 0.576281 | 59.31% | 55.98% |
| RTN4R | 0.016010016 | 0.121643 | 18.00% | 28.74% |
| ASTN2 | 0.01669561 | 0.356192 | 41.62% | 19.69% |
| ATRNL1 | 0.017493974 | 0.476618 | 57.72% | 45.51% |
| SEMA6C | 0.018030325 | 0.136158 | 21.77% | 23.65% |
| SLC12A5 | 0.018092542 | 0.850817 | 57.68% | 35.91% |
| GPR88 | 0.018097555 | 0.602736 | 37.97% | 76.42% |
| EPHB2 | 0.018229314 | 0.238481 | 15.33% | 28.82% |
| NRXN2 | 0.018463989 | 0.556142 | 32.16% | 57.47% |
| SLC22A15 | 0.01869566 | 0.124534 | 26.80% | 38.70% |
| NPR2 | 0.019561805 | 0.252443 | 21.21% | 25.70% |
| TNFRSF19 | 0.020259707 | 0.375924 | 32.02% | 31.68% |
| GLP1R | 0.020563182 | 0.147739 | 70.03% | 43.53% |
| GSG1 | 0.021129435 | 0.564146 | 56.49% | 65.89% |
| SLC6A20 | 0.021140595 | 0.714618 | 47.68% | 45.44% |
| BAI1 | 0.021520112 | 0.637911 | 21.14% | 26.33% |
| HTR5A | 0.022219521 | 0.920426 | 49.48% | 76.13% |
| GPR176 | 0.022340355 | 0.266634 | 11.42% | 29.24% |
| OPRL1 | 0.023260551 | 0.445688 | 22.02% | 32.89% |
| CLDN20 | 0.024180702 | 0.493392 | 40.92% | 58.17% |
| ACVR2B | 0.025118042 | 0.154813 | 22.96% | 16.13% |
| PCDHA11 | 0.025467667 | 0.698385 | 49.37% | 37.21% |
| GPR142 | 0.025714425 | 0.669571 | 78.98% | 37.59% |
| NKAIN3 | 0.026098592 | 0.614376 | 66.92% | 86.32% |
| SCNN1G | 0.026356187 | 0.543443 | 24.24% | 11.22% |
| GRIN3A | 0.027575955 | 0.614257 | 36.93% | 26.26% |
| ATP4B | 0.027634792 | 0.821483 | 31.44% | 14.83% |
| PTPRK | 0.028783658 | 0.153733 | 27.07% | 16.89% |
| GPRC5B | 0.029487306 | 0.106481 | 19.99% | 10.62% |
| SCN1B | 0.030230421 | 0.362256 | 16.62% | 35.61% |
| TMEM132D | 0.031012987 | 0.430694 | 50.01% | 35.85% |
| PTCHD1 | 0.033026771 | 0.722026 | 49.78% | 65.37% |
| GJD2 | 0.033878057 | 0.921458 | 61.02% | 74.77% |
| PCDHA12 | 0.03449809 | 0.599527 | 20.26% | 33.03% |
| OR2B6 | 0.03470576 | 0.446892 | 22.92% | 55.09% |
| SLITRK4 | 0.03490951 | 0.377342 | 63.27% | 58.86% |
| CHRNA5 | 0.036073469 | 0.118688 | 26.36% | 17.26% |
| SLC5A5 | 0.037002817 | 0.669536 | 30.41% | 63.30% |
| RGMA | 0.037156731 | 0.298614 | 19.15% | 41.66% |
| SLC18A2 | 0.037365146 | 0.239139 | 36.68% | 12.65% |
| ADRA1B | 0.037604704 | 0.339989 | 49.70% | 59.94% |
| GABRA5 | 0.037612786 | 0.888907 | 48.42% | 37.32% |
| PCDHB15 | 0.038247212 | 0.457324 | 46.02% | 26.07% |
| GPR113 | 0.03910298 | 0.19598 | 33.87% | 39.20% |
| S1PR5 | 0.040027269 | 0.503571 | 20.86% | 24.99% |
| TNFRSF10D | 0.040563215 | 0.112778 | 16.78% | 40.69% |
| NAALADL1 | 0.041633372 | 0.32281 | 21.01% | 16.92% |
| SCN4B | 0.042046543 | 0.235185 | 33.82% | 53.76% |
| BAI2 | 0.044092778 | 0.186591 | 32.26% | 20.65% |
| PCDHB1 | 0.045495897 | 0.569972 | 47.98% | 39.52% |
| PTPRT | 0.046043953 | 0.220097 | 76.01% | 39.36% |
| PCDHGC5 | 0.046051223 | 0.428101 | 58.50% | 45.43% |
| LRRC37A3 | 0.047156678 | 0.308517 | 22.59% | 15.33% |
| GSG1L | 0.048008064 | 0.905281 | 41.76% | 84.18% |
| SLC24A5 | 0.048509853 | 0.586259 | 47.38% | 39.49% |
| HCRTR2 | 0.048826764 | 0.479918 | 57.57% | 85.53% |
| HTR6 | 0.04978644 | 0.780251 | 42.16% | 45.63% |
| SLC22A5 | 0.050327647 | 0.114529 | 13.12% | 16.93% |
| SLC26A1 | 0.050911603 | 0.248366 | 28.84% | 42.21% |
| GRIA1 | 0.051346761 | 0.876596 | 33.33% | 10.16% |
| GABBR2 | 0.051787928 | 0.694487 | 10.38% | 20.92% |
| LRRC19 | 0.052751568 | 0.651326 | 39.71% | 19.77% |
| SHISA7 | 0.055958554 | 0.809785 | 75.22% | 22.13% |
| GRIK2 | 0.057980021 | 0.595821 | 62.69% | 21.62% |
| IGSF3 | 0.058652192 | 0.246259 | 20.17% | 11.35% |
| CDHR2 | 0.06105779 | 0.690136 | 55.75% | 28.98% |
| CA14 | 0.062443476 | 0.231984 | 56.13% | 41.45% |
| CHRM2 | 0.064707827 | 0.149607 | 71.64% | 72.70% |
| SLC14A2 | 0.065856992 | 0.49556 | 17.85% | 48.46% |
| PRLHR | 0.066523779 | 0.824816 | 77.68% | 47.07% |
| UPK3A | 0.067023796 | 0.665629 | 17.69% | 23.50% |
| SEMA5B | 0.067534396 | 0.508539 | 25.36% | 44.52% |
| GPR63 | 0.07140615 | 0.596877 | 18.54% | 23.90% |
| OR5B21 | 0.071451987 | 0.717285 | 70.81% | 76.12% |
| OR2AG1 | 0.071653636 | 0.503131 | 77.81% | 100.00% |
| GABRA1 | 0.074937804 | 0.342208 | 52.03% | 98.93% |
| PCDHAC2 | 0.076426756 | 0.54551 | 33.89% | 17.38% |
| QRFPR | 0.078409284 | 0.842708 | 44.67% | 78.95% |
| ADRA1A | 0.079075418 | 0.530968 | 42.08% | 72.94% |
| NTRK1 | 0.08095532 | 0.493323 | 32.02% | 55.82% |
| OR2A4 | 0.086233397 | 1 | 61.00% | 87.76% |
| PCDHA9 | 0.086649666 | 0.650196 | 75.97% | 69.87% |
| PCDHA2 | 0.088809981 | 0.688657 | 39.93% | 33.01% |
| ATP1B4 | 0.089019416 | 0.760612 | 47.09% | 76.43% |
| PCDHA3 | 0.089955822 | 0.614149 | 42.37% | 22.09% |
| TMPRSS6 | 0.090784591 | 0.609584 | 25.66% | 11.54% |
| SCNN1D | 0.090955327 | 0.433076 | 30.79% | 40.04% |
| PCDHGA6 | 0.095085055 | 0.237052 | 48.07% | 49.45% |
| GPR26 | 0.098104123 | 0.214137 | 60.48% | 64.67% |
| SLC8A2 | 0.099351624 | 0.530086 | 50.23% | 47.26% |
| CRHR1 | 0.10113572 | 0.943891 | 73.68% | 25.96% |
| ITGA7 | 0.102129657 | 0.158528 | 23.70% | 25.43% |
| SLC4A1 | 0.10323747 | 0.728947 | 25.33% | 39.46% |
| CD164L2 | 0.103479618 | 0.460086 | 52.48% | 41.58% |
| NPBWR2 | 0.103565566 | 0.826678 | 100.00% | 49.33% |
| CACNG2 | 0.106254766 | 0.675472 | 46.75% | 49.31% |
| PCDHA5 | 0.108397926 | 0.755615 | 54.92% | 39.21% |
| GFRAL | 0.108527048 | 0.825913 | 30.89% | 78.44% |
| UPK1B | 0.109771779 | 0.654696 | 59.96% | 54.73% |
| GPR50 | 0.111874922 | 0.776348 | 92.25% | 49.71% |
| LTB4R2 | 0.112299423 | 0.288312 | 13.15% | 13.91% |
| NPFFR1 | 0.113729383 | 0.477906 | 38.50% | 63.16% |
| DCBLD2 | 0.117216578 | 0.109382 | 12.93% | 12.70% |
| OR2J3 | 0.12356801 | 1 | 55.96% | 84.85% |
| EDA2R | 0.12356868 | 0.324638 | 25.09% | 55.05% |
| TMEFF1 | 0.124681705 | 0.201329 | 14.34% | 19.06% |
| GJB6 | 0.128510814 | 0.276044 | 74.48% | 92.02% |
| OR11H4 | 0.131825914 | 0.863813 | 90.29% | 33.63% |
| IZUMO1 | 0.133641173 | 0.488788 | 51.99% | 26.81% |
| OR2AG2 | 0.133735541 | 0.1305 | 78.49% | 47.61% |
| ABCG4 | 0.133955414 | 0.646916 | 70.65% | 77.52% |
| ROR2 | 0.137353073 | 0.402098 | 18.79% | 10.64% |
| OR6B2 | 0.137356036 | 1 | 48.72% | 56.32% |
| VN1R2 | 0.138478415 | 0.62155 | 39.71% | 61.18% |
| FAT3 | 0.138738047 | 0.161872 | 23.59% | 41.22% |
| CDH18 | 0.140554535 | 0.952295 | 73.14% | 29.71% |
| OR4D2 | 0.140910482 | 1 | 100.00% | 73.35% |
| SLC28A2 | 0.145661401 | 0.456536 | 12.63% | 39.76% |
| OR8D4 | 0.146050474 | 1 | 100.00% | 51.01% |
| GPRC5C | 0.147380471 | 0.282132 | 18.71% | 10.76% |
| SLC13A1 | 0.150531099 | 0.518005 | 25.06% | 50.51% |
| SLC10A1 | 0.150710233 | 0.512691 | 35.13% | 46.42% |
| GALR2 | 0.152318302 | 0.843032 | 54.90% | 26.47% |
| CNR1 | 0.156618006 | 0.253929 | 25.88% | 52.12% |
| GPR52 | 0.15668353 | 0.672409 | 47.46% | 63.58% |
| ROBO2 | 0.159006583 | 0.479126 | 31.60% | 32.30% |
| LRIT2 | 0.160418783 | 0.892687 | 66.37% | 54.89% |
| NEO1 | 0.165814007 | 0.14697 | 13.21% | 21.67% |
| HTR1A | 0.168898456 | 0.856383 | 85.34% | 50.51% |
| GPR139 | 0.170003836 | 0.677189 | 84.95% | 17.37% |
| NTM | 0.174732405 | 0.326119 | 23.63% | 15.06% |
| OR2AT4 | 0.181563681 | 1 | 100.00% | 43.87% |
| GPR83 | 0.183530926 | 0.427539 | 41.47% | 35.86% |
| CRB2 | 0.185182959 | 0.841715 | 48.48% | 55.31% |
| PROKR2 | 0.188278637 | 0.876786 | 73.61% | 82.42% |
| OR5B2 | 0.18899064 | 0.305426 | 44.94% | 87.64% |
| KCNK5 | 0.190722136 | 0.208355 | 15.28% | 22.81% |
| GRAMD1B | 0.196913999 | 0.250318 | 22.64% | 21.29% |
| DCT | 0.201840313 | 0.946325 | 85.82% | 67.11% |
| ABCA4 | 0.206010528 | 0.390735 | 62.65% | 74.46% |
| SLCO4C1 | 0.206429019 | 0.356972 | 26.87% | 13.61% |
| TAS2R3 | 0.207462669 | 0.568402 | 49.74% | 61.60% |
| IGSF11 | 0.214244988 | 0.477973 | 57.29% | 34.47% |
| GRM6 | 0.215284295 | 0.28505 | 45.09% | 49.85% |
| DPP6 | 0.216902254 | 0.572704 | 47.49% | 50.87% |
| TMPRSS11B | 0.23063236 | 0.858116 | 80.12% | 78.23% |
| OR1M1 | 0.231785999 | 0.243261 | 100.00% | 66.73% |
| SLC6A5 | 0.242516959 | 0.887958 | 18.10% | 22.11% |
| CLDN19 | 0.243148596 | 0.322167 | 68.34% | 95.83% |
| CA4 | 0.247950175 | 0.323519 | 24.17% | 33.03% |
| OR6M1 | 0.253167148 | 0.795195 | 100.00% | 62.25% |
| MTNR1B | 0.255241956 | 0.736212 | 37.84% | 14.13% |
| AMIGO1 | 0.257127443 | 0.120298 | 17.73% | 16.18% |
| OR13C5 | 0.258888071 | 0.44996 | 59.55% | 82.45% |
| PCDHGA10 | 0.26478046 | 0.514533 | 36.66% | 42.78% |
| LHFPL1 | 0.264893521 | 0.532403 | 56.72% | 22.21% |
| MYADML2 | 0.266453215 | 0.582998 | 36.92% | 17.98% |
| GRIN2B | 0.26961851 | 0.549027 | 52.34% | 90.29% |
| CDHR5 | 0.270982511 | 0.401615 | 51.90% | 34.09% |
| MOG | 0.27177264 | 0.574946 | 35.47% | 72.82% |
| HAVCR1 | 0.272617302 | 0.775078 | 69.71% | 88.80% |
| SLC13A2 | 0.273916786 | 0.159327 | 77.07% | 82.46% |
| SLC36A2 | 0.277083546 | 0.85674 | 38.55% | 58.38% |
| OR1N2 | 0.278195396 | 0.826438 | 72.20% | 36.75% |
| CNTN4 | 0.282985902 | 0.359715 | 25.08% | 11.80% |
| SLC13A4 | 0.286149647 | 0.684887 | 39.63% | 35.48% |
| ABCG2 | 0.287618112 | 0.114709 | 18.94% | 36.29% |
| PCDHGB3 | 0.295723689 | 0.224143 | 49.39% | 31.22% |
| ERBB4 | 0.308853178 | 0.382778 | 63.88% | 22.20% |
| SCN4A | 0.316000285 | 0.821599 | 74.29% | 69.84% |
| GNRHR | 0.316907837 | 0.370845 | 45.29% | 36.49% |
| OR13C9 | 0.320154404 | 1 | 100.00% | 51.92% |
| NTSR1 | 0.323538841 | 0.59583 | 71.88% | 21.42% |
| RHCG | 0.3241437 | 0.589922 | 55.93% | 59.63% |
| TRHDE | 0.32461021 | 0.209551 | 38.53% | 51.77% |
| MC2R | 0.32824809 | 0.547605 | 28.09% | 57.17% |
| TAS2R39 | 0.329819205 | 1 | 100.00% | 21.20% |
| GABBR1 | 0.332121366 | 0.156281 | 16.06% | 18.13% |
| OR2B3 | 0.332675481 | 0.638935 | 100.00% | 68.26% |
| PCDHGA7 | 0.339674098 | 0.296684 | 50.30% | 40.61% |
| OR4D5 | 0.341888191 | 1 | 100.00% | 47.13% |
| OR2D3 | 0.342106411 | 1 | 100.00% | 46.89% |
| ABCB5 | 0.342783624 | 0.471764 | 35.66% | 50.00% |
| CLRN1 | 0.347252491 | 0.952459 | 60.52% | 98.20% |
| OR10A2 | 0.347589078 | 0.771626 | 100.00% | 53.29% |
| PCDHGB2 | 0.361183917 | 0.477259 | 40.15% | 18.37% |
| PCDHB12 | 0.364157737 | 0.281814 | 29.46% | 13.75% |
| GRIA3 | 0.369555756 | 0.698176 | 18.87% | 54.57% |
| MCHR1 | 0.370920252 | 0.382421 | 21.56% | 30.68% |
| LRP4 | 0.378169328 | 0.325013 | 22.85% | 11.48% |
| P2RX3 | 0.386829077 | 0.577645 | 53.99% | 60.97% |
| TSPAN9 | 0.387084489 | 0.135846 | 10.34% | 11.74% |
| KL | 0.39392264 | 0.275568 | 29.86% | 14.63% |
| SLC22A7 | 0.394749732 | 0.27349 | 51.46% | 29.12% |
| UNC5B | 0.399400787 | 0.158693 | 13.43% | 12.45% |
| OR13D1 | 0.404831148 | 0.897819 | 41.36% | 41.69% |
| GPR75 | 0.413547524 | 0.237937 | 20.28% | 21.19% |
| SLCO1C1 | 0.416225466 | 0.133394 | 42.35% | 59.99% |
| MUC22 | 0.440017184 | 0.601891 | 66.90% | 55.21% |
| OR6V1 | 0.442695436 | 0.555368 | 81.88% | 74.35% |
| MRGPRX4 | 0.44826783 | 0.484448 | 92.44% | 57.78% |
| SLC4A4 | 0.448708428 | 0.409485 | 16.13% | 18.64% |
| PCDHGA3 | 0.449935602 | 0.213279 | 40.07% | 32.75% |
| OR1J1 | 0.455981474 | 0.324754 | 58.42% | 23.70% |
| KCNG4 | 0.459670925 | 0.994065 | 98.79% | 98.87% |
| OR13C2 | 0.464840185 | 0.222588 | 100.00% | 45.32% |
| CASR | 0.466024726 | 0.962777 | 46.88% | 10.97% |
| PCDHGA8 | 0.469031739 | 0.387423 | 35.03% | 41.07% |
| VN1R1 | 0.472551441 | 0.117911 | 29.86% | 12.99% |
| OPN1SW | 0.473487393 | 0.45325 | 83.91% | 64.80% |
| OR9A2 | 0.476528085 | 0.359129 | 100.00% | 62.99% |
| CSPG4 | 0.477082831 | 0.120924 | 16.17% | 16.63% |
| DSG3 | 0.481302846 | 0.559901 | 57.02% | 39.80% |
| GP6 | 0.483885944 | 0.472588 | 92.84% | 26.29% |
| PCDHGB5 | 0.48553214 | 0.147241 | 52.04% | 36.16% |
| OR1D5 | 0.486429736 | 1 | 100.00% | 11.18% |
| AQP8 | 0.498490745 | 0.202887 | 46.22% | 19.49% |
| CLCA4 | 0.499403367 | 0.883559 | 99.99% | 89.75% |
| OR1L6 | 0.501994688 | 0.60964 | 100.00% | 49.06% |
| SLC22A8 | 0.502894037 | 0.994337 | 98.97% | 97.81% |
| GLRA1 | 0.50704449 | 0.976348 | 78.86% | 88.16% |
| SUSD4 | 0.508846555 | 0.102802 | 28.87% | 10.67% |
| TMPRSS11D | 0.509886637 | 0.742177 | 82.71% | 88.26% |
| SLC39A12 | 0.517221977 | 0.892125 | 79.08% | 85.94% |
| CLCA2 | 0.522402852 | 0.273613 | 77.24% | 66.05% |
| MFAP3L | 0.52417151 | 0.105193 | 40.02% | 16.15% |
| IMPG2 | 0.532722645 | 0.719932 | 83.25% | 81.73% |
| OR5212 | 0.539612371 | 0.69605 | 100.00% | 32.57% |
| LRIT1 | 0.541321911 | 0.929516 | 97.24% | 97.89% |
| KCNV2 | 0.544091315 | 0.566779 | 55.27% | 64.67% |
| OR1N1 | 0.547414613 | 0.798429 | 96.53% | 88.63% |
| CLDN22 | 0.551313616 | 0.662127 | 98.20% | 81.23% |
| SLC22A9 | 0.56629364 | 0.350639 | 71.92% | 41.29% |
| OR14C36 | 0.570417938 | 0.356931 | 100.00% | 61.64% |
| MRGPRX3 | 0.573764198 | 0.284008 | 15.99% | 27.21% |
| HCAR1 | 0.5826713 | 0.19976 | 25.11% | 26.17% |
| EPHA6 | 0.595670482 | 0.554812 | 80.43% | 26.33% |
| OR2Z1 | 0.612170105 | 1 | 29.55% | 39.09% |
| LCT | 0.61778556 | 0.373601 | 44.96% | 14.48% |
| PCDHA6 | 0.618019459 | 0.175603 | 60.70% | 17.24% |
| PCDHGA4 | 0.618662906 | 0.12113 | 32.29% | 13.15% |
| OR52W1 | 0.619688955 | 0.528846 | 49.71% | 17.53% |
| OR13H1 | 0.628874747 | 0.346694 | 100.00% | 22.41% |
| NMUR1 | 0.63483045 | 0.216403 | 33.89% | 12.71% |
| OR5211 | 0.657234086 | 0.642073 | 37.79% | 15.85% |
| TARM1 | 0.685020329 | 0.784951 | 12.28% | 46.26% |
| TM4SF5 | 0.694652044 | 0.340664 | 53.14% | 14.05% |
| GPR149 | 0.69635078 | 0.749785 | 10.71% | 26.22% |
| GFRA4 | 0.703845483 | 0.778663 | 62.37% | 17.40% |
| OR1B1 | 0.708505177 | 0.378375 | 34.98% | 55.46% |
| CHRND | 0.710960838 | 0.846842 | 76.70% | 81.34% |
| OR2F1 | 0.713967693 | 0.686266 | 69.59% | 17.75% |
| LY6G6C | 0.714657465 | 0.185904 | 16.76% | 28.41% |
| OR1L4 | 0.721171266 | 0.803927 | 31.58% | 15.86% |
| MMEL1 | 0.724478565 | 0.320165 | 40.57% | 19.03% |
| SSTR1 | 0.732605159 | 0.253167 | 22.25% | 11.70% |
| RPRML | 0.738811204 | 0.197745 | 27.90% | 16.19% |
| OR4D10 | 0.760540662 | 0.807299 | 38.40% | 20.73% |
| OR2T4 | 0.762070001 | 0.429879 | 100.00% | 33.52% |
| CHRNA9 | 0.765428876 | 0.323677 | 25.70% | 23.22% |
| SLC36A3 | 0.770560357 | 0.387518 | 37.75% | 41.67% |
| SLC16A7 | 0.774871818 | 0.643722 | 32.04% | 34.81% |
| GPR62 | 0.817990691 | 0.231936 | 26.97% | 15.37% |
| OR4F21 | 0.823925642 | 0.458609 | 76.75% | 19.24% |
| PCDHA10 | 0.825875628 | 0.185558 | 34.94% | 40.43% |
| ZPLD1 | 0.852730583 | 0.606046 | 72.78% | 37.88% |
| OR10A4 | 0.866305563 | 0.332936 | 13.24% | 49.47% |
| OR4D1 | 0.896989595 | 0.646802 | 55.15% | 30.11% |
| OR12D2 | 0.901044732 | 0.287823 | 39.59% | 57.47% |
| ABCB11 | 0.918969979 | 0.450684 | 19.43% | 13.04% |
| RAET1E | 0.921247241 | 0.10264 | 26.48% | 14.95% |
| OR51I2 | 0.964945223 | 0.433546 | 37.37% | 22.50% |
| TMEM145 | 9.22598E−14 | 0.655183 | 34.42% | 8.48% |
| TMEM63C | 7.09855E−12 | 0.777888 | 41.17% | 5.64% |
| APLP1 | 2.25526E−10 | 0.625369 | 26.50% | 1.87% |
| NCAM1 | 8.41352E−07 | 0.356423 | 35.62% | 2.38% |
| FAM171A2 | 1.78318E−06 | 0.382009 | 16.84% | 2.68% |
| LPHN1 | 2.84774E−06 | 0.404436 | 29.66% | 3.91% |
| CASD1 | 3.37792E−06 | 0.296332 | 19.59% | 3.45% |
| NRCAM | 1.33462E−05 | 0.40223 | 30.45% | 7.53% |
| FAM174B | 7.15263E−05 | 0.210908 | 19.06% | 1.62% |
| TRIL | 0.000107965 | 0.013682 | 16.90% | 50.29% |
| DLK1 | 0.000174424 | 0.704542 | 20.38% | 6.06% |
| KIAA0319 | 0.000239135 | 0.739105 | 33.78% | 4.33% |
| CATSPERG | 0.000885557 | 0.684191 | 49.75% | 4.40% |
| GPR3 | 0.000960266 | 0.522747 | 24.50% | 8.95% |
| DLK2 | 0.000978658 | 0.628065 | 28.52% | 0.45% |
| KITLG | 0.001094693 | 0.383245 | 21.14% | 7.43% |
| RGMB | 0.001584362 | 0.297021 | 12.49% | 2.24% |
| SLC17A7 | 0.00189443 | 0.828683 | 6.62% | 60.50% |
| CDH2 | 0.001994447 | 0.284006 | 10.71% | 5.15% |
| KIRREL3 | 0.002490788 | 0.66314 | 25.72% | 9.07% |
| BACE1 | 0.002659259 | 0.250099 | 17.96% | 4.95% |
| KCNJ4 | 0.003522998 | 0.500352 | 0.61% | 80.98% |
| GPR158 | 0.003849924 | 0.518036 | 48.45% | 2.89% |
| LTB4R | 0.003965967 | 0.350999 | 15.28% | 8.30% |
| HRH3 | 0.004660425 | 0.711668 | 29.43% | 2.01% |
| VSIG10 | 0.004738031 | 0.123191 | 17.79% | 9.45% |
| ACVR2A | 0.004792428 | 0.241655 | 13.98% | 9.04% |
| ELFN1 | 0.005088737 | 0.346393 | 3.99% | 49.61% |
| EPHA8 | 0.006531534 | 0.738639 | 63.04% | 6.78% |
| PIEZO2 | 0.007216505 | 0.405748 | 8.04% | 43.58% |
| GDPD5 | 0.00854805 | 0.042827 | 10.86% | 25.91% |
| TSPAN18 | 0.008579339 | 0.045618 | 13.06% | 37.82% |
| PANX2 | 0.009130224 | 0.502185 | 25.99% | 6.56% |
| PTPRD | 0.01030759 | 0.606429 | 50.63% | 0.74% |
| SCN5A | 0.011288902 | 0.013622 | 58.46% | 46.05% |
| CLSTN1 | 0.01129717 | 0.160739 | 7.07% | 8.18% |
| SLC12A6 | 0.01499558 | 0.182437 | 9.47% | 23.37% |
| ADAM23 | 0.015480096 | 0.495738 | 10.39% | 6.53% |
| DSCAM | 0.017423827 | 0.646519 | 40.36% | 3.58% |
| PLXNB1 | 0.017711547 | 0.094547 | 20.23% | 5.73% |
| ANKH | 0.0200025 | 0.208604 | 12.92% | 2.19% |
| LRRN1 | 0.02076384 | 0.219567 | 9.45% | 37.36% |
| MEGF8 | 0.02101423 | 0.19617 | 17.84% | 9.03% |
| SLC38A1 | 0.022598169 | 0.090327 | 16.01% | 1.49% |
| MAMDC4 | 0.027720219 | 0.356046 | 18.26% | 3.76% |
| PSEN1 | 0.031499183 | 0.106095 | 4.01% | 0.61% |
| TSPAN11 | 0.037174733 | 0.405398 | 29.57% | 3.30% |
| SLC5A3 | 0.038078188 | 0.115521 | 16.48% | 4.36% |
| ABCA5 | 0.04035996 | 0.313357 | 7.93% | 1.59% |
| FZD1 | 0.041069243 | 0.19179 | 5.44% | 2.70% |
| KIAA0247 | 0.046560714 | 0.146324 | 8.73% | 6.21% |

TABLE 5
Surface proteins associated with the SCLC-N subtype from the cell line dataset

| Surface Protein | ANOVA p value | N vs P | N vs I | N vs A |
|---|---|---|---|---|
| CMKLR1 | 2.10309E−10 | 0.994633 | 33.60% | 89.35% |
| LRFN1 | 3.89482E−10 | 0.295799 | 39.58% | 81.19% |
| SLC38A5 | 1.78419E−09 | 0.657236 | 63.58% | 73.57% |
| ADAM11 | 1.37199E−08 | 0.290907 | 23.10% | 69.87% |
| SYP | 2.98276E−08 | 0.467823 | 30.20% | 12.74% |
| SGCD | 9.04582E−08 | 0.742385 | 72.77% | 43.41% |
| SSTR2 | 1.4378E−07 | 0.350274 | 53.14% | 23.09% |
| SLC6A11 | 2.82888E−07 | 0.573283 | 42.82% | 81.88% |
| ADCYAP1R1 | 8.02109E−07 | 0.444487 | 43.26% | 66.45% |
| GSG1L | 1.32378E−06 | 0.994036 | 45.92% | 87.83% |
| SLC8A2 | 1.36584E−06 | 0.890928 | 33.16% | 32.79% |
| ITGA4 | 1.48775E−06 | 0.454622 | 33.54% | 45.75% |
| ATP1A3 | 1.6528E−06 | 0.445611 | 36.38% | 27.31% |
| ASIC1 | 1.69302E−06 | 0.187793 | 11.43% | 38.78% |
| CA12 | 2.09371E−06 | 0.778806 | 33.32% | 71.92% |
| SLC17A7 | 2.5024E−06 | 0.498776 | 32.85% | 56.07% |
| TYRO3 | 2.63407E−06 | 0.204461 | 11.24% | 38.18% |
| LPAR3 | 4.24829E−06 | 0.361683 | 33.54% | 73.21% |
| IGDCC3 | 5.00197E−06 | 0.307074 | 44.73% | 55.91% |
| RTN4R | 5.29657E−06 | 0.305786 | 57.86% | 70.96% |
| CHRNA1 | 5.88627E−06 | 0.689204 | 73.15% | 46.57% |
| ALPL | 6.92758E−06 | 0.615717 | 24.45% | 59.55% |
| SLC1A7 | 8.03356E−06 | 0.69823 | 45.19% | 81.69% |
| TSPAN18 | 9.24494E−06 | 0.686971 | 36.83% | 44.37% |
| SCN1B | 1.08198E−05 | 0.820102 | 52.22% | 61.39% |
| SLC6A1 | 2.74093E−05 | 0.78869 | 26.98% | 69.88% |
| NRP2 | 2.76937E−05 | 0.237101 | 32.55% | 35.10% |
| NTRK1 | 3.84528E−05 | 0.684687 | 75.66% | 72.50% |
| TTYH2 | 4.06324E−05 | 0.592126 | 26.28% | 32.44% |
| RTN4RL1 | 6.22085E−05 | 0.729972 | 10.86% | 57.97% |
| SLC24A2 | 6.4919E−05 | 0.875798 | 71.72% | 34.95% |
| SLC7A14 | 7.20474E−05 | 0.443543 | 61.36% | 12.11% |
| SLC18A3 | 7.63537E−05 | 0.147922 | 13.09% | 74.04% |
| DRD2 | 0.000112404 | 0.396977 | 59.58% | 19.18% |
| SHISA6 | 0.000134903 | 0.642699 | 31.33% | 93.18% |
| ADCY7 | 0.000180527 | 0.232925 | 12.65% | 21.78% |
| GRID2 | 0.000196834 | 0.538033 | 46.30% | 46.92% |
| CNTN2 | 0.000209409 | 0.66165 | 37.26% | 35.85% |
| KIRREL2 | 0.000295177 | 0.71616 | 43.48% | 54.88% |
| ATP2B2 | 0.00029904 | 0.329493 | 35.48% | 37.03% |
| GPR162 | 0.000423418 | 0.524142 | 13.19% | 22.53% |
| IGSF11 | 0.000487053 | 0.301721 | 43.64% | 33.49% |
| L1CAM | 0.000727162 | 0.510092 | 41.21% | 24.64% |
| LAYN | 0.000741021 | 0.459238 | 35.88% | 42.64% |
| KREMEN1 | 0.000814766 | 0.121875 | 24.25% | 16.72% |
| THSD7B | 0.000838425 | 0.384433 | 46.74% | 38.96% |
| ROBO3 | 0.000870343 | 0.367212 | 10.48% | 64.52% |
| GPR26 | 0.00088841 | 0.880061 | 100.00% | 70.29% |
| GSG1 | 0.000900923 | 0.361862 | 15.67% | 28.75% |
| GRIN2A | 0.00090401 | 0.75203 | 53.87% | 58.17% |
| EDA | 0.000940211 | 0.390899 | 45.06% | 50.17% |
| NGFR | 0.000960785 | 0.803926 | 50.04% | 78.38% |
| UNC5A | 0.001036332 | 0.487779 | 45.32% | 13.45% |
| GRIN2D | 0.001044269 | 0.704023 | 56.99% | 44.67% |
| LPPR5 | 0.001085015 | 0.354221 | 44.70% | 50.23% |
| ATP1B2 | 0.00108724 | 0.344437 | 29.53% | 49.63% |
| TMEM145 | 0.0011529 | 0.57685 | 15.99% | 13.34% |
| GPR112 | 0.001296029 | 0.61633 | 43.01% | 47.90% |
| IL2RG | 0.001403184 | 0.99565 | 60.99% | 55.93% |
| DPP4 | 0.001485449 | 0.505435 | 16.67% | 38.05% |
| GABRB1 | 0.001515832 | 0.382421 | 71.83% | 60.89% |
| LINGO1 | 0.001671438 | 0.175328 | 15.44% | 34.85% |
| NTNG2 | 0.001704225 | 0.762589 | 53.84% | 50.70% |
| SPNS2 | 0.001736529 | 0.255798 | 12.09% | 29.05% |
| CA4 | 0.001737829 | 0.996609 | 46.64% | 59.45% |
| DCC | 0.001763163 | 0.611819 | 43.98% | 29.89% |
| TRHDE | 0.001799068 | 0.383545 | 52.28% | 34.28% |
| CELSR2 | 0.002040013 | 0.121845 | 16.11% | 14.90% |
| TSPAN7 | 0.002083182 | 0.238869 | 49.83% | 24.13% |
| MC4R | 0.00208658 | 0.870939 | 55.25% | 37.07% |
| SEMA6B | 0.002106013 | 0.757251 | 14.52% | 34.40% |
| GABBR2 | 0.002129002 | 0.532527 | 41.64% | 31.47% |
| P2RX1 | 0.00237415 | 0.993415 | 85.54% | 67.36% |
| PTPRU | 0.002397912 | 0.466814 | 28.44% | 24.41% |
| LRTM2 | 0.002527202 | 0.717192 | 66.22% | 59.82% |
| GABRA1 | 0.002636514 | 0.474428 | 69.15% | 56.25% |
| NRXN2 | 0.002983103 | 0.376875 | 41.40% | 42.94% |
| RPRML | 0.003139945 | 0.306696 | 89.14% | 53.12% |
| SLC22A8 | 0.003165052 | 0.770627 | 37.60% | 78.30% |
| OR2H2 | 0.003310359 | 0.179019 | 26.05% | 71.59% |
| SLC13A2 | 0.003800078 | 0.992882 | 59.02% | 70.66% |
| SLC4A1 | 0.004556806 | 0.993162 | 15.74% | 67.70% |
| CADM3 | 0.004840547 | 0.213281 | 66.08% | 54.39% |
| CDHR1 | 0.00484115 | 0.459116 | 54.28% | 19.66% |
| KCNJ3 | 0.004842937 | 0.447606 | 42.17% | 42.06% |
| GABRA5 | 0.005154631 | 0.691099 | 49.46% | 45.06% |
| KCNA3 | 0.0051888 | 0.378066 | 69.22% | 24.06% |
| ABCC9 | 0.005193669 | 0.235473 | 12.88% | 23.40% |
| SLC5A10 | 0.005218323 | 0.764052 | 48.17% | 47.21% |
| CDH23 | 0.005255963 | 0.179502 | 18.66% | 32.68% |
| GRM4 | 0.005587357 | 0.359874 | 45.93% | 35.12% |
| DCT | 0.006553942 | 0.643339 | 40.79% | 29.24% |
| CHRND | 0.006605221 | 0.617916 | 40.04% | 65.48% |
| CDH7 | 0.006638763 | 0.618596 | 49.10% | 25.69% |
| SUSD4 | 0.007377705 | 0.179487 | 40.25% | 20.74% |
| SLC2A5 | 0.008238736 | 0.746701 | 22.84% | 64.33% |
| SCN3B | 0.008502617 | 0.243491 | 49.90% | 19.07% |
| RTN4RL2 | 0.008820049 | 0.143139 | 34.66% | 26.27% |
| HCAR3 | 0.008901462 | 0.68062 | 100.00% | 60.12% |
| GRIN1 | 0.009078367 | 0.438567 | 75.12% | 38.31% |
| NKAIN1 | 0.009642192 | 0.238997 | 28.49% | 39.00% |
| SLC6A15 | 0.011264412 | 0.311918 | 21.00% | 30.96% |
| CNTFR | 0.011542929 | 0.51033 | 17.29% | 38.92% |
| MDGA1 | 0.011585819 | 0.555585 | 14.59% | 27.33% |
| EFNB1 | 0.011717381 | 0.237674 | 18.21% | 31.38% |
| PCDH9 | 0.011810994 | 0.399979 | 24.32% | 21.27% |
| ADORA3 | 0.012479083 | 0.23585 | 45.66% | 75.94% |
| JAM2 | 0.014074375 | 0.182362 | 17.16% | 25.17% |
| PTCHD2 | 0.016059586 | 0.578433 | 31.55% | 17.98% |
| PTGFR | 0.017080087 | 0.418004 | 27.46% | 60.53% |
| HRH2 | 0.018605611 | 0.994053 | 19.38% | 55.05% |
| APCDD1 | 0.018901357 | 0.486097 | 36.09% | 42.53% |
| SUSD2 | 0.019612785 | 0.519213 | 34.52% | 49.03% |
| CDH5 | 0.019710644 | 0.995518 | 68.16% | 50.61% |
| SLCO4A1 | 0.02063237 | 0.258444 | 37.55% | 39.35% |
| CHRNB4 | 0.022963416 | 0.253687 | 21.69% | 48.54% |
| ADAM23 | 0.023793566 | 0.201117 | 25.79% | 12.26% |
| GLP1R | 0.023976932 | 0.384716 | 70.15% | 47.25% |
| MEGF10 | 0.024891723 | 0.55549 | 16.40% | 43.50% |
| GJD2 | 0.025351565 | 0.7403 | 85.67% | 59.13% |
| NFAM1 | 0.025487119 | 0.994087 | 58.15% | 41.45% |
| SLC46A2 | 0.025594673 | 0.323981 | 87.03% | 27.75% |
| SLAMF1 | 0.027767997 | 0.992634 | 62.66% | 74.52% |
| CHODL | 0.029090402 | 0.297406 | 60.12% | 21.76% |
| CACNA1C | 0.030212047 | 0.203719 | 39.22% | 14.86% |
| FNDC5 | 0.030917088 | 0.261243 | 27.07% | 16.69% |
| SEMA5B | 0.031412403 | 0.292277 | 23.64% | 34.84% |
| TMEFF1 | 0.033544969 | 0.373938 | 86.67% | 15.93% |
| ROR1 | 0.035371532 | 0.243011 | 22.08% | 23.12% |
| NPHS1 | 0.036023319 | 0.995355 | 35.92% | 27.66% |
| PRIMA1 | 0.036222923 | 0.326878 | 49.72% | 33.84% |
| NPBWR2 | 0.037312881 | 0.172744 | 36.82% | 47.18% |
| OR4F4 | 0.037514132 | 0.710837 | 44.21% | 63.19% |
| ANTXRL | 0.038021714 | 0.684813 | 19.46% | 66.63% |
| THSD1 | 0.038144284 | 0.105326 | 26.56% | 23.01% |
| GRM2 | 0.038277571 | 0.18788 | 49.84% | 11.24% |
| ISLR2 | 0.039237948 | 0.790982 | 62.45% | 10.29% |
| NTRK3 | 0.03961383 | 0.157584 | 38.91% | 50.97% |
| SLITRK2 | 0.040807522 | 0.996703 | 58.42% | 56.27% |
| ATP2B3 | 0.040933239 | 0.259398 | 37.97% | 23.95% |
| SLC6A18 | 0.041500349 | 0.990155 | 100.00% | 59.64% |
| CDH24 | 0.043112033 | 0.135478 | 15.85% | 16.32% |
| KCNJ4 | 0.043328163 | 0.488501 | 71.06% | 69.25% |
| HEPACAM | 0.043981912 | 0.529568 | 31.42% | 70.48% |
| OPRK1 | 0.044515264 | 0.482909 | 70.71% | 32.76% |
| ADAM7 | 0.045375168 | 0.640254 | 38.89% | 48.37% |
| P2RX3 | 0.046827172 | 0.710298 | 75.59% | 58.24% |
| NTSR1 | 0.047718717 | 0.701568 | 39.60% | 57.02% |
| EPHA8 | 0.04818322 | 0.425589 | 45.88% | 35.55% |
| GPR128 | 0.050343275 | 0.857379 | 30.70% | 57.17% |
| HTR3A | 0.050668866 | 0.994507 | 77.79% | 11.76% |
| GPR132 | 0.053112532 | 0.527031 | 100.00% | 63.50% |
| SCN10A | 0.053666478 | 0.745732 | 28.40% | 59.70% |
| KREMEN2 | 0.053743604 | 0.423298 | 57.48% | 56.54% |
| SLC17A6 | 0.054454318 | 0.500605 | 33.02% | 38.37% |
| SLC22A17 | 0.05610198 | 0.343078 | 28.69% | 34.59% |
| CLEC17A | 0.060527134 | 0.261234 | 46.72% | 62.53% |
| FOLR1 | 0.060997887 | 0.744579 | 100.00% | 39.12% |
| TMEM225 | 0.061326362 | 0.416335 | 84.90% | 64.68% |
| TMEM158 | 0.061563453 | 0.185954 | 15.25% | 25.74% |
| SLC6A3 | 0.063368804 | 0.761258 | 56.75% | 13.58% |
| CD163L1 | 0.063766131 | 0.190876 | 36.57% | 43.36% |
| P2RY2 | 0.064606611 | 0.358441 | 42.33% | 56.35% |
| OR10G6 | 0.066681201 | 0.337719 | 42.08% | 74.02% |
| HRH4 | 0.066896804 | 0.211265 | 26.40% | 26.54% |
| ESAM | 0.067925809 | 0.116218 | 50.16% | 36.09% |
| CD34 | 0.069053757 | 0.708813 | 17.97% | 34.88% |
| GABRB2 | 0.071058093 | 0.287005 | 46.49% | 27.13% |
| EQTN | 0.071170782 | 0.366985 | 61.97% | 39.63% |
| CCR4 | 0.072963763 | 0.681381 | 44.61% | 61.98% |
| FCGR3B | 0.075475097 | 0.398595 | 21.13% | 63.24% |
| TMEM132E | 0.078663019 | 0.890608 | 44.60% | 42.21% |
| CACNA1G | 0.079889527 | 0.656978 | 13.94% | 25.13% |
| SLC5A5 | 0.080196733 | 0.990552 | 54.33% | 59.06% |
| KCNJ12 | 0.082526325 | 0.601294 | 12.77% | 48.72% |
| MIP | 0.084603583 | 0.989031 | 100.00% | 29.44% |
| HTR7 | 0.085060074 | 0.597565 | 52.08% | 40.32% |
| GPR4 | 0.086853398 | 0.493402 | 25.78% | 45.76% |
| GFRA1 | 0.091389977 | 0.31121 | 30.99% | 45.59% |
| CHRNA4 | 0.092519633 | 0.463322 | 26.48% | 55.71% |
| ITGA9 | 0.098490005 | 0.358721 | 26.47% | 11.87% |
| GPR179 | 0.098980368 | 0.303806 | 25.92% | 24.65% |
| CD19 | 0.099054329 | 0.780064 | 64.49% | 46.73% |
| OR1F1 | 0.100497141 | 0.645749 | 33.17% | 42.09% |
| OR4D5 | 0.100897942 | 0.301125 | 77.60% | 69.39% |
| ADRA1B | 0.102760655 | 0.987997 | 13.63% | 69.32% |
| TSHR | 0.103255459 | 0.223669 | 21.66% | 23.71% |
| P2RX2 | 0.106303874 | 0.200903 | 79.16% | 55.06% |
| CD3G | 0.108333756 | 0.691807 | 100.00% | 48.15% |
| CATSPERD | 0.11451802 | 0.416627 | 30.67% | 14.70% |
| LILRB3 | 0.118444169 | 0.99204 | 20.15% | 31.06% |
| GPR62 | 0.119117633 | 0.171653 | 30.99% | 38.51% |
| RAETIL | 0.122171319 | 0.988156 | 100.00% | 60.74% |
| SCN5A | 0.125197844 | 0.12765 | 26.02% | 27.05% |
| NALCN | 0.126324161 | 0.288302 | 50.00% | 17.14% |
| CACNG5 | 0.126702297 | 0.801518 | 23.59% | 43.55% |
| TACR2 | 0.12872173 | 0.991203 | 79.94% | 50.99% |
| P2RX6 | 0.132137039 | 0.42925 | 58.55% | 41.37% |
| DISP2 | 0.136632357 | 0.368852 | 12.80% | 17.22% |
| ASIC4 | 0.137388371 | 0.603354 | 56.16% | 31.18% |
| CDH18 | 0.137540096 | 0.540003 | 20.13% | 32.63% |
| CD80 | 0.139564702 | 0.225716 | 31.93% | 10.98% |
| KIR2DL3 | 0.140628153 | 0.990679 | 49.43% | 62.79% |
| IL10RA | 0.142512933 | 0.143167 | 59.22% | 19.44% |
| GABRA4 | 0.142536482 | 0.188001 | 47.22% | 53.33% |
| SV2C | 0.144743159 | 0.636311 | 57.08% | 41.65% |
| GPR182 | 0.150943639 | 0.138552 | 55.49% | 41.75% |
| SLAMF6 | 0.154931463 | 0.99269 | 13.54% | 52.07% |
| ITGB2 | 0.156420219 | 0.988004 | 46.31% | 55.62% |
| MUC12 | 0.166002909 | 0.186138 | 16.87% | 24.63% |
| MSLNL | 0.167171263 | 0.197801 | 100.00% | 16.18% |
| CDH11 | 0.168646986 | 0.417411 | 27.47% | 38.02% |
| PLXNA4 | 0.169702081 | 0.460245 | 20.46% | 23.40% |
| IZUMO3 | 0.170663536 | 0.993242 | 35.87% | 44.19% |
| CLDN11 | 0.175655291 | 0.433906 | 32.20% | 13.43% |
| IL2RB | 0.175689643 | 0.721105 | 42.21% | 58.92% |
| RXFP2 | 0.178376998 | 0.217354 | 50.79% | 45.03% |
| GRIA2 | 0.184534204 | 0.380533 | 16.88% | 13.10% |
| ITGAM | 0.186462375 | 0.992467 | 32.71% | 34.46% |
| GABRA3 | 0.198028647 | 0.151829 | 34.41% | 13.18% |
| TMEM106A | 0.199951886 | 0.115534 | 16.91% | 18.10% |
| UNC5D | 0.202116639 | 0.648594 | 16.02% | 27.90% |
| GPBAR1 | 0.202194834 | 0.989678 | 30.39% | 35.56% |
| OPN1LW | 0.203298156 | 0.634641 | 74.45% | 59.93% |
| KCNV2 | 0.204382861 | 0.44157 | 21.37% | 30.13% |
| SLC29A4 | 0.204428529 | 0.195657 | 12.10% | 19.28% |
| CD28 | 0.207056522 | 0.99023 | 40.77% | 42.46% |
| LAMP5 | 0.212140025 | 0.629302 | 16.73% | 26.94% |
| CDH22 | 0.213523399 | 0.316892 | 42.90% | 29.01% |
| CD244 | 0.214199101 | 0.670223 | 43.02% | 62.53% |
| NRN1 | 0.21922131 | 0.413486 | 31.63% | 35.04% |
| SLC1A2 | 0.224572726 | 0.141188 | 23.08% | 21.40% |
| OR6T1 | 0.22917947 | 0.693875 | 100.00% | 51.35% |
| ABCA8 | 0.230173119 | 0.448978 | 23.60% | 22.18% |
| LRP3 | 0.232840961 | 0.635271 | 79.69% | 37.56% |
| GABRA6 | 0.237650836 | 0.448059 | 100.00% | 47.31% |
| SLC5A11 | 0.258308171 | 0.489338 | 22.42% | 25.77% |
| CACNA1I | 0.259023484 | 0.423733 | 31.86% | 39.64% |
| HTR3D | 0.272879718 | 0.629849 | 41.34% | 50.01% |
| GRIK5 | 0.276461966 | 0.132461 | 32.16% | 21.24% |
| OR8D4 | 0.28003768 | 0.984577 | 100.00% | 61.68% |
| KCNK5 | 0.28098548 | 0.120781 | 46.90% | 32.86% |
| CEACAM20 | 0.284553421 | 0.146899 | 100.00% | 13.70% |
| PRLHR | 0.29414723 | 0.509059 | 77.77% | 55.19% |
| CACNG3 | 0.295588554 | 0.99419 | 73.19% | 48.63% |
| EFNB3 | 0.315241589 | 0.165782 | 24.15% | 14.50% |
| GGT3P | 0.317782258 | 0.480757 | 51.23% | 42.03% |
| IL9R | 0.322374188 | 0.988545 | 48.86% | 18.96% |
| SLC13A1 | 0.324645648 | 0.177374 | 56.62% | 31.51% |
| IL21R | 0.326899933 | 0.989428 | 29.42% | 41.65% |
| IL12RB1 | 0.329517537 | 0.315774 | 39.97% | 41.71% |
| FNDC4 | 0.330023313 | 0.326219 | 16.44% | 50.37% |
| CD84 | 0.342037245 | 0.988013 | 15.16% | 54.32% |
| FCGR2B | 0.344726618 | 0.629271 | 33.76% | 45.64% |
| UNC5B | 0.3500249 | 0.283174 | 15.67% | 13.14% |
| LHFPL4 | 0.359516303 | 0.581902 | 33.68% | 27.93% |
| SLITRK1 | 0.360713323 | 0.459576 | 32.17% | 15.41% |
| FAM171A2 | 0.364242476 | 0.707878 | 26.71% | 13.84% |
| LRRC52 | 0.368908866 | 0.986423 | 64.18% | 52.75% |
| SORCS2 | 0.381578535 | 0.266461 | 29.39% | 11.33% |
| ATP1B4 | 0.406298514 | 0.987941 | 40.69% | 23.38% |
| PLD5 | 0.427057522 | 0.357964 | 23.81% | 12.14% |
| CD22 | 0.4272725 | 0.421516 | 28.85% | 53.43% |
| LRFN2 | 0.428594101 | 0.44242 | 44.27% | 23.40% |
| ENG | 0.428716467 | 0.251264 | 49.68% | 14.77% |
| BEST2 | 0.431081339 | 0.986523 | 56.01% | 22.91% |
| CEACAM3 | 0.433555458 | 0.633021 | 73.49% | 32.48% |
| SCARA5 | 0.434417137 | 0.250507 | 22.98% | 49.38% |
| GPR84 | 0.456789252 | 0.621749 | 32.17% | 11.01% |
| CACNG2 | 0.464836781 | 0.420652 | 38.08% | 25.22% |
| CNTN1 | 0.465118408 | 0.19038 | 28.56% | 15.45% |
| TMPRSS6 | 0.47137444 | 0.268083 | 39.11% | 17.21% |
| LAG3 | 0.472254409 | 0.209448 | 22.43% | 15.32% |
| SLC39A12 | 0.472461668 | 0.536181 | 32.26% | 46.72% |
| SLCO2B1 | 0.482772795 | 0.593375 | 78.08% | 31.80% |
| NKAIN3 | 0.488481981 | 0.388975 | 42.99% | 39.64% |
| EDA2R | 0.491285835 | 0.60597 | 17.88% | 39.87% |
| CHRNB3 | 0.494834883 | 0.993082 | 30.98% | 20.01% |
| LILRA2 | 0.497229088 | 0.979169 | 54.25% | 56.50% |
| MC5R | 0.502098427 | 0.990584 | 24.45% | 35.99% |
| CD177 | 0.524615525 | 0.524602 | 100.00% | 57.50% |
| PRPH2 | 0.530742822 | 0.601163 | 16.14% | 37.43% |
| GHRHR | 0.532953844 | 0.666291 | 22.38% | 37.02% |
| GRID1 | 0.540289247 | 0.416137 | 20.36% | 12.40% |
| TIE1 | 0.546505104 | 0.636402 | 13.85% | 24.83% |
| RGR | 0.55619227 | 0.705131 | 23.42% | 51.08% |
| OR5AK3P | 0.568639971 | 0.984924 | 100.00% | 30.48% |
| PIGR | 0.594033022 | 0.989134 | 44.92% | 19.66% |
| CNTN3 | 0.597952987 | 0.291838 | 26.77% | 17.96% |
| OR9K2 | 0.599544888 | 0.989015 | 50.83% | 24.90% |
| FLRT2 | 0.6014282 | 0.116162 | 13.27% | 11.54% |
| CD33 | 0.604088847 | 0.990909 | 53.91% | 43.89% |
| GPR12 | 0.604440163 | 0.288298 | 42.12% | 32.24% |
| OTOP2 | 0.630371681 | 0.43103 | 28.75% | 41.61% |
| CSF3R | 0.63851776 | 0.225314 | 31.46% | 36.91% |
| DRD4 | 0.64210982 | 0.17982 | 36.22% | 35.40% |
| SLC32A1 | 0.668412006 | 0.989054 | 33.30% | 18.78% |
| EMR1 | 0.688837005 | 0.609496 | 16.90% | 36.00% |
| OR51B6 | 0.690240883 | 0.285373 | 72.24% | 15.43% |
| SLC11A1 | 0.692583273 | 0.539562 | 32.32% | 44.84% |
| VNN3 | 0.693805196 | 0.980375 | 55.26% | 18.10% |
| OR5H1 | 0.697717759 | 0.975923 | 21.42% | 50.26% |
| SLAMF7 | 0.714983665 | 0.982987 | 44.47% | 25.40% |
| NCAM2 | 0.720550155 | 0.143248 | 28.13% | 13.53% |
| OPN5 | 0.733138726 | 0.476409 | 43.29% | 11.61% |
| SHISA7 | 0.735659952 | 0.359804 | 27.03% | 19.78% |
| OR11H2 | 0.740117488 | 0.322261 | 35.71% | 29.55% |
| LY9 | 0.766677765 | 0.985067 | 14.03% | 22.24% |
| PIEZO2 | 0.781736941 | 0.210634 | 24.06% | 13.69% |
| QRFPR | 0.790602115 | 0.661764 | 11.89% | 23.84% |
| KIR2DS4 | 0.810759799 | 0.454782 | 23.88% | 42.53% |
| FLT3LG | 0.816222367 | 0.437917 | 19.03% | 18.76% |
| C3AR1 | 0.830058634 | 0.205067 | 25.06% | 25.11% |
| CD200R1L | 0.844930358 | 0.108784 | 18.79% | 17.73% |
| EMCN | 0.873427457 | 0.158966 | 14.47% | 19.92% |
| SIGLEC9 | 0.874007162 | 0.515003 | 29.71% | 37.99% |
| AGTR2 | 0.884431885 | 0.599187 | 29.61% | 12.42% |
| CD1E | 0.88552805 | 0.977289 | 48.22% | 18.34% |
| GPR133 | 0.89195899 | 0.303044 | 21.46% | 13.99% |
| PEAR1 | 0.906066466 | 0.41424 | 23.50% | 14.18% |
| ACVRL1 | 0.917990908 | 0.468943 | 21.40% | 12.89% |
| OR7D2 | 0.935890303 | 0.393953 | 21.17% | 17.74% |
| AOC3 | 0.936843859 | 0.116334 | 32.48% | 12.88% |
| SEMA6D | 4.46585E−12 | 0.317069 | 42.23% | 4.32% |
| SLC5A6 | 1.07987E−09 | 0.107876 | 4.56% | 17.95% |
| IGDCC4 | 7.547E−07 | 0.101576 | 3.90% | 47.41% |
| NFASC | 1.19209E−06 | 0.357136 | 11.66% | 0.57% |
| SLC47A1 | 1.21018E−06 | 0.576145 | 9.13% | 47.16% |
| PCDH8 | 1.93596E−06 | 0.57831 | 64.52% | 9.05% |
| SCARB1 | 2.06522E−06 | 0.170988 | 3.76% | 24.15% |
| TSPAN13 | 2.29062E−06 | 0.044154 | 28.50% | 2.55% |
| SEZ6 | 4.4758E−06 | 0.397072 | 36.10% | 3.74% |
| GABRR3 | 5.44958E−06 | 0.099033 | 91.64% | 75.94% |
| FGFR2 | 6.71968E−06 | 0.070438 | 11.05% | 41.79% |
| UNC5C | 1.44512E−05 | 0.232361 | 1.69% | 58.33% |
| PROCR | 2.31358E−05 | 0.857643 | 5.20% | 66.86% |
| PTPRN | 2.42204E−05 | 0.640559 | 79.11% | 9.87% |
| FZD7 | 3.49328E−05 | 0.230408 | 9.41% | 38.30% |
| ITGA7 | 3.61828E−05 | 0.226449 | 3.72% | 45.06% |
| SLC6A8 | 4.25538E−05 | 0.0428 | 9.56% | 22.43% |
| IFNAR2 | 4.4031E−05 | 0.048092 | 13.05% | 8.93% |
| ANKH | 5.17024E−05 | 0.114017 | 22.51% | 4.72% |
| CHRNA7 | 7.47711E−05 | 0.09178 | 23.63% | 38.77% |
| KIRREL | 0.000102951 | 0.051516 | 3.48% | 55.10% |
| SLC16A1 | 0.000109136 | 0.017519 | 2.32% | 13.17% |
| LRP8 | 0.000122092 | 0.08106 | 7.32% | 10.02% |
| GRIK3 | 0.00014315 | 0.473278 | 58.39% | 6.05% |
| HAVCR1 | 0.000231816 | 0.437585 | 8.48% | 48.67% |
| SLC24A4 | 0.00029119 | 0.588864 | 4.20% | 61.94% |
| ABCC1 | 0.000367086 | 0.082036 | 2.51% | 12.09% |
| ADAM22 | 0.000686889 | 0.043486 | 17.07% | 0.36% |
| SLC26A4 | 0.000702325 | 0.07637 | 30.76% | 33.93% |
| QSOX2 | 0.000738608 | 0.180563 | 9.75% | 1.17% |
| CD4 | 0.000771855 | 0.021375 | 17.30% | 47.77% |
| ACVR2A | 0.000817634 | 0.070081 | 13.08% | 1.09% |
| SLC46A1 | 0.000843757 | 0.157026 | 14.50% | 7.70% |
| GPM6B | 0.000858494 | 0.040771 | 16.26% | 6.57% |
| TSPAN9 | 0.001071511 | 0.39753 | 0.12% | 36.21% |
| SEZ6L2 | 0.001073125 | 0.324529 | 12.66% | 3.52% |
| SYNPR | 0.001438554 | 0.278522 | 83.05% | 9.99% |
| ABCC4 | 0.001463226 | 0.028498 | 12.30% | 23.91% |
| SLC29A2 | 0.001528701 | 0.359304 | 0.27% | 22.31% |
| P2RY11 | 0.002550368 | 0.361858 | 2.61% | 18.43% |
| GABRB3 | 0.002714039 | 0.421604 | 32.05% | 9.71% |
| AMIGO1 | 0.002827694 | 0.123021 | 7.88% | 21.64% |
| CXCR6 | 0.002924358 | 0.014913 | 14.00% | 15.51% |
| FZD3 | 0.003412621 | 0.008765 | 21.25% | 1.48% |
| RNF149 | 0.003809296 | 0.014678 | 4.00% | 6.88% |
| ATP1B3 | 0.003954213 | 0.091451 | 3.59% | 5.25% |
| NEO1 | 0.003978507 | 0.07129 | 10.47% | 13.65% |
| MFSD2A | 0.004074467 | 0.260661 | 33.29% | 2.25% |
| SLC39A10 | 0.004413382 | 0.018327 | 4.18% | 6.96% |
| IGSF9B | 0.005258068 | 0.423308 | 23.72% | 4.41% |
| ROBO2 | 0.005392348 | 0.486235 | 8.14% | 22.18% |
| SLC2A2 | 0.005957685 | 0.088545 | 2.16% | 30.39% |
| PODXL2 | 0.007807708 | 0.049172 | 21.38% | 9.04% |
| CHRNA5 | 0.007954016 | 0.025146 | 19.48% | 5.25% |
| TMEM132A | 0.009928634 | 0.15216 | 1.63% | 17.23% |
| ATP13A2 | 0.010573131 | 0.08218 | 0.45% | 14.87% |
| RAMP2 | 0.01343539 | 0.615989 | 4.39% | 27.87% |
| SLC2A4 | 0.014307518 | 0.72817 | 6.00% | 44.04% |
| HM13 | 0.015066921 | 0.091903 | 1.80% | 6.67% |
| CSPG5 | 0.01783607 | 0.056108 | 12.28% | 18.88% |
| GPR113 | 0.02295274 | 0.036293 | 13.97% | 16.62% |
| SLC12A5 | 0.024254771 | 0.337997 | 16.82% | 4.50% |
| TMEM63C | 0.024839859 | 0.316701 | 23.23% | 4.36% |
| SLC44A3 | 0.025003433 | 0.001446 | 24.67% | 1.76% |
| ADCY5 | 0.02720106 | 0.556833 | 10.50% | 4.57% |
| EPHA5 | 0.027531537 | 0.348035 | 4.62% | 29.40% |
| MPZL1 | 0.027615682 | 0.072514 | 1.03% | 6.14% |
| ABCA4 | 0.029199146 | 0.741878 | 5.96% | 36.13% |
| GPC6 | 0.02990218 | 0.100929 | 22.95% | 6.15% |
| TAS2R16 | 0.030836847 | 0.361952 | 3.85% | 72.28% |
| PTGFRN | 0.031848681 | 0.002821 | 4.08% | 11.19% |
| TGFBR1 | 0.032281711 | 0.09396 | 7.26% | 5.44% |
| GRIA4 | 0.032638813 | 0.294853 | 31.84% | 7.00% |
| ANO5 | 0.03292595 | 0.173622 | 24.39% | 2.17% |
| GPR63 | 0.033174637 | 0.023425 | 30.21% | 10.90% |
| TMEFF2 | 0.033210008 | 0.019234 | 41.02% | 16.98% |
| TSPAN5 | 0.035010843 | 0.052429 | 26.50% | 21.62% |
| IMPG2 | 0.03890453 | 0.366464 | 6.37% | 15.96% |
| GPR173 | 0.041864014 | 0.11082 | 26.11% | 7.01% |
| IGF1R | 0.042444353 | 0.037877 | 4.58% | 9.60% |
| CLSTN2 | 0.042626103 | 0.072209 | 37.69% | 9.79% |
| GPR3 | 0.043933031 | 0.26419 | 2.25% | 13.32% |
| LDLRAD3 | 0.045160096 | 0.018361 | 10.73% | 12.48% |
| SLC39A8 | 0.045735119 | 0.068626 | 21.31% | 13.59% |
| RNF150 | 0.045853947 | 0.077915 | 8.40% | 17.20% |

TABLE 6
Surface proteins associated with the SCLC-N subtype from the Sato dataset

| Surface Protein | ANOVA p value | N vs A | N vs P | N vs I |
|---|---|---|---|---|
| CHRNA9 | 5.05448E−08 | 0.120157 | 45.912% | 72.111% |
| GRM8 | 3.96474E−07 | 0.355071 | 80.993% | 76.876% |
| SEZ6 | 1.03337E−06 | 0.139193 | 76.746% | 75.267% |
| LRFN5 | 1.81258E−06 | 0.158124 | 66.122% | 55.978% |
| UMODL1 | 1.82838E−06 | 0.38338 | 34.606% | 43.204% |
| SLC17A6 | 3.22336E−06 | 0.524894 | 73.828% | 67.211% |
| ADRA2A | 6.51148E−06 | 0.115274 | 60.917% | 56.850% |
| LYPD1 | 9.67515E−06 | 0.100733 | 54.238% | 45.481% |
| CHRNA3 | 9.68903E−06 | 0.21799 | 64.120% | 48.008% |
| SLCO4C1 | 2.61247E−05 | 0.185723 | 26.358% | 18.216% |
| FZD6 | 3.18865E−05 | 0.140046 | 13.137% | 16.090% |
| DLK1 | 0.000116332 | 0.193184 | 62.136% | 47.937% |
| PTCHD1 | 0.000228436 | 0.632619 | 63.818% | 66.797% |
| SEMA6A | 0.001402726 | 0.289802 | 50.996% | 32.395% |
| LRP12 | 0.001655218 | 0.108132 | 17.170% | 29.620% |
| CLSTN2 | 0.00193009 | 0.144353 | 52.121% | 50.970% |
| NRG1 | 0.003235572 | 0.154046 | 60.232% | 25.135% |
| DCC | 0.003547465 | 0.566922 | 46.824% | 63.911% |
| GPR161 | 0.003619516 | 0.21023 | 18.249% | 17.966% |
| ADAM23 | 0.005311949 | 0.204287 | 63.098% | 25.644% |
| SSTR2 | 0.006876376 | 0.190847 | 30.143% | 29.927% |
| DSCAML1 | 0.007401821 | 0.133409 | 38.334% | 54.470% |
| NRP1 | 0.007847671 | 0.525123 | 32.142% | 32.762% |
| TRHDE | 0.007956922 | 0.492265 | 56.800% | 44.898% |
| GRAMD1B | 0.008010425 | 0.159664 | 36.605% | 30.847% |
| TMEM106A | 0.008322271 | 0.414556 | 32.818% | 40.949% |
| RPRM | 0.008765948 | 0.139974 | 27.699% | 34.039% |
| LY6H | 0.008792535 | 0.253085 | 57.908% | 44.754% |
| TNFRSF19 | 0.009987767 | 0.192521 | 35.433% | 14.841% |
| CLRN1 | 0.010076298 | 0.285813 | 52.537% | 55.581% |
| GPR55 | 0.01070931 | 0.646909 | 47.699% | 34.856% |
| NTNG2 | 0.010800069 | 0.455904 | 55.852% | 39.647% |
| EPHB2 | 0.011331235 | 0.235891 | 28.950% | 24.949% |
| SGCD | 0.011746661 | 0.350728 | 45.133% | 48.681% |
| MMP16 | 0.014956634 | 0.334138 | 50.321% | 61.710% |
| OR8G2 | 0.015622399 | 0.105404 | 50.108% | 59.996% |
| PLXNB1 | 0.018092138 | 0.145416 | 37.690% | 57.022% |
| CNR1 | 0.020145322 | 0.217248 | 50.401% | 45.292% |
| PCDHAC1 | 0.022162682 | 0.107299 | 20.347% | 55.591% |
| CD200R1 | 0.023823591 | 0.514934 | 29.122% | 72.538% |
| PRIMA1 | 0.025581156 | 0.254529 | 40.877% | 25.621% |
| MRGPRX1 | 0.025819972 | 0.421503 | 20.543% | 12.984% |
| THSD7B | 0.026001554 | 0.193007 | 69.592% | 35.961% |
| DRD4 | 0.026572063 | 0.139979 | 36.941% | 40.366% |
| CDHR1 | 0.031047336 | 0.50565 | 71.178% | 38.867% |
| GRIA2 | 0.032074602 | 0.368383 | 83.751% | 86.154% |
| SLC19A2 | 0.032547901 | 0.106657 | 11.397% | 13.372% |
| SEMA5B | 0.037498693 | 0.243647 | 42.878% | 31.330% |
| GPR12 | 0.038568437 | 0.117854 | 35.447% | 33.159% |
| EPHA6 | 0.041171946 | 0.348497 | 60.615% | 50.138% |
| ASTN1 | 0.041246289 | 0.316287 | 55.840% | 52.046% |
| GPR88 | 0.04132599 | 0.462697 | 54.393% | 63.525% |
| ALK | 0.042289593 | 0.31447 | 40.233% | 40.512% |
| EFNB3 | 0.050148985 | 0.130481 | 49.443% | 27.884% |
| BVES | 0.053667518 | 0.27505 | 26.212% | 33.684% |
| PTK7 | 0.05449027 | 0.19632 | 14.300% | 23.241% |
| CLDN4 | 0.056211309 | 0.136289 | 20.372% | 13.782% |
| TRIL | 0.062735791 | 0.356276 | 32.099% | 24.340% |
| GPR26 | 0.068259231 | 0.706576 | 46.731% | 60.279% |
| LRTM2 | 0.070396159 | 0.312631 | 45.241% | 50.649% |
| NLGN4X | 0.071490795 | 0.211887 | 32.272% | 58.343% |
| PCNXL2 | 0.074558267 | 0.138128 | 19.956% | 23.921% |
| TACR1 | 0.078481263 | 0.430424 | 55.460% | 38.901% |
| GRM2 | 0.080083393 | 0.236109 | 58.405% | 44.184% |
| SCNN1B | 0.080376427 | 0.290862 | 55.831% | 31.538% |
| SLC5A4 | 0.08070531 | 0.17415 | 38.647% | 37.976% |
| MEGF10 | 0.085450419 | 0.438442 | 36.192% | 49.937% |
| CHRNA1 | 0.087817204 | 0.181296 | 28.089% | 46.130% |
| APLP1 | 0.093640889 | 0.113433 | 63.459% | 52.045% |
| ROR2 | 0.096618214 | 0.397502 | 39.646% | 50.793% |
| SCNN1G | 0.096629118 | 0.357045 | 53.447% | 38.709% |
| MCHR1 | 0.099118137 | 0.296733 | 43.637% | 56.272% |
| LRP4 | 0.100891439 | 0.117016 | 34.838% | 26.299% |
| FZD10 | 0.101992191 | 0.256895 | 44.522% | 18.144% |
| CNTNAP5 | 0.10602115 | 0.333789 | 40.073% | 45.894% |
| FZD7 | 0.106798967 | 0.315139 | 14.175% | 14.988% |
| NPFFR2 | 0.108494803 | 0.484079 | 51.538% | 46.733% |
| DLL1 | 0.110675906 | 0.105097 | 12.264% | 27.898% |
| DUOXA1 | 0.119642284 | 0.460531 | 28.932% | 48.096% |
| CNTN1 | 0.136886879 | 0.264896 | 43.750% | 29.543% |
| TAS2R7 | 0.138565531 | 0.270276 | 49.610% | 50.050% |
| KIAA1324 | 0.141632729 | 0.162504 | 16.513% | 42.199% |
| F3 | 0.147728305 | 0.285252 | 25.133% | 10.791% |
| GABRG3 | 0.152392437 | 0.279615 | 40.861% | 31.331% |
| GPR125 | 0.156687696 | 0.150356 | 11.711% | 20.317% |
| GPR143 | 0.174935715 | 0.374312 | 10.405% | 22.707% |
| SLC16A1 | 0.178516379 | 0.210151 | 24.273% | 25.377% |
| CSPG5 | 0.179218288 | 0.204093 | 36.023% | 37.678% |
| GPR158 | 0.192363478 | 0.208089 | 43.456% | 51.035% |
| GABRD | 0.217250984 | 0.621264 | 73.978% | 55.365% |
| GABBR2 | 0.234181726 | 0.270761 | 51.035% | 49.768% |
| OR2C3 | 0.236689791 | 0.180238 | 20.191% | 45.481% |
| SLC4A4 | 0.251108889 | 0.114065 | 31.605% | 26.837% |
| TSPAN1 | 0.252406369 | 0.182981 | 50.202% | 18.189% |
| GPR162 | 0.254149923 | 0.119056 | 47.981% | 22.067% |
| GPRC5D | 0.265890768 | 0.127619 | 36.879% | 10.294% |
| SLC24A2 | 0.270254978 | 0.347414 | 48.070% | 50.961% |
| RXFP3 | 0.271016239 | 0.264443 | 28.202% | 26.946% |
| ROR1 | 0.275779075 | 0.304678 | 23.880% | 37.387% |
| ENPP1 | 0.283169434 | 0.192028 | 38.926% | 45.657% |
| OR1J4 | 0.290985808 | 0.300929 | 18.488% | 45.087% |
| CDH4 | 0.291596488 | 0.27611 | 18.370% | 59.757% |
| FAM174B | 0.297106361 | 0.104782 | 18.327% | 12.588% |
| AGER | 0.304827 | 0.295823 | 39.246% | 40.755% |
| TSPAN5 | 0.305897682 | 0.175816 | 30.716% | 13.622% |
| FNDC5 | 0.317906978 | 0.292368 | 52.050% | 45.160% |
| SLC12A2 | 0.318871853 | 0.18774 | 18.953% | 14.405% |
| TMEFF2 | 0.340686347 | 0.364396 | 47.881% | 48.679% |
| TAS1R2 | 0.357917894 | 0.425702 | 29.500% | 33.763% |
| SLC44A3 | 0.375565664 | 0.141601 | 13.061% | 10.402% |
| PCDHA2 | 0.377575079 | 0.154232 | 43.069% | 28.105% |
| FFAR1 | 0.380611634 | 0.303569 | 28.179% | 44.525% |
| MST1R | 0.390798181 | 0.117365 | 14.891% | 19.384% |
| IL23R | 0.394638216 | 0.333865 | 24.822% | 42.711% |
| PRLHR | 0.406872394 | 0.372499 | 44.210% | 44.449% |
| OPRM1 | 0.446154303 | 0.121851 | 38.802% | 30.008% |
| CHRNB2 | 0.451283795 | 0.121364 | 23.267% | 32.050% |
| SV2C | 0.473484792 | 0.221116 | 26.873% | 41.279% |
| TMEM255A | 0.477567647 | 0.181819 | 17.251% | 31.592% |
| BAI2 | 0.487715326 | 0.179821 | 23.929% | 28.985% |
| TSHR | 0.50492401 | 0.170709 | 32.340% | 58.390% |
| GJB5 | 0.50950575 | 0.360938 | 51.067% | 21.608% |
| TMEM114 | 0.518438848 | 0.233104 | 23.751% | 27.489% |
| PCDHB3 | 0.537684013 | 0.333606 | 16.661% | 13.730% |
| DUOX2 | 0.557156536 | 0.403027 | 28.678% | 23.353% |
| ABCA12 | 0.604360884 | 0.135086 | 36.284% | 27.536% |
| SLC13A2 | 0.612876222 | 0.371814 | 35.208% | 19.777% |
| SLC22A15 | 0.615019122 | 0.181757 | 11.823% | 14.681% |
| SGCZ | 0.635672356 | 0.382806 | 18.361% | 23.228% |
| TACR3 | 0.641433487 | 0.184102 | 36.043% | 17.226% |
| SCNN1D | 0.684813576 | 0.297107 | 16.944% | 14.482% |
| HCRTR1 | 0.69535971 | 0.148244 | 21.805% | 37.521% |
| SIRPB2 | 0.705934206 | 0.352815 | 18.177% | 25.873% |
| DUOX1 | 0.716800806 | 0.321059 | 29.171% | 17.478% |
| C11orf87 | 0.736573623 | 0.105579 | 44.340% | 52.865% |
| PCDHA3 | 0.739183518 | 0.191675 | 30.937% | 20.869% |
| EDA | 0.745140252 | 0.130576 | 22.578% | 13.102% |
| ELFN2 | 0.754601024 | 0.171733 | 13.515% | 12.772% |
| ADAM2 | 0.771326494 | 0.287737 | 28.638% | 40.110% |
| HRH4 | 0.772160105 | 0.179724 | 22.041% | 35.437% |
| NALCN | 0.775858453 | 0.253893 | 15.739% | 23.483% |
| ASIC5 | 0.817348593 | 0.240306 | 17.544% | 21.065% |
| OR2W1 | 0.818607943 | 0.204991 | 13.862% | 25.608% |
| UPK3A | 0.849783625 | 0.238744 | 26.503% | 20.476% |
| GLRA2 | 0.915505273 | 0.14121 | 17.162% | 23.684% |
| CHRNA5 | 1.27891E−11 | 0.107333 | 4.726% | 17.538% |
| GFRA1 | 1.01361E−09 | 0.437949 | 62.453% | 59.433% |
| IGSF9 | 1.05941E−09 | 0.081147 | 5.303% | 18.822% |
| SUCO | 1.7538E−09 | 0.006319 | 2.667% | 5.183% |
| QSOX2 | 4.81129E−08 | 0.015085 | 23.385% | 21.711% |
| NETO2 | 6.18953E−07 | 0.101151 | 2.041% | 8.087% |
| CELSR3 | 8.74373E−07 | 0.030931 | 35.137% | 37.436% |
| NRXN1 | 9.86924E−07 | 0.057663 | 68.892% | 56.752% |
| NLGN1 | 2.29956E−06 | 0.083058 | 65.490% | 50.420% |
| SLC6A3 | 2.5439E−06 | 0.006027 | 53.750% | 35.221% |
| NFASC | 2.14888E−05 | 0.021436 | 46.811% | 52.966% |
| GRIK3 | 2.77401E−05 | 0.089955 | 56.445% | 45.232% |
| DRD2 | 6.23064E−05 | 0.037653 | 26.987% | 33.380% |
| GRIK2 | 8.96329E−05 | 0.075612 | 73.445% | 60.604% |
| SLC26A6 | 0.000174847 | 0.228561 | 34.337% | 29.550% |
| LMBRD2 | 0.000258161 | 0.00234 | 7.877% | 11.539% |
| SLC7A14 | 0.000344826 | 0.008064 | 68.080% | 67.113% |
| LHFPL4 | 0.000348227 | 0.048872 | 52.622% | 62.477% |
| FAM171B | 0.000740595 | 0.034657 | 26.799% | 23.189% |
| NEGR1 | 0.000824165 | 0.210725 | 53.127% | 43.838% |
| LRIG2 | 0.001169129 | 0.082719 | 8.313% | 20.367% |
| SEMA6D | 0.0012735 | 0.040362 | 50.591% | 34.111% |
| TMEM67 | 0.001575966 | 0.099503 | 23.407% | 23.525% |
| CADM1 | 0.002123513 | 0.027474 | 24.056% | 14.192% |
| SLC22A17 | 0.003569576 | 0.059665 | 49.628% | 40.694% |
| OR1D2 | 0.004206712 | 7.29E−05 | 13.936% | 28.752% |
| EFNA5 | 0.004523034 | 0.077912 | 30.287% | 27.034% |
| KIAA1324L | 0.004582258 | 0.090634 | 14.271% | 6.425% |
| SLC24A3 | 0.005248239 | 0.174392 | 34.976% | 25.743% |
| TMEM145 | 0.006607062 | 0.015526 | 30.501% | 33.812% |
| STS | 0.006752526 | 0.077968 | 23.221% | 7.572% |
| TTYH3 | 0.00758668 | 0.147819 | 19.115% | 4.698% |
| PLXNA3 | 0.00877675 | 0.069302 | 16.423% | 22.207% |
| IGSF9B | 0.009159819 | 0.066643 | 40.401% | 20.370% |
| FZD8 | 0.010714137 | 0.089724 | 22.144% | 20.530% |
| CLSTN1 | 0.011222174 | 0.085149 | 17.772% | 15.478% |
| PCDHB15 | 0.012801464 | 0.008644 | 34.701% | 24.854% |
| CRB2 | 0.013313783 | 0.013022 | 45.575% | 40.953% |
| OR12D2 | 0.0135988 | 0.195798 | 23.328% | 23.286% |
| ADCY3 | 0.016815836 | 0.124477 | 7.248% | 7.985% |
| SLC2A11 | 0.016939645 | 0.161686 | 17.921% | 34.713% |
| SDC2 | 0.017142998 | 0.196627 | 20.358% | 20.772% |
| GPR153 | 0.018102302 | 0.060658 | 22.565% | 29.072% |
| ELFN1 | 0.018211234 | 0.041309 | 22.816% | 8.376% |
| EFNB1 | 0.018798692 | 0.166495 | 24.622% | 21.840% |
| CLSTN3 | 0.019026418 | 0.150045 | 22.445% | 26.636% |
| TRABD2B | 0.019362447 | 0.235516 | 58.989% | 46.049% |
| FZD1 | 0.020353875 | 0.087526 | 15.726% | 2.555% |
| TMEM62 | 0.024359108 | 0.178813 | 12.545% | 9.872% |
| NGFR | 0.024698937 | 0.201476 | 53.037% | 25.619% |
| MUC4 | 0.025668431 | 0.259347 | 10.987% | 1.052% |
| PTPRU | 0.029043504 | 0.025649 | 20.275% | 18.220% |
| GPR98 | 0.029427265 | 0.076611 | 25.721% | 2.844% |
| CASD1 | 0.030340316 | 0.053706 | 13.113% | 16.993% |
| PTPRK | 0.031622952 | 0.100806 | 5.618% | 11.728% |
| LGR4 | 0.037668939 | 0.017517 | 16.978% | 5.782% |
| EBP | 0.043128127 | 0.103045 | 14.244% | 14.328% |
| SEMA5A | 0.048368106 | 0.112467 | 26.387% | 17.440% |
| SHISA9 | 0.049409959 | 0.090641 | 3.616% | 26.685% |
| C14orf132 | 0.04949203 | 0.01393 | 29.133% | 24.324% |

TABLE 7
Surface proteins associated with the SCLC-P subtype from the George et al. dataset

| Surface Protein | ANOVA P value | P vs N | P vs I | P vs A |
|---|---|---|---|---|
| EFNA4 | 8.4107E−11 | 0.665145 | 42.38% | 76.47% |
| TNFSF8 | 2.83139E−09 | 0.822427 | 29.54% | 76.55% |
| CD46 | 5.37238E−09 | 0.330505 | 21.80% | 17.81% |
| SMO | 6.58432E−09 | 0.138195 | 23.32% | 62.86% |
| ART3 | 2.90097E−08 | 0.98914 | 96.86% | 98.78% |
| ANO7 | 3.85277E−08 | 0.894624 | 73.08% | 88.78% |
| OLR1 | 5.43841E−08 | 0.759019 | 16.04% | 65.49% |
| ITGB4 | 6.66957E−08 | 0.720817 | 65.15% | 76.48% |
| AGTR1 | 7.26116E−08 | 0.922769 | 80.24% | 90.16% |
| GJC3 | 1.19386E−07 | 0.675332 | 79.11% | 42.66% |
| OR2T33 | 1.39955E−07 | 0.979554 | 88.94% | 96.48% |
| EFNA1 | 1.52055E−07 | 0.394795 | 29.10% | 22.96% |
| MICA | 2.36218E−07 | 0.486327 | 14.22% | 66.29% |
| IL1RL2 | 2.77546E−07 | 0.844712 | 50.71% | 81.16% |
| OR2W3 | 3.9506E−07 | 0.94874 | 80.54% | 95.40% |
| OR2T8 | 4.6941E−07 | 0.986425 | 97.90% | 96.02% |
| TMEM63A | 4.99873E−07 | 0.459291 | 21.80% | 38.99% |
| TLR5 | 2.23372E−06 | 0.505671 | 10.88% | 40.05% |
| TNFRSF21 | 4.34983E−06 | 0.388026 | 23.79% | 14.33% |
| SLC2A10 | 7.30936E−06 | 0.429897 | 25.45% | 50.62% |
| TMEM87A | 8.43103E−06 | 0.23469 | 13.97% | 16.86% |
| OMG | 9.95594E−06 | 0.838163 | 80.60% | 87.79% |
| NOTCH1 | 1.17007E−05 | 0.245943 | 18.54% | 48.25% |
| TLR9 | 1.19364E−05 | 0.666505 | 29.82% | 63.49% |
| TMPRSS5 | 2.16647E−05 | 0.682055 | 72.61% | 78.22% |
| ADRB1 | 2.52632E−05 | 0.79476 | 64.06% | 59.85% |
| SLC11A1 | 2.61715E−05 | 0.610752 | 12.36% | 37.17% |
| PVRL4 | 2.68139E−05 | 0.632457 | 32.75% | 56.31% |
| FAM171A1 | 2.97092E−05 | 0.294072 | 27.96% | 15.16% |
| ABCB9 | 3.13633E−05 | 0.509671 | 51.18% | 42.12% |
| DSG1 | 3.20907E−05 | 0.94698 | 91.97% | 88.74% |
| HTR3E | 3.29423E−05 | 0.997206 | 89.60% | 99.09% |
| TAS1R3 | 3.47021E−05 | 0.760305 | 16.46% | 65.48% |
| GAS1 | 3.58406E−05 | 0.467904 | 10.81% | 58.01% |
| EPHA1 | 5.58326E−05 | 0.537752 | 24.02% | 53.60% |
| CLEC5A | 5.65009E−05 | 0.589582 | 12.12% | 61.60% |
| ITGB6 | 8.66066E−05 | 0.692233 | 33.36% | 39.12% |
| IL13RA1 | 9.60468E−05 | 0.303483 | 11.93% | 13.26% |
| CD9 | 9.68695E−05 | 0.258285 | 16.13% | 15.78% |
| OR14A16 | 0.000103109 | 0.961317 | 98.01% | 99.51% |
| TEX101 | 0.000137167 | 0.86682 | 91.40% | 34.75% |
| PTPRJ | 0.000140474 | 0.277882 | 25.66% | 37.68% |
| EPHB4 | 0.00015882 | 0.216584 | 17.54% | 43.25% |
| KIAA0922 | 0.000170286 | 0.34579 | 18.62% | 21.31% |
| FOLH1 | 0.000178323 | 0.542073 | 53.28% | 72.90% |
| EPHB3 | 0.000224589 | 0.340999 | 39.45% | 31.73% |
| ITGB5 | 0.000241464 | 0.326944 | 11.45% | 25.41% |
| CLEC17A | 0.000284055 | 0.845813 | 14.15% | 81.27% |
| CHPT1 | 0.000361697 | 0.252501 | 15.30% | 27.59% |
| AQP5 | 0.000440324 | 0.568598 | 53.40% | 70.14% |
| LILRA2 | 0.000448315 | 0.817182 | 24.72% | 78.33% |
| IL1RAP | 0.000452298 | 0.669933 | 51.78% | 71.24% |
| CHRM1 | 0.000454436 | 0.808455 | 82.09% | 89.84% |
| LRIT3 | 0.00057546 | 0.397658 | 59.39% | 77.72% |
| SLC6A9 | 0.000586836 | 0.486214 | 24.42% | 44.73% |
| LRP5 | 0.000608646 | 0.239165 | 27.54% | 20.39% |
| GPR110 | 0.000740268 | 0.795993 | 44.62% | 85.39% |
| RNF43 | 0.000793841 | 0.383204 | 37.91% | 57.82% |
| GPR143 | 0.000814447 | 0.213691 | 52.06% | 42.03% |
| SLC6A7 | 0.000838794 | 0.627173 | 20.99% | 72.61% |
| KCNMB3 | 0.001025188 | 0.511001 | 58.81% | 41.27% |
| PRLR | 0.001144253 | 0.888585 | 61.88% | 94.68% |
| DSC2 | 0.001261101 | 0.487046 | 45.61% | 63.42% |
| ATP2B4 | 0.001790531 | 0.10117 | 10.46% | 26.99% |
| BACE2 | 0.001864832 | 0.414236 | 14.77% | 12.43% |
| LRP6 | 0.001951906 | 0.115569 | 23.77% | 15.84% |
| CD1E | 0.002157992 | 0.759057 | 17.91% | 77.03% |
| TNFSF11 | 0.002210775 | 0.679738 | 32.97% | 68.24% |
| NRG4 | 0.002285591 | 0.398112 | 51.25% | 52.91% |
| IL1RAPL2 | 0.002505288 | 0.457987 | 95.43% | 88.32% |
| IL1RL1 | 0.002745563 | 0.764687 | 31.62% | 73.26% |
| CNTN6 | 0.002751463 | 0.935146 | 77.71% | 73.55% |
| OR2A7 | 0.00289799 | 0.432388 | 58.76% | 65.25% |
| SLC29A2 | 0.002907563 | 0.296952 | 28.61% | 12.92% |
| VTCN1 | 0.003312723 | 0.77516 | 62.99% | 88.59% |
| SLC46A2 | 0.003323087 | 0.704155 | 13.31% | 43.70% |
| EPHA4 | 0.003346467 | 0.440623 | 27.09% | 14.41% |
| TRPV5 | 0.003567806 | 0.670506 | 70.53% | 77.68% |
| EGF | 0.003757905 | 0.556532 | 54.25% | 89.67% |
| SLC12A8 | 0.00377477 | 0.639211 | 37.13% | 58.14% |
| AQP2 | 0.004139175 | 0.82412 | 90.21% | 87.42% |
| HFE2 | 0.004236521 | 0.786573 | 78.33% | 77.85% |
| JAG1 | 0.004751604 | 0.219891 | 20.70% | 17.58% |
| ANO6 | 0.004763639 | 0.23775 | 17.03% | 11.49% |
| CEACAM19 | 0.004826399 | 0.50418 | 57.14% | 29.05% |
| TREM1 | 0.005602779 | 0.644712 | 22.52% | 26.77% |
| EVC2 | 0.005611979 | 0.405536 | 34.36% | 40.37% |
| PCSK5 | 0.005693818 | 0.583504 | 42.85% | 46.11% |
| SLC43A3 | 0.006162496 | 0.211633 | 15.76% | 18.03% |
| TYRP1 | 0.006263061 | 0.760874 | 45.57% | 44.20% |
| AGER | 0.0066642 | 0.722123 | 14.66% | 46.37% |
| BTC | 0.007271929 | 0.645059 | 44.81% | 56.34% |
| ERBB3 | 0.007379771 | 0.262827 | 33.67% | 32.52% |
| MSLN | 0.008068245 | 0.745137 | 29.16% | 47.69% |
| GPR133 | 0.008127868 | 0.565386 | 30.80% | 43.01% |
| SLC16A4 | 0.008452332 | 0.357789 | 15.24% | 44.01% |
| NGFR | 0.008472324 | 0.204603 | 13.17% | 55.54% |
| PKHD1 | 0.008499116 | 0.866847 | 69.66% | 45.70% |
| ITGA8 | 0.008556682 | 0.497222 | 11.06% | 45.40% |
| LYVE1 | 0.008799947 | 0.587227 | 24.74% | 46.19% |
| AMN | 0.009052556 | 0.60465 | 28.56% | 53.72% |
| KCNS1 | 0.009065972 | 0.722646 | 70.64% | 80.52% |
| F2RL1 | 0.009251378 | 0.302104 | 26.28% | 31.68% |
| TRPV6 | 0.01019293 | 0.145089 | 58.54% | 86.59% |
| CHRNA7 | 0.010193192 | 0.230165 | 41.52% | 72.70% |
| ANO2 | 0.010456449 | 0.133929 | 57.11% | 65.11% |
| SLC16A5 | 0.010880559 | 0.348931 | 15.95% | 38.28% |
| CHODL | 0.010930067 | 0.671464 | 81.38% | 74.88% |
| ROS1 | 0.011061816 | 0.742415 | 17.58% | 29.67% |
| SLC9A2 | 0.01119155 | 0.825633 | 77.12% | 54.51% |
| AQP4 | 0.011337328 | 0.6693 | 21.99% | 37.02% |
| ELFN2 | 0.011558687 | 0.13072 | 35.27% | 86.35% |
| SGCA | 0.01188718 | 0.645452 | 40.43% | 29.40% |
| TTYH1 | 0.011952592 | 0.847065 | 89.14% | 89.74% |
| CORIN | 0.012523347 | 0.562165 | 38.78% | 44.19% |
| LPAR3 | 0.012837212 | 0.559451 | 54.49% | 82.84% |
| SLC6A16 | 0.013156814 | 0.437973 | 62.00% | 66.96% |
| ADORA1 | 0.013572428 | 0.148568 | 35.58% | 61.66% |
| CD177 | 0.013943537 | 0.80316 | 80.76% | 36.40% |
| ADRA2B | 0.014117225 | 0.193016 | 30.92% | 57.33% |
| TSPAN2 | 0.015405045 | 0.158347 | 24.13% | 39.67% |
| FGFRL1 | 0.015601802 | 0.452219 | 29.65% | 53.08% |
| GJA3 | 0.015961745 | 0.915415 | 81.06% | 86.61% |
| MME | 0.016403426 | 0.676529 | 46.78% | 26.60% |
| RRH | 0.01684885 | 0.350216 | 56.50% | 52.34% |
| ENPP6 | 0.01703951 | 0.742891 | 50.38% | 67.09% |
| MUC13 | 0.017125072 | 0.89308 | 79.58% | 18.22% |
| DSC3 | 0.017909978 | 0.429529 | 53.45% | 83.21% |
| DCHS2 | 0.018051482 | 0.545891 | 88.85% | 86.04% |
| EFNB2 | 0.018374811 | 0.208394 | 12.40% | 21.32% |
| OR1G1 | 0.019567153 | 0.800716 | 64.39% | 86.33% |
| GPRC5A | 0.020114473 | 0.526023 | 22.32% | 31.99% |
| SCN7A | 0.020505778 | 0.64189 | 29.04% | 34.32% |
| ESYT3 | 0.020876712 | 0.354858 | 40.51% | 12.29% |
| ADRB3 | 0.020966445 | 0.614002 | 24.08% | 68.18% |
| ERBB2 | 0.021055046 | 0.280321 | 22.63% | 22.99% |
| ADCY6 | 0.02117691 | 0.168773 | 31.19% | 20.17% |
| OR7A5 | 0.021286953 | 0.950561 | 80.72% | 95.35% |
| GPR156 | 0.022083402 | 0.231931 | 42.06% | 64.35% |
| SCTR | 0.023375499 | 0.62339 | 29.68% | 24.92% |
| SLC7A10 | 0.026205032 | 0.721705 | 84.47% | 81.12% |
| GPRC5D | 0.026497893 | 0.188734 | 42.94% | 37.86% |
| SLC1A1 | 0.026903816 | 0.349672 | 10.61% | 27.55% |
| SLC24A4 | 0.028137202 | 0.528737 | 67.10% | 89.70% |
| ACE2 | 0.028432856 | 0.479573 | 21.69% | 46.59% |
| GUCY2C | 0.028712571 | 0.305401 | 18.38% | 57.20% |
| GPR17 | 0.030094261 | 0.876818 | 83.58% | 93.97% |
| PLP1 | 0.030391865 | 0.598853 | 67.18% | 81.10% |
| LRP8 | 0.030965801 | 0.1222 | 34.17% | 27.74% |
| CCR3 | 0.033322703 | 0.774373 | 51.33% | 78.62% |
| NPY2R | 0.034661438 | 0.959179 | 97.39% | 97.85% |
| ANO3 | 0.035055085 | 0.682266 | 53.81% | 58.82% |
| SLCO6A1 | 0.036341673 | 0.725141 | 71.48% | 44.17% |
| OR51I1 | 0.037933366 | 0.818566 | 82.86% | 71.72% |
| MMP17 | 0.038688354 | 0.446805 | 38.26% | 54.76% |
| TYRO3 | 0.040249495 | 0.125142 | 19.95% | 33.37% |
| NPY1R | 0.041073669 | 0.676799 | 12.45% | 78.71% |
| MRGPRD | 0.041485825 | 0.341437 | 71.93% | 76.06% |
| CLDN10 | 0.042522253 | 0.514514 | 76.49% | 48.08% |
| NOTCH3 | 0.043919721 | 0.203597 | 15.43% | 23.51% |
| KCNMB4 | 0.044449222 | 0.319162 | 32.24% | 26.41% |
| OXGR1 | 0.045050137 | 0.689548 | 43.03% | 62.11% |
| EPHB6 | 0.045424814 | 0.254378 | 12.66% | 55.93% |
| OR1E1 | 0.045916035 | 0.888355 | 67.73% | 84.42% |
| CX3CR1 | 0.048647041 | 0.776186 | 38.40% | 77.43% |
| OR7C1 | 0.049394983 | 0.740652 | 63.17% | 83.97% |
| SLC5A9 | 0.049668309 | 0.573112 | 31.52% | 44.80% |
| GPC4 | 0.051678748 | 0.313392 | 20.86% | 30.10% |
| GJB7 | 0.05273889 | 0.712129 | 61.87% | 43.01% |
| ATP1A2 | 0.057053662 | 0.65187 | 62.91% | 49.30% |
| SLC34A2 | 0.057359102 | 0.472189 | 11.80% | 10.23% |
| TACSTD2 | 0.058819435 | 0.366904 | 17.51% | 30.18% |
| MUC16 | 0.060526781 | 0.407618 | 65.55% | 86.69% |
| SLC19A3 | 0.060674687 | 0.685987 | 35.54% | 31.08% |
| PTH1R | 0.06247867 | 0.49763 | 23.42% | 18.05% |
| VSIG2 | 0.062792439 | 0.357679 | 38.39% | 40.58% |
| ROR1 | 0.063145223 | 0.175749 | 41.48% | 42.48% |
| PTK7 | 0.064276357 | 0.19172 | 16.76% | 21.59% |
| TAS2R38 | 0.064796129 | 0.852551 | 93.97% | 79.30% |
| SLC6A4 | 0.065880531 | 0.844789 | 32.53% | 56.72% |
| PROM2 | 0.066147525 | 0.215337 | 42.47% | 39.03% |
| DSG2 | 0.066454771 | 0.194862 | 28.70% | 21.48% |
| HTR3B | 0.066612063 | 0.477303 | 85.44% | 71.38% |
| AVPR2 | 0.067733761 | 0.275832 | 48.53% | 49.11% |
| TAS2R9 | 0.068395293 | 0.463287 | 76.01% | 74.39% |
| TM4SF4 | 0.070625087 | 0.922386 | 89.76% | 84.08% |
| SHISA6 | 0.070637439 | 0.452698 | 87.07% | 92.28% |
| TMEM171 | 0.072804998 | 0.575858 | 28.52% | 17.58% |
| MEGF10 | 0.073549936 | 0.257342 | 79.53% | 96.08% |
| IL13RA2 | 0.075093846 | 0.679916 | 39.52% | 64.57% |
| TMEM211 | 0.07532478 | 0.849341 | 89.72% | 71.53% |
| S1PR3 | 0.07964697 | 0.270132 | 15.51% | 18.27% |
| SLC39A2 | 0.081436028 | 0.758829 | 75.78% | 97.87% |
| OR2M3 | 0.081772548 | 0.702289 | 86.93% | 76.87% |
| ITGB8 | 0.083510372 | 0.183599 | 31.49% | 32.20% |
| DPCR1 | 0.083941913 | 0.67573 | 36.31% | 68.62% |
| ABCC9 | 0.085480661 | 0.176252 | 21.87% | 16.19% |
| ATP13A5 | 0.085632442 | 0.382861 | 56.91% | 80.27% |
| SLC10A2 | 0.085821319 | 0.884712 | 80.44% | 87.16% |
| ABCB4 | 0.086819783 | 0.544165 | 35.97% | 68.09% |
| PTGFRN | 0.087535731 | 0.150178 | 19.58% | 14.59% |
| SLC2A4 | 0.087649817 | 0.543777 | 38.05% | 26.55% |
| SLCO2A1 | 0.088181013 | 0.116896 | 11.41% | 29.48% |
| NLGN3 | 0.092107476 | 0.268587 | 53.37% | 51.79% |
| CHRNE | 0.092608577 | 0.345935 | 15.86% | 45.25% |
| ABCA8 | 0.093124763 | 0.588744 | 48.62% | 55.18% |
| PTPRQ | 0.094850636 | 0.701677 | 18.98% | 35.27% |
| HTR2A | 0.095218297 | 0.572068 | 23.28% | 58.67% |
| FGFR4 | 0.097040066 | 0.587395 | 36.66% | 38.46% |
| FLRT3 | 0.097754914 | 0.388623 | 26.86% | 43.47% |
| NRG2 | 0.09799727 | 0.536332 | 71.87% | 75.05% |
| MAG | 0.098341934 | 0.594878 | 72.86% | 78.35% |
| P2RX2 | 0.099264374 | 0.571596 | 18.61% | 71.53% |
| CEACAM1 | 0.099670836 | 0.487904 | 55.32% | 33.66% |
| GABRP | 0.099691256 | 0.322992 | 37.31% | 62.97% |
| EMR3 | 0.099973368 | 0.719821 | 33.57% | 50.23% |
| EFNA3 | 0.100561442 | 0.224619 | 32.69% | 28.55% |
| TAS2R1 | 0.102303043 | 0.618894 | 53.75% | 70.86% |
| SVOPL | 0.10469247 | 0.691319 | 58.68% | 29.67% |
| GPR126 | 0.105320886 | 0.824585 | 49.19% | 32.83% |
| OR2C3 | 0.105426763 | 0.93233 | 87.34% | 96.90% |
| LRRN4CL | 0.105470732 | 0.50418 | 39.22% | 54.38% |
| LPAR4 | 0.105903885 | 0.725048 | 76.72% | 88.79% |
| TAS2R4 | 0.107228562 | 0.160054 | 63.62% | 50.39% |
| OR2L2 | 0.107945954 | 0.890086 | 100.00% | 63.35% |
| MPZ | 0.115877849 | 0.350074 | 33.52% | 25.02% |
| OR2AK2 | 0.120352195 | 0.85132 | 82.30% | 54.54% |
| BTNL9 | 0.127231925 | 0.115219 | 11.28% | 43.09% |
| OR2H2 | 0.127823089 | 0.577927 | 60.73% | 54.18% |
| UPK1A | 0.127948226 | 0.581606 | 68.03% | 43.60% |
| NPY5R | 0.130569764 | 0.786431 | 12.13% | 88.92% |
| TAS2R10 | 0.131962692 | 0.434925 | 69.51% | 64.23% |
| MTNR1A | 0.136531947 | 0.77608 | 46.03% | 50.89% |
| MMP16 | 0.137405514 | 0.343624 | 55.40% | 27.58% |
| TAS1R2 | 0.137648238 | 0.968373 | 98.39% | 96.85% |
| CDH17 | 0.137679582 | 0.792077 | 75.06% | 76.23% |
| OR10H1 | 0.140328417 | 0.675839 | 78.70% | 86.57% |
| TMPRSS11E | 0.141515309 | 0.869983 | 82.81% | 69.78% |
| OR52N2 | 0.145823719 | 0.822447 | 36.49% | 26.20% |
| ZP4 | 0.149279396 | 1 | 92.86% | 97.83% |
| GJB3 | 0.149663808 | 0.492994 | 46.99% | 55.77% |
| MFSD6L | 0.151785406 | 0.473599 | 46.92% | 20.73% |
| LPPR5 | 0.155375697 | 0.331811 | 88.30% | 88.33% |
| MC5R | 0.155724755 | 0.199239 | 50.79% | 71.24% |
| CDH16 | 0.156400295 | 0.997076 | 99.06% | 97.74% |
| OR3A1 | 0.159340672 | 0.766853 | 72.53% | 72.42% |
| ITGB3 | 0.160963176 | 0.23081 | 34.70% | 40.06% |
| SLC5A4 | 0.161854327 | 0.396068 | 50.95% | 61.50% |
| SLC26A8 | 0.163162981 | 0.483082 | 55.01% | 33.53% |
| CLDN16 | 0.164648344 | 0.426791 | 44.67% | 67.09% |
| NPR3 | 0.165148184 | 0.487012 | 10.71% | 27.82% |
| GABRA6 | 0.165706626 | 0.793245 | 86.08% | 71.01% |
| CD300LD | 0.165804434 | 0.958912 | 64.88% | 93.86% |
| LHCGR | 0.166864269 | 0.179474 | 64.50% | 86.48% |
| GHRHR | 0.169093244 | 0.11812 | 71.63% | 87.84% |
| MUSK | 0.175886446 | 0.656954 | 10.24% | 52.42% |
| AVPR1A | 0.17732047 | 0.270305 | 35.14% | 50.57% |
| CDH19 | 0.177565353 | 0.896903 | 63.29% | 85.95% |
| CLDN2 | 0.178639236 | 0.656958 | 43.46% | 44.38% |
| TRPV4 | 0.181421521 | 0.177181 | 28.21% | 66.97% |
| SLC1A6 | 0.187838755 | 0.884619 | 94.00% | 87.71% |
| OPCML | 0.188996811 | 0.114824 | 63.15% | 61.82% |
| PCDHGA11 | 0.191438613 | 0.5688 | 70.61% | 62.14% |
| PCDHGB7 | 0.191500813 | 0.420732 | 19.72% | 40.33% |
| OR56A1 | 0.193811563 | 0.899266 | 96.75% | 91.92% |
| OR2D2 | 0.194279947 | 0.710616 | 34.58% | 70.56% |
| OR5AK2 | 0.194720213 | 0.843427 | 83.52% | 68.30% |
| TAS2R30 | 0.197744614 | 0.702948 | 89.07% | 79.87% |
| SLC3A1 | 0.198821818 | 0.368173 | 44.28% | 29.66% |
| EDNRB | 0.199593812 | 0.37187 | 26.10% | 41.12% |
| OR52E6 | 0.20203476 | 1 | 39.14% | 15.56% |
| TRHR | 0.206396211 | 0.206114 | 18.56% | 79.22% |
| CEACAM8 | 0.207601332 | 0.445245 | 68.62% | 85.69% |
| SCN2B | 0.208183103 | 0.443652 | 40.18% | 61.08% |
| GPR87 | 0.210507679 | 0.71056 | 79.26% | 72.67% |
| NIPAL4 | 0.210619893 | 0.477055 | 23.27% | 41.40% |
| PCDH1 | 0.215677745 | 0.179956 | 27.49% | 16.78% |
| ZP2 | 0.221450708 | 0.844645 | 66.81% | 38.44% |
| LPL | 0.225272333 | 0.213236 | 21.72% | 13.14% |
| TAS2R14 | 0.231893619 | 0.159452 | 36.82% | 24.55% |
| RGR | 0.235820069 | 0.572266 | 67.41% | 94.45% |
| GRIA4 | 0.239230762 | 0.952162 | 75.07% | 58.06% |
| SLC22A1 | 0.239271045 | 0.333878 | 43.18% | 42.43% |
| OR13A1 | 0.243147239 | 0.385063 | 45.19% | 12.82% |
| OR3A3 | 0.248306294 | 0.562802 | 92.37% | 92.85% |
| TAS2R20 | 0.253720488 | 0.18062 | 30.82% | 20.27% |
| TREH | 0.257881966 | 0.884718 | 77.11% | 54.76% |
| LYPD2 | 0.266284276 | 0.118249 | 52.31% | 77.30% |
| OR10AD1 | 0.266518291 | 0.414722 | 60.90% | 37.05% |
| SLC6A15 | 0.266704162 | 0.57333 | 41.10% | 38.48% |
| SPACA1 | 0.269350373 | 0.945545 | 100.00% | 74.77% |
| IL22RA1 | 0.270446327 | 0.247689 | 33.30% | 33.17% |
| TGFA | 0.270467452 | 0.350554 | 31.84% | 35.48% |
| XPNPEP2 | 0.271584399 | 0.544586 | 40.62% | 55.07% |
| FFAR1 | 0.273068174 | 0.6556 | 65.33% | 62.51% |
| OR51B4 | 0.281462593 | 0.927919 | 69.49% | 21.91% |
| SELE | 0.28581219 | 0.503158 | 18.06% | 35.70% |
| HLA-DRB5 | 0.289245803 | 0.307817 | 10.35% | 24.63% |
| ITGA6 | 0.290825792 | 0.133514 | 13.24% | 15.19% |
| DUOXA1 | 0.290944834 | 0.275387 | 31.60% | 31.21% |
| LEPR | 0.295103641 | 0.368354 | 24.64% | 23.25% |
| CDH3 | 0.301911808 | 0.253595 | 22.64% | 20.82% |
| IGDCC4 | 0.303287665 | 0.17724 | 15.81% | 46.68% |
| OR5M11 | 0.308950386 | 0.916373 | 39.38% | 73.52% |
| TAS2R8 | 0.31169874 | 0.739599 | 55.64% | 76.01% |
| OR56A3 | 0.324780124 | 0.941395 | 55.34% | 25.53% |
| TPO | 0.325197567 | 0.83885 | 83.14% | 38.48% |
| OR6K3 | 0.326247755 | 0.897422 | 47.56% | 52.44% |
| FSHR | 0.332841152 | 0.553428 | 100.00% | 92.10% |
| HTR2C | 0.335918418 | 0.999735 | 99.97% | 72.20% |
| SLCO1B3 | 0.336276389 | 0.86063 | 92.82% | 69.90% |
| HTR3D | 0.337826954 | 0.459008 | 88.42% | 39.63% |
| PCDHGA2 | 0.341933737 | 0.276588 | 51.88% | 26.31% |
| PMEPA1 | 0.347625064 | 0.161026 | 20.53% | 16.49% |
| RHAG | 0.349959661 | 0.526784 | 83.06% | 61.50% |
| MDGA2 | 0.35062656 | 0.970358 | 89.32% | 63.82% |
| MRGPRG | 0.350733511 | 0.284351 | 94.91% | 75.73% |
| TAS1R1 | 0.364489067 | 0.347182 | 57.30% | 66.01% |
| SLC5A8 | 0.371037101 | 0.609705 | 13.17% | 21.11% |
| OR7G2 | 0.373781402 | 0.775875 | 100.00% | 29.43% |
| GPR128 | 0.374950878 | 0.998037 | 99.72% | 55.47% |
| EREG | 0.383464698 | 0.315331 | 10.39% | 69.81% |
| SLC39A5 | 0.39030688 | 0.386952 | 53.11% | 42.58% |
| OR56A4 | 0.390513457 | 0.860972 | 100.00% | 50.01% |
| SLC10A5 | 0.391236381 | 0.199345 | 30.43% | 10.24% |
| TM4SF20 | 0.394075952 | 0.209688 | 55.41% | 25.14% |
| BTN1A1 | 0.398177117 | 0.496319 | 46.37% | 14.84% |
| GPR22 | 0.398560689 | 0.826058 | 93.32% | 39.56% |
| SCARA5 | 0.39886845 | 0.57999 | 56.65% | 60.70% |
| TNFRSF25 | 0.400610662 | 0.153979 | 20.21% | 16.25% |
| MUC4 | 0.407044062 | 0.272589 | 24.35% | 44.32% |
| PCDHB7 | 0.409713081 | 0.319223 | 35.63% | 15.31% |
| PCDH7 | 0.434676508 | 0.508357 | 27.30% | 21.02% |
| TDGF1 | 0.444145245 | 0.560496 | 35.33% | 28.94% |
| RTN4RL2 | 0.44922469 | 0.176964 | 15.00% | 26.77% |
| SORCS1 | 0.449766671 | 0.789279 | 75.16% | 62.12% |
| NTRK2 | 0.454201266 | 0.205316 | 29.43% | 52.05% |
| OR52N1 | 0.454661384 | 0.849011 | 67.95% | 64.44% |
| OR7E24 | 0.456797167 | 0.869033 | 51.89% | 58.91% |
| TMEM27 | 0.466431493 | 0.193446 | 19.46% | 22.74% |
| SLC26A4 | 0.47014402 | 0.283031 | 40.83% | 52.24% |
| OR10Q1 | 0.477575038 | 0.479528 | 100.00% | 32.02% |
| GABRG2 | 0.478414967 | 0.699959 | 37.19% | 30.08% |
| BTNL3 | 0.480748272 | 0.613497 | 21.03% | 41.83% |
| TMEM213 | 0.483184799 | 0.402734 | 57.32% | 49.02% |
| UMOD | 0.49405328 | 0.737087 | 54.73% | 56.20% |
| TMPRSS13 | 0.496987784 | 0.301886 | 33.55% | 44.43% |
| SLC6A8 | 0.500550185 | 0.117882 | 10.01% | 19.14% |
| ERVV-1 | 0.50134262 | 0.2678 | 61.26% | 44.17% |
| CACHD1 | 0.508789618 | 0.16718 | 13.01% | 18.37% |
| OR2C1 | 0.512670511 | 0.137443 | 32.18% | 34.31% |
| OR3A2 | 0.531951762 | 0.271244 | 86.86% | 88.31% |
| SLC1A7 | 0.552627356 | 0.126664 | 50.58% | 65.17% |
| SLC5A11 | 0.573269145 | 0.211991 | 87.84% | 30.69% |
| OR2M4 | 0.580555576 | 0.73969 | 100.00% | 34.32% |
| VIPR2 | 0.592772318 | 0.487689 | 51.39% | 27.96% |
| GPR20 | 0.594023087 | 0.345519 | 16.94% | 26.54% |
| SLC17A8 | 0.603554458 | 0.358808 | 61.62% | 59.02% |
| TPSG1 | 0.604243563 | 0.506422 | 43.40% | 14.99% |
| ABCA13 | 0.613374042 | 0.363266 | 30.31% | 53.52% |
| OR2T3 | 0.622433332 | 0.571684 | 100.00% | 27.56% |
| SLC22A12 | 0.623032751 | 0.169417 | 100.00% | 24.19% |
| OR11L1 | 0.624516845 | 0.407419 | 59.54% | 78.07% |
| OR2K2 | 0.633368729 | 0.366968 | 44.71% | 51.31% |
| KIR3DL3 | 0.659474269 | 0.661364 | 39.74% | 15.36% |
| SLC18A3 | 0.6599718 | 0.306886 | 37.11% | 40.94% |
| OR10A3 | 0.67529466 | 0.788284 | 62.84% | 18.54% |
| CNTN1 | 0.679674951 | 0.145236 | 36.97% | 19.45% |
| OR11H12 | 0.703757044 | 0.230856 | 74.21% | 47.76% |
| PTGER3 | 0.710831509 | 0.394932 | 61.96% | 42.77% |
| SERINC5 | 0.721823022 | 0.112287 | 19.21% | 12.95% |
| MLNR | 0.724905932 | 0.17268 | 39.04% | 16.80% |
| DUOX1 | 0.727206229 | 0.122774 | 18.37% | 21.26% |
| LRRC4 | 0.742301207 | 0.123438 | 10.33% | 19.47% |
| OR2A2 | 0.754863653 | 0.489595 | 34.11% | 58.86% |
| ERVFRD-1 | 0.783008795 | 0.355158 | 31.97% | 25.50% |
| GRM5 | 0.787467035 | 0.291893 | 55.92% | 23.65% |
| GPM6A | 0.798661537 | 0.446923 | 41.10% | 26.42% |
| PCDH15 | 0.80956288 | 0.414102 | 49.67% | 41.97% |
| CDH6 | 0.819645372 | 0.173842 | 22.98% | 11.76% |
| GPR78 | 0.875975682 | 0.394652 | 58.66% | 61.84% |
| SLC22A13 | 0.906218245 | 0.109789 | 17.26% | 20.63% |
| LY6K | 0.9128643 | 0.219513 | 23.59% | 27.02% |
| RXFP4 | 0.918452229 | 0.211565 | 11.51% | 30.11% |
| OR5K1 | 0.939867811 | 0.221892 | 56.54% | 46.71% |
| CLDN1 | 0.963511597 | 0.111213 | 13.19% | 11.12% |
| IL27RA | 3.80835E−10 | 0.491909 | 1.57% | 38.05% |
| TMEM37 | 2.07883E−08 | 0.401771 | 2.69% | 38.32% |
| FZD7 | 1.05725E−07 | 0.291174 | 5.62% | 56.74% |
| TNFRSF1A | 2.97304E−07 | 0.293602 | 5.05% | 20.43% |
| SIRPB2 | 1.66205E−06 | 0.738437 | 7.51% | 69.80% |
| TFPI | 6.13469E−06 | 0.476096 | 8.02% | 24.64% |
| PTGDR | 6.70351E−06 | 0.656201 | 0.24% | 61.19% |
| CD70 | 7.94973E−06 | 0.778936 | 0.47% | 67.60% |
| UNC5C | 3.31841E−05 | 0.679531 | 7.70% | 57.95% |
| CD55 | 3.34123E−05 | 0.369519 | 8.42% | 15.47% |
| PDGFRA | 9.46902E−05 | 0.48919 | 1.73% | 35.02% |
| ICAM4 | 0.000118391 | 0.685901 | 1.99% | 56.94% |
| AOC3 | 0.000250065 | 0.417545 | 2.29% | 10.73% |
| CX3CL1 | 0.000260353 | 0.311673 | 6.99% | 32.06% |
| GPR125 | 0.000292712 | 0.019715 | 21.19% | 16.00% |
| LTBR | 0.000322781 | 0.230382 | 5.91% | 8.51% |
| EMP1 | 0.000423485 | 0.276759 | 7.15% | 24.47% |
| PROM1 | 0.000556949 | 0.358625 | 56.74% | 3.99% |
| RNFT1 | 0.000595944 | 0.157005 | 1.32% | 5.40% |
| CRIM1 | 0.000752526 | 0.211906 | 0.79% | 24.63% |
| QSOX1 | 0.000949294 | 0.225639 | 7.44% | 12.53% |
| NOTCH2 | 0.001391497 | 0.432679 | 9.91% | 35.44% |
| LDLR | 0.002098538 | 0.30669 | 23.44% | 1.44% |
| SLC29A1 | 0.002665488 | 0.197281 | 15.85% | 4.54% |
| TMEM87B | 0.002787639 | 0.150347 | 0.88% | 1.18% |
| NRP1 | 0.003486853 | 0.189763 | 5.00% | 34.02% |
| TM7SF3 | 0.004471624 | 0.150418 | 5.20% | 3.64% |
| DUOX2 | 0.004501498 | 0.063127 | 47.16% | 61.35% |
| LYPD3 | 0.004916422 | 0.36759 | 35.87% | 9.05% |
| IGF1R | 0.005824442 | 0.001382 | 18.15% | 23.42% |
| DAGLB | 0.006306386 | 0.101116 | 2.85% | 18.77% |
| ATP1A1 | 0.006719033 | 0.138506 | 8.71% | 5.99% |
| PPAP2B | 0.006922599 | 0.215878 | 4.79% | 15.20% |
| SLC12A2 | 0.007080979 | 0.050994 | 19.62% | 22.26% |
| ABCC4 | 0.007336235 | 0.419182 | 7.37% | 40.77% |
| SLC11A2 | 0.007926953 | 0.216816 | 12.87% | 8.52% |
| SHISA9 | 0.007978787 | 0.001578 | 40.78% | 9.21% |
| TMEM9B | 0.008107124 | 0.112142 | 1.90% | 0.63% |
| GRPR | 0.008826539 | 0.09859 | 76.15% | 68.85% |
| OR52N4 | 0.008933724 | 0.790776 | 6.85% | 67.48% |
| HEG1 | 0.009239977 | 0.17681 | 0.66% | 27.88% |
| LRRN2 | 0.009827145 | 0.081034 | 24.82% | 42.49% |
| FURIN | 0.009833891 | 0.165856 | 5.20% | 6.86% |
| CDCP1 | 0.009930141 | 0.323386 | 17.32% | 9.31% |
| ROBO3 | 0.010179135 | 0.016396 | 14.70% | 61.69% |
| KCNMB1 | 0.010785151 | 0.339065 | 2.12% | 30.07% |
| IGSF9 | 0.011837538 | 0.03483 | 1.96% | 27.93% |
| ADAM15 | 0.011971108 | 0.296194 | 9.59% | 15.63% |
| IL17RB | 0.012038765 | 0.093433 | 30.02% | 12.59% |
| P2RY1 | 0.012285131 | 0.264869 | 6.51% | 51.61% |
| FGFR1 | 0.013687141 | 0.464362 | 15.19% | 3.87% |
| ENPP5 | 0.014027223 | 0.162987 | 27.72% | 7.02% |
| GPR55 | 0.015085835 | 0.072386 | 5.22% | 58.55% |
| GPR157 | 0.016995435 | 0.552128 | 3.58% | 32.33% |
| PKD2 | 0.017914637 | 0.0393 | 6.18% | 18.66% |
| MFI2 | 0.018053005 | 0.0917 | 34.51% | 13.46% |
| ADAM32 | 0.020247857 | 0.64185 | 9.50% | 46.15% |
| GLDN | 0.021536127 | 0.503465 | 53.98% | 3.46% |
| MANSC4 | 0.021762601 | 0.499754 | 1.05% | 55.22% |
| CD109 | 0.022506005 | 0.031266 | 27.38% | 50.34% |
| CALHM2 | 0.024631579 | 0.114388 | 3.25% | 23.65% |
| TMEM62 | 0.026593448 | 0.193535 | 6.37% | 9.31% |
| S1PR2 | 0.027717445 | 0.210772 | 15.84% | 9.68% |
| MUC1 | 0.034007566 | 0.370313 | 14.97% | 5.45% |
| AQP1 | 0.03425542 | 0.240582 | 4.53% | 22.90% |
| SEMA5A | 0.035410634 | 0.073182 | 21.71% | 42.09% |
| LAYN | 0.036306834 | 0.005031 | 2.33% | 23.91% |
| TM9SF4 | 0.036561803 | 0.076365 | 2.75% | 0.55% |
| ICAM5 | 0.037135955 | 0.43688 | 1.74% | 28.60% |
| FAT1 | 0.041493222 | 0.233061 | 14.15% | 5.17% |
| GPR116 | 0.042591638 | 0.227676 | 0.72% | 20.74% |
| ADAM10 | 0.044380072 | 0.109811 | 9.08% | 10.06% |
| CDON | 0.045400463 | 0.088132 | 27.52% | 24.83% |
| CD58 | 0.049115226 | 0.244785 | 4.97% | 16.26% |
| GABRE | 0.049254216 | 0.087662 | 31.34% | 62.00% |
| NOX4 | 0.049597451 | 0.431235 | 2.21% | 33.10% |

TABLE 8
Surface proteins associated with the SCLC-P subtype from the cell line dataset

| | ANOVA P | | | |
|---|---|---|---|---|
| Surface Protein | value | P vs N | P vs I | P vs A |
| OR2W3 | 1.84063E−09 | 0.973307 | 78.97% | 90.63% |
| TNFRSF25 | 3.18647E−08 | 0.403317 | 35.77% | 15.18% |
| P2RY8 | 1.69851E−06 | 0.886062 | 100.00% | 94.18% |
| ANO9 | 2.00245E−06 | 0.609042 | 92.17% | 10.81% |
| CDCP1 | 3.27424E−06 | 0.561207 | 66.07% | 14.00% |
| OR2T8 | 6.0221E−06 | 0.927161 | 85.65% | 84.59% |
| IL17RB | 2.00076E−05 | 0.184279 | 19.91% | 13.25% |
| SIGIRR | 2.27068E−05 | 0.699622 | 68.09% | 15.97% |
| NIPAL1 | 3.47959E−05 | 0.583635 | 57.00% | 22.38% |
| OR2A1 | 4.98952E−05 | 0.949456 | 67.14% | 84.51% |
| ART3 | 8.81977E−05 | 0.55051 | 58.96% | 45.47% |
| PCSK5 | 0.000193993 | 0.4293 | 71.49% | 40.80% |
| GJC3 | 0.000224967 | 0.297753 | 25.50% | 13.12% |
| NRG2 | 0.000241036 | 0.104315 | 32.85% | 58.77% |
| MPZL2 | 0.000303634 | 0.420672 | 68.20% | 15.96% |
| IL13RA1 | 0.000321744 | 0.385055 | 27.47% | 21.82% |
| OR2AJ1 | 0.000451239 | 0.887388 | 72.04% | 87.04% |
| ACE2 | 0.000500139 | 0.422543 | 63.73% | 38.24% |
| OR2L8 | 0.000551402 | 0.80432 | 52.51% | 83.54% |
| OR2AK2 | 0.000569755 | 0.758023 | 73.85% | 79.80% |
| HTR1E | 0.000596264 | 0.217146 | 87.73% | 79.00% |
| CD8A | 0.000633729 | 0.823627 | 85.03% | 82.00% |
| OR2A7 | 0.000801993 | 0.920764 | 70.45% | 68.49% |
| TAS2R38 | 0.000848049 | 0.839025 | 100.00% | 52.91% |
| CLEC5A | 0.001010686 | 0.490292 | 62.28% | 14.91% |
| NRP1 | 0.001075071 | 0.374064 | 12.26% | 11.35% |
| CD8B | 0.001192305 | 0.616522 | 87.57% | 66.30% |
| TAS2R8 | 0.001258191 | 0.50259 | 26.49% | 20.55% |
| ERBB3 | 0.001304319 | 0.419577 | 43.11% | 22.79% |
| PPAP2C | 0.001484947 | 0.697998 | 52.35% | 25.32% |
| OR10W1 | 0.001670769 | 0.838678 | 56.47% | 94.68% |
| EPHA1 | 0.001699042 | 0.371204 | 24.11% | 59.20% |
| GPR141 | 0.001776037 | 0.850228 | 100.00% | 83.30% |
| CRB3 | 0.001848833 | 0.666753 | 67.25% | 16.84% |
| TAS2R9 | 0.001858952 | 0.53866 | 13.89% | 12.22% |
| GJB7 | 0.001915966 | 0.360237 | 31.48% | 18.53% |
| ITGA8 | 0.00249817 | 0.515019 | 39.38% | 63.52% |
| OR2L3 | 0.00338085 | 0.808428 | 65.83% | 82.10% |
| OR6V1 | 0.003486184 | 0.379756 | 16.40% | 18.61% |
| DCHS2 | 0.003868332 | 0.551525 | 40.71% | 53.84% |
| OR1N1 | 0.003946122 | 0.716867 | 86.87% | 63.89% |
| BACE2 | 0.003948012 | 0.520011 | 29.78% | 13.29% |
| PVR | 0.003952176 | 0.140649 | 16.24% | 22.99% |
| OR1S1 | 0.003978803 | 0.991257 | 71.42% | 90.88% |
| OR1N2 | 0.004171406 | 0.682637 | 86.09% | 56.31% |
| OR2A2 | 0.004429299 | 0.995638 | 68.50% | 63.58% |
| OR2L2 | 0.005055368 | 0.824659 | 61.11% | 77.99% |
| TAS2R3 | 0.005721531 | 0.242471 | 28.24% | 13.37% |
| OR1B1 | 0.005829807 | 0.694422 | 89.24% | 59.70% |
| OR2L5 | 0.00646865 | 0.890388 | 67.95% | 75.11% |
| TMEM27 | 0.006509446 | 0.230813 | 37.03% | 15.62% |
| FOLH1 | 0.006676013 | 0.625949 | 39.07% | 67.97% |
| RELT | 0.007030806 | 0.149871 | 67.44% | 20.00% |
| OR9Q2 | 0.007379378 | 0.990776 | 100.00% | 85.83% |
| SCN4A | 0.007459589 | 0.606443 | 66.83% | 66.40% |
| CLDN1 | 0.007493518 | 0.720816 | 22.10% | 23.10% |
| ABCG5 | 0.007626799 | 0.249168 | 19.37% | 11.07% |
| OR1L1 | 0.007730612 | 0.628269 | 92.37% | 61.76% |
| GJB2 | 0.008920645 | 0.921522 | 36.79% | 51.62% |
| OR5B21 | 0.009408114 | 0.935063 | 50.68% | 75.83% |
| TLR5 | 0.011131903 | 0.201083 | 49.00% | 28.05% |
| OR1L3 | 0.011147399 | 0.681512 | 70.15% | 45.38% |
| FGFRL1 | 0.011434613 | 0.37889 | 19.30% | 56.73% |
| ANO7 | 0.011733181 | 0.627077 | 70.57% | 60.74% |
| FAT1 | 0.012132693 | 0.339811 | 17.16% | 14.90% |
| OR1Q1 | 0.013140994 | 0.565595 | 74.42% | 44.35% |
| CEACAM19 | 0.013216645 | 0.304763 | 46.49% | 32.64% |
| OR9A4 | 0.013839863 | 0.647544 | 100.00% | 34.65% |
| OR2A14 | 0.015508627 | 0.993415 | 81.58% | 67.65% |
| TAS2R10 | 0.016026464 | 0.326119 | 15.83% | 12.88% |
| GPR110 | 0.016469961 | 0.726944 | 59.84% | 60.86% |
| CX3CL1 | 0.017536946 | 0.117163 | 100.00% | 71.11% |
| TAS2R4 | 0.0185871 | 0.201529 | 29.12% | 14.53% |
| XKR3 | 0.018628148 | 0.545371 | 18.87% | 70.91% |
| EPHB3 | 0.018940533 | 0.502897 | 29.63% | 47.52% |
| TMPRSS5 | 0.018969675 | 0.589436 | 71.78% | 30.85% |
| OR9Q1 | 0.018983602 | 0.917247 | 81.20% | 81.27% |
| TLR2 | 0.019100789 | 0.470826 | 51.75% | 53.64% |
| OR9A2 | 0.019317845 | 0.516833 | 31.23% | 28.47% |
| DSG1 | 0.019795728 | 0.567852 | 54.76% | 55.28% |
| OR13A1 | 0.021116533 | 0.716623 | 84.64% | 47.81% |
| FAT3 | 0.021138231 | 0.289766 | 23.34% | 25.15% |
| OR1L8 | 0.022372362 | 0.643219 | 80.77% | 51.71% |
| IL4R | 0.023899289 | 0.335102 | 30.77% | 58.12% |
| ENPP6 | 0.025088121 | 0.335612 | 21.96% | 75.90% |
| EFNB2 | 0.025356024 | 0.169393 | 22.74% | 26.45% |
| FGFR4 | 0.0262064 | 0.296085 | 37.36% | 53.94% |
| SLC9A2 | 0.027190369 | 0.365608 | 41.31% | 19.11% |
| CDH3 | 0.027397592 | 0.453031 | 51.62% | 30.17% |
| CR2 | 0.028556562 | 0.642956 | 68.14% | 62.41% |
| ADRA1A | 0.028845367 | 0.426984 | 85.82% | 73.90% |
| OR1S2 | 0.02916823 | 0.717935 | 100.00% | 83.34% |
| MME | 0.030756491 | 0.591549 | 14.03% | 36.32% |
| KLRB1 | 0.032475047 | 0.213607 | 15.92% | 10.26% |
| SLC10A2 | 0.03275455 | 0.851805 | 70.74% | 75.16% |
| DSC2 | 0.033233433 | 0.520117 | 46.48% | 28.57% |
| TAS2R7 | 0.033841624 | 0.614803 | 26.32% | 33.24% |
| OR13C9 | 0.034277568 | 0.740651 | 100.00% | 28.60% |
| PDGFRA | 0.034416122 | 0.451549 | 10.86% | 46.23% |
| OR2A25 | 0.03552652 | 0.938721 | 100.00% | 71.03% |
| OR2A12 | 0.035687425 | 0.994012 | 60.25% | 62.80% |
| IL23R | 0.036369788 | 0.613067 | 58.45% | 64.06% |
| OR9A1P | 0.038328013 | 0.69407 | 100.00% | 48.42% |
| OR1J4 | 0.038914619 | 0.549154 | 67.58% | 46.38% |
| MMP17 | 0.04021141 | 0.281181 | 22.52% | 51.55% |
| SLC46A3 | 0.041716091 | 0.389513 | 10.14% | 29.11% |
| HLA-DPB1 | 0.043641859 | 0.395253 | 56.66% | 67.43% |
| SUCNR1 | 0.047035846 | 0.660814 | 91.66% | 65.69% |
| HLA-DMA | 0.049805633 | 0.336833 | 11.35% | 39.34% |
| OR1J2 | 0.050489198 | 0.664251 | 67.92% | 53.01% |
| OR9G4 | 0.050824372 | 0.91413 | 100.00% | 85.55% |
| CEACAM1 | 0.052876583 | 0.346306 | 21.52% | 11.56% |
| CRHR2 | 0.055144286 | 0.264782 | 63.08% | 29.91% |
| ERVMER34-1 | 0.055572169 | 0.318853 | 33.42% | 41.85% |
| MTNR1A | 0.056140081 | 0.777653 | 76.36% | 64.45% |
| OR1J1 | 0.056598819 | 0.712029 | 47.77% | 53.37% |
| SLC17A8 | 0.05829662 | 0.679933 | 45.27% | 41.92% |
| CNTNAP3 | 0.058407089 | 0.161318 | 39.24% | 29.89% |
| OR4M2 | 0.059603738 | 0.528109 | 100.00% | 75.41% |
| RGMA | 0.061505925 | 0.272183 | 21.14% | 41.97% |
| TRPV6 | 0.06204423 | 0.413929 | 43.54% | 64.49% |
| CACNG7 | 0.066494282 | 0.2808 | 10.98% | 82.33% |
| UPK1A | 0.066974912 | 0.663818 | 40.32% | 75.89% |
| SLCO2A1 | 0.06741531 | 0.576558 | 42.03% | 13.55% |
| MFI2 | 0.072748757 | 0.215102 | 30.66% | 22.15% |
| GABRR2 | 0.07317984 | 0.47354 | 34.99% | 40.99% |
| TNFRSF1A | 0.073812744 | 0.364976 | 10.21% | 13.39% |
| OR56A1 | 0.07399119 | 0.858549 | 53.00% | 34.91% |
| MST1R | 0.074404792 | 0.591335 | 58.89% | 15.83% |
| OR2L13 | 0.07695085 | 0.757735 | 81.87% | 73.75% |
| IL1RAPL2 | 0.0776838 | 0.523302 | 58.36% | 46.99% |
| CYBB | 0.078629877 | 0.342058 | 57.60% | 30.36% |
| PCDHB4 | 0.084692394 | 0.302877 | 25.12% | 16.26% |
| SPACA1 | 0.085012316 | 0.584819 | 34.07% | 74.13% |
| SLC12A8 | 0.08538709 | 0.326941 | 38.88% | 35.42% |
| GPRC5D | 0.085529285 | 0.465035 | 38.48% | 24.62% |
| LYPD3 | 0.085802705 | 0.759723 | 58.79% | 54.25% |
| AQP4 | 0.089780677 | 0.549586 | 59.14% | 38.39% |
| ESYT3 | 0.091559678 | 0.178229 | 21.46% | 15.31% |
| VTCN1 | 0.095112743 | 0.185368 | 33.51% | 46.82% |
| ERVV-2 | 0.096038814 | 0.666382 | 55.12% | 70.28% |
| IL18R1 | 0.096127678 | 0.551192 | 73.42% | 64.50% |
| PCDHB16 | 0.098000564 | 0.331829 | 16.51% | 15.08% |
| LRRN2 | 0.098105712 | 0.127241 | 39.40% | 35.39% |
| GRIK2 | 0.098857286 | 0.203627 | 32.25% | 19.54% |
| NRG4 | 0.099399089 | 0.260411 | 18.13% | 20.67% |
| CD86 | 0.100551892 | 0.663328 | 59.42% | 11.47% |
| OR5G3 | 0.102371633 | 0.676355 | 100.00% | 67.27% |
| PCDHB11 | 0.103815249 | 0.26072 | 11.17% | 16.28% |
| KCNMB4 | 0.105038502 | 0.222219 | 22.01% | 22.40% |
| CNR1 | 0.10571149 | 0.213543 | 35.83% | 31.06% |
| F2RL1 | 0.106186666 | 0.484344 | 54.69% | 35.26% |
| GFRAL | 0.107140783 | 0.170322 | 29.31% | 48.29% |
| GPRC5B | 0.108411012 | 0.10721 | 23.37% | 10.41% |
| GJB6 | 0.109506084 | 0.862423 | 50.38% | 61.87% |
| PCDHB6 | 0.112838679 | 0.364807 | 20.99% | 15.03% |
| SLC22A15 | 0.113416061 | 0.378399 | 51.89% | 35.40% |
| PCDHB5 | 0.114159032 | 0.334816 | 17.97% | 19.27% |
| TRPV4 | 0.114643774 | 0.146926 | 26.99% | 42.94% |
| TNFSF4 | 0.115249636 | 0.371655 | 17.33% | 43.20% |
| DSG2 | 0.116000019 | 0.202068 | 39.80% | 20.91% |
| KEL | 0.120910405 | 0.365646 | 19.53% | 38.30% |
| PTPRT | 0.130093482 | 0.368033 | 72.16% | 31.30% |
| SUSD3 | 0.133668009 | 0.783583 | 85.95% | 47.18% |
| TENM4 | 0.134065874 | 0.226163 | 32.49% | 20.84% |
| CHRM2 | 0.13554 | 0.371524 | 49.51% | 20.81% |
| SLC26A1 | 0.138435991 | 0.390587 | 39.94% | 52.26% |
| ITGB6 | 0.139238334 | 0.594235 | 52.04% | 24.87% |
| FPR3 | 0.139804913 | 0.502996 | 44.66% | 35.31% |
| ADRB1 | 0.141201256 | 0.607753 | 22.55% | 42.52% |
| ERVV-1 | 0.145860715 | 0.507579 | 36.44% | 65.81% |
| DSC3 | 0.146403903 | 0.648925 | 28.03% | 34.75% |
| TNFSF8 | 0.14698994 | 0.618329 | 80.13% | 31.95% |
| CD55 | 0.150135424 | 0.211569 | 16.98% | 11.83% |
| SLCO1B7 | 0.150952106 | 0.915267 | 57.51% | 69.94% |
| OR52I1 | 0.154537609 | 0.513911 | 50.16% | 20.58% |
| GLRA2 | 0.156600032 | 0.14043 | 19.23% | 21.63% |
| CD96 | 0.157863716 | 0.477956 | 52.90% | 44.88% |
| OR2M7 | 0.15876197 | 0.916036 | 39.01% | 52.33% |
| PRLR | 0.159286417 | 0.420592 | 28.09% | 53.15% |
| ANO2 | 0.16030019 | 0.252646 | 63.13% | 40.91% |
| CD69 | 0.164815239 | 0.690481 | 55.14% | 55.37% |
| ADAM20 | 0.166413187 | 0.243452 | 15.92% | 14.82% |
| MET | 0.169015296 | 0.235704 | 14.02% | 14.11% |
| OR2T33 | 0.174236616 | 0.812728 | 57.23% | 62.11% |
| OR5K2 | 0.174666738 | 0.422613 | 73.39% | 17.10% |
| SLC7A4 | 0.17803141 | 0.2785 | 100.00% | 32.07% |
| LILRB1 | 0.181261011 | 0.723975 | 42.58% | 70.18% |
| TNFRSF21 | 0.182948506 | 0.154049 | 23.81% | 13.26% |
| IL1R2 | 0.185112377 | 0.627093 | 29.48% | 68.51% |
| SLC26A8 | 0.19056878 | 0.337852 | 34.50% | 18.91% |
| NCMAP | 0.19327846 | 0.607444 | 100.00% | 43.91% |
| CDH8 | 0.194710737 | 0.354918 | 50.62% | 16.22% |
| ATRNL1 | 0.195716156 | 0.245131 | 23.42% | 12.15% |
| OR2K2 | 0.196488959 | 0.721186 | 77.89% | 54.47% |
| TMEM132C | 0.202964813 | 0.633432 | 91.89% | 47.15% |
| OR5B2 | 0.203401678 | 0.810152 | 65.45% | 74.87% |
| TLR9 | 0.20361623 | 0.3674 | 72.90% | 19.20% |
| OR14A16 | 0.203932798 | 0.771262 | 50.32% | 67.38% |
| TNFRSF10A | 0.204795169 | 0.65951 | 44.10% | 46.49% |
| OR6C76 | 0.207984379 | 0.9901 | 80.75% | 65.14% |
| SLC37A2 | 0.209902696 | 0.292212 | 21.10% | 50.60% |
| TNFRSF14 | 0.213815918 | 0.645521 | 49.63% | 47.81% |
| SLC22A9 | 0.217506966 | 0.220773 | 71.16% | 47.72% |
| SPN | 0.217712541 | 0.339599 | 49.88% | 62.67% |
| SLC36A2 | 0.220665533 | 0.326637 | 77.16% | 69.33% |
| OR5H6 | 0.224152204 | 0.795929 | 100.00% | 67.57% |
| TRPV5 | 0.224234664 | 0.510244 | 27.48% | 56.10% |
| SLC5A1 | 0.224586241 | 0.432877 | 61.43% | 10.40% |
| CLDN24 | 0.230121474 | 0.158046 | 12.00% | 54.84% |
| LPAR5 | 0.230286127 | 0.158757 | 25.14% | 59.51% |
| CORIN | 0.235163153 | 0.339445 | 25.11% | 26.74% |
| FLT3 | 0.236239688 | 0.507759 | 54.60% | 48.73% |
| VSIG10L | 0.239025236 | 0.287458 | 27.07% | 50.65% |
| SLC26A9 | 0.244667472 | 0.20879 | 55.73% | 20.72% |
| PCDH20 | 0.24473481 | 0.406825 | 69.21% | 24.93% |
| IL22RA1 | 0.247480624 | 0.528809 | 48.70% | 41.01% |
| HTR1F | 0.25262321 | 0.426215 | 38.01% | 39.52% |
| SCTR | 0.269360584 | 0.585706 | 41.15% | 53.18% |
| AREG | 0.285365133 | 0.988205 | 40.33% | 34.77% |
| TMPRSS11E | 0.287289737 | 0.757176 | 50.76% | 35.24% |
| GPR27 | 0.287369334 | 0.129887 | 15.05% | 41.52% |
| OR2M3 | 0.287883414 | 0.723642 | 49.61% | 25.37% |
| CHRM3 | 0.292760552 | 0.429913 | 35.99% | 28.84% |
| OR5212 | 0.293589602 | 0.565417 | 100.00% | 25.71% |
| AMICA1 | 0.294366502 | 0.430957 | 54.26% | 61.89% |
| CNTNAP3B | 0.304664468 | 0.162365 | 37.98% | 27.46% |
| MMP16 | 0.305192815 | 0.148228 | 32.67% | 21.30% |
| DSC1 | 0.30666097 | 0.465046 | 29.64% | 37.02% |
| EPHA6 | 0.307795383 | 0.117628 | 33.80% | 21.89% |
| SLC40A1 | 0.308943972 | 0.314302 | 12.16% | 25.02% |
| PTPRJ | 0.314666079 | 0.141104 | 15.58% | 14.29% |
| OR11H4 | 0.317603218 | 0.560032 | 29.95% | 35.99% |
| CDH4 | 0.318696407 | 0.389524 | 46.11% | 20.95% |
| OR2M2 | 0.322368988 | 0.708985 | 74.12% | 62.75% |
| CLDN10 | 0.327363727 | 0.159067 | 16.77% | 25.90% |
| P2RY10 | 0.333403021 | 0.665451 | 77.17% | 77.98% |
| ULBP2 | 0.337577227 | 0.29396 | 35.12% | 22.53% |
| OR2T12 | 0.342472629 | 0.714293 | 75.25% | 61.56% |
| PVRL3 | 0.353586641 | 0.250931 | 10.89% | 19.79% |
| OR2M5 | 0.354120997 | 0.779831 | 44.43% | 50.29% |
| EPHA3 | 0.362160887 | 0.138787 | 23.92% | 33.99% |
| NMBR | 0.362182681 | 0.541793 | 29.88% | 28.24% |
| OR56B4 | 0.369114629 | 0.558478 | 58.38% | 30.35% |
| OPRM1 | 0.372173832 | 0.186038 | 64.22% | 32.52% |
| SLC5A4 | 0.376301607 | 0.129465 | 20.37% | 22.08% |
| OPCML | 0.389676505 | 0.294599 | 69.53% | 35.03% |
| PCDHA1 | 0.391064646 | 0.157543 | 35.28% | 10.59% |
| GPR116 | 0.394508978 | 0.247755 | 27.35% | 34.25% |
| KLRF1 | 0.399078459 | 0.674108 | 19.83% | 47.89% |
| SLC6A14 | 0.4026801 | 0.566357 | 52.21% | 40.86% |
| HTR2C | 0.409046112 | 0.498029 | 59.44% | 51.72% |
| NPSR1 | 0.414882712 | 0.461735 | 60.16% | 37.20% |
| OR5H2 | 0.420245553 | 0.905735 | 71.84% | 66.85% |
| SLC6A19 | 0.421923475 | 0.536126 | 78.46% | 16.05% |
| HBEGF | 0.422130674 | 0.127216 | 10.82% | 17.99% |
| RNF43 | 0.423679314 | 0.396023 | 26.70% | 32.10% |
| SLC22A13 | 0.425250729 | 0.510741 | 16.86% | 32.17% |
| ABCA9 | 0.4277513 | 0.275948 | 31.58% | 27.72% |
| MFAP3L | 0.428727582 | 0.199564 | 11.18% | 24.09% |
| IFNLR1 | 0.433412386 | 0.294 | 20.30% | 13.23% |
| FZD10 | 0.437181281 | 0.622028 | 47.63% | 24.64% |
| UNC93A | 0.438775001 | 0.649639 | 82.06% | 19.41% |
| PROM2 | 0.441833696 | 0.393812 | 54.47% | 37.29% |
| SLCO1B3 | 0.442776927 | 0.854388 | 42.64% | 61.95% |
| SLC28A2 | 0.444899935 | 0.217122 | 25.65% | 12.37% |
| TAS1R1 | 0.45291952 | 0.626463 | 43.90% | 20.96% |
| OR4F21 | 0.454580119 | 0.210305 | 28.56% | 24.89% |
| OR2A5 | 0.470985473 | 0.837936 | 100.00% | 63.44% |
| SLC22A20 | 0.471884901 | 0.31203 | 13.44% | 13.14% |
| ABCG2 | 0.480513416 | 0.329798 | 23.97% | 42.04% |
| SLC4A5 | 0.481157567 | 0.128431 | 13.80% | 11.15% |
| PCDH19 | 0.489035885 | 0.189855 | 28.68% | 25.74% |
| SEMA5A | 0.500223818 | 0.364455 | 23.62% | 38.59% |
| VIPR2 | 0.510572731 | 0.660258 | 51.81% | 33.31% |
| LTK | 0.519512543 | 0.311795 | 13.06% | 38.57% |
| LY75 | 0.522290382 | 0.249467 | 31.45% | 12.70% |
| EFNA3 | 0.529633845 | 0.27612 | 16.80% | 26.97% |
| EPHB6 | 0.539794027 | 0.597071 | 40.31% | 50.22% |
| ERBB4 | 0.56107755 | 0.172151 | 13.10% | 17.25% |
| CA14 | 0.571308258 | 0.128152 | 26.81% | 12.06% |
| CNTN5 | 0.581581196 | 0.300722 | 27.49% | 29.98% |
| OR2M4 | 0.582020963 | 0.580173 | 66.24% | 36.75% |
| FCER1A | 0.591025159 | 0.493507 | 100.00% | 67.56% |
| OR5H15 | 0.593724479 | 0.689702 | 55.59% | 66.75% |
| KLRG1 | 0.596205981 | 0.142958 | 27.45% | 20.35% |
| OR13H1 | 0.598162745 | 0.51299 | 22.13% | 31.26% |
| PCDHA4 | 0.60134538 | 0.218153 | 23.26% | 15.97% |
| PCDHGA4 | 0.602655137 | 0.163238 | 10.30% | 11.58% |
| LYVE1 | 0.61113917 | 0.268007 | 15.56% | 14.62% |
| CDH13 | 0.613502521 | 0.342099 | 40.25% | 18.34% |
| CD93 | 0.627459193 | 0.224708 | 100.00% | 12.24% |
| EMB | 0.629582122 | 0.247293 | 10.10% | 13.93% |
| TNFRSF17 | 0.630768688 | 0.245416 | 45.29% | 13.44% |
| CTLA4 | 0.642812168 | 0.393482 | 26.78% | 58.56% |
| SELE | 0.6433245 | 0.390103 | 54.55% | 35.02% |
| OR11H1 | 0.649357164 | 0.269127 | 32.41% | 38.72% |
| PTGER3 | 0.665517257 | 0.197934 | 39.76% | 15.31% |
| IL1RAPL1 | 0.688183296 | 0.231912 | 41.10% | 35.67% |
| GPR157 | 0.69036096 | 0.537657 | 39.32% | 51.47% |
| OR2F2 | 0.696878987 | 0.401806 | 45.07% | 62.41% |
| DYNAP | 0.706197838 | 0.567356 | 15.43% | 36.68% |
| OR4D2 | 0.720626162 | 0.14698 | 43.04% | 39.72% |
| FLT1 | 0.729697246 | 0.194547 | 15.15% | 23.07% |
| OXGR1 | 0.743841162 | 0.288636 | 25.39% | 33.57% |
| HTR3B | 0.779009395 | 0.278209 | 29.90% | 45.18% |
| OTOA | 0.785937295 | 0.267347 | 16.95% | 18.12% |
| PTGER2 | 0.793703486 | 0.121655 | 15.34% | 25.18% |
| SLCO1A2 | 0.802250978 | 0.196178 | 17.31% | 10.80% |
| OR6B1 | 0.802485941 | 0.46091 | 72.71% | 52.28% |
| NOX4 | 0.821136337 | 0.266074 | 45.25% | 20.04% |
| MSR1 | 0.828371516 | 0.151105 | 41.81% | 24.54% |
| C5AR1 | 0.832412178 | 0.310529 | 16.58% | 32.90% |
| MUC13 | 0.834813066 | 0.322597 | 19.84% | 16.00% |
| SLCO1B1 | 0.836752168 | 0.260668 | 20.64% | 11.76% |
| MDGA2 | 0.836864692 | 0.210291 | 24.66% | 16.08% |
| NPY5R | 0.867710216 | 0.388816 | 58.16% | 41.80% |
| IGSF1 | 0.872718716 | 0.121582 | 16.61% | 19.65% |
| HLA-DMB | 0.89272369 | 0.20179 | 29.71% | 17.38% |
| IL18RAP | 0.907294485 | 0.425764 | 54.57% | 31.49% |
| OR56B1 | 0.926460274 | 0.397007 | 27.69% | 39.20% |
| OR2F1 | 0.943961981 | 0.443042 | 41.18% | 41.24% |
| DUOX2 | 0.959949085 | 0.184136 | 19.80% | 13.30% |
| SGCZ | 0.969958844 | 0.14357 | 32.02% | 10.60% |
| RAETIG | 0.973088186 | 0.139034 | 16.11% | 14.57% |
| EPCAM | 2.02382E−18 | 0.345566 | 44.27% | 0.08% |
| MPZL3 | 4.99593E−16 | 0.534769 | 76.83% | 9.06% |
| DLL1 | 1.74009E−10 | 0.427292 | 44.74% | 1.01% |
| CDH1 | 7.07699E−10 | 0.523184 | 58.95% | 6.24% |
| KIT | 6.69101E−09 | 0.378968 | 31.12% | 5.66% |
| ENPP4 | 1.95292E−08 | 0.189435 | 11.85% | 2.46% |
| SLC10A5 | 2.96282E−08 | 0.292874 | 28.00% | 7.67% |
| P2RX4 | 2.27518E−07 | 0.193772 | 6.62% | 1.20% |
| TMEM87A | 1.22819E−06 | 0.135133 | 6.80% | 11.55% |
| ENPP5 | 1.15581E−05 | 0.319918 | 45.40% | 9.41% |
| GRM7 | 5.91371E−05 | 0.468273 | 50.67% | 2.29% |
| SMO | 6.68432E−05 | 0.088063 | 26.67% | 63.85% |
| MEGF9 | 7.56124E−05 | 0.12105 | 18.55% | 7.64% |
| TPBG | 8.56861E−05 | 0.424973 | 4.17% | 2.82% |
| F2R | 0.000113079 | 0.149416 | 2.29% | 53.20% |
| LPPR1 | 0.000118726 | 0.045147 | 57.69% | 32.92% |
| CD37 | 0.000139435 | 0.143309 | 7.38% | 59.39% |
| SLC15A2 | 0.000255311 | 0.239744 | 15.61% | 2.70% |
| FREM2 | 0.000256065 | 0.338356 | 31.55% | 8.91% |
| ITGB8 | 0.000279081 | 0.298247 | 26.47% | 4.82% |
| SLC29A1 | 0.000295873 | 0.046854 | 0.65% | 14.91% |
| GPR160 | 0.000380265 | 0.192355 | 23.31% | 6.96% |
| KLRK1 | 0.000413482 | 0.995288 | 50.03% | 9.98% |
| TMEM87B | 0.000510537 | 0.112924 | 10.02% | 1.40% |
| LPHN3 | 0.000698088 | 0.060092 | 46.51% | 4.75% |
| BACE1 | 0.000810086 | 0.065745 | 9.24% | 0.71% |
| ALCAM | 0.001043198 | 0.246204 | 20.28% | 7.57% |
| NCR3LG1 | 0.001245492 | 0.08902 | 26.24% | 5.19% |
| PKHD1L1 | 0.001307845 | 0.29306 | 45.18% | 8.13% |
| TMEM123 | 0.001447703 | 0.102344 | 11.43% | 4.56% |
| TMEM182 | 0.001478348 | 0.051119 | 17.60% | 3.19% |
| PLET1 | 0.001595024 | 0.171932 | 14.69% | 5.60% |
| SLC6A16 | 0.001833695 | 0.0162 | 26.65% | 48.39% |
| LINGO2 | 0.001895523 | 0.19192 | 55.82% | 1.39% |
| KIAA0922 | 0.001914469 | 0.158753 | 9.78% | 11.92% |
| HEPACAM2 | 0.002317196 | 0.164385 | 51.46% | 7.43% |
| SLC12A2 | 0.00242017 | 0.045658 | 4.79% | 10.35% |
| SLC1A1 | 0.003394852 | 0.191851 | 19.87% | 1.43% |
| CCKBR | 0.003725192 | 0.342195 | 90.32% | 3.31% |
| CD160 | 0.00389868 | 0.170698 | 6.32% | 2.52% |
| NOTCH1 | 0.004020153 | 0.147541 | 4.23% | 40.57% |
| SVOPL | 0.004081823 | 0.341754 | 34.83% | 3.91% |
| SLC23A1 | 0.004213537 | 0.25089 | 7.30% | 7.41% |
| SUCO | 0.004675952 | 0.07198 | 1.44% | 3.00% |
| ITGB4 | 0.004906184 | 0.514201 | 8.75% | 55.46% |
| ANO6 | 0.005524992 | 0.172207 | 3.00% | 6.65% |
| GP1BA | 0.005945809 | 0.002759 | 41.14% | 17.46% |
| RNFT1 | 0.006873387 | 0.099814 | 2.44% | 8.05% |
| ENTPD3 | 0.007279018 | 0.165306 | 20.28% | 1.10% |
| TMPRSS11D | 0.009674279 | 0.175342 | 28.07% | 2.94% |
| UGT8 | 0.012270284 | 0.11394 | 32.92% | 6.71% |
| GNRHR2 | 0.012379872 | 0.081632 | 5.38% | 0.92% |
| SLC22A23 | 0.013490751 | 0.160706 | 14.88% | 9.74% |
| NUP210 | 0.013666093 | 0.092027 | 15.09% | 5.60% |
| P2RY1 | 0.013698663 | 0.034256 | 10.70% | 47.88% |
| SLC7A2 | 0.014618404 | 0.053093 | 53.75% | 12.30% |
| VSIG2 | 0.01579597 | 0.092862 | 36.95% | 37.47% |
| ZP2 | 0.016556352 | 0.078104 | 58.40% | 52.67% |
| BRS3 | 0.01697724 | 0.042971 | 59.71% | 0.74% |
| FGFR3 | 0.020297633 | 0.083726 | 7.22% | 25.79% |
| MXRA8 | 0.020404014 | 0.462705 | 7.14% | 67.07% |
| SLC16A6 | 0.02390724 | 0.030914 | 3.61% | 16.38% |
| CD46 | 0.026096598 | 0.07699 | 6.34% | 6.70% |
| IL20RA | 0.026755353 | 0.560022 | 45.79% | 2.92% |
| TMX3 | 0.027176233 | 0.063082 | 5.61% | 2.68% |
| ASTN2 | 0.027524861 | 0.040549 | 20.44% | 2.51% |
| SLC22A4 | 0.029046522 | 0.119051 | 0.70% | 2.14% |
| SLCO5A1 | 0.030227009 | 0.050288 | 30.43% | 6.75% |
| TGOLN2 | 0.03273918 | 0.083115 | 2.38% | 3.33% |
| ITGA2 | 0.034090169 | 0.308722 | 5.49% | 18.58% |
| CLDN2 | 0.035184898 | 0.416548 | 14.58% | 7.96% |
| SLC41A1 | 0.03927828 | 0.120731 | 2.92% | 15.18% |
| TMEM25 | 0.039486944 | 0.125839 | 14.06% | 5.13% |
| CNNM4 | 0.042901454 | 0.02802 | 6.38% | 9.24% |
| TGFA | 0.045427395 | 0.410837 | 39.09% | 9.43% |
| PCDHB13 | 0.047145464 | 0.20157 | 2.20% | 3.73% |
| CLEC2D | 0.048823542 | 0.176045 | 6.71% | 7.68% |
| GPR55 | 0.04916716 | 0.439132 | 100.00% | 4.48% |
| MFSD8 | 0.049379748 | 0.059857 | 3.94% | 1.37% |

TABLE 9
Surface proteins associated with the SCLC-P subtype from the Sato dataset

| Surface Protein | ANOVA p value | P vs N | P vs I | P vs A |
|---|---|---|---|---|
| ART3 | 1.2998E−05 | 0.662148 | 70.204% | 62.747% |
| PROM1 | 2.76663E−05 | 0.25483 | 57.251% | 14.357% |
| NOTCH1 | 4.87508E−05 | 0.123338 | 17.436% | 19.492% |
| TNFSF4 | 0.000199207 | 0.244973 | 25.491% | 19.693% |
| PRLR | 0.000840064 | 0.260746 | 46.405% | 52.358% |
| MME | 0.001908314 | 0.473581 | 24.432% | 50.273% |
| OR2B3 | 0.004715832 | 0.586817 | 29.496% | 26.796% |
| LDLRAD4 | 0.005285089 | 0.258651 | 21.737% | 14.572% |
| CCR5 | 0.005464222 | 0.408635 | 10.927% | 21.751% |
| TIGIT | 0.012805575 | 0.49146 | 13.370% | 19.357% |
| MFI2 | 0.012826303 | 0.156787 | 27.083% | 25.959% |
| TEX101 | 0.013201621 | 0.527416 | 21.996% | 13.154% |
| FAP | 0.022727492 | 0.242762 | 18.279% | 16.091% |
| JAG1 | 0.023646265 | 0.192893 | 12.006% | 18.315% |
| CDH22 | 0.027697679 | 0.341067 | 52.483% | 29.156% |
| CD6 | 0.030125671 | 0.543197 | 14.206% | 27.522% |
| PRTG | 0.035274209 | 0.189406 | 22.644% | 29.456% |
| BTN3A1 | 0.040328712 | 0.186453 | 12.742% | 14.610% |
| ERVFRD-1 | 0.04249732 | 0.437423 | 58.257% | 44.479% |
| SVOPL | 0.043554717 | 0.547867 | 29.708% | 36.161% |
| HLA-DQB1 | 0.044608653 | 0.538001 | 41.789% | 53.641% |
| SLC28A3 | 0.058109964 | 0.166732 | 11.442% | 49.439% |
| GPR32 | 0.059841001 | 0.326674 | 48.851% | 11.051% |
| PKHD1L1 | 0.060329735 | 0.73797 | 21.830% | 25.166% |
| LGR5 | 0.06858935 | 0.160077 | 62.650% | 15.182% |
| VCAM1 | 0.070368953 | 0.358085 | 17.366% | 25.644% |
| CXCR3 | 0.085582802 | 0.327426 | 10.335% | 22.699% |
| LRRC15 | 0.090682069 | 0.306011 | 13.012% | 23.493% |
| IL20RA | 0.111415007 | 0.596385 | 37.625% | 41.667% |
| TNFRSF4 | 0.129959253 | 0.459243 | 18.426% | 39.519% |
| VTCN1 | 0.151308506 | 0.226516 | 20.306% | 37.608% |
| C3orf80 | 0.15220129 | 0.302032 | 17.846% | 27.087% |
| P2RY10 | 0.157726973 | 0.446814 | 18.430% | 42.652% |
| GABRP | 0.171236688 | 0.234794 | 48.794% | 42.461% |
| SIRPG | 0.172099411 | 0.669871 | 11.428% | 26.020% |
| TLR1 | 0.233778951 | 0.248104 | 13.499% | 23.407% |
| DRD3 | 0.265995847 | 0.54176 | 40.561% | 33.405% |
| LY75 | 0.268294766 | 0.281054 | 11.438% | 20.845% |
| GLRA1 | 0.290102441 | 0.174587 | 52.418% | 16.091% |
| EPHA4 | 0.304526498 | 0.249193 | 19.504% | 18.679% |
| VLDLR | 0.308025733 | 0.259313 | 13.836% | 11.308% |
| SIGLEC10 | 0.359314534 | 0.32217 | 12.112% | 12.512% |
| TNFRSF9 | 0.368884213 | 0.246724 | 15.606% | 17.552% |
| CCRL2 | 0.380422323 | 0.41643 | 10.540% | 21.366% |
| EFNA3 | 0.394772627 | 0.225541 | 21.263% | 18.815% |
| FCRL4 | 0.457873258 | 0.405458 | 17.592% | 16.385% |
| CORIN | 0.508566636 | 0.395145 | 30.486% | 28.618% |
| NOX4 | 0.514923909 | 0.139361 | 23.313% | 17.446% |
| CEACAM1 | 0.546893035 | 0.424365 | 10.824% | 17.432% |
| AMN | 0.554657767 | 0.365207 | 28.224% | 33.180% |
| BTLA | 0.576763569 | 0.303658 | 14.473% | 21.401% |
| CDH17 | 0.586890385 | 0.506942 | 35.203% | 19.240% |
| ZPLD1 | 0.628796415 | 0.135294 | 10.391% | 29.853% |
| SUCNR1 | 0.631460481 | 0.336939 | 15.209% | 18.889% |
| GSG1 | 0.633640739 | 0.269539 | 33.426% | 34.495% |
| CCR3 | 0.675585655 | 0.363197 | 13.957% | 23.492% |
| TRHR | 0.68036082 | 0.133491 | 10.296% | 12.604% |
| TAS2R8 | 0.834484916 | 0.400714 | 23.592% | 12.855% |
| HEPACAM2 | 2.17258E−11 | 0.097628 | 53.731% | 0.744% |
| KCNMB3 | 3.76264E−09 | 0.38387 | 2.573% | 5.931% |
| KIT | 6.26736E−08 | 0.087065 | 42.303% | 9.946% |
| EPCAM | 8.43294E−08 | 0.035613 | 0.017% | 0.734% |
| SLC44A1 | 3.48196E−07 | 0.078006 | 5.172% | 6.303% |
| CHPT1 | 1.69432E−06 | 0.216974 | 5.321% | 17.991% |
| TMEM87A | 3.1838E−06 | 0.145235 | 1.119% | 11.110% |
| TMX3 | 4.2271E−06 | 0.049912 | 5.689% | 0.692% |
| FZD3 | 4.80862E−06 | 0.132925 | 11.171% | 1.990% |
| EFNA4 | 3.69156E−05 | 0.154554 | 1.872% | 44.668% |
| ACVR2B | 5.85767E−05 | 0.090462 | 17.760% | 4.970% |
| ICOS | 0.000618045 | 0.411374 | 7.269% | 51.703% |
| SLC5A3 | 0.000720572 | 0.091042 | 9.897% | 2.103% |
| TNFRSF25 | 0.000813338 | 0.130748 | 23.393% | 1.481% |
| DSG2 | 0.000887054 | 0.17887 | 2.184% | 16.799% |
| BTN3A3 | 0.001043248 | 0.278059 | 22.301% | 27.320% |
| SLAMF7 | 0.001190124 | 0.466535 | 6.076% | 22.392% |
| BTN2A2 | 0.001547106 | 0.066278 | 7.428% | 9.763% |
| IL27RA | 0.001800748 | 0.644602 | 9.623% | 39.850% |
| TNFSF13B | 0.001949925 | 0.346322 | 8.821% | 18.479% |
| HLA-E | 0.001990978 | 0.149901 | 2.007% | 14.554% |
| RNFT1 | 0.002140771 | 0.108749 | 3.437% | 5.442% |
| TMEM123 | 0.002288474 | 0.035826 | 2.558% | 4.067% |
| HIAT1 | 0.00290717 | 0.007914 | 2.513% | 1.009% |
| CD84 | 0.005249768 | 0.336669 | 6.300% | 17.497% |
| PDGFRA | 0.005478881 | 0.355302 | 12.580% | 25.318% |
| SLC12A8 | 0.005777932 | 0.305535 | 5.688% | 39.048% |
| RNF43 | 0.005791168 | 0.10495 | 1.723% | 28.760% |
| CD40LG | 0.006348126 | 0.634674 | 8.149% | 31.415% |
| MILR1 | 0.007520632 | 0.359341 | 16.872% | 39.426% |
| LILRA4 | 0.007791151 | 0.294002 | 13.465% | 35.727% |
| IL15RA | 0.008020583 | 0.221492 | 2.696% | 15.234% |
| TMEM108 | 0.008314283 | 0.115159 | 36.188% | 4.178% |
| BTN2A1 | 0.010034365 | 0.056814 | 15.270% | 8.897% |
| FPR3 | 0.011425995 | 0.276224 | 3.203% | 14.234% |
| GPC4 | 0.01154952 | 0.24206 | 6.118% | 26.140% |
| SIGLEC8 | 0.013371752 | 0.550992 | 19.753% | 22.763% |
| BTN3A2 | 0.014909281 | 0.193761 | 16.264% | 24.178% |
| C16orf52 | 0.015057781 | 0.067421 | 7.449% | 1.318% |
| CD80 | 0.016477818 | 0.25146 | 8.800% | 5.681% |
| CD96 | 0.017797972 | 0.381662 | 8.632% | 26.825% |
| TLR7 | 0.018041997 | 0.517391 | 1.441% | 22.758% |
| TNFRSF17 | 0.018050766 | 0.559379 | 5.926% | 37.133% |
| CLEC5A | 0.020084646 | 0.377996 | 0.631% | 47.287% |
| SLC10A3 | 0.021207072 | 0.066508 | 1.616% | 18.374% |
| CRTAM | 0.021898481 | 0.584179 | 9.660% | 20.736% |
| LRP1B | 0.024412537 | 0.333244 | 78.679% | 45.629% |
| GPR171 | 0.02441676 | 0.473553 | 1.048% | 29.203% |
| IL18R1 | 0.024602517 | 0.537454 | 9.455% | 30.993% |
| ADORA3 | 0.02718111 | 0.334552 | 10.534% | 41.849% |
| ITGB7 | 0.031076277 | 0.459 | 28.290% | 38.349% |
| FCGR2B | 0.033423689 | 0.373126 | 7.167% | 12.491% |
| SLC43A3 | 0.036494082 | 0.098964 | 17.614% | 15.471% |
| PTGER1 | 0.037929605 | 0.282717 | 7.504% | 11.009% |
| MPZL3 | 0.040723592 | 0.188717 | 1.284% | 4.597% |
| HLA-C | 0.045437467 | 0.07974 | 2.116% | 10.147% |
| CXCR6 | 0.045599702 | 0.359267 | 22.747% | 29.545% |
| HAVCR2 | 0.049116133 | 0.298879 | 12.792% | 34.416% |

TABLE 10
Surface proteins associated with the SCLC-I subtype
from the George et al. dataset

| Surface | ANOVA | | | |
|---|---|---|---|---|
| Protein | p value | I vs N | I vs P | I vs A |
| SLAMF6 | 2.0489E−14 | 83.15% | 52.34% | 70.83% |
| HLA-DOB | 8.40165E−13 | 68.03% | 23.50% | 53.58% |
| VNN2 | 1.10838E−12 | 82.20% | 44.26% | 62.27% |
| IL15RA | 1.68281E−11 | 66.66% | 27.77% | 50.91% |
| CD180 | 1.80221E−11 | 68.23% | 33.79% | 58.50% |
| CD38 | 2.43721E−11 | 72.74% | 40.18% | 56.98% |
| EVI2B | 4.76043E−11 | 60.28% | 24.38% | 47.72% |
| HAVCR2 | 5.12432E−11 | 56.33% | 24.39% | 44.74% |
| CD84 | 5.79077E−11 | 75.16% | 42.13% | 69.38% |
| CCRL2 | 7.2792E−11 | 71.71% | 36.02% | 55.07% |
| CLEC12A | 7.86442E−11 | 83.14% | 43.21% | 70.31% |
| GPR65 | 8.06116E−11 | 71.95% | 32.19% | 61.57% |
| P2RY10 | 8.30508E−11 | 78.01% | 47.92% | 68.33% |
| SIGLEC8 | 1.09725E−10 | 75.90% | 48.17% | 63.12% |
| SLAMF1 | 1.3785E−10 | 77.14% | 43.32% | 64.24% |
| CD6 | 1.51793E−10 | 73.31% | 43.59% | 61.77% |
| CD96 | 1.63341E−10 | 74.93% | 40.55% | 62.12% |
| TMEM150B | 1.72326E−10 | 74.06% | 35.42% | 59.69% |
| CD86 | 2.11085E−10 | 71.44% | 40.10% | 58.35% |
| PDCD1LG2 | 2.11378E−10 | 70.19% | 40.35% | 58.80% |
| S1PR4 | 2.41467E−10 | 72.20% | 31.30% | 60.31% |
| TNFSF13B | 2.72077E−10 | 69.87% | 34.27% | 51.78% |
| CD48 | 3.15615E−10 | 59.36% | 26.28% | 43.26% |
| CD97 | 3.35334E−10 | 60.69% | 28.88% | 49.28% |
| SEMA4A | 3.98484E−10 | 65.90% | 21.09% | 58.06% |
| SIGLEC14 | 4.04895E−10 | 77.99% | 33.06% | 58.28% |
| CLEC7A | 4.37288E−10 | 67.40% | 27.08% | 62.62% |
| FCRL5 | 4.61194E−10 | 82.65% | 43.09% | 72.34% |
| SLAMF7 | 4.83623E−10 | 61.39% | 33.32% | 47.06% |
| CD53 | 5.25442E−10 | 52.99% | 25.06% | 41.56% |
| TLR8 | 5.4449E−10 | 75.93% | 42.60% | 66.11% |
| ITGAX | 5.50841E−10 | 57.54% | 23.53% | 50.63% |
| PILRA | 5.65337E−10 | 56.54% | 30.80% | 49.61% |
| LY9 | 6.62498E−10 | 81.47% | 43.18% | 66.66% |
| CD300LF | 6.82801E−10 | 63.97% | 35.65% | 50.65% |
| GPR171 | 6.89105E−10 | 78.14% | 41.29% | 59.99% |
| CCR5 | 7.96872E−10 | 70.44% | 39.09% | 61.44% |
| HLA-DMA | 8.65142E−10 | 39.67% | 15.03% | 29.81% |
| SIGLEC7 | 1.09414E−09 | 68.42% | 38.66% | 58.99% |
| CD33 | 1.11754E−09 | 71.71% | 31.20% | 61.52% |
| EVI2A | 1.12892E−09 | 69.84% | 23.16% | 54.93% |
| ICOS | 1.13456E−09 | 76.36% | 43.86% | 63.09% |
| HLA-DMB | 1.3426E−09 | 43.05% | 19.42% | 34.63% |
| CD52 | 1.37278E−09 | 44.74% | 13.23% | 32.50% |
| CEACAM21 | 1.38519E−09 | 70.34% | 35.60% | 67.69% |
| ITGAL | 1.42536E−09 | 68.25% | 37.67% | 58.94% |
| HLA-DOA | 1.42768E−09 | 54.89% | 25.47% | 45.14% |
| CD2 | 1.52338E−09 | 62.65% | 29.09% | 48.37% |
| CXCR6 | 1.54737E−09 | 72.14% | 36.93% | 53.34% |
| CD80 | 1.57981E−09 | 69.61% | 47.19% | 57.54% |
| P2RY13 | 1.64544E−09 | 70.69% | 35.02% | 61.92% |
| CD5 | 1.66421E−09 | 73.72% | 48.40% | 62.63% |
| TNFRSF17 | 1.69606E−09 | 76.86% | 43.38% | 62.56% |
| AMICA1 | 1.76006E−09 | 64.84% | 27.46% | 56.14% |
| SIGLEC9 | 1.87646E−09 | 67.54% | 30.70% | 59.15% |
| IL18RAP | 1.87988E−09 | 81.56% | 41.86% | 67.76% |
| CD37 | 1.88527E−09 | 60.25% | 23.07% | 49.57% |
| CCR1 | 1.96273E−09 | 58.07% | 26.96% | 46.26% |
| SLC1A3 | 1.98011E−09 | 63.13% | 36.42% | 69.71% |
| TNFRSF9 | 2.03278E−09 | 70.70% | 51.48% | 65.47% |
| GPR174 | 2.04938E−09 | 75.64% | 37.31% | 64.35% |
| LILRB1 | 2.0837E−09 | 73.98% | 41.14% | 64.29% |
| FCRL3 | 2.16991E−09 | 81.56% | 55.75% | 70.41% |
| TRAT1 | 2.28791E−09 | 77.68% | 41.67% | 57.98% |
| PTAFR | 2.34959E−09 | 69.70% | 33.29% | 63.81% |
| PDCD1 | 2.50224E−09 | 73.30% | 39.80% | 61.43% |
| DPEP2 | 2.56288E−09 | 69.85% | 25.38% | 56.00% |
| CD19 | 2.57633E−09 | 84.60% | 58.66% | 72.71% |
| TIGIT | 2.65466E−09 | 70.79% | 37.24% | 54.95% |
| PTPRC | 2.70821E−09 | 61.71% | 31.26% | 49.42% |
| IL12RB1 | 2.80163E−09 | 69.06% | 49.59% | 58.58% |
| IFNAR2 | 3.15989E−09 | 32.28% | 12.09% | 16.47% |
| CD4 | 3.20309E−09 | 59.32% | 30.52% | 47.89% |
| FCGR1A | 3.20713E−09 | 65.89% | 27.17% | 47.96% |
| CD3G | 3.22345E−09 | 72.35% | 32.31% | 58.83% |
| SPN | 3.41185E−09 | 69.27% | 20.65% | 66.77% |
| SELPLG | 3.46916E−09 | 61.50% | 29.76% | 50.56% |
| CRTAM | 3.54528E−09 | 73.66% | 43.76% | 59.94% |
| CSF2RB | 3.66491E−09 | 62.28% | 34.16% | 54.27% |
| KLRB1 | 4.01281E−09 | 68.44% | 19.72% | 49.09% |
| TREM2 | 4.10337E−09 | 57.59% | 13.36% | 47.72% |
| IL2RG | 4.37455E−09 | 56.39% | 26.84% | 43.19% |
| FPR3 | 4.51633E−09 | 51.16% | 23.59% | 41.35% |
| IL7R | 4.72697E−09 | 66.87% | 27.59% | 54.52% |
| IL6R | 4.76571E−09 | 71.47% | 19.88% | 59.72% |
| CD244 | 4.86836E−09 | 76.78% | 44.67% | 65.83% |
| FCRL2 | 4.87908E−09 | 84.74% | 45.76% | 68.67% |
| HLA-DRA | 5.19295E−09 | 28.51% | 10.30% | 21.97% |
| CD27 | 5.30164E−09 | 64.15% | 34.91% | 48.06% |
| SLAMF8 | 5.46295E−09 | 55.90% | 29.68% | 44.05% |
| TLR2 | 5.65819E−09 | 58.78% | 13.70% | 47.53% |
| KLRK1 | 5.67699E−09 | 68.45% | 27.81% | 54.95% |
| VNN1 | 5.91542E−09 | 77.04% | 47.28% | 74.73% |
| BTN3A3 | 6.75949E−09 | 45.96% | 10.20% | 40.78% |
| SIGLEC10 | 7.09795E−09 | 74.41% | 54.78% | 59.94% |
| LPAR5 | 7.15917E−09 | 66.34% | 30.43% | 60.49% |
| CD79A | 7.28226E−09 | 68.55% | 41.55% | 54.44% |
| CYBB | 7.43534E−09 | 50.62% | 23.26% | 45.78% |
| LILRB3 | 7.55178E−09 | 67.34% | 35.78% | 57.41% |
| SELL | 7.602E−09 | 70.89% | 42.56% | 60.71% |
| ITGB2 | 7.98337E−09 | 53.23% | 27.03% | 44.31% |
| IL2RB | 8.46731E−09 | 62.64% | 37.10% | 51.24% |
| GPNMB | 8.74489E−09 | 41.54% | 16.42% | 33.77% |
| LILRB5 | 8.82366E−09 | 70.26% | 33.34% | 64.94% |
| LAIR1 | 9.24736E−09 | 57.93% | 27.07% | 47.15% |
| TLR7 | 9.90723E−09 | 66.60% | 34.48% | 61.77% |
| IL10RA | 1.04823E−08 | 52.92% | 28.25% | 45.80% |
| PTCRA | 1.05765E−08 | 77.67% | 44.69% | 66.83% |
| CD69 | 1.09854E−08 | 69.62% | 26.38% | 54.66% |
| BTLA | 1.12215E−08 | 80.35% | 57.52% | 70.30% |
| IGSF6 | 1.42487E−08 | 51.11% | 32.39% | 45.74% |
| CD28 | 1.56819E−08 | 66.31% | 50.44% | 64.11% |
| TLR6 | 1.64202E−08 | 68.10% | 32.24% | 58.62% |
| CD79B | 1.65147E−08 | 70.32% | 39.13% | 60.77% |
| FLVCR2 | 1.74986E−08 | 61.62% | 24.72% | 61.91% |
| CXCR3 | 1.88085E−08 | 70.31% | 51.83% | 64.47% |
| IL2RA | 1.90577E−08 | 65.31% | 41.64% | 49.39% |
| SIRPG | 2.06264E−08 | 77.69% | 48.60% | 61.76% |
| GLIPR1 | 2.06265E−08 | 50.89% | 19.99% | 38.23% |
| CMKLR1 | 2.09689E−08 | 67.64% | 38.13% | 61.77% |
| SIRPA | 2.33553E−08 | 42.57% | 10.02% | 49.47% |
| FASLG | 2.98975E−08 | 74.10% | 42.34% | 62.51% |
| LILRA6 | 3.07924E−08 | 64.72% | 27.01% | 55.29% |
| ICAM3 | 3.13145E−08 | 49.22% | 27.61% | 40.97% |
| CD68 | 3.15674E−08 | 41.96% | 19.38% | 32.34% |
| SLC15A3 | 3.21496E−08 | 47.63% | 17.63% | 33.84% |
| LILRB2 | 3.40809E−08 | 66.57% | 33.84% | 54.00% |
| IL21R | 3.53519E−08 | 74.08% | 53.86% | 66.33% |
| MS4A6A | 3.55458E−08 | 52.47% | 21.52% | 40.34% |
| HLA-DPB1 | 4.62258E−08 | 38.92% | 18.02% | 31.78% |
| CCR2 | 4.90605E−08 | 73.47% | 47.05% | 67.86% |
| ITGB7 | 5.18847E−08 | 56.49% | 36.44% | 47.69% |
| SLC17A9 | 5.22376E−08 | 59.55% | 24.59% | 42.20% |
| CD74 | 5.89811E−08 | 29.88% | 13.21% | 22.43% |
| GPR132 | 6.0616E−08 | 59.76% | 42.91% | 62.90% |
| CD300C | 6.06254E−08 | 65.62% | 34.25% | 59.30% |
| CD3D | 6.07523E−08 | 64.84% | 34.19% | 46.67% |
| GPR18 | 6.85491E−08 | 76.57% | 51.70% | 65.17% |
| FCGR1B | 6.94693E−08 | 75.07% | 39.92% | 60.42% |
| LRRC25 | 7.27699E−08 | 59.15% | 34.35% | 45.84% |
| FCGR2A | 7.7703E−08 | 45.19% | 14.35% | 34.99% |
| CXCL16 | 7.7909E−08 | 47.98% | 17.01% | 38.67% |
| SIRPB1 | 7.9852E−08 | 77.34% | 22.01% | 69.69% |
| CCR4 | 8.58863E−08 | 73.03% | 43.61% | 68.09% |
| SLC47A1 | 1.16018E−07 | 44.09% | 28.31% | 61.89% |
| CD200R1 | 1.18058E−07 | 71.15% | 31.27% | 56.01% |
| FCGR3A | 1.22443E−07 | 51.83% | 23.22% | 38.61% |
| LAG3 | 1.30277E−07 | 49.93% | 40.94% | 43.91% |
| SIGLEC1 | 1.31718E−07 | 60.56% | 31.75% | 54.07% |
| C3AR1 | 1.40605E−07 | 51.68% | 23.33% | 41.57% |
| NCR3 | 1.48023E−07 | 76.23% | 48.12% | 67.49% |
| CYSLTR1 | 1.50401E−07 | 63.99% | 20.11% | 55.17% |
| CD226 | 1.52294E−07 | 67.39% | 43.16% | 56.67% |
| SLC37A2 | 1.53783E−07 | 54.44% | 27.67% | 53.23% |
| VCAM1 | 1.64606E−07 | 55.58% | 27.17% | 47.12% |
| HLA-DQA1 | 1.69908E−07 | 44.45% | 18.49% | 39.29% |
| FCRL6 | 1.83468E−07 | 66.99% | 45.54% | 63.45% |
| CD1B | 1.86058E−07 | 82.55% | 51.56% | 81.07% |
| HLA-DPA1 | 2.02404E−07 | 38.54% | 15.02% | 29.25% |
| CD7 | 2.08808E−07 | 65.34% | 36.98% | 46.83% |
| GPR183 | 2.13468E−07 | 55.32% | 14.67% | 38.78% |
| SLC2A9 | 2.31902E−07 | 54.77% | 22.86% | 42.88% |
| P2RX1 | 2.38536E−07 | 69.10% | 37.58% | 65.96% |
| CD14 | 2.64212E−07 | 47.87% | 21.59% | 35.09% |
| MPEG1 | 2.85089E−07 | 47.79% | 26.15% | 37.94% |
| BTN3A1 | 2.86212E−07 | 41.14% | 10.50% | 33.62% |
| SLC6A12 | 3.14038E−07 | 65.20% | 49.93% | 70.78% |
| MS4A1 | 3.19834E−07 | 84.56% | 48.96% | 68.67% |
| HLA-DRB1 | 3.23095E−07 | 35.56% | 12.43% | 27.58% |
| CLEC12B | 3.60286E−07 | 80.14% | 38.48% | 80.54% |
| P2RY8 | 3.66932E−07 | 61.14% | 26.35% | 58.14% |
| CD22 | 3.88315E−07 | 83.86% | 44.82% | 74.78% |
| CR1 | 4.07631E−07 | 74.29% | 47.59% | 70.77% |
| FCGR2B | 4.12418E−07 | 72.23% | 33.70% | 57.18% |
| MRC1 | 5.15829E−07 | 76.75% | 24.80% | 61.36% |
| SLC46A3 | 5.17993E−07 | 47.42% | 11.22% | 40.55% |
| MSR1 | 5.37688E−07 | 54.65% | 21.26% | 47.91% |
| GPR141 | 5.93184E−07 | 72.74% | 38.06% | 68.40% |
| HLA-F | 7.44915E−07 | 40.21% | 17.95% | 33.64% |
| CD40LG | 7.59849E−07 | 75.62% | 39.77% | 63.81% |
| CD274 | 7.68211E−07 | 64.26% | 45.06% | 51.94% |
| SLCO2B1 | 7.72444E−07 | 52.65% | 27.47% | 47.05% |
| ITGAM | 8.53209E−07 | 66.32% | 29.99% | 61.49% |
| HLA-DQB1 | 1.06577E−06 | 47.90% | 15.89% | 44.25% |
| TNFRSF1B | 1.17968E−06 | 43.41% | 25.94% | 38.64% |
| TLR10 | 1.1797E−06 | 84.76% | 49.94% | 71.99% |
| TLR1 | 1.24504E−06 | 56.51% | 23.91% | 42.70% |
| GYPC | 1.54539E−06 | 45.92% | 15.50% | 38.38% |
| MMP25 | 1.62565E−06 | 59.73% | 41.45% | 54.94% |
| TMEM106A | 1.6803E−06 | 48.20% | 20.02% | 39.41% |
| TRPV2 | 1.70835E−06 | 41.99% | 27.93% | 35.63% |
| CXCR5 | 1.78382E−06 | 79.94% | 48.27% | 69.34% |
| TNFRSF4 | 1.86433E−06 | 46.04% | 19.92% | 36.95% |
| NFAM1 | 1.96793E−06 | 51.99% | 34.12% | 52.55% |
| CD300A | 2.01384E−06 | 45.49% | 28.39% | 32.29% |
| RHBDF2 | 2.14921E−06 | 45.46% | 21.34% | 36.56% |
| P2RY6 | 2.37473E−06 | 66.51% | 39.46% | 52.37% |
| SLC2A5 | 2.5299E−06 | 59.27% | 21.91% | 54.89% |
| TMEM26 | 2.67638E−06 | 54.26% | 42.84% | 49.87% |
| MFSD7 | 2.77692E−06 | 49.38% | 14.60% | 40.74% |
| CCR8 | 3.01745E−06 | 70.08% | 49.68% | 60.91% |
| FCRL1 | 3.04241E−06 | 89.43% | 65.60% | 80.86% |
| EMB | 3.21177E−06 | 42.78% | 18.43% | 43.72% |
| TNFRSF8 | 3.2862E−06 | 67.45% | 48.20% | 64.49% |
| CLEC2D | 3.36044E−06 | 40.47% | 18.69% | 50.70% |
| CSF2RA | 3.46349E−06 | 63.54% | 42.71% | 66.91% |
| CD163 | 3.57333E−06 | 56.37% | 25.46% | 48.79% |
| ADCY7 | 3.83263E−06 | 40.11% | 22.48% | 41.89% |
| ICAM1 | 4.07088E−06 | 43.01% | 12.45% | 27.30% |
| SUSD3 | 4.32085E−06 | 54.21% | 22.25% | 40.81% |
| TNFRSF13B | 4.52712E−06 | 77.23% | 48.44% | 69.06% |
| BTN3A2 | 4.77524E−06 | 41.86% | 11.41% | 33.89% |
| IL1R1 | 4.81042E−06 | 48.07% | 12.41% | 35.98% |
| BTN2A2 | 5.21307E−06 | 40.02% | 20.07% | 43.54% |
| LY75 | 5.2498E−06 | 56.23% | 24.32% | 41.26% |
| CSF1 | 5.39237E−06 | 47.67% | 25.46% | 46.20% |
| LILRA5 | 6.25423E−06 | 68.43% | 25.32% | 48.48% |
| CCR7 | 6.57438E−06 | 61.96% | 40.88% | 53.70% |
| IL1R2 | 6.59636E−06 | 80.57% | 76.21% | 74.88% |
| FCRL4 | 6.65091E−06 | 91.45% | 69.44% | 85.13% |
| SECTM1 | 8.18885E−06 | 49.89% | 26.48% | 35.57% |
| ITGAE | 8.40917E−06 | 32.33% | 10.40% | 16.67% |
| GPR114 | 8.49098E−06 | 69.54% | 51.83% | 57.62% |
| P2RY12 | 8.59531E−06 | 76.77% | 38.26% | 72.21% |
| TNFRSF14 | 8.85907E−06 | 27.17% | 14.42% | 16.31% |
| SIT1 | 9.05091E−06 | 57.64% | 35.78% | 43.08% |
| IGFLR1 | 9.13735E−06 | 32.55% | 23.56% | 26.08% |
| SIGLEC5 | 9.26414E−06 | 65.93% | 54.83% | 64.56% |
| MEP1A | 1.14997E−05 | 68.03% | 29.53% | 72.27% |
| GPR15 | 1.24945E−05 | 77.03% | 38.22% | 71.99% |
| UNC93B1 | 1.33021E−05 | 30.30% | 10.53% | 20.11% |
| ADORA3 | 1.46671E−05 | 53.72% | 30.14% | 57.04% |
| ADAM8 | 1.48264E−05 | 54.48% | 26.24% | 37.66% |
| MR1 | 1.51261E−05 | 53.64% | 15.66% | 44.40% |
| PTGER4 | 1.53174E−05 | 56.08% | 25.44% | 42.22% |
| FAS | 1.5901E−05 | 49.21% | 26.79% | 46.37% |
| AQP9 | 1.76825E−05 | 71.52% | 14.35% | 49.20% |
| SLC12A3 | 1.96002E−05 | 72.81% | 61.48% | 71.42% |
| VSIG10L | 2.28557E−05 | 51.54% | 29.87% | 49.65% |
| SLC40A1 | 2.51598E−05 | 30.08% | 17.39% | 24.45% |
| TNFRSF18 | 2.58362E−05 | 58.20% | 29.59% | 53.59% |
| HLA-DQB2 | 2.58684E−05 | 48.71% | 28.82% | 47.26% |
| TLR3 | 2.90136E−05 | 58.10% | 14.88% | 43.45% |
| TGFBR2 | 3.26298E−05 | 38.64% | 11.16% | 34.37% |
| P2RX7 | 3.59739E−05 | 61.65% | 54.30% | 56.68% |
| ITGAD | 3.72153E−05 | 85.15% | 40.41% | 67.95% |
| CSF1R | 4.03411E−05 | 45.95% | 24.19% | 38.39% |
| CD1D | 4.26485E−05 | 59.23% | 32.18% | 49.17% |
| XCR1 | 5.54676E−05 | 79.11% | 54.65% | 76.04% |
| LILRA1 | 5.69984E−05 | 66.82% | 41.35% | 59.53% |
| KIR3DL1 | 5.81227E−05 | 89.65% | 80.94% | 75.57% |
| EMR2 | 5.84273E−05 | 68.15% | 10.27% | 66.38% |
| CD8B | 6.94531E−05 | 51.36% | 28.04% | 65.09% |
| TMEM140 | 7.26214E−05 | 24.11% | 14.36% | 20.25% |
| GPR84 | 7.29165E−05 | 63.61% | 42.80% | 55.83% |
| HLA-DQA2 | 7.38048E−05 | 47.14% | 36.66% | 44.75% |
| ENTPD1 | 7.54083E−05 | 27.10% | 20.45% | 38.74% |
| LILRA4 | 8.71569E−05 | 62.17% | 46.58% | 55.78% |
| CTLA4 | 0.000108268 | 70.11% | 40.83% | 38.14% |
| GPR34 | 0.000133066 | 52.05% | 25.35% | 44.91% |
| CR2 | 0.000147094 | 88.33% | 47.00% | 74.57% |
| CEACAM4 | 0.000222012 | 60.09% | 40.71% | 55.50% |
| IL3RA | 0.000222018 | 36.92% | 25.30% | 37.17% |
| GGT1 | 0.000228935 | 45.05% | 26.02% | 40.66% |
| FPR1 | 0.000251402 | 58.07% | 29.81% | 45.35% |
| ATP1A4 | 0.00025478 | 81.26% | 52.62% | 85.12% |
| GP1BA | 0.000270596 | 55.79% | 41.63% | 62.82% |
| FOLR2 | 0.000275979 | 46.80% | 28.68% | 39.22% |
| CPM | 0.000280529 | 53.62% | 31.81% | 44.24% |
| FCGRT | 0.000306889 | 19.04% | 11.01% | 23.37% |
| KIR3DL2 | 0.000307374 | 79.16% | 57.65% | 74.23% |
| OSTM1 | 0.000310326 | 20.12% | 10.29% | 18.85% |
| CRLF2 | 0.000311888 | 78.15% | 23.11% | 81.39% |
| KIR2DL3 | 0.000314959 | 88.50% | 65.54% | 63.34% |
| NCR1 | 0.000316966 | 76.30% | 60.02% | 67.58% |
| MFSD2A | 0.000320659 | 38.77% | 37.18% | 11.39% |
| ADRB2 | 0.000335867 | 58.23% | 18.87% | 48.44% |
| CDH11 | 0.000336463 | 34.61% | 12.12% | 28.96% |
| CLEC1A | 0.000407114 | 39.57% | 13.63% | 45.47% |
| GPR161 | 0.000476523 | 15.95% | 14.47% | 51.73% |
| SLC29A3 | 0.000479328 | 34.04% | 25.87% | 34.32% |
| CNR2 | 0.000483208 | 70.16% | 60.56% | 70.03% |
| CCR6 | 0.000497955 | 63.03% | 35.28% | 52.50% |
| TNFSF4 | 0.000502367 | 43.05% | 21.95% | 44.87% |
| SCARF1 | 0.000509252 | 44.83% | 24.71% | 45.88% |
| PLXDC2 | 0.000510073 | 36.97% | 16.58% | 35.60% |
| KLRC1 | 0.000516275 | 86.58% | 57.26% | 53.41% |
| AXL | 0.000517776 | 41.12% | 18.20% | 36.67% |
| CD1C | 0.000539098 | 66.68% | 18.75% | 64.49% |
| SLC38A5 | 0.000552214 | 51.02% | 17.13% | 43.23% |
| IL9R | 0.00057445 | 41.17% | 32.66% | 70.34% |
| KCNA3 | 0.000578449 | 26.41% | 47.26% | 54.24% |
| PTGER2 | 0.000585255 | 59.39% | 36.43% | 48.74% |
| SLC16A6 | 0.00061927 | 26.64% | 18.33% | 52.08% |
| SLC2A14 | 0.000630181 | 56.86% | 17.42% | 77.35% |
| DPP4 | 0.000668206 | 49.59% | 14.30% | 43.92% |
| SLC2A6 | 0.000702092 | 35.42% | 47.33% | 28.85% |
| ILAR | 0.00071199 | 35.84% | 14.71% | 36.55% |
| SLC2A3 | 0.000761924 | 30.92% | 10.89% | 25.42% |
| TMIGD2 | 0.000874526 | 53.95% | 58.88% | 61.89% |
| CHRNA6 | 0.001107356 | 76.82% | 35.67% | 68.42% |
| ACVRL1 | 0.001131442 | 33.96% | 12.05% | 33.08% |
| SELP | 0.001357549 | 58.69% | 15.73% | 49.17% |
| HFE | 0.001374258 | 52.60% | 12.01% | 44.34% |
| KIR2DL4 | 0.001433465 | 87.86% | 39.81% | 56.35% |
| FPR2 | 0.001710602 | 72.46% | 45.59% | 52.64% |
| PTGIR | 0.001858841 | 42.31% | 11.48% | 25.24% |
| GPR25 | 0.002113398 | 55.44% | 40.36% | 57.82% |
| GPR31 | 0.002152634 | 72.19% | 51.40% | 79.69% |
| IL31RA | 0.002201478 | 59.85% | 39.16% | 73.59% |
| FAP | 0.00224563 | 38.08% | 10.25% | 26.50% |
| EMR1 | 0.00232614 | 77.04% | 50.07% | 67.34% |
| HCAR3 | 0.002438521 | 63.30% | 35.35% | 41.75% |
| FLT3 | 0.002458482 | 70.14% | 46.12% | 54.61% |
| ITGA4 | 0.002477499 | 32.80% | 14.23% | 31.39% |
| HLA-G | 0.002606559 | 50.00% | 26.50% | 55.48% |
| KIR2DS4 | 0.002665903 | 77.92% | 55.01% | 75.58% |
| SUCNR1 | 0.002967653 | 74.23% | 12.13% | 23.35% |
| DPEP1 | 0.003052523 | 58.76% | 37.01% | 66.21% |
| ABCA6 | 0.003078104 | 52.19% | 10.72% | 44.27% |
| CD300E | 0.003196233 | 56.76% | 17.16% | 51.67% |
| TSPAN33 | 0.003331691 | 21.37% | 19.20% | 35.25% |
| HEPACAM | 0.003396402 | 35.45% | 39.38% | 86.07% |
| VSTM1 | 0.003398374 | 82.60% | 42.51% | 57.88% |
| TLR4 | 0.003544398 | 54.48% | 29.70% | 33.53% |
| PIK3IP1 | 0.004121404 | 24.75% | 18.38% | 14.20% |
| SIDT1 | 0.004231394 | 50.33% | 34.16% | 32.55% |
| ALPP | 0.005265665 | 76.73% | 29.75% | 70.52% |
| ENG | 0.005443173 | 24.33% | 12.68% | 24.92% |
| ICAM2 | 0.005643045 | 37.41% | 21.80% | 14.41% |
| LRP10 | 0.005665413 | 22.05% | 10.72% | 11.98% |
| KLRC3 | 0.005849581 | 73.21% | 40.32% | 28.55% |
| MYOF | 0.006175117 | 33.29% | 12.36% | 30.62% |
| GPR68 | 0.006417327 | 32.13% | 30.75% | 39.46% |
| S1PR1 | 0.006676834 | 29.22% | 13.12% | 26.73% |
| CLMP | 0.00668654 | 40.35% | 10.68% | 47.81% |
| HCAR2 | 0.006754245 | 50.86% | 20.17% | 49.09% |
| SLC28A3 | 0.007405939 | 32.97% | 33.32% | 83.21% |
| FCGR3B | 0.007501269 | 49.97% | 21.56% | 50.60% |
| ADAM19 | 0.00784852 | 13.08% | 16.14% | 29.94% |
| SIGLEC6 | 0.008217117 | 73.67% | 13.29% | 69.79% |
| CLEC9A | 0.008600165 | 73.55% | 18.08% | 53.08% |
| CD40 | 0.010588927 | 33.25% | 19.23% | 20.22% |
| RAMP3 | 0.010713307 | 31.56% | 15.49% | 27.07% |
| LRRC3B | 0.010915448 | 64.58% | 68.48% | 77.56% |
| CYSLTR2 | 0.011086624 | 40.48% | 34.20% | 38.41% |
| KLRC2 | 0.011660162 | 79.89% | 31.98% | 23.46% |
| PLXNC1 | 0.011819309 | 17.19% | 37.67% | 37.53% |
| TSPAN4 | 0.012634994 | 25.08% | 26.26% | 18.13% |
| HTR2B | 0.012971039 | 43.95% | 15.14% | 43.83% |
| ALPPL2 | 0.013258468 | 87.82% | 26.61% | 86.32% |
| TNFSF18 | 0.014089112 | 51.13% | 43.48% | 52.69% |
| GPR1 | 0.014149687 | 77.95% | 53.47% | 91.14% |
| HEPH | 0.014584616 | 45.69% | 14.43% | 31.02% |
| ART4 | 0.016075366 | 64.98% | 12.39% | 54.73% |
| TMEM158 | 0.016971813 | 17.84% | 43.98% | 20.77% |
| GPR155 | 0.018031969 | 40.02% | 37.62% | 37.00% |
| OR4C6 | 0.01906316 | 89.60% | 81.29% | 54.61% |
| EFNA2 | 0.022332929 | 10.03% | 59.65% | 75.45% |
| CD163L1 | 0.024429535 | 35.12% | 14.29% | 38.33% |
| SLC22A2 | 0.026703114 | 58.18% | 61.27% | 94.41% |
| EDAR | 0.028623147 | 48.77% | 32.68% | 79.09% |
| SPNS3 | 0.030240964 | 47.63% | 34.39% | 56.12% |
| CD82 | 0.031670685 | 20.63% | 18.57% | 27.18% |
| GPBAR1 | 0.031808223 | 53.16% | 43.66% | 22.55% |
| KIR2DL1 | 0.033881652 | 72.08% | 77.67% | 64.43% |
| FGFR3 | 0.03601052 | 10.85% | 23.25% | 56.31% |
| DPEP3 | 0.036810148 | 55.54% | 51.38% | 69.85% |
| ABCB1 | 0.037449053 | 42.34% | 33.34% | 47.50% |
| APCDD1 | 0.038885148 | 20.45% | 33.79% | 14.00% |
| SIDT2 | 0.039726648 | 19.41% | 15.55% | 12.19% |
| ITGA11 | 0.041348494 | 36.32% | 16.69% | 30.65% |
| LRRC15 | 0.044608166 | 62.33% | 29.44% | 43.40% |
| SSTR3 | 0.045237944 | 24.52% | 71.22% | 61.35% |
| SLC43A2 | 0.04579749 | 20.01% | 12.15% | 15.42% |
| CD8A | 0.048186112 | 50.12% | 53.07% | 48.81% |
| FCAMR | 0.048672388 | 89.94% | 70.70% | 80.27% |
| SEMA4D | 0.049671578 | 13.78% | 15.77% | 11.34% |
| PCDH11Y | 0.05249787 | 55.00% | 61.40% | 72.57% |
| CD83 | 0.053170623 | 21.81% | 14.61% | 12.71% |
| HRH2 | 0.05425675 | 30.56% | 15.85% | 50.20% |
| OR9K2 | 0.055346648 | 50.14% | 100.00% | 72.80% |
| ACE | 0.057091438 | 23.59% | 16.05% | 19.58% |
| KLRF1 | 0.0624496 | 49.67% | 14.42% | 21.97% |
| HBEGF | 0.063083869 | 26.82% | 12.85% | 22.82% |
| AREG | 0.063379385 | 59.46% | 11.05% | 48.78% |
| IL17RA | 0.069735707 | 16.60% | 15.60% | 18.15% |
| FAM26E | 0.076139775 | 34.14% | 14.07% | 24.37% |
| CLEC4M | 0.076825144 | 84.98% | 67.86% | 53.74% |
| SLC14A1 | 0.078179092 | 26.35% | 35.35% | 66.22% |
| STAB2 | 0.080166637 | 61.02% | 58.90% | 49.80% |
| KEL | 0.081362044 | 25.76% | 22.89% | 41.76% |
| GPR150 | 0.085508538 | 53.96% | 33.70% | 22.25% |
| GHR | 0.086428647 | 50.68% | 39.88% | 75.13% |
| THBD | 0.0889064 | 31.42% | 10.05% | 12.78% |
| MAS1L | 0.089717898 | 90.73% | 41.49% | 72.12% |
| CA12 | 0.092735208 | 42.57% | 15.66% | 44.36% |
| CTNS | 0.095491713 | 20.76% | 11.87% | 12.29% |
| ANPEP | 0.095821779 | 33.92% | 16.74% | 31.87% |
| FLT3LG | 0.099824876 | 26.64% | 23.49% | 18.55% |
| GPR82 | 0.106862956 | 30.15% | 45.56% | 42.85% |
| IL23R | 0.10892897 | 51.72% | 30.38% | 57.90% |
| GPR151 | 0.114981165 | 75.93% | 87.60% | 51.46% |
| PCDH11X | 0.11793424 | 18.31% | 91.98% | 48.10% |
| FCER1A | 0.129568313 | 52.45% | 14.65% | 67.52% |
| FCAR | 0.137064204 | 37.35% | 53.89% | 62.14% |
| ANTXR2 | 0.138531686 | 24.23% | 24.79% | 25.13% |
| SDC1 | 0.142896081 | 17.67% | 18.48% | 11.30% |
| RAET1G | 0.143670101 | 21.34% | 28.99% | 46.24% |
| LIM2 | 0.145921766 | 68.41% | 72.52% | 60.71% |
| LRRN4 | 0.152287624 | 51.00% | 17.34% | 26.10% |
| TMEM204 | 0.160249453 | 21.96% | 10.99% | 15.89% |
| OR10G2 | 0.162520657 | 84.20% | 89.46% | 35.19% |
| F2RL3 | 0.163598915 | 28.96% | 25.29% | 25.86% |
| OR6S1 | 0.165047248 | 67.09% | 94.81% | 73.91% |
| PCDHGA12 | 0.168434817 | 23.20% | 15.87% | 46.95% |
| PARM1 | 0.173585911 | 24.09% | 26.04% | 12.02% |
| CD1A | 0.187558898 | 34.09% | 13.56% | 60.83% |
| SIGLEC12 | 0.188071133 | 51.48% | 41.93% | 44.17% |
| TMEFF2 | 0.191127136 | 31.05% | 62.28% | 10.41% |
| SIGLEC15 | 0.191489067 | 32.80% | 65.06% | 24.43% |
| SIGLEC11 | 0.198069651 | 45.66% | 17.01% | 49.12% |
| OR52D1 | 0.203182103 | 85.29% | 20.00% | 73.48% |
| ADAM7 | 0.218058488 | 65.43% | 75.61% | 88.53% |
| PRND | 0.21958522 | 19.64% | 45.17% | 69.95% |
| DDR2 | 0.220941876 | 29.15% | 17.37% | 34.66% |
| LRRC52 | 0.223360079 | 70.15% | 35.54% | 35.83% |
| CD248 | 0.225295624 | 19.59% | 15.38% | 16.88% |
| NRP2 | 0.226983312 | 17.46% | 26.93% | 25.77% |
| OR5AU1 | 0.230862414 | 52.98% | 81.82% | 56.11% |
| HTR7 | 0.231150739 | 50.54% | 13.38% | 67.83% |
| TREML2 | 0.236355659 | 60.54% | 16.68% | 35.74% |
| SLCO1B1 | 0.23811508 | 80.02% | 97.52% | 49.43% |
| OR51B5 | 0.247380328 | 94.86% | 72.58% | 69.40% |
| ALPI | 0.248773362 | 62.79% | 100.00% | 91.77% |
| OR11H6 | 0.252091671 | 62.39% | 100.00% | 70.28% |
| OR4N2 | 0.269899128 | 83.72% | 63.78% | 89.22% |
| OR4M2 | 0.277627911 | 84.31% | 62.19% | 100.00% |
| ITGA1 | 0.297561615 | 11.24% | 10.41% | 15.12% |
| SSTR4 | 0.301516412 | 62.14% | 91.83% | 84.42% |
| SORCS3 | 0.30598007 | 15.56% | 92.47% | 12.11% |
| ICOSLG | 0.308851901 | 15.47% | 25.06% | 16.39% |
| ITLN1 | 0.32944986 | 59.45% | 25.32% | 77.42% |
| OR9A4 | 0.342397173 | 59.73% | 33.38% | 73.64% |
| EPHA5 | 0.375869842 | 52.30% | 64.02% | 54.66% |
| TNFRSF10C | 0.37873391 | 32.63% | 28.83% | 10.65% |
| PCDHA4 | 0.400014956 | 30.91% | 73.41% | 14.42% |
| PTGFR | 0.411624358 | 19.46% | 40.61% | 50.59% |
| CNTNAP4 | 0.411697707 | 52.50% | 72.17% | 54.19% |
| OR10C1 | 0.420933008 | 53.34% | 100.00% | 57.06% |
| SLAMF9 | 0.429164603 | 41.21% | 22.82% | 12.79% |
| OR1E2 | 0.438456241 | 87.23% | 34.19% | 66.34% |
| LY6D | 0.444188916 | 12.69% | 19.90% | 66.86% |
| HRH1 | 0.445940141 | 29.53% | 22.48% | 21.70% |
| LRP2 | 0.453436945 | 51.77% | 41.99% | 28.99% |
| CD101 | 0.453721123 | 29.37% | 20.81% | 25.74% |
| PLB1 | 0.459993867 | 24.60% | 32.48% | 16.06% |
| SLC6A19 | 0.473661425 | 42.74% | 94.78% | 62.75% |
| P2RY14 | 0.474983931 | 27.51% | 11.39% | 26.45% |
| ASGR1 | 0.476297454 | 22.57% | 22.52% | 11.36% |
| PRSS41 | 0.525049211 | 85.27% | 87.12% | 59.26% |
| ERVV-2 | 0.533637306 | 44.35% | 73.25% | 40.47% |
| CLEC1B | 0.536328757 | 51.51% | 47.43% | 48.59% |
| ACHE | 0.543275241 | 22.41% | 37.72% | 26.09% |
| SUSD2 | 0.554481684 | 22.06% | 10.61% | 10.54% |
| TM4SF18 | 0.565251826 | 15.14% | 14.23% | 27.96% |
| LPPR1 | 0.587730816 | 40.02% | 21.26% | 18.01% |
| OR10J3 | 0.588745272 | 62.98% | 100.00% | 18.60% |
| MUC15 | 0.607910314 | 46.90% | 46.41% | 13.02% |
| OR11G2 | 0.627970732 | 31.28% | 100.00% | 60.46% |
| CEACAM6 | 0.644987142 | 15.24% | 28.56% | 12.57% |
| MAS1 | 0.657858756 | 11.04% | 78.07% | 18.14% |
| OR52E4 | 0.658683792 | 80.82% | 27.43% | 49.82% |
| OR1L8 | 0.670604998 | 19.29% | 46.31% | 13.57% |
| LRRC38 | 0.671819613 | 46.01% | 55.67% | 35.07% |
| SCN10A | 0.673755981 | 40.92% | 26.29% | 40.05% |
| RAET1L | 0.699603878 | 20.71% | 58.98% | 40.36% |
| USH2A | 0.704907869 | 35.06% | 87.97% | 27.88% |
| PHEX | 0.708815533 | 14.96% | 17.62% | 25.27% |
| LY6G6D | 0.730040354 | 11.19% | 50.33% | 39.54% |
| CLEC4G | 0.737227214 | 37.27% | 53.92% | 22.23% |
| GRID2 | 0.739784265 | 57.21% | 22.35% | 47.45% |
| GRIN3B | 0.749536105 | 21.78% | 34.50% | 12.92% |
| SLC34A1 | 0.757860005 | 17.97% | 30.88% | 51.28% |
| GLP2R | 0.790996461 | 11.82% | 34.33% | 46.60% |
| TMIGD1 | 0.827554573 | 53.42% | 51.05% | 46.69% |
| CNTNAP3 | 0.931090379 | 13.25% | 15.57% | 16.86% |
| OR5V1 | 0.969185091 | 48.09% | 24.79% | 30.25% |
| HLA-E | 2.88005E−09 | 27.48% | 7.86% | 20.92% |
| IL18R1 | 4.43558E−09 | 69.21% | 9.27% | 64.20% |
| GPR137B | 4.23233E−08 | 25.19% | 0.04% | 22.24% |
| RNF149 | 5.54774E−08 | 21.62% | 5.38% | 17.17% |
| BST2 | 5.80782E−08 | 39.21% | 8.78% | 23.70% |
| CSF3R | 6.05325E−07 | 65.07% | 7.53% | 45.69% |
| EMP3 | 9.52059E−07 | 28.36% | 8.98% | 26.96% |
| HLA-B | 2.06027E−06 | 20.48% | 5.68% | 15.10% |
| KLRG1 | 2.11931E−06 | 44.42% | 1.32% | 45.70% |
| C5AR1 | 5.32274E−06 | 46.82% | 7.56% | 38.22% |
| SEMA7A | 9.19462E−06 | 55.11% | 9.25% | 44.94% |
| SSPN | 1.34226E−05 | 55.84% | 0.73% | 43.29% |
| CD44 | 1.41112E−05 | 36.61% | 4.37% | 48.78% |
| FCGR2C | 1.52087E−05 | 66.73% | 7.85% | 61.92% |
| BST1 | 1.72928E−05 | 41.62% | 5.00% | 34.79% |
| VASN | 3.53555E−05 | 36.74% | 6.15% | 20.72% |
| CNTN2 | 7.12044E−05 | 2.23% | 80.14% | 68.63% |
| IFNGR1 | 0.000134821 | 17.09% | 6.22% | 10.21% |
| OTOA | 0.000136446 | 63.84% | 3.69% | 57.64% |
| NAGPA | 0.000140162 | 27.25% | 8.52% | 19.70% |
| SLC39A8 | 0.000171504 | 44.02% | 3.70% | 28.51% |
| ABCC3 | 0.000257432 | 47.97% | 8.33% | 46.07% |
| OSMR | 0.000282849 | 43.26% | 2.31% | 35.46% |
| PIEZO1 | 0.000286913 | 33.28% | 4.50% | 32.78% |
| MPZL1 | 0.000372352 | 16.14% | 0.71% | 14.32% |
| HLA-C | 0.00039901 | 18.48% | 3.34% | 12.32% |
| TMEM150A | 0.000447531 | 33.23% | 7.75% | 17.85% |
| SLC41A2 | 0.000472854 | 9.54% | 40.29% | 6.68% |
| IFNGR2 | 0.000478988 | 12.59% | 4.66% | 1.74% |
| HLA-A | 0.000493636 | 18.13% | 6.57% | 10.88% |
| ACP2 | 0.000547946 | 20.59% | 8.52% | 12.85% |
| LPAR6 | 0.000648364 | 37.34% | 0.60% | 37.86% |
| MFSD5 | 0.001016103 | 20.69% | 10.02% | 6.42% |
| RNF130 | 0.001069597 | 9.34% | 3.07% | 14.16% |
| PLAUR | 0.001308991 | 31.56% | 8.17% | 21.49% |
| SLC6A11 | 0.00136016 | 1.69% | 54.06% | 89.45% |
| HM13 | 0.001389185 | 12.67% | 3.33% | 3.36% |
| LAMP3 | 0.001608543 | 34.39% | 9.59% | 7.20% |
| SPPL2A | 0.002034623 | 15.46% | 6.93% | 12.87% |
| PSEN2 | 0.002203809 | 24.28% | 11.57% | 6.05% |
| M6PR | 0.002212382 | 12.05% | 1.34% | 6.23% |
| MMP14 | 0.00227536 | 13.27% | 4.88% | 21.98% |
| MPZL2 | 0.002618202 | 25.80% | 0.15% | 28.43% |
| TNFRSF10A | 0.002652269 | 39.18% | 4.47% | 39.36% |
| CD59 | 0.004884834 | 19.06% | 0.68% | 3.24% |
| MRC2 | 0.005121681 | 29.09% | 6.18% | 22.18% |
| PROCR | 0.005162919 | 22.38% | 1.75% | 29.40% |
| CD63 | 0.005308545 | 11.81% | 4.59% | 6.44% |
| ADAM9 | 0.005410379 | 18.21% | 4.09% | 7.40% |
| RPRM | 0.006224452 | 4.85% | 45.62% | 3.38% |
| MRGPRF | 0.006327273 | 50.89% | 9.61% | 46.35% |
| OR56B1 | 0.006634671 | 79.41% | 5.25% | 82.89% |
| CD302 | 0.006691516 | 30.25% | 8.64% | 4.49% |
| ABCA1 | 0.006722944 | 14.83% | 4.77% | 31.22% |
| ITM2B | 0.007028054 | 10.38% | 2.15% | 6.28% |
| ITGA5 | 0.007467691 | 23.83% | 4.99% | 20.93% |
| GPA33 | 0.008626779 | 84.23% | 7.89% | 83.48% |
| TCIRG1 | 0.010323314 | 22.33% | 6.69% | 15.04% |
| F2R | 0.01097726 | 15.75% | 3.70% | 19.37% |
| SLC31A1 | 0.012409667 | 13.91% | 7.82% | 8.51% |
| ABCA9 | 0.013132101 | 44.70% | 2.49% | 41.00% |
| CALCRL | 0.014087908 | 33.78% | 1.70% | 21.77% |
| MYADM | 0.015217168 | 26.50% | 0.97% | 1.61% |
| RECK | 0.015625369 | 8.76% | 16.91% | 31.17% |
| LINGO3 | 0.018052603 | 8.62% | 70.63% | 18.43% |
| MXRA8 | 0.0181563 | 24.65% | 7.89% | 17.18% |
| NCSTN | 0.018785421 | 6.80% | 0.43% | 0.92% |
| FRRS1 | 0.01931061 | 36.23% | 2.11% | 38.38% |
| PLA2R1 | 0.019413426 | 41.93% | 4.22% | 38.90% |
| LY6E | 0.021661514 | 20.04% | 9.83% | 16.19% |
| PRNP | 0.022136049 | 25.41% | 7.09% | 19.59% |
| NPC1 | 0.022850945 | 12.00% | 5.32% | 12.99% |
| PKD1L1 | 0.025432154 | 20.04% | 2.45% | 44.08% |
| CD93 | 0.031807592 | 19.54% | 1.39% | 23.40% |
| RNF13 | 0.03288098 | 13.52% | 4.90% | 3.07% |
| FFAR3 | 0.035469445 | 59.77% | 7.83% | 44.34% |
| PPAP2A | 0.035890417 | 16.16% | 3.05% | 15.66% |
| STAB1 | 0.036106728 | 25.31% | 7.64% | 23.96% |
| RTN4RL1 | 0.036943614 | 3.64% | 8.55% | 57.38% |
| TTYH3 | 0.037151495 | 1.22% | 11.13% | 9.71% |
| FGFR2 | 0.042451549 | 3.97% | 23.92% | 51.89% |
| CLEC14A | 0.045282141 | 20.89% | 7.13% | 19.54% |
| TEK | 0.049971964 | 29.29% | 4.41% | 37.31% |

TABLE 11
Surface proteins associated with the SCLC-I subtype from the cell line dataset

| Surface Protein | ANOVA P value | I vs N | I vs P | I vs A |
|---|---|---|---|---|
| PDGFRB | 9.70855E−13 | 41.82% | 87.73% | 89.46% |
| SCARF2 | 1.14401E−06 | 28.00% | 55.46% | 87.84% |
| AXL | 1.41233E−06 | 42.54% | 60.57% | 47.87% |
| S1PR3 | 1.00165E−05 | 34.43% | 50.87% | 76.36% |
| MRC2 | 1.16913E−05 | 40.41% | 66.21% | 64.45% |
| GPR124 | 1.53035E−05 | 24.18% | 16.47% | 33.93% |
| SSPN | 3.15744E−05 | 47.13% | 20.08% | 73.38% |
| GPR176 | 4.11096E−05 | 18.82% | 28.91% | 39.70% |
| ITGA11 | 5.90876E−05 | 51.71% | 99.77% | 64.24% |
| EMP3 | 6.40989E−05 | 29.27% | 73.47% | 61.72% |
| ELFN1 | 7.084E−05 | 20.66% | 99.69% | 59.94% |
| OR2W1 | 7.51498E−05 | 47.62% | 53.74% | 90.16% |
| ABCC10 | 8.64139E−05 | 16.88% | 24.08% | 15.80% |
| SIRPG | 0.000125124 | 54.50% | 20.54% | 87.91% |
| CD248 | 0.000140839 | 51.80% | 88.49% | 80.94% |
| ERBB2 | 0.000158538 | 31.30% | 17.83% | 28.08% |
| IL15RA | 0.000180892 | 86.00% | 24.09% | 65.39% |
| EPHB4 | 0.000183815 | 36.27% | 35.64% | 58.50% |
| NOTCH3 | 0.000184805 | 51.42% | 28.11% | 54.94% |
| VCAM1 | 0.000185193 | 75.66% | 12.66% | 73.35% |
| TMEM63B | 0.000202062 | 11.31% | 11.86% | 13.46% |
| GAS1 | 0.000229798 | 63.61% | 28.89% | 84.65% |
| EMP1 | 0.000251881 | 56.89% | 39.27% | 67.12% |
| DAG1 | 0.000268459 | 14.58% | 10.17% | 10.80% |
| SIRPB1 | 0.000285131 | 41.23% | 34.75% | 73.19% |
| ITGA5 | 0.000291127 | 14.15% | 39.97% | 38.83% |
| GJA3 | 0.000343217 | 70.40% | 71.58% | 89.41% |
| HLA-E | 0.000345515 | 53.04% | 44.15% | 73.70% |
| CCR10 | 0.000404984 | 21.44% | 58.04% | 54.58% |
| TP53I13 | 0.000464936 | 11.36% | 33.85% | 53.00% |
| EMR2 | 0.000465156 | 35.27% | 35.02% | 66.99% |
| SLC2A3 | 0.000480693 | 14.22% | 50.21% | 59.18% |
| LY6E | 0.000548484 | 23.98% | 77.93% | 59.03% |
| BTNL9 | 0.000576928 | 48.11% | 57.89% | 80.48% |
| BTN2A3P | 0.00062003 | 23.96% | 16.19% | 49.19% |
| EPHA2 | 0.000630155 | 25.40% | 20.37% | 53.25% |
| SLC1A6 | 0.000699746 | 63.96% | 45.32% | 83.48% |
| TMEM26 | 0.00074033 | 62.21% | 99.65% | 84.53% |
| CHRNG | 0.000779166 | 26.61% | 69.85% | 71.18% |
| HLA-C | 0.000829317 | 67.10% | 20.53% | 39.03% |
| SECTM1 | 0.001033177 | 99.40% | 59.17% | 71.79% |
| GYPC | 0.001048073 | 20.53% | 99.05% | 93.07% |
| PTPRQ | 0.001228831 | 46.76% | 71.68% | 37.91% |
| SIRPA | 0.0012998 | 23.00% | 19.42% | 53.09% |
| CSPG4 | 0.001382179 | 16.05% | 64.93% | 56.40% |
| VASN | 0.001678081 | 49.90% | 62.89% | 45.92% |
| CD36 | 0.001802107 | 44.34% | 57.34% | 43.04% |
| ELFN2 | 0.001814298 | 13.24% | 26.98% | 68.75% |
| SLC16A5 | 0.001827075 | 88.43% | 55.60% | 76.18% |
| MALRD1 | 0.001941222 | 50.13% | 37.60% | 58.76% |
| LRRC15 | 0.002610256 | 53.05% | 76.41% | 81.17% |
| CD97 | 0.002967346 | 35.16% | 38.68% | 53.52% |
| EDNRA | 0.002984467 | 61.85% | 31.26% | 56.19% |
| IL1R1 | 0.003251884 | 79.09% | 22.85% | 63.05% |
| SLC38A4 | 0.003300738 | 31.52% | 31.04% | 21.41% |
| OLR1 | 0.003597158 | 69.69% | 22.47% | 78.10% |
| OR2J3 | 0.003661151 | 53.38% | 45.61% | 73.72% |
| LY6K | 0.003696562 | 76.50% | 81.73% | 68.27% |
| CSF2RA | 0.003860425 | 40.84% | 50.18% | 68.11% |
| MICA | 0.004093527 | 82.55% | 50.87% | 76.35% |
| GJA1 | 0.004139472 | 34.81% | 44.90% | 39.66% |
| HFE | 0.004759886 | 58.52% | 44.98% | 39.99% |
| FSHR | 0.004852942 | 27.62% | 99.42% | 72.48% |
| OR2J2 | 0.005516914 | 49.92% | 46.85% | 78.53% |
| ZPLD1 | 0.00554278 | 26.30% | 87.74% | 62.18% |
| SIGLEC10 | 0.005610828 | 59.09% | 10.73% | 57.72% |
| LPHN2 | 0.005757306 | 23.37% | 28.32% | 22.89% |
| CRLF2 | 0.006602335 | 74.81% | 88.17% | 75.13% |
| NLGN2 | 0.007183664 | 11.51% | 15.52% | 22.20% |
| NT5E | 0.007910095 | 46.08% | 23.07% | 32.38% |
| FAM26E | 0.008056667 | 81.74% | 85.99% | 69.92% |
| CALCR | 0.008328862 | 69.39% | 34.94% | 40.61% |
| SCNN1D | 0.008695163 | 31.70% | 35.19% | 45.85% |
| GPNMB | 0.009107969 | 42.84% | 43.29% | 35.71% |
| TMEM150A | 0.009184623 | 21.96% | 28.47% | 41.19% |
| SLC6A7 | 0.009465125 | 19.01% | 99.21% | 71.82% |
| NIPAL4 | 0.010403691 | 11.82% | 53.50% | 39.59% |
| AGTR1 | 0.011008851 | 65.46% | 21.84% | 64.33% |
| ADAM19 | 0.011294672 | 12.43% | 16.75% | 18.07% |
| CACNG8 | 0.011445992 | 69.51% | 43.60% | 82.00% |
| CDH10 | 0.011463268 | 22.18% | 77.86% | 36.14% |
| HLA-DRA | 0.011810381 | 90.27% | 64.96% | 74.19% |
| FZD2 | 0.011825025 | 40.01% | 75.15% | 46.91% |
| MMP14 | 0.011895604 | 43.04% | 41.58% | 61.49% |
| ITGB5 | 0.011906104 | 25.09% | 15.21% | 45.53% |
| LRRC4B | 0.012282734 | 50.44% | 86.50% | 68.81% |
| ATP1A2 | 0.01356725 | 40.87% | 75.14% | 70.15% |
| HLA-B | 0.015591244 | 68.55% | 28.55% | 54.29% |
| SEMA7A | 0.016400864 | 13.93% | 14.32% | 49.75% |
| F2RL2 | 0.016703706 | 44.63% | 36.55% | 66.00% |
| NAGPA | 0.016942233 | 10.82% | 28.27% | 26.65% |
| SLC2A10 | 0.017091586 | 51.60% | 54.82% | 65.32% |
| MICB | 0.017256735 | 62.98% | 57.16% | 57.14% |
| ZP3 | 0.017350621 | 25.93% | 39.64% | 37.85% |
| LRFN3 | 0.017451257 | 11.83% | 43.72% | 28.58% |
| HYAL2 | 0.017616091 | 35.96% | 49.22% | 28.55% |
| IL31RA | 0.018781884 | 46.13% | 68.78% | 60.05% |
| OR7A5 | 0.019891214 | 32.52% | 99.35% | 76.26% |
| SUSD5 | 0.019979736 | 28.91% | 45.10% | 41.66% |
| EGFR | 0.020072443 | 43.36% | 44.48% | 24.78% |
| GHR | 0.021406512 | 59.67% | 32.33% | 50.02% |
| TEK | 0.021476624 | 33.06% | 60.81% | 45.15% |
| HCRTR2 | 0.022721512 | 44.85% | 56.05% | 70.83% |
| CD180 | 0.024188886 | 86.84% | 99.44% | 46.09% |
| LPAR4 | 0.025833382 | 61.07% | 12.43% | 68.02% |
| SLC43A2 | 0.028164393 | 15.56% | 49.17% | 17.98% |
| PTGDR | 0.028701973 | 67.64% | 27.36% | 27.10% |
| CEACAM8 | 0.032014126 | 52.16% | 99.48% | 64.33% |
| CD163 | 0.03389116 | 11.84% | 15.55% | 60.96% |
| HLA-F | 0.034348946 | 51.77% | 52.74% | 59.52% |
| SLC12A9 | 0.035177737 | 22.62% | 25.00% | 38.24% |
| HTR1B | 0.035875203 | 77.67% | 99.42% | 75.68% |
| PCDH7 | 0.038481146 | 41.72% | 43.17% | 32.77% |
| HLA-H | 0.04201114 | 78.24% | 50.87% | 61.85% |
| LMAN2 | 0.042283864 | 11.64% | 22.53% | 15.98% |
| CD109 | 0.043629781 | 39.60% | 49.60% | 42.38% |
| HLA-DRB1 | 0.043948828 | 99.34% | 72.03% | 73.64% |
| RHBDF2 | 0.046645232 | 12.81% | 28.73% | 49.67% |
| GPER1 | 0.04742007 | 13.13% | 39.53% | 41.14% |
| XPNPEP2 | 0.049087924 | 59.32% | 99.34% | 74.80% |
| IL6R | 0.049745524 | 54.93% | 15.57% | 57.60% |
| TMEM161A | 0.050201605 | 19.59% | 35.40% | 37.79% |
| CACNG4 | 0.05073865 | 17.62% | 71.93% | 18.62% |
| CELSR1 | 0.051217298 | 28.36% | 14.10% | 10.73% |
| CSF1 | 0.051958401 | 38.68% | 26.49% | 53.37% |
| ZAN | 0.052351116 | 11.67% | 72.23% | 43.70% |
| MFSD5 | 0.053591241 | 18.80% | 24.83% | 17.04% |
| S1PR5 | 0.053635072 | 30.73% | 22.83% | 47.94% |
| ELTD1 | 0.058172278 | 40.56% | 57.07% | 68.80% |
| TTYH3 | 0.064064238 | 16.47% | 17.10% | 14.58% |
| LRP10 | 0.064141968 | 20.58% | 17.03% | 22.91% |
| CHRNE | 0.069609585 | 22.06% | 20.42% | 26.67% |
| SLC26A6 | 0.071529916 | 11.20% | 31.98% | 15.40% |
| NPY6R | 0.072231424 | 25.59% | 76.42% | 46.99% |
| ULBP3 | 0.072772617 | 34.55% | 17.67% | 47.19% |
| CXCR5 | 0.073347916 | 27.46% | 18.95% | 32.42% |
| GPIHBP1 | 0.07686716 | 82.68% | 51.07% | 68.18% |
| GABRE | 0.07851393 | 14.17% | 39.09% | 52.18% |
| BTN3A3 | 0.079711513 | 23.74% | 13.99% | 25.87% |
| OR11A1 | 0.080154435 | 14.49% | 56.48% | 67.08% |
| GRIN2B | 0.082424733 | 16.96% | 32.50% | 44.93% |
| TGFBR3 | 0.083141392 | 25.41% | 12.89% | 22.76% |
| S1PR2 | 0.084817954 | 26.85% | 99.06% | 61.04% |
| SPRN | 0.086293416 | 70.70% | 99.18% | 66.86% |
| DPEP3 | 0.086424214 | 33.32% | 69.13% | 72.82% |
| TMEM179B | 0.086534095 | 14.21% | 12.08% | 10.21% |
| CD70 | 0.087034742 | 88.22% | 17.81% | 73.03% |
| S1PR1 | 0.08969823 | 19.82% | 99.70% | 33.44% |
| EVC2 | 0.089894424 | 53.35% | 36.67% | 39.32% |
| PCDH11Y | 0.090602169 | 55.66% | 99.69% | 32.42% |
| SLC2A6 | 0.091936867 | 17.91% | 48.87% | 19.90% |
| PTAFR | 0.098445711 | 33.11% | 44.85% | 39.69% |
| P2RX7 | 0.102272577 | 23.50% | 53.02% | 28.38% |
| ADAM18 | 0.104336724 | 84.11% | 24.64% | 49.22% |
| BST2 | 0.104466458 | 90.31% | 61.70% | 71.82% |
| BTNL3 | 0.105508382 | 57.74% | 63.97% | 77.52% |
| UPK3B | 0.107067928 | 48.51% | 35.35% | 24.50% |
| MR1 | 0.108011944 | 56.39% | 32.77% | 39.47% |
| CALY | 0.110304713 | 65.48% | 99.28% | 48.57% |
| KCNMB1 | 0.110447558 | 64.76% | 53.40% | 69.55% |
| NPR3 | 0.111672739 | 60.72% | 32.32% | 35.24% |
| PTCHD3 | 0.111985187 | 31.80% | 58.61% | 55.01% |
| GPR108 | 0.119031557 | 27.66% | 52.58% | 30.36% |
| MRVI1 | 0.121894675 | 19.91% | 13.06% | 34.23% |
| TMEM95 | 0.126037075 | 42.49% | 67.41% | 69.99% |
| FAP | 0.127343392 | 28.33% | 66.10% | 38.01% |
| BCAN | 0.127394461 | 34.65% | 80.21% | 26.67% |
| SLC52A2 | 0.127746183 | 15.00% | 27.59% | 28.44% |
| SIDT2 | 0.128965846 | 13.44% | 11.07% | 10.23% |
| IL3RA | 0.131274959 | 29.56% | 57.02% | 32.51% |
| LRFN4 | 0.131499594 | 27.62% | 16.19% | 37.23% |
| DPCR1 | 0.138274816 | 29.90% | 48.99% | 38.89% |
| FCRL5 | 0.138531663 | 36.22% | 99.20% | 63.34% |
| MYADM | 0.142338163 | 40.96% | 32.08% | 43.94% |
| SDC1 | 0.143847536 | 21.00% | 18.25% | 20.56% |
| CDH6 | 0.146632816 | 42.00% | 76.80% | 26.39% |
| TSPAN4 | 0.147567311 | 18.66% | 28.91% | 30.74% |
| OR8B4 | 0.149381345 | 87.90% | 62.52% | 67.17% |
| OR8B12 | 0.150200686 | 48.66% | 77.61% | 76.50% |
| OR8B8 | 0.150658993 | 87.02% | 99.35% | 68.61% |
| SLC14A2 | 0.156210262 | 19.95% | 58.56% | 14.06% |
| GHSR | 0.156234158 | 71.26% | 19.08% | 54.68% |
| SLC6A13 | 0.164696133 | 60.61% | 74.74% | 40.26% |
| KLRC1 | 0.165423349 | 70.79% | 11.45% | 40.18% |
| OR1F2P | 0.167047968 | 34.59% | 80.15% | 52.00% |
| IL7R | 0.168491361 | 67.99% | 99.41% | 70.50% |
| PTPRC | 0.169864738 | 42.59% | 25.65% | 28.29% |
| UNC93B1 | 0.170029747 | 50.14% | 13.72% | 37.99% |
| PLXNB2 | 0.17020525 | 10.05% | 10.74% | 10.67% |
| SLC26A2 | 0.171178969 | 11.76% | 10.61% | 11.28% |
| SIGLEC7 | 0.174623553 | 55.62% | 98.98% | 78.48% |
| RXFP1 | 0.175323639 | 58.44% | 52.19% | 40.28% |
| HTR6 | 0.181627387 | 15.43% | 99.11% | 39.39% |
| MMP25 | 0.185402559 | 22.21% | 16.96% | 30.63% |
| SLCO1C1 | 0.193949469 | 45.99% | 54.07% | 44.47% |
| KIR3DL1 | 0.194145863 | 44.82% | 77.27% | 67.27% |
| NPY2R | 0.209153189 | 46.30% | 45.28% | 67.38% |
| THY1 | 0.210844572 | 18.36% | 19.04% | 44.53% |
| IGSF8 | 0.212197037 | 29.16% | 24.02% | 17.22% |
| TMEM219 | 0.212203145 | 21.10% | 22.50% | 18.22% |
| MCOLN1 | 0.219563384 | 10.47% | 23.21% | 17.28% |
| PTPRR | 0.225118894 | 41.96% | 31.59% | 33.56% |
| LRRC3B | 0.226893135 | 13.85% | 78.36% | 54.04% |
| SLC7A10 | 0.228337953 | 15.75% | 37.57% | 29.84% |
| IL1RL2 | 0.231235711 | 33.38% | 77.33% | 63.99% |
| IL27RA | 0.238484435 | 18.83% | 43.82% | 38.56% |
| OR13G1 | 0.243162251 | 77.09% | 78.16% | 66.47% |
| TLR7 | 0.249528057 | 46.12% | 56.71% | 46.49% |
| ENPEP | 0.251772489 | 47.95% | 10.97% | 37.35% |
| FGFR1 | 0.265314907 | 11.64% | 21.89% | 17.16% |
| OR14K1 | 0.265862673 | 91.17% | 99.19% | 54.88% |
| PCDHGA3 | 0.267301318 | 26.59% | 21.80% | 21.68% |
| DSG3 | 0.267594902 | 50.64% | 42.01% | 41.91% |
| GPR1 | 0.273842346 | 22.84% | 70.37% | 24.36% |
| SLC10A3 | 0.277025033 | 14.69% | 15.82% | 18.76% |
| TGFBR2 | 0.278270861 | 27.44% | 40.77% | 29.32% |
| OR7C1 | 0.279025692 | 61.63% | 99.14% | 67.90% |
| ABCB11 | 0.281867014 | 12.42% | 39.31% | 38.80% |
| OR6J1 | 0.284944765 | 18.47% | 50.76% | 60.28% |
| TFPI | 0.291695477 | 12.54% | 16.98% | 31.45% |
| CLCA2 | 0.292814974 | 51.42% | 45.41% | 25.99% |
| TM4SF5 | 0.299823016 | 24.97% | 98.70% | 61.73% |
| OR8A1 | 0.302696206 | 73.58% | 80.33% | 67.47% |
| OR3A2 | 0.306415526 | 89.65% | 99.05% | 67.27% |
| CD40 | 0.308489803 | 68.74% | 11.91% | 40.40% |
| GPC1 | 0.312044176 | 12.15% | 24.86% | 13.06% |
| SLC51B | 0.320090119 | 65.92% | 98.93% | 53.99% |
| PCDHGA8 | 0.321583365 | 22.02% | 10.43% | 20.96% |
| OR8B2 | 0.322781396 | 66.66% | 63.56% | 63.86% |
| HLA-DOB | 0.325697853 | 58.00% | 47.99% | 53.49% |
| FCRL2 | 0.329327269 | 28.22% | 59.81% | 65.65% |
| GDPD2 | 0.330216847 | 30.99% | 36.13% | 20.08% |
| MC1R | 0.333798247 | 11.38% | 23.30% | 19.40% |
| BDKRB2 | 0.335990089 | 32.48% | 82.83% | 46.24% |
| TYRP1 | 0.340270627 | 28.06% | 24.09% | 19.40% |
| SLC6A2 | 0.340516161 | 54.47% | 98.88% | 66.54% |
| GRIA3 | 0.349815304 | 23.36% | 19.76% | 33.49% |
| GABRR1 | 0.351236381 | 48.31% | 22.01% | 38.96% |
| FCRL4 | 0.352669005 | 41.45% | 66.92% | 62.88% |
| HLA-A | 0.354772295 | 17.19% | 10.20% | 17.68% |
| ROS1 | 0.365755385 | 30.56% | 12.12% | 42.04% |
| SLC5A7 | 0.367674471 | 16.57% | 83.78% | 37.48% |
| GPA33 | 0.371405824 | 30.27% | 98.81% | 55.27% |
| MYOF | 0.385629323 | 21.19% | 41.11% | 21.06% |
| ADAM2 | 0.395455857 | 55.32% | 72.58% | 36.43% |
| CD151 | 0.39706503 | 31.10% | 16.38% | 25.88% |
| OR52E6 | 0.404255084 | 67.86% | 79.46% | 14.39% |
| FCGR2C | 0.404723052 | 27.46% | 32.10% | 47.30% |
| SHISA4 | 0.404857855 | 36.58% | 35.62% | 36.20% |
| ERVFRD-1 | 0.411868828 | 10.96% | 11.62% | 11.12% |
| NLGN4Y | 0.414016011 | 55.61% | 54.26% | 30.85% |
| CALCRL | 0.416735924 | 28.36% | 10.24% | 23.47% |
| TMPRSS11B | 0.419430434 | 44.65% | 66.33% | 38.57% |
| ACKR2 | 0.420171485 | 39.10% | 10.84% | 15.55% |
| ITLN1 | 0.431710976 | 42.83% | 99.00% | 39.89% |
| LPAR1 | 0.437577179 | 40.17% | 23.56% | 24.92% |
| GPRC5A | 0.443911646 | 25.96% | 36.66% | 42.75% |
| RHO | 0.449401165 | 35.23% | 70.26% | 50.31% |
| CD82 | 0.451520502 | 33.50% | 54.70% | 17.28% |
| ASGR2 | 0.453889237 | 38.09% | 17.99% | 60.73% |
| BTN1A1 | 0.457469508 | 17.26% | 75.38% | 36.90% |
| VSTM4 | 0.470409176 | 46.10% | 29.19% | 37.90% |
| SIGLEC5 | 0.471798433 | 33.14% | 98.80% | 50.91% |
| CR1 | 0.472408291 | 21.88% | 34.07% | 31.65% |
| TNFSF18 | 0.47248415 | 39.04% | 99.11% | 50.55% |
| ITGAD | 0.477965365 | 35.44% | 76.09% | 41.32% |
| CSF1R | 0.482231444 | 25.84% | 61.18% | 29.47% |
| PCDHGA12 | 0.487188389 | 18.84% | 10.66% | 12.80% |
| ITGAX | 0.492293391 | 33.77% | 62.72% | 44.85% |
| HRH1 | 0.496070302 | 13.78% | 77.21% | 14.54% |
| LTB4R2 | 0.499245999 | 33.67% | 32.50% | 29.70% |
| ABCA1 | 0.501257794 | 15.16% | 15.98% | 10.10% |
| PCDH18 | 0.50445456 | 12.44% | 43.11% | 12.32% |
| OR1C1 | 0.504532204 | 74.30% | 99.04% | 58.03% |
| TPSG1 | 0.513791919 | 15.34% | 57.52% | 36.54% |
| TECTB | 0.524070714 | 35.21% | 56.48% | 61.23% |
| MS4A15 | 0.55496264 | 98.39% | 33.58% | 22.56% |
| HLA-DQB1 | 0.560863505 | 66.88% | 33.66% | 58.50% |
| CLEC7A | 0.566991408 | 61.95% | 23.70% | 25.62% |
| CCR3 | 0.578683946 | 31.30% | 60.27% | 55.63% |
| SLC10A6 | 0.580091405 | 19.54% | 39.67% | 34.23% |
| OR8D1 | 0.596270694 | 24.63% | 79.33% | 43.53% |
| SLC10A4 | 0.602488524 | 12.23% | 15.04% | 15.86% |
| CCRL2 | 0.604393418 | 43.96% | 98.88% | 21.95% |
| ASIC5 | 0.608054386 | 54.30% | 81.59% | 40.83% |
| ADRA2C | 0.616104389 | 15.05% | 33.74% | 29.29% |
| CLEC9A | 0.617473862 | 38.03% | 35.89% | 48.96% |
| DUOXA1 | 0.619770714 | 13.09% | 36.92% | 36.36% |
| OR10D3 | 0.620509038 | 34.29% | 71.33% | 45.38% |
| OR8D2 | 0.632808285 | 64.17% | 58.19% | 59.91% |
| CLEC12A | 0.633663241 | 40.08% | 98.80% | 46.02% |
| MEP1A | 0.647686397 | 20.07% | 98.50% | 22.15% |
| GJB4 | 0.648355542 | 21.58% | 98.41% | 42.69% |
| FZD9 | 0.64889927 | 50.45% | 48.65% | 47.69% |
| SLC44A2 | 0.649439099 | 12.58% | 12.55% | 12.01% |
| OR14A2 | 0.660245293 | 60.97% | 99.10% | 41.17% |
| OR8B3 | 0.663064669 | 35.02% | 58.48% | 47.13% |
| IL13RA2 | 0.667080328 | 31.79% | 30.27% | 34.08% |
| MILR1 | 0.674176317 | 41.25% | 34.83% | 18.69% |
| SORCS1 | 0.695659133 | 27.17% | 63.09% | 20.35% |
| GPR149 | 0.697360216 | 44.76% | 27.14% | 23.83% |
| CLEC1A | 0.69855076 | 16.16% | 98.34% | 39.08% |
| OR8G1 | 0.700029247 | 48.42% | 59.96% | 45.23% |
| ITGAL | 0.700284334 | 15.57% | 57.11% | 11.69% |
| KIR2DL4 | 0.711538473 | 73.52% | 55.15% | 55.76% |
| OR8G5 | 0.726783101 | 55.19% | 39.67% | 46.27% |
| OR2AT4 | 0.730479448 | 57.56% | 29.53% | 35.85% |
| CCR2 | 0.738631773 | 23.19% | 98.17% | 25.66% |
| CNTNAP4 | 0.752168179 | 18.26% | 35.49% | 25.87% |
| SLC22A3 | 0.754846789 | 26.52% | 58.93% | 31.07% |
| OR4D1 | 0.755277997 | 33.10% | 64.97% | 37.15% |
| OR6F1 | 0.755553415 | 75.53% | 97.76% | 26.49% |
| GPM6A | 0.755936769 | 16.72% | 14.76% | 22.54% |
| UPK3A | 0.758561782 | 27.49% | 98.19% | 25.25% |
| MUC21 | 0.784791847 | 60.17% | 32.54% | 45.31% |
| ASGR1 | 0.789223379 | 15.16% | 50.12% | 22.04% |
| MUSK | 0.794634299 | 28.41% | 23.46% | 17.41% |
| PCDH15 | 0.802189331 | 12.88% | 38.54% | 17.19% |
| NPFFR2 | 0.803972336 | 18.04% | 64.73% | 15.18% |
| GRIN3B | 0.805884492 | 34.69% | 14.30% | 28.02% |
| VNN1 | 0.807570724 | 47.83% | 59.74% | 36.35% |
| CD74 | 0.814203565 | 34.46% | 29.95% | 26.52% |
| GRM6 | 0.818292499 | 25.79% | 61.95% | 41.09% |
| BOC | 0.820910863 | 12.71% | 12.06% | 13.40% |
| KIR2DL1 | 0.823617205 | 71.14% | 53.01% | 31.27% |
| EREG | 0.824067105 | 43.12% | 61.54% | 30.64% |
| SLC22A2 | 0.829422311 | 38.37% | 65.89% | 32.44% |
| CNTN6 | 0.833575047 | 26.98% | 13.27% | 16.50% |
| ADORA2A | 0.834148243 | 12.33% | 50.83% | 30.69% |
| LRRC32 | 0.849594703 | 18.03% | 11.26% | 37.22% |
| OR1E2 | 0.851206648 | 71.67% | 98.19% | 49.96% |
| HLA-DRB5 | 0.857348426 | 50.84% | 97.96% | 45.82% |
| FCRL1 | 0.863158359 | 44.76% | 98.03% | 31.54% |
| ABCC3 | 0.89599808 | 11.50% | 28.19% | 12.20% |
| CDH19 | 0.897963086 | 31.79% | 38.89% | 26.58% |
| OR52B6 | 0.908122928 | 39.77% | 21.13% | 17.03% |
| SLC17A1 | 0.915852827 | 24.27% | 16.53% | 32.31% |
| AQP8 | 0.91910142 | 14.67% | 37.05% | 29.28% |
| TM4SF4 | 0.919889102 | 39.50% | 36.56% | 40.08% |
| OR8K3 | 0.927735573 | 50.61% | 55.79% | 31.66% |
| FCGR2A | 0.96423416 | 11.89% | 17.21% | 15.01% |
| SLC22A25 | 0.971153274 | 11.53% | 35.59% | 16.21% |
| SLC19A3 | 0.973316054 | 10.44% | 30.64% | 12.49% |
| KITLG | 1.22912E−09 | 33.22% | 8.72% | 1.73% |
| EPOR | 3.64388E−07 | 37.77% | 52.03% | 1.36% |
| LRP1 | 1.43654E−05 | 22.89% | 9.74% | 22.90% |
| SLC1A3 | 1.70439E−05 | 0.47% | 52.15% | 58.29% |
| CRIM1 | 2.3671E−05 | 10.46% | 3.26% | 19.75% |
| PIEZO1 | 3.72517E−05 | 6.01% | 11.45% | 17.13% |
| SCAP | 4.45561E−05 | 2.45% | 8.05% | 13.10% |
| DDR2 | 6.74876E−05 | 2.63% | 28.27% | 31.13% |
| SLC38A2 | 7.23319E−05 | 10.61% | 7.33% | 4.92% |
| CNTNAP1 | 7.33744E−05 | 8.44% | 11.28% | 16.73% |
| GPR126 | 0.000115686 | 56.76% | 7.36% | 40.95% |
| TMEM67 | 0.000207487 | 13.84% | 6.96% | 1.98% |
| TCTN2 | 0.000294838 | 12.12% | 7.00% | 1.70% |
| HEG1 | 0.000324881 | 1.32% | 13.80% | 26.29% |
| CD276 | 0.000332245 | 5.66% | 9.27% | 15.55% |
| SLC41A2 | 0.000375485 | 5.29% | 28.71% | 3.36% |
| OSMR | 0.000429575 | 17.80% | 2.08% | 15.09% |
| ATP13A1 | 0.000649164 | 2.17% | 11.47% | 11.98% |
| FAT4 | 0.001200168 | 8.87% | 29.26% | 30.85% |
| TMEM63A | 0.001200493 | 33.38% | 5.87% | 22.36% |
| ADAM9 | 0.001366603 | 12.74% | 5.63% | 9.12% |
| BTN2A1 | 0.001550834 | 11.85% | 8.34% | 13.02% |
| SERINC1 | 0.001952639 | 5.37% | 4.67% | 0.33% |
| DLK2 | 0.00209582 | 6.12% | 22.46% | 21.41% |
| OPRL1 | 0.002200766 | 0.01% | 6.60% | 53.82% |
| FAM189B | 0.002623661 | 1.29% | 13.06% | 13.27% |
| ANPEP | 0.002631649 | 7.72% | 57.25% | 56.01% |
| TCTN3 | 0.003283122 | 8.06% | 8.80% | 9.73% |
| BTN2A2 | 0.003593958 | 5.87% | 5.53% | 23.92% |
| PLA2R1 | 0.003601681 | 31.45% | 1.46% | 45.84% |
| EFNA4 | 0.004659915 | 52.46% | 3.85% | 54.17% |
| ITGB1 | 0.005116504 | 10.13% | 4.32% | 6.51% |
| SPPL2A | 0.0079037 | 6.19% | 6.57% | 4.24% |
| ATG9A | 0.00823111 | 3.26% | 5.11% | 11.31% |
| BMPR1A | 0.008696054 | 6.96% | 6.18% | 7.15% |
| SLC39A14 | 0.010300373 | 5.92% | 13.63% | 11.43% |
| PPAP2A | 0.010349324 | 2.84% | 7.97% | 6.97% |
| TIMD4 | 0.010952103 | 9.98% | 72.04% | 56.92% |
| BTN3A1 | 0.011807557 | 21.66% | 4.62% | 21.92% |
| ABCB9 | 0.012872125 | 3.69% | 16.39% | 20.94% |
| PSEN2 | 0.013607372 | 1.57% | 44.97% | 16.40% |
| NOTCH2 | 0.015085852 | 23.85% | 9.53% | 28.46% |
| FAS | 0.015303925 | 41.01% | 7.09% | 37.76% |
| TM9SF4 | 0.015507074 | 2.08% | 4.85% | 6.30% |
| TSPAN3 | 0.015549376 | 0.03% | 4.22% | 4.12% |
| PRND | 0.016101818 | 4.40% | 66.53% | 65.83% |
| IGF2R | 0.016362752 | 9.92% | 5.44% | 3.71% |
| TMEM104 | 0.017780302 | 1.39% | 22.68% | 4.67% |
| FAIM2 | 0.0194138 | 5.67% | 79.10% | 16.13% |
| NPR2 | 0.020748914 | 5.84% | 26.26% | 6.55% |
| TREH | 0.021038328 | 35.00% | 8.77% | 6.40% |
| ADCY3 | 0.023753612 | 0.57% | 3.65% | 5.53% |
| SLC19A1 | 0.024259375 | 5.18% | 18.97% | 25.28% |
| TNFRSF10D | 0.024866438 | 41.92% | 9.94% | 52.01% |
| UPK2 | 0.027646553 | 0.52% | 40.88% | 72.80% |
| ECE1 | 0.027824102 | 4.17% | 14.54% | 0.41% |
| TENM3 | 0.029749637 | 19.17% | 5.48% | 2.74% |
| STT3B | 0.030311224 | 6.09% | 5.49% | 5.80% |
| LRIG2 | 0.030916383 | 0.57% | 0.97% | 3.72% |
| NCSTN | 0.035326878 | 8.01% | 11.55% | 10.17% |
| CACHD1 | 0.036374936 | 1.32% | 2.27% | 12.48% |
| SLC15A3 | 0.037803739 | 9.28% | 28.53% | 25.09% |
| AMN | 0.038022559 | 3.05% | 12.27% | 9.08% |
| AMIGO2 | 0.040462608 | 42.52% | 33.54% | 1.11% |
| LRP5 | 0.04064938 | 11.33% | 0.99% | 15.79% |
| NPC1 | 0.040734377 | 4.41% | 10.32% | 4.47% |
| PLXDC2 | 0.041556302 | 48.29% | 9.83% | 34.02% |
| HLA-DPA1 | 0.041968046 | 32.97% | 4.78% | 61.81% |
| APLP2 | 0.042589097 | 10.07% | 4.84% | 4.06% |
| NLGN3 | 0.042745598 | 1.92% | 1.58% | 18.82% |
| SLC17A5 | 0.044873172 | 8.75% | 5.89% | 5.21% |
| LAMP1 | 0.046646461 | 10.03% | 8.94% | 3.99% |
| OR52W1 | 0.048413228 | 75.90% | 21.33% | 5.51% |

TABLE 12
Surface proteins associated with the SCLC-I subtype from the Sato dataset

| Surface Protein | ANOVA p value | I vs N | I vs P | I vs A |
|---|---|---|---|---|
| ULBP2 | 0.000401771 | 33.616% | 21.160% | 30.038% |
| LAMP3 | 0.000500427 | 27.061% | 18.820% | 23.810% |
| IL1R2 | 0.001123023 | 48.534% | 16.083% | 59.376% |
| FREM2 | 0.001290176 | 27.403% | 29.302% | 21.104% |
| ABCC4 | 0.001291636 | 10.574% | 21.181% | 30.267% |
| CEACAM6 | 0.001572035 | 28.896% | 48.775% | 24.840% |
| CTLA4 | 0.00235198 | 59.992% | 12.364% | 34.041% |
| LPAR6 | 0.004382294 | 29.854% | 13.805% | 26.729% |
| TMEM116 | 0.008232977 | 15.832% | 18.672% | 11.796% |
| SLC6A14 | 0.010025829 | 38.415% | 23.327% | 44.788% |
| QRFPR | 0.010389646 | 22.644% | 58.781% | 28.888% |
| ITGB6 | 0.010994872 | 13.902% | 11.241% | 39.858% |
| SLC26A9 | 0.011576247 | 19.328% | 51.036% | 13.183% |
| SLC44A5 | 0.012341309 | 26.223% | 23.536% | 22.349% |
| CDH26 | 0.012561895 | 31.647% | 14.247% | 14.401% |
| ROS1 | 0.014075255 | 50.362% | 23.774% | 22.464% |
| TMPRSS13 | 0.016875706 | 29.883% | 25.000% | 46.376% |
| MPZL2 | 0.016942684 | 20.914% | 27.805% | 32.087% |
| IL1RAP | 0.019303137 | 37.041% | 16.450% | 54.210% |
| NIPAL4 | 0.019309994 | 36.139% | 48.262% | 44.384% |
| SLC2A1 | 0.028160352 | 15.621% | 10.434% | 13.744% |
| GPR110 | 0.030651959 | 23.740% | 37.659% | 39.189% |
| MUC1 | 0.032222447 | 18.344% | 14.318% | 11.590% |
| CD109 | 0.038542669 | 11.875% | 17.350% | 22.645% |
| CDH3 | 0.043188459 | 25.570% | 45.250% | 47.256% |
| CLCA2 | 0.043321363 | 42.009% | 73.048% | 58.739% |
| GJB6 | 0.047670625 | 51.153% | 52.840% | 80.502% |
| SLC51A | 0.054500968 | 51.515% | 36.209% | 49.467% |
| ZAN | 0.063702876 | 19.138% | 39.480% | 10.196% |
| PVRL3 | 0.065439262 | 14.855% | 33.512% | 30.180% |
| SLC7A5 | 0.072533224 | 29.273% | 18.383% | 18.077% |
| CLDN1 | 0.073714631 | 32.382% | 27.791% | 36.580% |
| AVPR2 | 0.075099561 | 50.256% | 30.796% | 50.875% |
| ENTPD3 | 0.076708202 | 18.680% | 36.303% | 23.105% |
| GJB2 | 0.078372255 | 58.528% | 26.796% | 53.764% |
| LPAR3 | 0.081401485 | 11.129% | 30.020% | 39.629% |
| ADAM20 | 0.091064095 | 46.719% | 28.273% | 24.993% |
| CEACAM7 | 0.094983069 | 37.838% | 47.755% | 10.661% |
| OR51M1 | 0.10617364 | 28.967% | 38.831% | 27.455% |
| SDC1 | 0.108283365 | 23.959% | 27.889% | 28.630% |
| TMC7 | 0.127266725 | 28.041% | 10.121% | 15.949% |
| DSG3 | 0.138965552 | 59.627% | 55.316% | 74.495% |
| CLDN6 | 0.140494484 | 53.224% | 33.893% | 30.715% |
| PANX2 | 0.146302225 | 18.780% | 30.390% | 28.326% |
| EGF | 0.147163821 | 50.558% | 10.109% | 42.195% |
| ATP13A5 | 0.16067304 | 22.588% | 26.785% | 39.892% |
| PCDHB5 | 0.184159617 | 27.607% | 38.288% | 41.172% |
| ABCA13 | 0.189178704 | 21.495% | 48.736% | 40.243% |
| UPK1B | 0.199342196 | 31.540% | 30.963% | 27.315% |
| LYPD3 | 0.202574616 | 34.065% | 26.625% | 42.489% |
| OXGR1 | 0.206375308 | 58.893% | 26.740% | 26.748% |
| TACSTD2 | 0.208454138 | 18.494% | 22.452% | 35.639% |
| PCDH20 | 0.211076831 | 15.426% | 44.983% | 16.357% |
| GPRC5A | 0.213072322 | 13.133% | 30.278% | 44.221% |
| SLC46A2 | 0.217209365 | 46.731% | 43.778% | 35.347% |
| EFNB2 | 0.22377892 | 10.721% | 16.446% | 19.745% |
| TREM1 | 0.223947474 | 33.399% | 21.515% | 32.476% |
| ERVMER34-1 | 0.229064798 | 19.235% | 14.167% | 16.286% |
| GFRA3 | 0.232751993 | 35.323% | 28.622% | 27.143% |
| CALCR | 0.233111903 | 45.738% | 40.242% | 37.457% |
| CLDN20 | 0.238970005 | 14.562% | 20.459% | 26.028% |
| TMPRSS11D | 0.239442354 | 23.926% | 31.485% | 30.009% |
| ACE2 | 0.240101964 | 47.376% | 36.245% | 36.245% |
| PTPRO | 0.250845719 | 21.768% | 22.274% | 18.421% |
| SLC52A3 | 0.269986489 | 36.261% | 13.813% | 46.402% |
| LYPD6B | 0.272299557 | 37.875% | 40.037% | 33.835% |
| PTPRZ1 | 0.291648271 | 23.355% | 45.840% | 24.104% |
| SLCO1B3 | 0.298279793 | 68.423% | 12.303% | 40.314% |
| TYRP1 | 0.302111078 | 24.148% | 32.252% | 36.444% |
| GPR87 | 0.310279746 | 26.904% | 53.279% | 54.040% |
| F2RL2 | 0.332184561 | 37.288% | 41.031% | 26.633% |
| MSLN | 0.343970634 | 46.256% | 18.829% | 33.190% |
| HLA-DQB2 | 0.350158887 | 20.153% | 15.104% | 18.533% |
| SLAMF9 | 0.353137509 | 14.036% | 23.481% | 11.639% |
| GPR68 | 0.353981958 | 47.305% | 19.060% | 20.809% |
| VN1R3 | 0.362208892 | 44.630% | 39.140% | 31.468% |
| UNC93B1 | 0.369803275 | 16.255% | 14.871% | 28.872% |
| EPHA1 | 0.377588291 | 21.526% | 16.347% | 21.495% |
| HTR2B | 0.383339042 | 51.236% | 22.463% | 24.653% |
| SLITRK1 | 0.414452608 | 53.002% | 56.783% | 21.849% |
| EMR1 | 0.414508571 | 44.855% | 14.646% | 17.070% |
| OR7A5 | 0.429503421 | 44.713% | 15.734% | 36.237% |
| SLC34A2 | 0.43388428 | 10.692% | 35.292% | 13.931% |
| HTR2C | 0.448988824 | 38.674% | 33.834% | 48.501% |
| MUC15 | 0.464271203 | 28.149% | 38.156% | 53.016% |
| GPR45 | 0.472734149 | 42.147% | 26.431% | 21.258% |
| CNTN5 | 0.479426869 | 58.117% | 25.530% | 24.467% |
| ADORA2B | 0.490531691 | 16.395% | 25.203% | 21.145% |
| NRG4 | 0.505959614 | 33.911% | 31.350% | 32.512% |
| OR2F2 | 0.521732819 | 39.682% | 12.472% | 15.795% |
| TMEM171 | 0.52708684 | 31.488% | 12.212% | 18.162% |
| RXFP1 | 0.54822571 | 32.665% | 27.142% | 30.082% |
| MDGA2 | 0.554126454 | 42.113% | 36.030% | 41.024% |
| OR6B1 | 0.565764307 | 15.997% | 37.251% | 10.699% |
| MUC16 | 0.574609577 | 51.836% | 32.324% | 43.961% |
| FFAR4 | 0.586185845 | 30.353% | 22.593% | 35.908% |
| VNN3 | 0.609937374 | 48.825% | 40.015% | 42.229% |
| CR1 | 0.610551241 | 44.176% | 11.561% | 22.088% |
| LRRN4 | 0.620032487 | 43.633% | 26.532% | 28.864% |
| GNRHR | 0.734837204 | 14.775% | 32.424% | 26.310% |
| ACKR4 | 0.740067283 | 18.813% | 22.240% | 12.871% |
| TMPRSS11B | 0.756391224 | 23.647% | 27.261% | 36.061% |
| OR5V1 | 0.764212185 | 37.067% | 14.598% | 20.162% |
| PRSS8 | 0.803107736 | 15.101% | 10.536% | 13.219% |
| MEP1B | 0.804628935 | 36.581% | 22.287% | 11.916% |
| SLC22A11 | 0.885014214 | 35.337% | 26.901% | 13.936% |
| ABCC10 | 1.00807E−07 | 14.767% | 1.216% | 2.741% |
| DCBLD1 | 1.1404E−06 | 31.620% | 4.903% | 14.499% |
| F11R | 1.36563E−06 | 9.197% | 2.673% | 6.087% |
| ADAM17 | 1.60763E−05 | 12.457% | 4.381% | 7.925% |
| ITGAE | 1.73594E−05 | 7.132% | 0.502% | 3.595% |
| MICA | 1.92641E−05 | 28.382% | 5.300% | 25.966% |
| SEMA4C | 5.72638E−05 | 14.299% | 6.514% | 5.206% |
| EPHA2 | 0.000198743 | 41.813% | 26.823% | 46.253% |
| GPR107 | 0.00023609 | 9.022% | 4.047% | 6.809% |
| SLC11A2 | 0.000340249 | 12.708% | 7.690% | 7.153% |
| TSPAN31 | 0.000343171 | 23.140% | 3.247% | 4.969% |
| LAMP1 | 0.000384984 | 9.557% | 2.627% | 6.110% |
| ABCA1 | 0.000407256 | 14.279% | 2.386% | 13.243% |
| SLC12A7 | 0.000429879 | 6.601% | 3.175% | 1.590% |
| CD46 | 0.000586389 | 6.835% | 2.602% | 6.828% |
| DSC2 | 0.000621513 | 16.234% | 3.214% | 38.646% |
| SLC31A1 | 0.000940061 | 26.990% | 6.241% | 21.250% |
| CD274 | 0.000951345 | 40.294% | 3.023% | 21.822% |
| PCDH1 | 0.001082683 | 15.109% | 27.859% | 28.287% |
| TMEM9 | 0.001377575 | 1.684% | 5.723% | 6.547% |
| EMP2 | 0.001477302 | 4.856% | 12.404% | 2.868% |
| ATRAID | 0.001636825 | 12.721% | 6.131% | 10.313% |
| CXCL16 | 0.003528987 | 35.088% | 3.172% | 16.673% |
| ITGB5 | 0.004208038 | 11.972% | 4.089% | 17.520% |
| ZDHHC5 | 0.004653623 | 7.929% | 6.247% | 7.814% |
| SLC37A3 | 0.005332971 | 3.113% | 5.188% | 2.832% |
| RNF149 | 0.005531618 | 14.775% | 4.299% | 14.857% |
| SLAMF8 | 0.005551306 | 49.786% | 0.477% | 21.436% |
| SLC33A1 | 0.005647423 | 7.888% | 2.271% | 2.174% |
| TSPAN13 | 0.006210859 | 1.847% | 7.745% | 0.906% |
| THBD | 0.006315115 | 26.931% | 21.150% | 21.213% |
| P2RY6 | 0.006366337 | 50.048% | 6.705% | 27.836% |
| CDH1 | 0.006482738 | 9.663% | 7.428% | 7.370% |
| C3AR1 | 0.007003309 | 34.122% | 1.010% | 17.107% |
| GPR157 | 0.007599707 | 19.989% | 2.341% | 22.021% |
| SLC39A4 | 0.007876928 | 14.161% | 4.295% | 4.092% |
| EFNA1 | 0.007891944 | 20.804% | 0.404% | 24.316% |
| CDCP1 | 0.007958915 | 1.822% | 1.423% | 6.232% |
| TNFSF15 | 0.009410143 | 14.459% | 1.449% | 11.172% |
| LPAR5 | 0.010109622 | 33.939% | 7.455% | 35.447% |
| TSPAN33 | 0.01063962 | 17.422% | 7.100% | 19.477% |
| CD9 | 0.013244926 | 9.064% | 2.699% | 17.328% |
| IL13RA1 | 0.013356457 | 14.127% | 2.085% | 13.021% |
| IL17RA | 0.013615554 | 17.979% | 4.282% | 15.084% |
| LYSMD3 | 0.013667996 | 6.733% | 0.769% | 3.200% |
| MR1 | 0.014310513 | 22.268% | 7.679% | 35.081% |
| TMEM37 | 0.01458688 | 54.056% | 15.680% | 34.939% |
| MET | 0.016631502 | 29.117% | 11.653% | 3.321% |
| SEMA4B | 0.01729249 | 7.237% | 4.817% | 21.461% |
| LRIG3 | 0.019103303 | 1.499% | 21.173% | 14.097% |
| ADAM9 | 0.019713191 | 12.943% | 6.388% | 12.784% |
| LTBR | 0.021083671 | 9.975% | 9.724% | 16.849% |
| LMAN2L | 0.021967872 | 9.752% | 3.135% | 3.320% |
| SLC47A1 | 0.022622797 | 40.991% | 7.648% | 25.502% |
| DGCR2 | 0.023431268 | 12.239% | 7.817% | 5.004% |
| TLR3 | 0.024401176 | 26.580% | 4.189% | 30.641% |
| SLC12A6 | 0.02474767 | 22.363% | 9.449% | 26.572% |
| TM4SF1 | 0.026267868 | 7.239% | 1.953% | 17.463% |
| ESYT3 | 0.026675173 | 8.354% | 15.233% | 5.129% |
| BCAN | 0.027012643 | 33.601% | 8.132% | 2.357% |
| LRRC4 | 0.028490024 | 34.757% | 46.633% | 38.560% |
| PLB1 | 0.029078035 | 32.743% | 11.511% | 19.376% |
| IFNGR1 | 0.034174512 | 10.475% | 5.439% | 11.688% |
| TM9SF2 | 0.034950372 | 5.576% | 3.487% | 6.981% |
| GLIPR1 | 0.035521861 | 32.326% | 7.468% | 18.401% |
| C14orf37 | 0.035801097 | 63.457% | 13.510% | 28.378% |
| FLVCR2 | 0.037917827 | 22.459% | 8.245% | 7.603% |
| STEAP4 | 0.038626049 | 17.663% | 28.655% | 22.804% |
| CLDN12 | 0.040350372 | 5.558% | 5.867% | 3.123% |
| TMEM9B | 0.043371434 | 9.870% | 7.121% | 5.370% |
| ALCAM | 0.045036666 | 3.578% | 10.628% | 2.237% |
| SCN11A | 0.045107412 | 14.980% | 9.254% | 15.953% |
| ADCY7 | 0.045393791 | 22.209% | 1.888% | 16.249% |
| SLC5A6 | 0.04680909 | 22.261% | 4.969% | 15.821% |
| ABCC3 | 0.049733245 | 41.071% | 15.109% | 38.927% |

### Example 2—Analysis of Therapeutic Surface Targets

To investigate cell surface targets broadly, the inventors examined expression of genes that encode known targets of therapeutic monoclonal antibodies, CARs, or ADCs across each subtype in SCLC tumor and cell line data sets.

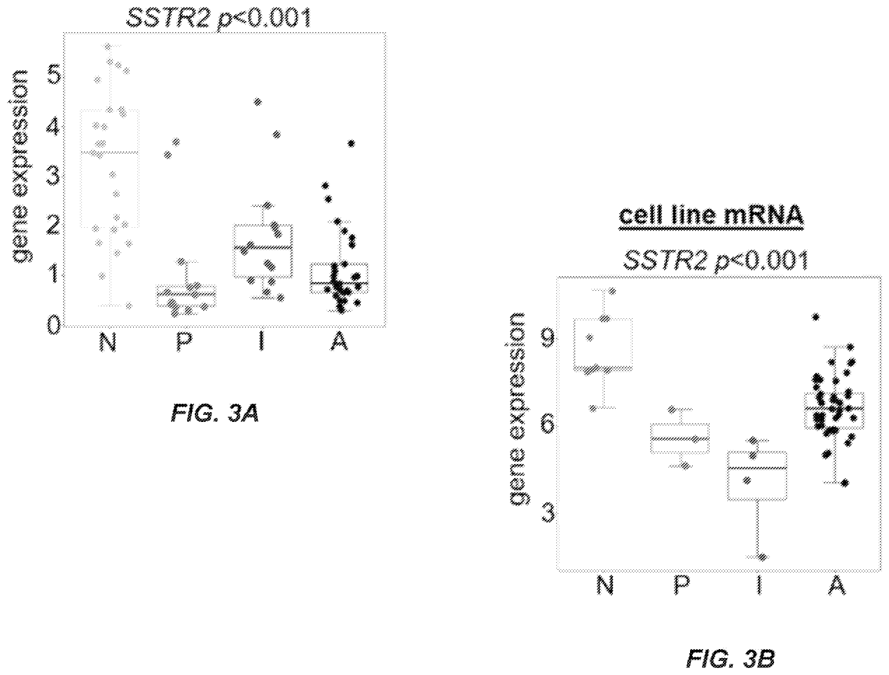

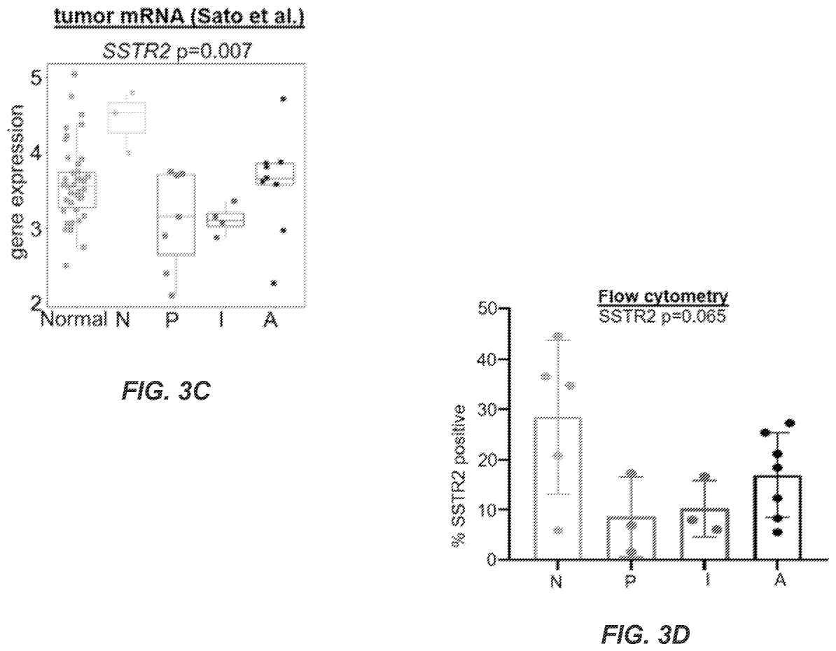

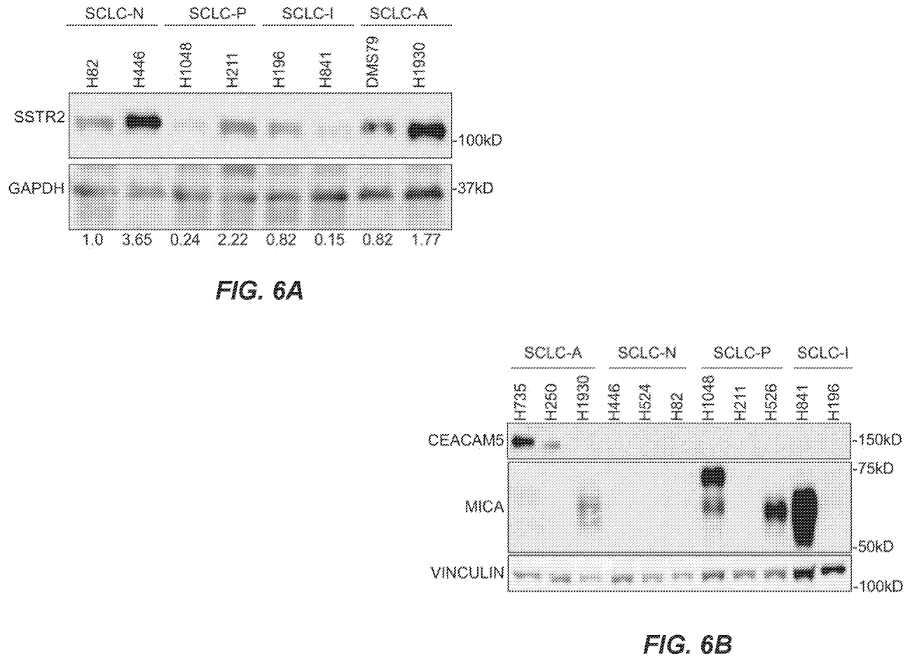

The inventors identified several surface protein-encoding genes with consistent relative expression patterns among our three data sets and with therapeutics already in development. For example, somatostatin receptor 2 (SSTR2) is a well-established target expressed in low- and intermediate-grade neuroendocrine tumors (NETs), in which somatostatin analogues, such as octreotide and lanreotide, which bind SSTR2, are routinely used therapeutically. SSTR2 is also the target of an ADC, PEN-221, already under development for less aggressive NETs^{26,27}. While SSTR2 is not broadly expressed in all SCLCs, it was observed to be highly expressed in both SCLC-N tumors and cell lines (FIGS. 3A-3C). Further, flow cytometric analyses confirmed robust expression of SSTR2 in SCLC-N cell lines and supported the trend toward greater relative expression in SCLC-N (FIG. 3D). In addition, Western blot analysis of cell lines shows that SSTR2 is preferentially expressed in SCLC-N (FIG. 6A).

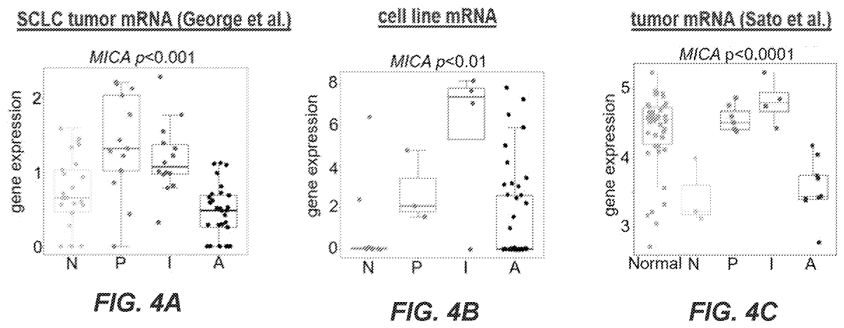

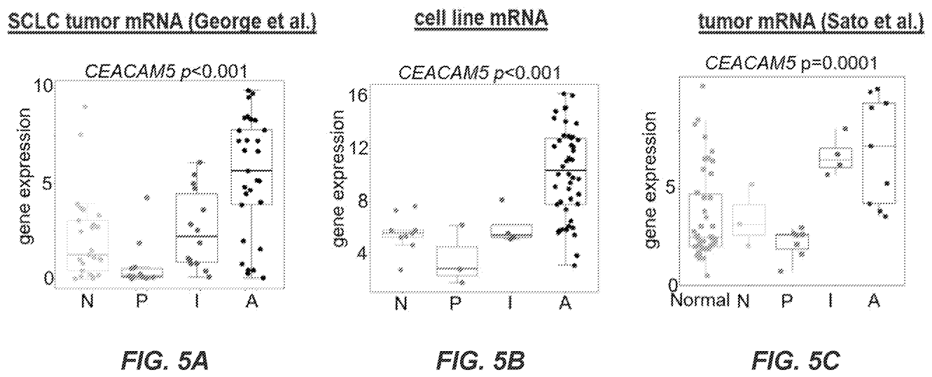

For SCLC-P and SCLC-I, MICA, the gene which encodes MHC class I polypeptide-related sequence A, was identified as highly expressed in both of these subtypes (FIGS. 4A-4C). In addition, Western blot analysis of cell lines shows that on a global scale, there is differential protein expression across the subtypes of MICA, expressed in SCLC-I and -P (FIG. 6B). MICA normally acts as the ligand for Natural Killer Group 2 (NKG2D) receptor activation, however prolonged NKG2D activation can ultimately suppress Natural Killer (NK) cell and CD8+ T-cell activity, allowing for immune evasion. MICA is the target of a molecule (IPH43) currently in preclinical development, with proposed dual mechanism of blocking MICA's interaction with NKG2D, while also designed as an ADC targeting MICA-expressing cells²⁸. As for the remaining subtype, SCLC-A, carcinoembryonic antigen-related cell adhesion molecule 5 (CEACAM5), a cell adhesion molecule overexpressed in gastrointestinal and breast cancers as well as in NSCLC, is the target of labetuzumab govitecan, an ADC in clinical investigation for patients with refractory metastatic colorectal cancer, as well as a CAR T-cell²⁹⁻³². While this molecule has not been previously described as a target for SCLC-specific therapies, the inventors observed differential expression in the datasets, with significantly higher CEACAM5 expression in SCLC-A (FIGS. 5A-5C). In addition, Western blot analysis of cell lines shows that on a global scale, there is differential protein expression across the subtypes of CEACAM5, expressed in SCLC-A (FIG. 6B).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Govindan, R. et al. Changing epidemiology of small-cell lung cancer in the United States over the last 30 years: Analysis of the surveillance, epidemiologic, and end results database. J. Clin. Oncol. 24, 4539— 4544 (2006).
2. Karachaliou, N. et al. Cellular and molecular biology of small cell lung cancer: An overview. Translational Lung Cancer Research 5, 2-15 (2016).
3. Byers, L. A. & Rudin, C. M. Small cell lung cancer: Where do we go from here? Cancer 121, 664-672 (2015).
4. Eshoo, A. G. H.R.733-112th Congress (2011-2012): Recalcitrant Cancer Research Act of 2012. (2012).
5. Peifer, M. et al. Integrative genome analyses identify key somatic driver mutations of small-cell lung cancer. Nat. Genet. 44, 1104-1110 (2012).
6. Demedts, I. K., Vermaelen, K. Y. & Van Meerbeeck, J. P. Treatment of extensive-stage small cell lung carcinoma: Current status and future prospects. European Respiratory Journal 202-215 (2010).
7. Alvarado-Luna, G. & Morales-Espinosa, D. Treatment for small cell lung cancer, where are we now?-A review. Translational Lung Cancer Research 5, 26-38 (2016).
8. Paz-Ares, L. et al. Durvalumab plus platinum-etoposide versus platinum-etoposide in first-line treatment of extensive-stage small-cell lung cancer (CASPIAN): a randomised, controlled, open-label, phase 3 trial. Lancet 394, 1929-1939 (2019).
9. Horn, L. et al. First-line atezolizumab plus chemotherapy in extensive-stage small-cell lung cancer. N. Engl. J. Med. 379, 2220-2229 (2018).
10. Trigo, J. et al. Lurbinectedin as second-line treatment for patients with small-cell lung cancer: a singlearm, open-label, phase 2 basket trial. Lancet Oncol. 21, 645-654 (2020).
11. FDA grants accelerated approval to lurbinectedin for metastatic small cell lung cancer I FDA. Available on the World Wide Web at fda.gov/drugs/drug-approvals-and-databases/fda-grants-accelerated-approval-lurbinectedin-metastatic-small-cell-lung-cancer. (Accessed: 20th July 2020)
12. Waqar, S. N. & Morgensztern, D. Treatment advances in small cell lung cancer (SCLC). Pharmacology and Therapeutics 180, 16-23 (2017).
13. Herbst, R. S., Morgensztern, D. & Boshoff, C. The biology and management of non-small cell lung cancer. Nature 553, 446-454 (2018).
14. Duma, N., Santana-Davila, R. & Molina, J. R. Non-Small Cell Lung Cancer: Epidemiology, Screening, Diagnosis, and Treatment. Mayo Clinic Proceedings 94, 1623-1640 (2019).
15. Cardnell, R. J. et al. Protein expression of TTF1 and cMYC define distinct molecular subgroups of small cell lung cancer with unique vulnerabilities to aurora kinase inhibition, DLL3 targeting, and other targeted therapies. Oncotarget 8, 73419-73432 (2017).
16. Stewart, C. A. et al. Single-cell analyses reveal increased intratumoral heterogeneity after the onset of therapy resistance in small-cell lung cancer. Nat. Cancer 1, 423-436 (2020).
17. George, J. et al. Comprehensive genomic profiles of small cell lung cancer. Nature 524, 47-53 (2015).
18. McColl, K. et al. Reciprocal expression of INSM1 and YAP1 defines subgroups in small cell lung cancer. Oncotarget 8, 73745-73756 (2017).
19. Yarchoan, M., Hopkins, A. & Jaffee, E. M. Tumor mutational burden and response rate to PD-1 inhibition. New England Journal of Medicine 377, 2500-2501 (2017).
20. Carretero-Gonzalez, A. et al. Comparative safety analysis of immunotherapy combined with chemotherapy versus monotherapy in solid tumors: A meta-analysis of randomized clinical trials. Oncotarget 10, 3294-3301 (2019).
21. Rizvi, N. A. et al. Mutational landscape determines sensitivity to PD-1 blockade in non-small cell lung cancer. Science (80-.). 348, 124-128 (2015).
22. Byers, L. A. et al. Proteomic profiling identifies dysregulated pathways in small cell lung cancer and novel therapeutic targets including PARP1. Cancer Discov. 2, 798-811 (2012).
23. Byers, L. A., Chiappori, A. & Smit, M.-A. D. Phase 1 study of AMG 119, a chimeric antigen receptor (CAR) T cell therapy targeting DLL3, in patients with relapsed/refractory small cell lung cancer (SCLC). J. Clin. Oncol. 37, TPS8576-TPS8576 (2019).
24. Owen, D. H. et al. DLL3: An emerging target in small cell lung cancer. Journal of Hematology and Oncology 12, 61 (2019).
25. Owonikoko, T. K. et al. Phase I study of AMG 757, a half-life extended bispecific T-cell engager (HLE BITE immune therapy) targeting DLL3, in patients with small cell lung cancer (SCLC). J. Clin. Oncol. 38, TPS9080-TPS9080 (2020).
26. Whalen, K. A., White, B. H., Quinn, J. M., Kriksciukaite, K., Alargova, R., Au Yeung, T. P., Bazinet, P., Brockman, A., DuPont, M. M., 0Iler, H., et al. (2019). Targeting the Somatostatin Receptor 2 with the Miniaturized Drug Conjugate, PEN-221: A Potent and Novel Therapeutic for the Treatment of Small Cell Lung Cancer. Mol Cancer Ther 18, 1926-1936.
27. White, B. H., Whalen, K., Kriksciukaite, K., Alargova, R., Au Yeung, T., Bazinet, P., Brockman, A., DuPont, M., 0Iler, H., Lemelin, C. A., et al. (2019). Discovery of an SSTR2-Targeting Maytansinoid Conjugate (PEN-221) with Potent Activity in Vitro and in Vivo. J Med Chem 62, 2708-2719.
28. Bonnafous, C. P., Valentine; Trichard, Sylvia; Perrot, Ivan; Cornen, Stephanie; Morel, Ariane; Breso, Violette; Morel, Yannis; Rossi, Benjamin; Paturel, Carine; Gauthier, Laurent; Blery, Mathieu (2013). Abstract 5037: Targeting MICA with therapeutic antibodies for the treatment of cancer. Journal for ImmunoTherapy of Cancer 1, P41.
29. Das, M. (2017). Labetuzumab govitecan in metastatic colorectal cancer. Lancet Oncol 18, e563.
30. Dotan, E., Cohen, S. J., Starodub, A. N., Lieu, C. H., Messersmith, W. A., Simpson, P. S., Guarino, M. J., Marshall, J. L., Goldberg, R. M., Hecht, J. R., et al. (2017). Phase I/II Trial of Labetuzumab Govitecan (Anti-CEACAM5/SN-38 Antibody-Drug Conjugate) in Patients With Refractory or Relapsing Metastatic Colorectal Cancer. J Clin Oncol 35, 3338-3346.
31. Sharkey, R. M., Govindan, S. V., Cardillo, T. M., Donnell, J., Xia, J., Rossi, E. A., Chang, C. H., and Goldenberg, D. M. (2018). Selective and Concentrated Accretion of SN-38 with a CEACAM5-Targeting Antibody-Drug Conjugate (ADC), Labetuzumab Govitecan (IMMU-130). Mol Cancer Ther 17, 196-203.
32. Thistlethwaite, F. C., Gilham, D. E., Guest, R. D., Rothwell, D. G., Pillai, M., Burt, D. J., Byatte, A. J., Kirillova, N., Valle, J. W., Sharma, S. K., et al. (2017). The clinical efficacy of first-generation carcinoembryonic antigen (CEACAM5)-specific CAR T cells is limited by poor persistence and transient pre-conditioning-dependent respiratory toxicity. Cancer Immunol Immunother 66, 1425-1436.

What is claimed:

1. A method for treating a subject for small cell lung cancer (SCLC), the method comprising administering a targeting agent capable of specifically binding to one or more of the proteins of Table 1, Table 2, or Table 3 to a subject determined to have SCLC-A.

2. The method of claim 1, wherein the one or more proteins are one or more proteins of Table 1.

3. The method of claim 1, wherein the one or more proteins are one or more proteins of Table 2.

4. The method of claim 1, wherein the one or more proteins are one or more proteins of Table 3.

5. The method of any of claims 1-4, wherein the targeting agent is capable of specifically binding to DLL3.

6. The method of any of claims 1-4, wherein the targeting agent is capable of specifically binding to CEACAM5.

7. The method of any of claims 1-4, wherein the targeting agent is capable of specifically binding to SCNN1A.

8. The method of any of claims 1-7, wherein the subject was determined to have SCLC-A by detecting expression of ASCL1 from cancer cells from the subject.

9. The method of any of claims 1-8, wherein the targeting agent comprises an antibody or fragment thereof.

10. The method of any of claims 1-8, wherein the targeting agent is a bispecific T-cell engager.

11. The method of any of claims 1-10, wherein a cell comprising the targeting agent is administered to the subject.

12. The method of claim 11, wherein the cell is an immune cell.

13. The method of claim 12, wherein the immune cell is a T cell.

14. The method of claim 12, wherein the immune cell is an NK cell.

15. The method of any of claims 11-13, wherein the targeting agent is a chimeric antigen receptor.

16. The method of any of claims 11-13, wherein the targeting agent is a T cell receptor.

17. The method of any of claims 1-16, wherein the targeting agent is operatively linked to a therapeutic agent.

18. The method of claim 17, wherein the therapeutic agent is a chemotherapeutic.

19. The method of claim 17, wherein the therapeutic agent is a toxin.

20. The method of claim 17, wherein the therapeutic agent is a therapeutic nucleic acid.

21. The method of any of claims 1-20, wherein the targeting agent is an antibody-drug conjugate.

22. The method of any of claims 1-20, wherein the targeting agent is an antibody-oligonucleotide conjugate.

23. A method for treating a subject for SCLC, the method comprising administering a targeting agent capable of specifically binding to one or more of the proteins of Table 4, Table 5, or Table 6 to a subject determined to have SCLC-N.

24. The method of claim 23, wherein the one or more proteins are one or more proteins of Table 4.

25. The method of claim 23, wherein the one or more proteins are one or more proteins of Table 5.

26. The method of claim 23, wherein the one or more proteins are one or more proteins of Table 6.

27. The method of any of claims 23-26, wherein the targeting agent is capable of specifically binding to SSTR2.

28. The method of any of claims 23-26, wherein the targeting agent is capable of specifically binding to SEMA6D.

29. The method of any of claims 23-26, wherein the targeting agent is capable of specifically binding to SGCD.

30. The method of any of claims 23-29, wherein the subject was determined to have SCLC-N by detecting expression of NEUROD1 from cancer cells from the subject.

31. The method of any of claims 23-30, wherein the targeting agent comprises an antibody or fragment thereof.

32. The method of any of claims 23-30, wherein the targeting agent is a bispecific T-cell engager.

33. The method of any of claims 23-32, wherein a cell comprising the targeting agent is administered to the subject.

34. The method of claim 33, wherein the cell is an immune cell.

35. The method of claim 34, wherein the immune cell is a T cell.

36. The method of claim 34, wherein the immune cell is an NK cell.

37. The method of any of claims 33-35, wherein the targeting agent is a chimeric antigen receptor.

38. The method of any of claims 33-35, wherein the targeting agent is a T cell receptor.

39. The method of any of claims 23-38, wherein the targeting agent is operatively linked to a therapeutic agent.

40. The method of claim 39, wherein the therapeutic agent is a chemotherapeutic.

41. The method of claim 39, wherein the therapeutic agent is a toxin.

42. The method of claim 39, wherein the therapeutic agent is a therapeutic nucleic acid.

43. The method of any of claims 23-42, wherein the targeting agent is an antibody-drug conjugate.

44. The method of any of claims 23-42, wherein the targeting agent is an antibody-oligonucleotide conjugate.

45. A method for treating a subject for SCLC, the method comprising administering a targeting agent capable of specifically binding to one or more of the proteins of Table 7, Table 8, or Table 9 to a subject determined to have SCLC-P.

46. The method of claim 45, wherein the one or more proteins are one or more proteins of Table 7.

47. The method of claim 45, wherein the one or more proteins are one or more proteins of Table 8.

48. The method of claim 45, wherein the one or more proteins are one or more proteins of Table 9.

49. The method of any of claims 45-48, wherein the targeting agent is capable of specifically binding to MICA.

50. The method of any of claims 45-48, wherein the targeting agent is capable of specifically binding to TMEM87A.

51. The method of any of claims 45-48, wherein the targeting agent is capable of specifically binding to ART3.

52. The method of any of claims 45-51, wherein the subject was determined to have SCLC-P by detecting expression of POU2F3 from cancer cells from the subject.

53. The method of any of claims 45-52, wherein the targeting agent comprises an antibody or fragment thereof.

54. The method of any of claims 45-52, wherein the targeting agent is a bispecific T-cell engager.

55. The method of any of claims 45-54, wherein a cell comprising the targeting agent is administered to the subject.

56. The method of claim 55, wherein the cell is an immune cell.

57. The method of claim 56, wherein the immune cell is a T cell.

58. The method of claim 56, wherein the immune cell is an NK cell.

59. The method of any of claims 55-57, wherein the targeting agent is a chimeric antigen receptor.

60. The method of any of claims 55-57, wherein the targeting agent is a T cell receptor.

61. The method of any of claims 45-60, wherein the targeting agent is operatively linked to a therapeutic agent.

62. The method of any of claim 61, wherein the therapeutic agent is a chemotherapeutic.

63. The method of any of claim 61, wherein the therapeutic agent is a toxin.

64. The method of any of claim 61, wherein the therapeutic agent is a therapeutic nucleic acid.

65. The method of any of claims 45-64, wherein the targeting agent is an antibody-drug conjugate.

66. The method of any of claims 45-64, wherein the targeting agent is an antibody-oligonucleotide conjugate.

67. A method for treating a subject for SCLC, the method comprising administering a targeting agent capable of specifically binding to one or more of the proteins of Table 10, Table 11, or Table 12 to a subject determined to have SCLC-I.

68. The method of claim 67, wherein the one or more proteins are one or more proteins of Table 10.

69. The method of claim 67, wherein the one or more proteins are one or more proteins of Table 11.

70. The method of claim 67, wherein the one or more proteins are one or more proteins of Table 12.

71. The method of any of claims 67-70, wherein the targeting agent is capable of specifically binding to SLAMF8.

72. The method of claim 67, wherein the targeting agent is capable of specifically binding to MRC2.

73. The method of claim 67, wherein the targeting agent is capable of specifically binding to PIEZO1.

74. The method of any of claims 67-73, wherein the subject was determined to have SCLC-I by determining cancer cells from the subject not to express any of ASCL1, NEUROD1, or POU2F3.

75. The method of any of claims 67-74, wherein the targeting agent comprises an antibody or fragment thereof.

76. The method of any of claims 67-74, wherein the targeting agent is a bispecific T-cell engager.

77. The method of any of claims 67-76, wherein a cell comprising the targeting agent is administered to the subject.

78. The method of claim 77, wherein the cell is an immune cell.

79. The method of claim 78 wherein the immune cell is a T cell.

80. The method of claim 78, wherein the immune cell is an NK cell.

81. The method of any of claims 77-79, wherein the targeting agent is a chimeric antigen receptor.

82. The method of any of claims 77-79, wherein the targeting agent is a T cell receptor.

83. The method of any of claims 67-82, wherein the targeting agent is operatively linked to a therapeutic agent.

84. The method of claim 83, wherein the therapeutic agent is a chemotherapeutic.

85. The method of claim 83, wherein the therapeutic agent is a toxin.

86. The method of claim 83, wherein the therapeutic agent is a therapeutic nucleic acid.

87. The method of any of claims 67-86, wherein the targeting agent is an antibody-drug conjugate.

88. The method of any of claims 67-86, wherein the targeting agent is an antibody-oligonucleotide conjugate.

89. A method for treating a subject for SCLC, the method comprising administering a DLL3-binding protein to a subject determined to have SCLC-A.

90. A method for treating a subject for SCLC, the method comprising administering a CEACAM5-binding protein to a subject determined to have SCLC-A.

91. A method for treating a subject for SCLC, the method comprising administering a SCNN1A-binding protein to a subject determined to have SCLC-A.

92. A method for treating a subject for SCLC, the method comprising administering a SSTR2-binding protein to a subject determined to have SCLC-N.

93. A method for treating a subject for SCLC, the method comprising administering a SEMA6D-binding protein to a subject determined to have SCLC-N.

94. A method for treating a subject for SCLC, the method comprising administering a SGCD-binding protein to a subject determined to have SCLC-N.

95. A method for treating a subject for SCLC, the method comprising administering a MICA-binding protein to a subject determined to have SCLC-P.

96. A method for treating a subject for SCLC, the method comprising administering a TMEM87A-binding protein to a subject determined to have SCLC-P.

97. A method for treating a subject for SCLC, the method comprising administering a ART3-binding protein to a subject determined to have SCLC-P.

98. A method for treating a subject for SCLC, the method comprising administering a SLAMF8-binding protein to a subject determined to have SCLC-I.

99. A method for treating a subject for SCLC, the method comprising administering a MRC2-binding protein to a subject determined to have SCLC-I.

100. A method for treating a subject for SCLC, the method comprising administering a PIEZO1-binding protein to a subject determined to have SCLC-I.
